SEQUENCE LISTING (219 sequences)

SEQ ID NO: 1 - protein, 137 aa, synthetic construct
MVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLN

SEQ ID NO: 2 - protein, 63 aa, synthetic construct
SIVAQLSRPDPALAALTNDHLVALACLGGRPAMDAVKKGLPHAPELIRRVNRRIGERTSHRVA

SEQ ID NO: 3 - protein, 34 aa, synthetic construct
LTPDQVVAIASNNGGKQALETVQRLLPVLCQDHG

SEQ ID NO: 4 - protein, 34 aa, synthetic construct
LTPDQVVAIASHDGGKQALETVQRLLPVLCQDHG

SEQ ID NO: 5 - protein, 34 aa, synthetic construct
LTPDQVVAIASNGGGKQALETVQRLLPVLCQDHG

SEQ ID NO: 6 - protein, 34 aa, synthetic construct; identical to SEQ ID NO: 3

SEQ ID NO: 7 - protein, 34 aa, synthetic construct; identical to SEQ ID NO: 3

SEQ ID NO: 8 - protein, 34 aa, synthetic construct
LTPDQVVAIASNIGGKQALETVQRLLPVLCQDHG

SEQ ID NO: 9 - protein, 34 aa, synthetic construct; identical to SEQ ID NO: 5

SEQ ID NO: 10 - protein, 34 aa, synthetic construct; identical to SEQ ID NO: 3

SEQ ID NO: 11 - protein, 34 aa, synthetic construct; identical to SEQ ID NO: 4

SEQ ID NO: 12 - protein, 34 aa, synthetic construct; identical to SEQ ID NO: 5

SEQ ID NO: 13 - protein, 34 aa, synthetic construct; identical to SEQ ID NO: 5

SEQ ID NO: 14 - protein, 34 aa, synthetic construct; identical to SEQ ID NO: 5

SEQ ID NO: 15 - protein, 34 aa, synthetic construct; identical to SEQ ID NO: 3

SEQ ID NO: 16 - protein, 34 aa, synthetic construct; identical to SEQ ID NO: 8

SEQ ID NO: 17 - protein, 34 aa, synthetic construct; identical to SEQ ID NO: 3

SEQ ID NO: 18 - protein, 34 aa, synthetic construct; identical to SEQ ID NO: 5

SEQ ID NO: 19 - protein, 20 aa, synthetic construct
LTPDQVVAIASNIGGKQALE

SEQ ID NO: 20 - protein, 34 aa, synthetic construct; identical to SEQ ID NO: 3

SEQ ID NO: 21 - protein, 34 aa, synthetic construct; identical to SEQ ID NO: 4

SEQ ID NO: 22 - protein, 34 aa, synthetic construct; identical to SEQ ID NO: 4

SEQ ID NO: 23 - protein, 34 aa, synthetic construct; identical to SEQ ID NO: 5

SEQ ID NO: 24 - protein, 34 aa, synthetic construct; identical to SEQ ID NO: 5

SEQ ID NO: 25 - protein, 34 aa, synthetic construct; identical to SEQ ID NO: 5

SEQ ID NO: 26 - protein, 34 aa, synthetic construct; identical to SEQ ID NO: 5

SEQ ID NO: 27 - protein, 34 aa, synthetic construct; identical to SEQ ID NO: 3

SEQ ID NO: 28 - protein, 34 aa, synthetic construct; identical to SEQ ID NO: 5

SEQ ID NO: 29 - protein, 34 aa, synthetic construct; identical to SEQ ID NO: 4

SEQ ID NO: 30 - protein, 34 aa, synthetic construct; identical to SEQ ID NO: 4

SEQ ID NO: 31 - protein, 34 aa, synthetic construct; identical to SEQ ID NO: 8

SEQ ID NO: 32 - protein, 34 aa, synthetic construct; identical to SEQ ID NO: 8

SEQ ID NO: 33 - protein, 34 aa, synthetic construct; identical to SEQ ID NO: 3

SEQ ID NO: 34 - protein, 34 aa, synthetic construct; identical to SEQ ID NO: 3

SEQ ID NO: 35 - protein, 34 aa, synthetic construct; identical to SEQ ID NO: 8

SEQ ID NO: 36 - protein, 229 aa, Rattus norvegicus [sequence omitted]

SEQ ID NO: 37 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 38 - protein, 188 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 39 - protein, 213 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSG

SEQ ID NO: 40 - protein, 121 aa, synthetic construct
SHRCYVLFELKRRGERRACFWGYAVNKPQSGTERGIHAEIFSIRKVEEYLRDNPGQFTINWYSSWSPCADCAEKILEWYNQELRGNGHTLKIEACKLYYEKNARNQIGLQNLRDNGVGLNV

SEQ ID NO: 41 - protein, 198 aa, Homo sapiens [sequence omitted]

SEQ ID NO: 42 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 43 - protein, 194 aa, Rhinopithecus roxellana
MKPQIRDHRPNPMEAMYPHIFYFHFENLEKAYGRNETWLCFTVEIIKQYLPVPWKKGVFRNQVDPETHCHAEKCFLSWFCNNTLSPKKNYQVTWYTSWSPCPECAGEVAEFLAEHSNVKLTIYTARLYYFWDTDYQEGLRSLSEEGASVEIMDYEDFQYCWENFVYDDGEPFKRWKGLKYNFQSLTRRLREILQ

SEQ ID NO: 44 - protein, 192 aa, Alligator mississippiensis
MADSSEKMRGQYISRDTFEKNYKPIDGTKEAHLLCEIKWGKYGKPWLHWCQNQRMNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRLLDIFWESKCRSPNPW

SEQ ID NO: 45 - protein, 280 aa, Sus scrofa
MDPQRLRQWPGPGPASRGGYGQRPRIRNPEEWFHELSPRTFSFHFRNLRFASGRNRSYICCQVEGKNCFFQGIFQNQVPPDPPCHAELCFLSWFQSWGLSPDEHYYVTWFISWSPCCECAAKVAQFLEENRNVSLSLSAARLYYFWKSESREGLRRLSDLGAQVGIMSFQDFQHCWNNFVHNLGMPFQPWKKLHKNYQRLVTELKQILREEPATYGSPQAQGKVRIGSTAAGLRHSHSHTRSEAHLRPNHSSRQHRILNPPREARARTCVLVDASWICYR

SEQ ID NO: 46 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 47 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 48 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 49 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 50 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 51 - protein, 182 aa, Homo sapiens [sequence omitted]

SEQ ID NO: 52 - protein, 197 aa, Homo sapiens
MEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 53 - protein, 161 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKL

SEQ ID NO: 54 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 55 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 56 - protein, 403 aa, Homo sapiens
MHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 57 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 58 - protein, 167 aa, Escherichia coli
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 59 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 60 - protein, 196 aa, synthetic construct
QLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 61 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVATKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 62 - protein, 196 aa, synthetic construct
QLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 63 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVATKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 64 - protein, 837 aa, Mus musculus
MGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFH

SEQ ID NO: 65 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 66 - protein, 445 aa, Mus musculus
MSSGMAYTSDRDRNKARAYSHCHKDHMKGRASKRRCSKVYCSVTKTSKYRWNRTTTSVDASGKVVVTAGHCGSVMGSNGTVYTGDRAKGASRMHSGGRVKDSVYDTTCDRYSRCRGVRSWVTRSHHVVWNCKAAYGYYTNSGVVHVDKDMKNMDHHTTDRNTHACRHKACWNKCGTSNKTAHTSKSTMWGRTRKTNVVRTGSSYRACSHSSSKDSYCVNVYNVVGTVDKVMDVKCRSSVKYKGKKRARTHDSDDDDDTRHKVYTSMKADRSGGCKASVWSSANDCSNSDSGTSGGGSTVNADDVDWVKRRDTGCHSSTGGSSKCSDSKCSDSKCSDSDGDSTHSSNSSSTHTDGSGWDSCDTVSSKSGGDSTSNKGAYKKKSSASDACDTHCDKSRAVNGACVDTSGRKSKTSSTRADSSSSDSTATHCYRKATGSVVKRKCSDS

SEQ ID NO: 67 - protein, 291 aa, Escherichia phage
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 68 - protein, 83 aa, Bacillus subtilis bacteriophage PBS1
TNLSDIIEKETGKQLVIQESILMLPEEVEEVIGNKPESDILVHTAYDESTDENVMLLTSDAPEYKPWALVIQDSNGENKIKML

SEQ ID NO: 69 - DNA, 1987 bp, synthetic construct
  source 1..1987: mol_type = other DNA
tgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgatcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgca

SEQ ID NO: 70 - DNA, 678 bp, synthetic construct
  source 1..678: mol_type = other DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctga

SEQ ID NO: 71 - DNA, 891 bp, synthetic construct
  source 1..891: mol_type = other DNA
cctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgacctcaacacaacatatacaaaacaaacgaatctcaagcaatcaagcattctacttctattgcagcaatttaaatcatttcttttaaagcaaaagcaattttctgaaaattttcaccatttacgaacgata

SEQ ID NO: 72 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 73 - DNA, 258 bp, synthetic construct
  source 1..258: mol_type = other DNA
gaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

SEQ ID NO: 74 - DNA, 635 bp, synthetic construct
  source 1..635: mol_type = other DNA
agagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 75 - DNA, 175 bp, synthetic construct
  source 1..175: mol_type = other DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 76 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 77 - protein, 30 aa, synthetic construct
MLSRAVCGTSRQLAPVLGYLGSRQKHSLPD

SEQ ID NO: 78 - protein, 25 aa, synthetic construct
MSVLTPLLLRGLTGSARRLPVPRAK

SEQ ID NO: 79 - protein, 49 aa, synthetic construct
MAPTVMMASSATAVAPFQGLKSAASLPVARRSTRSLGNVSNGGRIRCMQ

SEQ ID NO: 80 - protein, 144 aa, Sorangium cellulosum
MAPDSLVWFDPLGLIVLQQVPYNDHPLFGAVSEFIQGKSRSDLRGRNVAAVLLDDGTVIVRASEGGGNHAERVLMGLSEVDPAKVVAVYTERSPCTGRINCHDLLDSSLGADVPVYYTHEMIRGQEGKTAQQIEADRNQFCRGG

SEQ ID NO: 81 - protein, 208 aa, Stackebrandtia nassauensis
MSASAQLNTYLAAIGNSTTTVEAQPEAAPPPAAAESLDSTPRLPDGGIDFHALAKRLGLLEARPTEQPPFDPRRFNPACWQGLKPYDQAGTAEGNLFIAPGKRWNTRPMQASKLEVGPQSDLHPQWRSRKAPWHIEGKIAAYMRQKGFTDGCVYLNARPCSGPDGCARNLPDLLPVGSTLHVHARYIDRTGETRFYYREYRGTGKALT

SEQ ID NO: 82 - protein, 212 aa, Stackebrandtia nassauensis
MLDAMDAYLSEIAGGNAPARAGPKAPEPKQPGGSSSPRARDGRIDFRALLERLKAQGVVGLEGRSDDPIPDFDPKKQNPACYQGLAPRQKGKPVRGNLFFPDGRRWNDVALESSRGEPAFDLNIIKPEYRSLSPARGHLEGNVAAWMRSTFHQEMVLYINESPCRKHGKGCLYTLEHFLPRGYVLHVWSRNDRGEWRGNTFRGSGEAFTEGA

SEQ ID NO: 83 - protein, 226 aa, Stackebrandtia nassauensis
MVETRDKIIAAKSRSDAGLLAFQQATNGSIDSRPAEAIANLQRAKTHLDEAQRLVANSDAAVDNYINAILGGASAATAQPSAVIPASKPSRFKPMRTDPAKADEIRPHVGKDRAVATLWDADGNRVLGLHSADDDGPAATAAWKPPWRDYVRLRRHVEAHAAARMHQDGHKTMVMYINLPPCKYFDGCKLNLEDILPKGSTLWMHRVFQNGGTKIYQFNGTGRAYV

SEQ ID NO: 84 - protein, 168 aa, Acionosynnema mirum
MLEAVRARLIGEGGGPGAVPEGGDGPPAVPAEEVERLRGELPPPVVPGTGQKTHGRWIGPDGRVRAIVSGRDEDAALVHAQLAAKGIPDEPTRNSDVEQKLAAHMVANGIRHVTLVINHRPCRGFDDSCDTLVPIILPEGCTLTVHGQTDKGMRVRVRYTGGARPWWS

SEQ ID NO: 85 - protein, 166 aa, Acionosynnema mirum
MLDAALGAVRRIIAALGTSGAERASPGANGSERVDELAERLPPTVVPNTSAKTHGWWFTGQGAAQELISGEGPDARAAYEALREEGYPRPGMPFVAMHVEIKLAAHMRRNDIEHATVVINNIPCPLVWGCENLIGVVLPEGSSLTVHGSNGYERTFTGGRKPPWPR

SEQ ID NO: 86 - protein, 167 aa, Acionosynnema mirum
MLLTPPPRPAAPPTTRPKPLVARTGDAYPPGTEWALPLIVQPHPPVGGTVPVEGHVRALRPESQISHVFHPGGGHWTEQARARLRVLPGFGWAVNLGHHVELQIAAWMTACGIHHAELVLNRPPCGERYGLGCHQALPVLLPRGYRLTVSSTRGGPQPYQHHYEGKA

SEQ ID NO: 87 - protein, 196 aa, Flavobacterium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 88 - protein, 196 aa, Flavobacterium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 89 - protein, 564 aa, synthetic construct
LTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALE

SEQ ID NO: 90 - protein, 564 aa, synthetic construct
LTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALE

SEQ ID NO: 91 - protein, 632 aa, synthetic construct
LTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALE

SEQ ID NO: 92 - protein, 496 aa, synthetic construct
LTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALE

SEQ ID NO: 93 - protein, 530 aa, synthetic construct
LTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALE

SEQ ID NO: 94 - protein, 598 aa, synthetic construct
LTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALE

SEQ ID NO: 95 - protein, 598 aa, synthetic construct
LTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALE

SEQ ID NO: 96 - protein, 632 aa, synthetic construct
LTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALE

SEQ ID NO: 97 - protein, 564 aa, synthetic construct
LTPDQVVAIASNGGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPEQVVAIASHDGGRPALE

SEQ ID NO: 98 - protein, 564 aa, synthetic construct
LTPDQVVAIASHDGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNNGGRPALE

SEQ ID NO: 99 - protein, 462 aa, synthetic construct
LTPDQVVAIASNGGGKQALETVQRLLPVLCQDHGLTPDQVVAIASNGGGKQALETVQRLLPVLCQDHGLTPDQVVAIASNIGGKQAVETVQRLLPVLCQAHGLTPAQVVAIASHDGGKQAVETVQRLLPVLCQDHGLTPDQVVAIASHDGGKQAVETVQRLLPVLCQDHGLTPDQVVAIASNIGGKQALETLQRLLPVLCQAHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNIGGKQALETVQRLLPVLCQAHGLTPDQVVAIASNGGGKQAVETVQRLLPVLCQAHGLTPAQVVAIASNNGGKQAVETVQRLLPVLCQAHGLTPAQVVAIASNIGGKQAVETVQRLLPVLCQDHGLTPEQVVAIASNGGGKQALE

SEQ ID NO: 100 - protein, 564 aa, synthetic construct
LTPAQVVAIASHDGGKQALETVQRLLPVLCQAHGLTPDQVVAIASHDGGKQAVETVQRLLPVLCQDHGLTPDQVVAIASNIGGKQAVETVQRLLPVLCQAHGLTPAQVVAIASNIGGKQAVETVQRLLPVLCQDHGLTPDQVVAIASHDGGKQALETLQRLLPVLCQDHGLTPDQVVAIASNNGGKQALETVQRLLPVLCQAHGLTPAQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQAVETVQRLLPVLCQDHGLTPDQVVAIASNIGGKQAVETVQRLLPVLCQDHGLTPDQVVAIASNIGGKQAVETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPAQVVAIASNGGGKQALETVQRLLPVLCQDHGLTPEQVVAIASNGGGKQALE

SEQ ID NO: 101 - protein, 326 aa, synthetic construct
LTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALE

SEQ ID NO: 102 - protein, 360 aa, synthetic construct
LTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALE

SEQ ID NO: 103 - protein, 496 aa, synthetic construct
LTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGRPALE

SEQ ID NO: 104 - protein, 428 aa, synthetic construct
LTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGRPALE

SEQ ID NO: 105 - protein, 530 aa, synthetic construct
LTPEQVVAIASNIGGKQALETVQALLPVLCQAHGLTPQQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNNGGKQALETVQALLPVLCQAHGLTPEQVVAIASNGGGKQALETVQALLPVLCQAHGLTPEQVVAIASNNGGKQALETVQRLLPVLCQAHGLTPEQVVAIASHDGGKQALETVQALLPVLCQAHGLTPEQVVAIASNNGGKQALETVQRLLPVLCQAHGLTPEQVVAIASNNGGKQALETVQALLPVLCQAHGLTPEQVVAIASHDGGKQALETVQALLPVLCQAHGLTPEQVVAIASNGGGKQALETVQALLPVLCQAHGLTPEQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPQQVVAIASNIGGRPALE

SEQ ID NO: 106 - protein, 529 aa, synthetic construct
LTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNIGGKQALETVQALLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNNGGKQALETVQALLPVLCQAHGLTPEQVVAIASNIGGKQLETVQRLLPVLCQAHGLTPQQVVAIASHDGGKQALETVQRLLPVLCQAHGLTPEQVVAIASNNGGKQALETVQALLPVLCQAHGLTPQQVVAIASHDGGRPALE

SEQ ID NO: 107 - protein, 530 aa, synthetic construct
LTPEQVVAIASHDGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASHDGGKQALETVQALLPVLCQAHGLTPEQVVAIASNIGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNNGGKQALETVQALLPVLCQAHGLTPEQVVAIASHDGGKQALETVQALLPVLCQAHGLTPEQVVAIASNIGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGRPALE

SEQ ID NO: 108 - protein, 564 aa, synthetic construct
LTPEQVVAIASHDGGKQALETVQALLPVLCQAHGLTPQQVVAIASHDGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNNGGKQALETVQALLPVLCQAHGLTPEQVVAIASNNGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNIGGKQALETVQALLPVLCQAHGLTPEQVVAIASNNGGKQALETVQRLLPVLCQAHGLTPQQVVAIASNNGGKQALETVQRLLPVLCQAHGLTPEQVVAIASNGGGKQALETVQALLPVLCQAHGLTPEQVVAIASNGGGKQALETVQALLPVLCQAHGLTPQQVVAIASHDGGRPALE

SEQ ID NO: 109 - protein, 462 aa, synthetic construct
LTPEQVVAIASHDGGKQALETVQALLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNGGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNGGGKQALETVQALLPVLCQAHGLTPEQVVAIASHDGGKQALETVQALLPVLCQAHGLTPEQVVAIASNGGGKQALETVQALLPVLCQAHGLTPEQVVAIASNIGGKQALETVQALLPVLCQAHGLTPQQVVAIASNNGGRPALE

SEQ ID NO: 110 - protein, 394 aa, synthetic construct
LTPEQVVAIASNGGGKQALETVQALLPVLCQAHGLTPQQVVAIASNNGGKQALETVQRLLPVLCQAHGLTPQQVVAIASNIGGKQALETVQRLLPVLCQAHGLTPEQVVAIASNNGGKQALETVQALLPVLCQAHGLTPEQVVAIASNGGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNNGGKQALETVQALLPVLCQAHGLTPEQVVAIASNNGGKQALETVQALLPVLCQAHGLTPQQVVAIASHDGGRPALE

SEQ ID NO: 111 - protein, 530 aa, synthetic construct
LTPEQVVAIASHDGGKQALETVQALLPVLCQAHGLTPQQVVAIASHDGGKQALETVQRLLPVLCQAHGLTPQQVVAIASHDGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNNGGKQALETVQALLPVLCQAHGLTPEQVVAIASHDGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNNGGKQALETVQALLPVLCQAHGLTPEQVVAIASNNGGKQALETVQRLLPVLCQAHGLTPQQVVAIASNIGGKQALETVQRLLPVLCQAHGLTPEQVVAIASNGGGKQALETVQALLPVLCQAHGLTPQQVVAIASNIGGRPALE

SEQ ID NO: 112 - protein, 530 aa, synthetic construct
LTPEQVVAIASNGGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNIGGKQALETVQALLPVLCQAHGLTPEQVVAIASNGGGKQALETVQALLPVLCQAHGLTPEQVVAIASNIGGKQALETVQRLLPVLCQAHGLTPEQVVAIASNGGGKQALETVQALLPVLCQAHGLTPEQVVAIASNIGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNIGGKQALETVQALLPVLCQAHGLTPEQVVAIASHDGGKQALETVQRLLPVLCQAHGLTPQQVVAIASNIGGKQALETVQRLLPVLCQAHGLTPEQVVAIASNNGGKQALETVQALLPVLCQAHGLTPQQVVAIASHDGGRPALE

SEQ ID NO: 113 - protein, 17 aa, synthetic construct
NSGSETPGTSESATPES

SEQ ID NO: 114 - protein, 48 aa, synthetic construct
SGSETPGTSESATPESSGGSSGGSSGSETPGTSESATPESSGGSSGGS

SEQ ID NO: 115 - protein, 16 aa, synthetic construct
SGSETPGTSESATPES

SEQ ID NO: 116 - protein, 14 aa, synthetic construct
SGGGSGGSGGSGGS

SEQ ID NO: 117 - protein, 11 aa, synthetic construct
SGGSGGSGGSS

SEQ ID NO: 118 - protein, 4 aa, synthetic construct
SGGS

SEQ ID NO: 119 - protein, 152 aa, Barley Stripe Mosaic Virus
MMATFSCVCCGTLTTSTYCGKRCERKHVYSETRNKRLELYKKYLLEPQKCALNGIVGHSCGMPCSIAEEACDQLPIVSRFCGQKHADLYDSLLKRSEQELLLEFLQKKMQELKLSHIVKMAKLESEVNAIRKSVASSFEDSVGCDDSSSVSK

SEQ ID NO: 120 - protein, 2023 aa, synthetic construct
MDYKDHDGDYKDHDIDYKDDDDKMAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 121 - protein, 2023 aa, synthetic construct
MDYKDHDGDYKDHDIDYKDDDDKMAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 122 - protein, 2023 aa, synthetic construct
MDYKDHDGDYKDHDIDYKDDDDKMAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 123 - protein, 317 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESTNLSDIIEKETGKQLVIQESILMLPEEVEEVIGNKPESDILVHTAYDESTDENVMLLTSDAPEYKPWALVIQDSNGENKIKML

SEQ ID NO: 124 - protein, 286 aa, synthetic construct
MKRTADGSEFESPKKKRKVLEAVRARLIGEGGGPGAVPEGGDGPPAVPAEEVERLRGELPPPVVPGTGQKTHGRWIGPDGRVRAIVSGRDEDAALVHAQLAAKGIPDEPTRNSDVEQKLAAHMVANGIRHVTLVINHRPCRGFDDSCDTLVPIILPEGCTLTVHGQTDKGMRVRVRYTGGARPWWSNSGSETPGTSESATPESTNLSDIIEKETGKQLVIQESILMLPEEVEEVIGNKPESDILVHTAYDESTDENVMLLTSDAPEYKPWALVIQDSNGENKIKML

SEQ ID NO: 125 - protein, 856 aa, synthetic construct
MGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFHMKRTADGSEFESPKKKRKV

SEQ ID NO: 126 - protein, 255 aa, synthetic construct
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEAMKRTADGSEFESPKKKRKV

SEQ ID NO: 127 - protein, 2269 aa, synthetic construct
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 128 - protein, 2269 aa, synthetic construct
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 129 - protein, 856 aa, synthetic construct; identical to SEQ ID NO: 125

SEQ ID NO: 130 - protein, 255 aa, synthetic construct; identical to SEQ ID NO: 126

SEQ ID NO: 131 - protein, 102 aa, synthetic construct
MKRTADGSEFESPKKKRKVTNLSDIIEKETGKQLVIQESILMLPEEVEEVIGNKPESDILVHTAYDESTDENVMLLTSDAPEYKPWALVIQDSNGENKIKML

SEQ ID NO: 132 - protein, 2362 aa, synthetic construct
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 133 - protein, 2362 aa, synthetic construct
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 134 - protein, 856 aa, synthetic construct; identical to SEQ ID NO: 125

SEQ ID NO: 135 - protein, 255 aa, synthetic construct; identical to SEQ ID NO: 126

SEQ ID NO: 136 - protein, 3218 aa, synthetic construct
  REGION 1..2362: note = cassette1
  REGION 2363..3218: note = cassette2
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFSGGSGGSGGSTNLSDIIEKETGKQLVIQESILMLPEEVEEVIGNKPESDILVHTAYDESTDENVMLLTSDAPEYKPWALVIQDSNGENKIKMLMGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFHMKRTADGSEFESPKKKRKV

SEQ ID NO: 137 - protein, 3218 aa, synthetic construct
  REGION 1..2362: note = cassette1
  REGION 2363..3218: note = cassette2
MDYKDHDGDYKDHDIDYKDDDDKMAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFSGGSGGSGGSTNLSDIIEKETGKQLVIQESILMLPEEVEEVIGNKPESDILVHTAYDESTDENVMLLTSDAPEYKPWALVIQDSNGENKIKMLMGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFHMKRTADGSEFESPKKKRKV

SEQ ID NO: 138 - protein, 764 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 139 - protein, 790 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 140 - protein, 764 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 141 - protein, 790 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 142 - protein, 868 aa, synthetic construct
MALSRAVCGTSRQLAPVLGYLGSRQKHSLPDMGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFH

SEQ ID NO: 143 - protein, 267 aa, synthetic construct
MALSRAVCGTSRQLAPVLGYLGSRQKHSLPDMSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 144 - protein, 229 aa, synthetic construct
MALSRAVCGTSRQLAPVLGYLGSRQKHSLPDEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 145 - protein, 198 aa, synthetic construct
MALSRAVCGTSRQLAPVLGYLGSRQKHSLPDLEAVRARLIGEGGGPGAVPEGGDGPPAVPAEEVERLRGELPPPVVPGTGQKTHGRWIGPDGRVRAIVSGRDEDAALVHAQLAAKGIPDEPTRNSDVEQKLAAHMVANGIRHVTLVINHRPCRGFDDSCDTLVPIILPEGCTLTVHGQTDKGMRVRVRYTGGARPWWS

SEQ ID NO: 146 - protein, 120 aa, synthetic construct
MALSRAVCGTSRQLAPVLGYLGSRQKHSLPDGSSGGSTNLSDIIEKETGKQLVIQESILMLPEEVEEVIGNKPESDILVHTAYDESTDENVMLLTSDAPEYKPWALVIQDSNGENKIKML

SEQ ID NO: 147 - protein, 1010 aa, synthetic construct
MALSRAVCGTSRQLAPVLGYLGSRQKHSLPDYPYDVPDYAGYPYDVPDYAGYPYDVPDYAEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESSGGSSGGSSGSETPGTSESATPESSGGSSGGSMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 148 - protein, 1074 aa, synthetic construct
MASVLTPLLLRGLTGSARRLPVPRAKIHSLDYKDHDGDYKDHDIDYKDDDDKMSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEASGSETPGTSESATPESSGGSSGGSSGSETPGTSESATPESSGGSSGGSMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 149 - protein, 1167 aa, synthetic construct
MASVLTPLLLRGLTGSARRLPVPRAKIHSLDYKDHDGDYKDHDIDYKDDDDKTNLSDIIEKETGKQLVIQESILMLPEEVEEVIGNKPESDILVHTAYDESTDENVMLLTSDAPEYKPWALVIQDSNGENKIKMLSGGSGGSGGSMSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEASGSETPGTSESATPESSGGSSGGSSGSETPGTSESATPESSGGSSGGSMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 150 - protein, 5 aa, synthetic construct
KKRKV

SEQ ID NO: 151 - protein, 7 aa, synthetic construct
PKKKRKV

SEQ ID NO: 152 - protein, 16 aa, synthetic construct
KRPAATKKAGQAKKKK

SEQ ID NO: 153 - protein, 373 aa, Mus musculus
MAETGEEETASAEASGFSDLSDSELVEFLDLEEAKESAVSLSKPGPSAELPGKDDKPVSLQNWKGGLDVLSPMERFHLKYLYVTDLCTQNWCELQMVYGKELPGSLTPEKAAVLDTGASIHLAKELELHDLVTVPIATKEDAWAVKFLNILAMIPALQSEGRVREFPVFGEVEGIFLVGVIDELHYTSKGELELAELKTRRRPVLPLPAQKKKDYFQVSLYKYIFDAMVQGKVTPASLIHHTKLCLDKPLGPSVLRHARQGGVSVKSLGDLMELVFLSLTLSDLPAIDTLKLEYIHQETATILGTEIVAFEEKEVKSKVQHYVAYWMGHRDPQGVDVEEAWKCRTCDYVDICEWRRGSGVLSSSWEPKAKKFK

SEQ ID NO: 154 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 155 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 156 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVATKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 157 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 158 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 159 - protein, 2005 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVATKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 160 - protein, 2311 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 161 - protein, 2311 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 162 - protein, 2311 aa, synthetic construct
MAPKKKRKVGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 163 - protein, 103 aa, synthetic construct
MTNLSDIIEKETGKQLVIQESILMLPEEVEEVIGNKPESDILVHTAYDESTDENVMLLTSDAPEYKPWALVIQDSNGENKIKMLMKRTADGSEFESPKKKRKV

SEQ ID NO: 164 - protein, 347 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKNSGSETPGTSESATPESTNLSDIIEKETGKQLVIQESILMLPEEVEEVIGNKPESDILVHTAYDESTDENVMLLTSDAPEYKPWALVIQDSNGENKIKML

SEQ ID NO: 165 - protein, 392 aa, synthetic construct
MAETGEEETASAEASGFSDLSDSELVEFLDLEEAKESAVSLSKPGPSAELPGKDDKPVSLQNWKGGLDVLSPMERFHLKYLYVTDLCTQNWCELQMVYGKELPGSLTPEKAAVLDTGASIHLAKELELHDLVTVPIATKEDAWAVKFLNILAMIPALQSEGRVREFPVFGEVEGIFLVGVIDELHYTSKGELELAELKTRRRPVLPLPAQKKKDYFQVSLYKYIFDAMVQGKVTPASLIHHTKLCLDKPLGPSVLRHARQGGVSVKSLGDLMELVFLSLTLSDLPAIDTLKLEYIHQETATILGTEIVAFEEKEVKSKVQHYVAYWMGHRDPQGVDVEEAWKCRTCDYVDICEWRRGSGVLSSSWEPKAKKFKMKRTADGSEFESPKKKRKV

SEQ ID NO: 166 - protein, 190 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSMKRTADGSEFESPKKKRKV

SEQ ID NO: 167 - protein, 1189 aa, synthetic construct
MKRTADGSEFESPKKKRKVMGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFHSGSETPGTSESATPESMKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESTNLSDIIEKETGKQLVIQESILMLPEEVEEVIGNKPESDILVHTAYDESTDENVMLLTSDAPEYKPWALVIQDSNGENKIKML

SEQ ID NO: 168 - protein, 1221 aa, synthetic construct
MKRTADGSEFESPKKKRKVMGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFHSGSETPGTSESATPESSGGSSGGSSGSETPGTSESATPESSGGSSGGSMKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESTNLSDIIEKETGKQLVIQESILMLPEEVEEVIGNKPESDILVHTAYDESTDENVMLLTSDAPEYKPWALVIQDSNGENKIKML

SEQ ID NO: 169 - protein, 2251 aa, synthetic construct
MAPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 170 - protein, 2281 aa, synthetic construct
MAPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 171 - protein, 2557 aa, synthetic construct
MAPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 172 - protein, 2587 aa, synthetic construct
MAPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESSGGSSGGSSGSETPGTSESATPESSGGSSGGSGIHGVPSRMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFRSGGGEGRGSLLTCGDVEENPGPRMDYKDHDGDYKDHDIDYKDDDDKMAPKKKRKVGIHGVPARMVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAGSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 173 - protein, 1001 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPEQVVAIASHDGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 174 - protein, 915 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNNGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 175 - protein, 763 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 176 - protein, 711 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 177 - protein, 899 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNGGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNGGGKQALETVQALLPVLCQAHGLTPEQVVAIASHDGGKQALETVQALLPVLCQAHGLTPEQVVAIASNGGGKQALETVQALLPVLCQAHGLTPEQVVAIASNIGGKQALETVQALLPVLCQAHGLTPQQVVAIASNNGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 178 - protein, 725 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQALLPVLCQAHGLTPQQVVAIASNNGGKQALETVQRLLPVLCQAHGLTPQQVVAIASNIGGKQALETVQRLLPVLCQAHGLTPEQVVAIASNNGGKQALETVQALLPVLCQAHGLTPEQVVAIASNGGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNNGGKQALETVQALLPVLCQAHGLTPEQVVAIASNNGGKQALETVQALLPVLCQAHGSIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 179 - protein, 967 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASHDGGKQALETVQALLPVLCQAHGLTPEQVVAIASNIGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNNGGKQALETVQALLPVLCQAHGLTPEQVVAIASHDGGKQALETVQALLPVLCQAHGLTPEQVVAIASNIGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 180 - protein, 915 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASHDGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNNGGKQALETVQALLPVLCQAHGLTPEQVVAIASNNGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNIGGKQALETVQALLPVLCQAHGLTPEQVVAIASNNGGKQALETVQRLLPVLCQAHGLTPQQVVAIASNNGGKQALETVQRLLPVLCQAHGLTPEQVVAIASNGGGKQALETVQALLPVLCQAHGLTPEQVVAIASNGGGKQALETVQALLPVLCQAHGLTPQQVVAIASHDGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 181 - protein, 967 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASHDGGKQALETVQRLLPVLCQAHGLTPQQVVAIASHDGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNNGGKQALETVQALLPVLCQAHGLTPEQVVAIASHDGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNNGGKQALETVQALLPVLCQAHGLTPEQVVAIASNNGGKQALETVQRLLPVLCQAHGLTPQQVVAIASNIGGKQALETVQRLLPVLCQAHGLTPEQVVAIASNGGGKQALETVQALLPVLCQAHGLTPQQVVAIASNIGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 182 - protein, 881 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNGGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNIGGKQALETVQALLPVLCQAHGLTPEQVVAIASNGGGKQALETVQALLPVLCQAHGLTPEQVVAIASNIGGKQALETVQRLLPVLCQAHGLTPEQVVAIASNGGGKQALETVQALLPVLCQAHGLTPEQVVAIASNIGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNIGGKQALETVQALLPVLCQAHGLTPEQVVAIASHDGGKQALETVQRLLPVLCQAHGLTPQQVVAIASNIGGKQALETVQRLLPVLCQAHGLTPEQVVAIASNNGGKQALETVQALLPVLCQAHGLTPQQVVAIASHDGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 183 - protein, 967 aa, synthetic construct
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNNGGKQALETVQALLPVLCQAHGLTPEQVVAIASNGGGKQALETVQALLPVLCQAHGLTPEQVVAIASNNGGKQALETVQRLLPVLCQAHGLTPEQVVAIASHDGGKQALETVQALLPVLCQAHGLTPEQVVAIASNNGGKQALETVQRLLPVLCQAHGLTPEQVVAIASNNGGKQALETVQALLPVLCQAHGLTPEQVVAIASHDGGKQALETVQALLPVLCQAHGLTPEQVVAIASNGGGKQALETVQALLPVLCQAHGLTPEQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPQQVVAIASNIGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 184 - protein, 880 aa, synthetic construct
MASVLTPLLLRGLTGSARRLPVPRAKIHSLDYKDHDGDYKDHDIDYKDDDDKMDIADLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNIGGKQALETVQALLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNNGGKQALETVQALLPVLCQAHGLTPEQVVAIASNIGGKQLETVQRLLPVLCQAHGLTPQQVVAIASHDGGKQALETVQRLLPVLCQAHGLTPEQVVAIASNNGGKQALETVQALLPVLCQAHGLTPQQVVAIASHDGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 185 - protein, 340 aa, synthetic construct
LTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHG

SEQ ID NO: 186 - protein, 578 aa, synthetic construct
LTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHG

SEQ ID NO: 187 - protein, 408 aa, synthetic construct
LTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHG

SEQ ID NO: 188 - DNA, 52 bp, Oryzasativa
  source 1..52: mol_type = other DNA
gctggatgctttgagtactttgcagatcttgcagaatccttggacaaaaggc

SEQ ID NO: 189 - DNA, 52 bp, Oryzasativa
  source 1..52: mol_type = other DNA
cgacctacgaaactcatgaaacgtctagaacgtcttaggaacctgttttccg

SEQ ID NO: 190 - DNA, 52 bp, Oryzasativa
  source 1..52: mol_type = other DNA
gcaaaagaccaaggtgcctcaattgttcttgcagctcatgctgcgacgagcc

SEQ ID NO: 191 - DNA, 52 bp, Oryzasativa
  source 1..52: mol_type = other DNA
cgttttctggttccacggagttaacaagaacgtcgagtacgacgctgctcgg

SEQ ID NO: 192 - DNA, 53 bp, Oryzasativa
  source 1..53: mol_type = other DNA
cctggaccgcgtccacgacggcgagctcaagctccgcgccgcggggctctggg

SEQ ID NO: 193 - DNA, 53 bp, Oryzasativa
  source 1..53: mol_type = other DNA
ggacctggcgcaggtgctgccgctcgagttcgaggcgcggcgccccgagaccc

SEQ ID NO: 194 - DNA, 47 bp, Homo sapiens
  source 1..47: mol_type = other DNA
cccctgacccccatgcctcaggatactcctcaatagccatcgctgta

SEQ ID NO: 195 - DNA, 47 bp, Homo sapiens
  source 1..47: mol_type = other DNA
ggggactgggggtacggagtcctatgaggagttatcggtagcgacat

SEQ ID NO: 196 - DNA, 55 bp, Oryzasativa
  source 1..55: mol_type = other DNA
ccaggacgacgtcggcggcctcgaggtcctcgtcgacggcgaatggcgccccgtc

SEQ ID NO: 197 - DNA, 55 bp, Oryzasativa
  source 1..55: mol_type = other DNA
ggtcctgctgcagccgccggagctccaggagcagctgccgcttaccgcggggcag

SEQ ID NO: 198 - DNA, 52 bp, Homo sapiens
  source 1..52: mol_type = other DNA
tacgcggcggggctgtcgccgtacgcggacaagggcaagtgcggcctcccgg

SEQ ID NO: 199 - DNA, 52 bp, Homo sapiens
  source 1..52: mol_type = other DNA
atgcgccgccccgacagcggcatgcgcctgttcccgttcacgccggagggcc

SEQ ID NO: 200 - DNA, 47 bp, Oryzasativa
  source 1..47: mol_type = other DNA
ttaccaaagatgatgaaaacgtaaactcacaaccatttatgcgttgg

SEQ ID NO: 201 - DNA, 47 bp, Oryzasativa
  source 1..47: mol_type = other DNA
aatggtttctactacttttgcatttgagtgttggtaaatacgcaacc

SEQ ID NO: 202 - DNA, 52 bp, Homo sapiens
  source 1..52: mol_type = other DNA
gacccccatgcctcaggatactcctcaatagccatcgctgtagtatatccaa

SEQ ID NO: 203 - DNA, 52 bp, Homo sapiens
  source 1..52: mol_type = other DNA
ctgggggtacggagtcctatgaggagttatcggtagcgacatcatataggtt

SEQ ID NO: 204 - DNA, 40 bp, Homo sapiens
  source 1..40: mol_type = other DNA
cctatttattctagccacctctagcctagccgtttactca

SEQ ID NO: 205 - DNA, 40 bp, Homo sapiens
  source 1..40: mol_type = other DNA
ggataaataagatcggtggagatcggatcggcaaatgagt

SEQ ID NO: 206 - DNA, 51 bp, Homo sapiens
  source 1..51: mol_type = other DNA
tctccacactagcagagaccaaccgaacccccttcgaccttgccgaagggg

SEQ ID NO: 207 - DNA, 51 bp, Homo sapiens
  source 1..51: mol_type = other DNA
agaggtgtgatcgtctctggttggcttgggggaagctggaacggcttcccc

SEQ ID NO: 208 - DNA, 49 bp, Homo sapiens
  source 1..49: mol_type = other DNA
acgagtgcggcttcgaccctatatcccccgcccgcgtccctttctccat

SEQ ID NO: 209 - DNA, 49 bp, Homo sapiens
  source 1..49: mol_type = other DNA
tgctcacgccgaagctgggatatagggggcgggcgcagggaaagaggta

SEQ ID NO: 210 - DNA, 49 bp, Homo sapiens
  source 1..49: mol_type = other DNA
ctagcctagccgtttactcaatcctctcatcagggtgagcatcaaactc

SEQ ID NO: 211 - DNA, 49 bp, Homo sapiens
  source 1..49: mol_type = other DNA
gatcggatcggcaaatgagttaggagactagtcccactcgtagtttgag

SEQ ID NO: 212 - DNA, 48 bp, Homo sapiens
  source 1..48: mol_type = other DNA
gctagtaaccacgttctcctgatcaaatatcactctcctacttacagg

SEQ ID NO: 213 - DNA, 48 bp, Homo sapiens
  source 1..48: mol_type = other DNA
cgatcattggtgcaagaggactagtttatagtgagaggatgaatgtcc

SEQ ID NO: 214 - DNA, 44 bp, Homo sapiens
  source 1..44: mol_type = other DNA
agcattagcaggaatacctttcctcacaggtttctactccaaag

SEQ ID NO: 215 - DNA, 44 bp, Homo sapiens
  source 1..44: mol_type = other DNA
tcgtaatcgtccttatggaaaggagtgtccaaagatgaggtttc

SEQ ID NO: 216 - DNA, 36 bp, synthetic construct
  source 1..36: mol_type = other DNA
gacccccatgcctcaggatactcctcaatagccatc

SEQ ID NO: 217 - DNA, 36 bp, synthetic construct
  source 1..36: mol_type = other DNA
ctgggggtacggagtcctatgaggagttatcggtag

SEQ ID NO: 218 - DNA, 49 bp, synthetic construct
  source 1..49: mol_type = other DNA
ccccatgcctcaggatactcctcaatagccatcgctgtagtatatccaa

SEQ ID NO: 219 - DNA, 49 bp, synthetic construct
  source 1..49: mol_type = other DNA
ggggtacggagtcctatgaggagttatcggtagcgacatcatataggtt

(19) United States
(12) Patent Application Publication
(10) Pub. No.: US 2025/0101469 A1
(43) Pub. Date: Mar. 27, 2025

(54) BASE EDITOR AND THE USE THEREOF

(71) Applicants: INSTITUTE OF GENETICS AND DEVELOPMENTAL BIOLOGY, CHINESE ACADEMY OF SCIENCES, Beijing (CN); SUZHOU QI BIODESIGN BIOTECHNOLOGY COMPANY LIMITED, Suzhou (CN)

(72) Inventors: Caixia GAO, Beijing (CN); Kevin T. ZHAO, Beijing (CN); Yu SUN, Beijing (CN); Jiacheng HU, Beijing (CN); Boshu LI, Beijing (CN)

(21) Appl. No.: 18/970,575

(22) Filed: Dec. 5, 2024

Related U.S. Application Data
(63) Continuation of application No. PCT/CN2023/135588, filed on Nov. 30, 2023.

(30) Foreign Application Priority Data
Dec. 15, 2022 (CN) 202211613160.4
Aug. 14, 2023 (CN) 202311017698.3

(51) Int. Cl. C12N 15/90; C12N 9/22; C12N 9/78; C12N 15/11

(52) U.S. Cl. CPC C12N 15/907; C12N 9/22; C12N 9/78; C12N 15/11; C12N 2310/20

(57) ABSTRACT

The present disclosure discloses a base editor and the use thereof. The present disclosure provides a nucleic acid base editor, specifically a base editor which is not based on CRISPR technology. The base editor comprises a sequence-specific DNA binding protein, a nickase, an exonuclease and a base-specific deaminase. This base editor is single-strand-specific, and as compared with conventional base editors, the base editor of the present disclosure has wide applicability in cells and is capable of functioning in the nucleus as well as in mitochondrial DNA and/or chloroplast DNA. This base editor has the characteristics of achieving base editing products with high purity and resulting in few indel byproducts while realizing efficient base editing, which is conducive to being used as an efficient and safe gene editing tool.

### CROSS-REFERENCE TO RELATED APPLICATION

The present application is a continuation of International Application. No. PCT/CN2023/135588, filed on Nov. 30, 2023, which claims priority to Chinese patent application 202211613160.4, filed on Dec. 15, 2022, and Chinese patent application 202311017698.3, filed on Aug. 14, 2023, the entire contents of which including the appendixes are each herein incorporated by reference.

### SEQUENCE LISTING

The instant application contains a Sequence Listing that has been submitted electronically and is hereby incorporated by reference in its entirety. The Sequence Listing was created on Dec. 5, 2024, is named “24-1276-US-CON_SequenceListing.xml,” and is 299,517 bytes in size.

### TECHNICAL FIELD

The present disclosure relates to the field of gene editing, specifically relates to a nucleic acid base editor, and particularly relates to a base editor comprising a sequence-specific DNA binding protein, a nickase, an exonuclease and a base-specific deaminase, and the use thereof.

### BACKGROUND

Mutations in genome and mitochondrial DNA are known to lead to various genetic diseases (Newby et al., 2021, Nature 595: 295-302), and correcting these mutations is expected to result in effective treatment or amelioration of some severe disease. In plants, some important agronomic traits are associated with the single nucleotide variation (SNV) occurring in the plant genome, plant mitochondrial genome or plant chloroplast genome; and introducing these SNVs into plants could promote plant performance, molecular breeding, restoring gene function to alleviate disease states, and the like.

Genome editing has shown great potential for genome modification; among the genome editing tools, base editing could achieve targeted base substitution without introducing DNA double-strand breaks (DSB) so as to realize more precise and more accurate editing (Gaudelli et al., 2017, Nature 551: 464-471; Komor et al., 2016, Nature 533: 420-424), thus holding great promise for disease treatment and crop improvement.

Cytosine base editor (CBE) (Komor et al., 2016, Nature 533: 420-424) and adenine base editor (ABE) (Gaudelli et al., 2017, Nature 551: 464-471) are the most widely used base editors. In the CBE system, CRISPR-Cas9 nickase (nCas9) with nicking activity on single-stranded DNA is guided to the target dsDNA by sgRNA, and the sgRNA-targeting strand is nicked by nCas9 to form an R-loop. Subsequently, the single-strand-specific cytidine deaminase converts cytosine (C) to uracil (U) within an approximately five-nucleotide window in the single-stranded DNA bubble-like structure created by nCas9, U is replaced by T after DNA repair, thereby resulting in the conversion from a C:G base pair to a T:A base pair. In addition, the addition of a uracil glycosylase inhibitor (UGI) with the function of impeding uracil excision and its downstream processes could improve the base editing efficiency and the purity of the product. Cytidine deaminases suitable for the Cas-mediated CBE systems include but are not limited to APOBEC1, hAID and hAPOBEC3A. Recently, some new deaminase systems have also been found to be suitable for the deaminase of the present disclosure (Huang, J. et al. Discovery of new deaminase functions by structure-based protein clustering. bioRxiv (2023).).

The ABE system is generated by fusing nCas9 to an artificially evolved single-stranded DNA adenosine deaminase TadA (Gaudelli et al., 2017, Nature 551: 464-471). The working principle of ABE is similar to that of CBE, nCas9 would nick the target strand of DNA under the guidance of sgRNA to generate a nick, and the adenosine deaminase TadA converts adenine (A) to inosine (I), which is replaced by G after DNA repair, resulting in the conversion of an A:T base pair to a G:C base pair. However, UGI is not required in the ABE system to improve its editing efficiency or the purity of the product, since no uracil intermediate is involved in the process.

ABE and CBE mentioned above are capable of working efficiently in the nucleus, but they could not work in chloroplasts or mitochondria, since the sgRNA in the CRISPR system could not be transferred into these organelles efficiently.

In 2020, researchers developed a non-CRISPR base editor system that is solely comprised of protein components. This novel base editor system was designated as DdCBE (Mok et al., 2020, Nature 583: 631-637). The core components of DdCBE include a double-stranded DNA cytidine deaminase DddA, which could convert C to U on the double-stranded DNA without the need for CRISPR-Cas9 to create a single-stranded DNA. However, intact DddA has cytotoxicity, therefore, it is split into two halves—DddA-N and DddA-C, which are fused to a pair of TALE proteins separately. DddA-N and DddA-C are guided to the target DNA sequence by the TALE pair and are recombined to restore the cytidine deaminase activity; similar to the CRISPR-based CBE system, this system is also capable of converting a C:G base pair to a T:A base pair; the addition of UGI could improve the base editing efficiency and the purity of the product of DdCBE. Due to the characteristics that the components of the DdCBE system are all protein components, the DdCBE system could not only work in the nucleus, but also could be translocated into chloroplasts and mitochondria to achieve targeted cytosine base editing in chloroplast DNA and mitochondrial DNA.

However, since DddA toxin is a cytidine deaminase, it could merely operate on a cytosine base in the CBE system, but could not operate on an adenine base as required by the ABE system, thus severely limiting its application ranges. In 2022, researchers fused an adenosine deaminase TadA-8e obtained by artificial directed evolution to DdCBE to generate the TALED system, and this system were capable of realizing the base editing of A-to-G conversion (Cho et al., 2022, Cell 185: 1764-1776). In TALED system, the adenosine deaminase TadA-8e is fused to one of the split DddAs, and this combination successfully induces C-to-T base conversion and A-to-G base conversion simultaneously in the mitochondrial DNA. In addition, when the deaminase activity of DddA is inactivated, the TadA-8e-mediated A-to-G base editing remains effective.

Although the DdCBE system and the TALED system have expanded the application range of base editing to mitochondrial DNA and/or chloroplast DNA, there are still some limitations. First, due to the intrinsic double-stranded DNA cytidine deaminase activity of DddA, deamination would occur for the cytosines in the deamination window on both strands, which means that deamination could not merely occur on a selected single strand, and thus would not be safe and precise enough to be used safely. Second, compared to the CBE-mediated base editing and ABE-mediated base editing in the nucleus, the base editing products of DddA contain a relative higher indel frequency, and the resulting products have lower purity. Third, it has been reported that a DddA-based mitochondrial base editor would induce extensive off-target mutations in the nucleus when performing mitochondrial base editing (Lei et al., 2022, Nature 606: 804-811). It is worth noting that most of the off-target mutations are TALE-independent and are caused by DddA. The substantial nuclear off-target mutations would result in significant adverse impact on the safety of using these base editors.

Therefore, there is an urgent need in the art to develop a novel base editor that is single-strand-specific and could function in the nucleus as well as in mitochondrial DNA and/or chloroplast DNA with high product purity.

### SUMMARY

In order to solve the above-mentioned technical problems, the present application provides a novel base editor that does not rely on CRISPR technology. This system is single-strand-specific, is capable of functioning in the nucleus as well as in mitochondrial DNA or chloroplast DNA, and could obtain editing products with high purity.

To be specific, the present disclosure provides a novel nucleic acid base editor protein composition, a recombinant expression construct encoding a novel synthetic nucleic acid base editor protein, a genetically engineered cell comprising one or more recombinant expression constructs encoding novel synthetic nucleic acid base editor proteins, as well as the application methods of the above-mentioned novel nucleic acid base editor protein, recombinant expression construct and genetically engineered cell.

The nucleic acid base editor of the present disclosure comprises: a sequence-specific DNA binding protein; a nickase; an exonuclease and a base-specific deaminase. In certain embodiments, the nucleic acid base editor further comprises a uracil glycosylase inhibitor. In a specific embodiment, the sequence-specific DNA binding protein, the nickase, the exonuclease and the base-specific deaminase form one or more fusion proteins. In an advantageous embodiment of the nucleic acid base editor provided by the present disclosure, the sequence-specific DNA binding protein is selected from a TALE protein, a ZFA protein, a Cas protein and a meganuclease. In certain specific embodiments, the sequence-specific DNA binding protein is preferably a TALE protein. In a specific embodiment of the nucleic acid base editor of the present disclosure, the nickase is an FokI nickase. In the nucleic acid base editor of the present disclosure, the deaminase is selected from a cytidine-specific deaminase and an adenosine-specific deaminase. In an advantageous embodiment of the nucleic acid base editor of the present disclosure comprising a cytidine-specific deaminase, the cytidine deaminase is selected from hAPOBEC3A, rAPOBEC1, hAID, pmCDAT and Sdd deaminase. In an advantageous embodiment of the nucleic acid base editor of the present disclosure comprising an adenosine-specific deaminase, the adenosine deaminase is TadA-8e.

In another preferred embodiment, the composition provided by the present disclosure comprises one or more recombinant expression constructs encoding a sequence-specific DNA binding protein, a nickase, an exonuclease and a base-specific deaminase, wherein each of the sequence-specific DNA binding protein, the nickase, the exonuclease and the base-specific deaminase is capable of being expressed in a cell. In certain embodiments, these nucleic acid compositions further comprise a recombinant expression construct encoding a uracil glycosylase inhibitor. In a specific embodiment, this composition comprises one or more recombinant expression constructs encoding a sequence-specific DNA binding protein, a nickase, an exonuclease and a base-specific deaminase as a fusion protein, wherein the fusion protein comprised thereof is capable of being expressed in a cell. In an advantageous embodiment of the nucleic acid base editor provided herein, the sequence-specific DNA binding protein is selected from a TALE protein, a ZFA protein, a Cas protein and a meganuclease, and in certain specific embodiments, the sequence-specific DNA binding protein is a TALE protein. In a specific embodiment of the nucleic acid base editor of the present disclosure, the nickase is an FokI nickase. The deaminase in the nucleic acid base editor of the present disclosure is selected from a cytidine-specific deaminase and an adenosine-specific deaminase, preferably, the deaminase is selected from the deaminase as set forth in sequences SEQ ID NO. 36-59 and 80-86. In an advantageous embodiment of the above-mentioned nucleic acid base editor comprising a cytidine-specific deaminase, the cytidine deaminase is selected from hAPOBEC3A, rAPOBEC1, hAID, pmCDAT and Sdd deaminase. In an embodiment of the nucleic acid base editor of the present disclosure comprising an adenosine-specific deaminase, the adenosine deaminase is TadA-8e.

In another preferred embodiment, the present disclosure also provides a recombinant cell, which comprises one or more recombinant expression constructs encoding a sequence-specific DNA binding protein, a nickase, an exonuclease and a base-specific deaminase; wherein each of the sequence-specific DNA binding protein, the nickase, the exonuclease and the base-specific deaminase is capable of being expressed in a cell. In certain embodiments, these recombinant cells comprise nucleic acid compositions that further comprise a recombinant expression construct encoding a uracil glycosylase inhibitor. In a specific embodiment, the recombinant cell comprises one or more recombinant expression constructs encoding a sequence-specific DNA binding protein, a nickase, an exonuclease and a base-specific deaminase as a fusion protein, wherein the fusion protein comprised thereof is capable of being expressed in a cell. In an advantageous embodiment of the recombinant cell provided herein, the sequence-specific DNA binding protein is selected from a TALE protein, a ZFA protein, a Cas protein and a meganuclease, and in certain specific embodiments, the sequence-specific DNA binding protein is a TALE protein. In a specific embodiment of the recombinant cell provided herein, the nickase is FokI. Further provided are the recombinant cell of the present disclosure, comprising one or more recombinant expression constructs encoding a deaminase, wherein the deaminase is a cytidine-specific deaminase or an adenosine-specific deaminase, preferably, the deaminase is selected from the deaminase as set forth in sequences SEQ ID NO. 36-59 and 80-86. An advantageous embodiment of the recombinant cell provided herein comprises one or more recombinant expression constructs encoding a cytidine-specific deaminase, wherein the cytidine deaminase is selected from hAPOBEC3A, rAPOBEC1, hAID, pmCDAT and Sdd deaminase in an advantageous embodiment. In additional advantageous embodiments, the recombinant cell comprises one or more recombinant expression constructs encoding an adenosine-specific deaminase, wherein the adenosine deaminase is TadA-8e in non-limiting examples.

In another preferred embodiment, the present disclosure also provides a method for performing base editing in a cell, comprising the step of introducing a nucleic acid base editor, or a recombinant expression construct encoding the nucleic acid base editor of the present disclosure, or a fusion protein encoding the nucleic acid base editor of the present disclosure into the cell. In the practice of the method set forth herein, base editing is performed at a target nucleic acid recognized by the specific binding protein, and results in the change of a cytosine residue or an adenine residue.

In another preferred embodiment, the present disclosure provides a nucleic acid base editor that is specific for the base editing activity in nucleus or organelles. Further, a nucleic acid base editor for nucleus may comprise a nuclear localization signal (NLS). Further, a base editor for mitochondrion or chloroplast may comprise a mitochondrial targeting sequence (MTS) or a chloroplast translocation peptide (CTP), respectively. In these Examples, NLS, MTS or CTP may be substituted with each other depending on different specific target organelles or base editors, which will be described in further detail herein.

Exemplary technical solutions of the present disclosure are as below.

The first object of the present disclosure is to provide a nucleic acid base editor, comprising the following elements: a) a sequence-specific DNA binding protein; b) a nickase; c) an exonuclease; and d) a base-specific deaminase.

Preferably, each element of the nucleic acid base editor exists alone, or constitutes one or more fusion proteins.

Preferably, the sequence-specific DNA binding protein is one or more selected from the group consisting of a TALE protein, a ZFA protein, a Cas protein and a meganuclease.

Preferably, the sequence-specific DNA binding protein is a TALE protein.

Preferably, the nickase is a dimer of a cleavage domain monomer of FokI (Cleavage Domain monomer of FokI, FokICD) or a mutant of the dimer, the dimer of the FokICD monomer or the mutant of the dimer is composed of a pair of interacting cleavage domain monomers of FokI, and the dimer of the FokICD monomer or the mutant of the dimer has one and only one FokICD monomer which has DNA endonuclease activity.

Preferably, the cleavage domain monomer of FokI is isolated from a mutant of a wild-type FokI protein, the mutant of the wild-type FokI protein has a mutation at position 450 and/or position 467, or has an amino acid sequence which has at least 85%, at least 90%, at least 92%, at least 95%, at least 96%, at least 97%, at least 98%, at least 99%, or at least 99.5% identity with that of the cleavage domain monomer of FokI.

Further preferably, the mutation causes the FokICD monomer to lose DNA endonuclease activity.

Preferably, the cleavage domain monomer of FokI (FokICD) is isolated from a mutant of a wild-type FokI protein, the mutation prevents the FokICD monomer from the self-polymerization with a FokICD monomer containing a mutation at a same site and the formation of a dimer.

Further preferably, a sequence of the FokICD monomer is selected from SEQ ID No.87-88.

Preferably, the amino acid sequence of the cleavage domain monomer of FokI (FokICD) is selected from SEQ No. 60-63.

Preferably, the base-specific deaminase is selected from a cytidine-specific deaminase and an adenosine-specific deaminase.

Further preferably, the deaminase is selected from the deaminase as set forth in sequences SEQ ID NO. 36-59 and 80-86.

Further preferably, the base-specific deaminase is a cytidine-specific deaminase.

Further preferably, the cytidine-specific deaminase is one or more selected from the group consisting of hAPOBEC3A, rAPOBEC1, hAID, pmCDAT and Sdd deaminase.

Further, the nucleic acid base editor further comprises:
e) a uracil glycosylase inhibitor (UGI); and
the uracil glycosylase inhibitor exists alone, or constitutes at least one fusion protein with other elements of the nucleic acid base editor.

Preferably, the base-specific deaminase is an adenosine-specific deaminase.

Preferably, the adenosine-specific deaminase is TadA-8e.

Further, the nucleic acid base editor further comprises:
f) γb;
the γb constitutes at least one fusion protein with other elements of the nucleic acid base editor.

The second object of the present disclosure is to provide a fusion protein that is a nucleic acid base editor, the fusion protein comprises a protein domain of the base editor as described in the first object.

Another object of the present disclosure is to provide a fusion protein that is a nucleic acid base editor, the fusion protein comprises in linear order from the protein's amino terminus an exonuclease, an XTEN linker peptide, a base-specific deaminase, an XTEN linker peptide, a uracil glycosylase inhibitor (UGI) and a nuclear localization signal.

Another object of the present disclosure is to provide a fusion protein that is a nucleic acid base editor, the fusion protein comprises in linear order from the protein's amino terminus an exonuclease, a 48-amino acid linker peptide, a base-specific deaminase, an XTEN linker peptide, a uracil glycosylase inhibitor (UGI) and a nuclear localization signal.

Another object of the present disclosure is to provide a composition of fusion proteins having nucleic acid base editor activity, the composition comprises:
a first fusion protein comprising a nuclear localization signal (NLS), a sequence-specific DNA binding protein and a base-specific deaminase;
a second fusion protein comprising an exonuclease and a nuclear localization signal (NLS); and
a third fusion protein comprising a uracil glycosylase inhibitor (UGI) and a nuclear localization signal (NLS).

Another object of the present disclosure is to provide a composition of fusion proteins having nucleic acid base editor activity, the composition comprises:
a first fusion protein comprising in linear order from the protein's amino terminus a nuclear localization signal (NLS), a base-specific deaminase, a TALE-L protein, an FokI-L_{D450A} protein, a T2A sequence, an NLS, a TALE-R protein and an FokI-R protein;
a second fusion protein comprising an exonuclease and a nuclear localization signal (NLS); and
a third fusion protein comprising a uracil glycosylase inhibitor (UGI) and a nuclear localization signal (NLS).

Another object of the present disclosure is to provide a composition of fusion proteins having nucleic acid base editor activity, the composition comprises:
a first fusion protein comprising in linear order from the protein's amino terminus a nuclear localization signal (NLS), a TALE-L protein, an FokI-L_{D450A} protein, a T2A sequence, an NLS, a base-specific deaminase, a 48-amino acid linker peptide, a TALE-R protein and an FokI-R protein;
a second fusion protein comprising an exonuclease and a nuclear localization signal (NLS); and
a third fusion protein comprising a uracil glycosylase inhibitor (UGI) and a nuclear localization signal (NLS).

Another object of the present disclosure is to provide a composition of fusion proteins having nucleic acid base editor activity, the composition comprises:
a first fusion protein comprising a nuclear localization signal (NLS), a sequence-specific DNA binding protein, a base-specific deaminase and a uracil glycosylase inhibitor (UGI); and
a second fusion protein comprising an exonuclease and a nuclear localization signal (NLS).

Another object of the present disclosure is to provide a composition of fusion proteins having nucleic acid base editor activity, the composition comprises:
a first fusion protein comprising in linear order from the protein's amino terminus a nuclear localization signal (NLS), a base-specific deaminase, a 48-amino acid linker peptide, a TALE-L protein, an FokI-L_{D450A} protein, a T2A sequence, an NLS, a TALE-R protein, an FokI-R protein, a 4-amino acid linker peptide and a uracil glycosylase inhibitor (UGI); and
a second fusion protein comprising an exonuclease and a nuclear localization signal (NLS).

Another object of the present disclosure is to provide a composition of fusion proteins having nucleic acid base editor activity, the composition comprises:
a first fusion protein comprising in linear order from the protein's amino terminus a nuclear localization signal (NLS), a uracil glycosylase inhibitor (UGI), a 4-amino acid linker peptide, a base-specific deaminase, a 48-amino acid linker peptide, a TALE-L protein, an FokI-L_{D450A} protein, a T2A sequence, an NLS, a TALE-R protein and an FokI-R protein; and
a second fusion protein comprising an exonuclease and a nuclear localization signal (NLS).

Another object of the present disclosure is to provide a composition of fusion proteins having nucleic acid base editor activity and capable of performing base editing in mitochondria, the composition comprises:
a first fusion protein comprising in linear order from the protein's amino terminus a mitochondrial targeting sequence (MTS), a TALE-L protein and an FokI-L_{D450A} protein;
a second fusion protein comprising in linear order from the protein's amino terminus a mitochondrial targeting sequence (MTS), a TALE-R protein and an FokI-R protein;
a third fusion protein comprising in linear order from the protein's amino terminus a mitochondrial targeting sequence (MTS) and an exonuclease;
a fourth fusion protein comprising in linear order from the protein's amino terminus a mitochondrial targeting sequence (MTS) and a base-specific deaminase; and
a fifth fusion protein comprising in linear order from the protein's amino terminus a mitochondrial targeting sequence (MTS) and a uracil glycosylase inhibitor (UGI).

Another object of the present disclosure is to provide a composition of fusion proteins having nucleic acid base editor activity and capable of performing base editing in mitochondria, the composition comprises:
a first fusion protein comprising in linear order from the protein's amino terminus a mitochondrial targeting sequence (MTS), a TALE-L protein and an FokI-L_{D450A} protein;
a second fusion protein comprising in linear order from the protein's amino terminus a mitochondrial targeting sequence (MTS), a TALE-R protein and an FokI-R protein;
a third fusion protein comprising in linear order from the protein's amino terminus a mitochondrial targeting sequence (MTS), γb and an exonuclease;
a fourth fusion protein comprising in linear order from the protein's amino terminus a mitochondrial targeting sequence (MTS) and a base-specific deaminase; and
a fifth fusion protein comprising in linear order from the protein's amino terminus a mitochondrial targeting sequence (MTS), γb and a uracil glycosylase inhibitor (UGI).

Another object of the present disclosure is to provide a composition of fusion proteins having nucleic acid base editor activity, the composition comprises:
a first fusion protein comprising a nuclear localization signal (NLS)/a chloroplast translocation peptide (CTP)/a mitochondrial targeting sequence (MTS), a sequence-specific DNA binding protein and a nickase;
a second fusion protein comprising an exonuclease and a nuclear localization signal (NLS)/a chloroplast translocation peptide (CTP)/a mitochondrial targeting sequence (MTS); and
a third fusion protein comprising a base-specific deaminase, a uracil glycosylase inhibitor (UGI) and a nuclear localization signal (NLS)/a chloroplast translocation peptide (CTP)/a mitochondrial targeting sequence (MTS).

Another object of the present disclosure is to provide a composition of fusion proteins having nucleic acid base editor activity, the composition comprises:
a first fusion protein comprising in linear order from the protein's amino terminus a nuclear localization signal (NLS)/a chloroplast translocation peptide (CTP)/a mitochondrial targeting sequence (MTS), a TALE-L protein, an FokI-L_{D450A} protein, a T2A sequence, an NLS, a TALE-R protein and an FokI-R protein, or comprising in linear order from the protein's amino terminus a nuclear localization signal (NLS)/a chloroplast translocation peptide (CTP)/a mitochondrial targeting sequence (MTS), a TALE-L protein, an FokI-L protein, a T2A sequence, an NLS, a TALE-R protein and an FokI-R_{D450A} protein;
a second fusion protein comprising in linear order from the protein's amino terminus a nuclear localization signal (NLS)/a chloroplast translocation peptide (CTP)/a mitochondrial targeting sequence (MTS) and an exonuclease; and
a third fusion protein comprising in linear order from the protein's amino terminus a nuclear localization signal (NLS)/a chloroplast translocation peptide (CTP)/a mitochondrial targeting sequence (MTS), a base-specific deaminase, an XTEN linker peptide and a uracil glycosylase inhibitor (UGI).

Another object of the present disclosure is to provide a composition of fusion proteins having nucleic acid base editor activity and capable of performing base editing in mitochondria, wherein the composition comprises:
a first fusion protein comprising in linear order from the protein's amino terminus a mitochondrial targeting sequence (MTS), a base-specific deaminase, a 48-amino acid linker peptide, a TALE-L protein, an FokI-L_{D450A} protein, an 11-amino acid linker peptide and a uracil glycosylase inhibitor (UGI); and
a second fusion protein comprising in linear order from the protein's amino terminus a mitochondrial targeting sequence (MTS), a 48-amino acid linker peptide, a TALE-R protein, a uracil glycosylase inhibitor (UGI), a 14-amino acid linker peptide and an FokI-R protein.

Another object of the present disclosure is to provide a recombinant expression construct for nucleic acid base editing, the recombinant expression construct is used to express the nucleic acid base editor of the first object mentioned above or the fusion protein or the composition of other objects mentioned above.

Another object of the present disclosure is to provide a genetically engineered cell, and the genetically engineered cell is used for the transformation of the recombinant expression construct of the above-mentioned objects.

Another object of the present disclosure is to provide a method of performing nucleic acid base editing in a cell, the nucleic acid base editor or the recombinant expression construct of the above-mentioned objects is introduced into the cell so as to edit a target gene.

Preferably, the target gene is selected from a nuclear genomic DNA, a mitochondrial genomic DNA and a chloroplast genomic DNA.

Further preferably, the target gene is a nuclear genomic DNA, and the nucleic acid base editor further comprises a nuclear localization signal (NLS).

Further preferably, the target gene is a mitochondrial genomic DNA, and the nucleic acid base editor further comprises a mitochondrial targeting sequence (MTS).

Further preferably, the target gene is a chloroplast genomic DNA, and the nucleic acid base editor further comprises a chloroplast translocation peptide (CTP).

Another object of the present disclosure is to allow γb to be fused to the terminus of each element.

Further preferably, γb is fused to UGI and Trex2, respectively.

Another object of the present disclosure is to provide the use of base editing technique in base editing, wherein the base editor, the fusion protein, the composition, the recombinant expression construct, the genetically engineered cell or the method of the above-mentioned object is used to perform base editing on a DNA in a cell, and the cell is a mammalian cell, a bacterium, a protist, a fungus, an insect cell, a yeast, a non-conventional yeast or a plant cell.

Preferably, the plant cell is derived from a whole plant of a monocotyledon or a dicotyledon, a seedling, a meristem, a ground tissue, a vascular tissue, a dermal tissue, a seed, a leaf, a root, a bud, a stem, a flower, a fruit, a stolon, a bulb, a tuber, a corm, an asexual terminal branch, a bud, a budlet, or a tumor tissue.

Preferably, the mammalian cell is selected from a germ cell, a neuron, a muscle cell, an endocrine/exocrine cell, an epithelial cell, a muscle cell, a tumor cell, an embryonic cell, a hematopoietic cell, an osteocyte, germplasm cell, a somatic cell, a stem cell, a pluripotent stem cell, an induced pluripotent stem cell, a progenitor cell, a meiotic cell and a mitotic cell of human.

Preferably, the editor is used to perform base editing on a nuclear genome or an organellar genome.

Preferably, the organelle is mitochondrion or chloroplast.

Another object of the present disclosure is to provide the use of the base editor, the fusion protein, the composition, the recombinant expression construct or the genetically engineered cell of the above-mentioned objects in preparation of a pharmaceutical composition for treating a disease in a subject in need thereof.

Another object of the present disclosure is to provide a pharmaceutical composition for treating a disease in a subject in need thereof, the pharmaceutical composition comprises the base editor, the fusion protein, the composition, the recombinant expression construct or the genetically engineered cell of the above-mentioned objects, and optionally, a pharmaceutically acceptable carrier.

Another object of the present disclosure is to provide a method for producing a genetically modified plant, wherein the method comprises introducing the base editor, the fusion protein, the composition, the recombinant expression construct or the genetically engineered cell of the above-mentioned objects into at least one of the plants.

The present disclosure provides a base editor and the use thereof, and the beneficial effects thereof are as follow.
(1) The base editor of the present disclosure merely causes the occurrence of base editing on a selected single strand, thereby exhibiting good safety and precision.
(2) The base editor of the present disclosure achieves editing products with high purity and shows low production rate of indel byproducts, thereby having excellent editing efficiency.
(3) The base editor of the present disclosure has a low off-target rate, thereby effectively enhancing its therapeutic effects and safety.
(4) The base editor of the present disclosure is not based on CRISPR technology, has a wider range of applications and application scenarios, and all of the elements of said base editor are capable of functioning in nucleus or an organelle such as mitochondrion and chloroplast.

### BRIEF DESCRIPTION OF THE DRAWINGS

In order to better understand the technical solutions described in the present disclosure, description is now made with reference to the following drawings.

FIG. 1 is a schematic diagram of the functioning of the nucleic acid base editor of the present disclosure, wherein firstly, a sequence-specific DNA binding protein (SSDBP) locates and binds to a target DNA sequence; secondly, a nickase nicks one DNA strand preferentially at the target site and thereafter an exonuclease digests the nicked DNA strand from the nick to the SSDBP binding site. This would expose an ssDNA fragment in the complementary chain, which then becomes a substrate for a deaminase to realize deamination, thus resulting in the conversion of corresponding bases (C:G pairing to T:A pairing or A:T pairing to G:C pairing, the type of conversion depends on the deaminase used) after DNA repair.

FIG. 2A and FIG. 2B show the application effects of the high-purity base editing of the nucleic acid base editor of the present disclosure in rice nuclear base editing. Among them, FIG. 2A shows the C>T base editing efficiency for the OsBADH2 site in rice protoplast in cases where different treatment methods are adopted, and FIG. 2B shows the C>T base editing efficiency and the frequency of generating indel byproducts for the OsBADH2 site in rice protoplast in cases where different treatment methods are adopted.

FIG. 3A and FIG. 3B show the analysis of the base editing window of the base editor of the present disclosure. The rice protoplast is transformed with the nucleic acid base editor of the present disclosure, DNA is then extracted and the target site is subjected to high-throughput sequencing, so as to obtain the editing efficiency for different bases on the target sequence. FIG. 3A shows the schematic diagram of the OsBADH2 target sequence. The gray sequences on both sides are the TALE binding sites, and the black region in the middle is the spacer sequence. FIG. 3B shows the base editing window of the base editor obtained according to the analysis of the high-throughput sequencing results, wherein CK is a blank control without the transformation of any plasmid, TALEN_{WT} and TALEN_{WT}+ExoI are those with the transformation of the wild-type TALEN or the transformation of a combination of TALEN and exonuclease ExoI, respectively, and these two treatments serve as negative control.

FIG. 4A and FIG. 4B show the editing efficiency of cytosine nucleotide at the target site (FIG. 4A) and the frequency of generating indel byproducts (FIG. 4B) analyzed by high-throughput sequencing after the rice protoplast is transformed with the base editor of the present disclosure to target OsDEP1, wherein CK is a blank control without the transformation of any plasmid, TALEN_{WT} and TALEN_{WT}+ExoI are those with the transformation of the wild-type TALEN or the transformation of a combination of TALEN and exonuclease ExoI, respectively, and these two treatments serve as negative control.

FIG. 5A and FIG. 5B show the application effects achieved by using base editors comprising combinations of different FokI nickases, different exonucleases and the cytidine deaminase. Different editing windows are generated when exonucleases with different digestion directions are used; and different DNA single strands at the target site are subjected to specific base editing when different nickases are used (FIG. 5A). The purity of the editing products and the frequency of generating byproducts of the base editor of the present disclosure achieved by different combinations are analyzed (FIG. 5B).

FIG. 6A and FIG. 6B show the base editing efficiency and the frequency of indel byproducts introduced into a target sequence (OsBADH2 in rice protoplast) by the base editor comprising a combination of a cytidine deaminase and an exonuclease of the present disclosure as determined by high-throughput sequencing, wherein the exonuclease is a 5′ exonuclease or a 3′ exonuclease.

FIG. 7A and FIG. 7B show the base editing efficiency introduced into a target sequence (OsBADH2 in rice protoplast) by the base editor comprising a combination of a different cytidine deaminase and an exonuclease of the present disclosure as determined by high-throughput sequencing, and show the editing window.

FIG. 8 shows the base editing efficiency introduced into a target sequence (OsCKX2 in rice protoplast) by the base editor comprising an adenosine deaminase of the present disclosure, as determined by high-throughput sequencing.

FIG. 9 is a schematic diagram of a base editor of the present disclosure, comprising a fusion protein of an exonuclease, a deaminase, a uracil DNA glycosylase inhibitor and a nuclear localization signal (NLS) separated by an XTEN linker peptide or a 48-amino acid linker peptide.

FIG. 10A and FIG. 10B show the base editing efficiency introduced into a target sequence (OsDEP1 in rice protoplast) by the base editors expressed by the different constructs of the present disclosure as determined by high-throughput sequencing (FIG. 10A) and show the editing windows of different base editors (FIG. 10B).

FIG. 11A and FIG. 11B are schematic diagrams of the base editors comprising a deaminase-TALE fusion protein as a vector of the present disclosure. In each embodiment, a fusion protein of an NLS-exonuclease and an NLS-uracil glycosylase inhibitor (UGI) are provided individually in separate vectors.

FIG. 12A and FIG. 12B are bar graphs showing the base editing rates and the indel rates introduced into target sequences (OsDEP1 in rice protoplast, FIG. 12A; OsCKX2 in rice protoplast, FIG. 12B) by the base editor (fusion protein) of the present disclosure. The results of the fusion protein of the deaminase-TALE-FokI-R_{nickase} protein are as shown in FIG. 12A, and the results of the fusion protein of the deaminase-TALE-FokI-L_{nickase} protein are as shown in FIG. 12B.

FIG. 13A and FIG. 13B are schematic diagrams of the base editors comprising a deaminase-TALE fusion protein of the present disclosure. In each embodiment, a fusion protein of an NLS and an exonuclease is provided in a separate vector.

FIG. 14 shows the base editing efficiency in a target sequence (OsDEP1 in rice protoplast) resulted from using the fusion proteins as shown in FIG. 13A and FIG. 13B or expressing each component individually as the base editor of the present disclosure.

FIG. 15A is a schematic diagram of a vector used in the base editor of the present disclosure in mitochondrial editing, comprising constructs expressing MTS-deaminase, MTS-UGI, MTS-TALE-R-FokI-R (or MTS-TALE-R-FokI-R_{D450A}), MTS-TALE-L-FokI-L_{D450A} (or MTS-TALE-L-FokI-L) nickase and MTS-exonuclease.

FIG. 15B is a schematic diagram showing a target sequence targeted by the base editor of the present disclosure using constructs as shown in FIG. 15A and showing the binding sites of TALE-R and TALE-L and cytosine residues targeted by certain nucleic acid base editors of the present disclosure, that is, a schematic diagram of mitochondrial ND6 target sequence and TALE binding sites.

FIG. 15C shows the efficiency of the base mutations introduced into the target sequence by the base editor of the present disclosure using constructs as shown in FIG. 15A.

FIG. 16A to FIG. 16E are representative illustrations of the recombinant expression constructs encoding the base editors used in the Examples set forth herein in rice. In FIG. 16A to FIG. 16E, FokK-L-nickase is equivalent to FOKI-L; and FokI-R is equivalent to FOKI-R (D450A/D467A).

FIG. 16A shows the recombinant expression construct encoding the wild-type TALEN used in Example 2 and other examples (the schematic diagram of the NLS-TALEN_{WT} vector, taking the TALE targeting OsBADH2 as an example). This vector could result in double-strand breaks and trigger indel mutations randomly in the target DNA, and is used as control in each example. In this construct, a stably expressed T-DNA vector having a UBI promoter derived from maize and a Nos terminator is used to drive the expression of the wild-type TALEN (including the TALE-L-FokI-L fusion protein and the TALE-R-FokI-R fusion protein, wherein FokI does not contain D450A or D467A mutation), wherein the N- and C-terminal regions of TALE comprise the corresponding truncations (ΔN152/C63), flanking the DNA-binding domain of TALE. The TALE-L-FokI-L fusion protein and the TALE-R-FokI-R fusion protein are linked via the T2A self-cleaving peptide. Other components shown in the Figure include a CaMV 35S promoter (a Cauliflower Mosaic Virus-derived promoter), the hygromycin resistance gene Hyg, the nopaline synthase terminator Nos of Agrobacterium tumefaciens, and the like.

FIG. 16B is a schematic representation of a recombinant expression construct comprising the sequence-specific DNA binding proteins (TALE-L, TALE-R) and the nickase (FokI nickase) (i.e., a schematic diagram of a vector containing a nickase, an exonuclease and a deaminase as parts of the vector, taking the TALE targeting OsBADH2 as an example; the corresponding coding sequence of TALE may be designed depending on the target sequence) and two additional constructs, i.e., NLS-deaminase-UGI and exonuclease-NLS. All of these constructs comprise a UBI promoter derived from maize and a Nos terminator, which drive the expression of the deaminase-UGI fusion protein and the exonuclease, respectively. UGI (a uracil-DNA glycosylase inhibitor derived from Bacillus subtilis bacteriophage) protects the uracil(s) in DNA by irreversibly inhibiting uracil-DNA glycosylase which is the key DNA repair enzyme. Other components shown in the Figure include a CaMV 35S promoter (a Cauliflower Mosaic Virus-derived promoter), the hygromycin resistance gene Hyg, the nopaline synthase terminator Nos of Agrobacterium tumefaciens, and a CaMV poly(A) signal terminator.

FIG. 16C is a schematic representation of a recombinant expression construct comprising the fusion protein of the sequence-specific DNA binding proteins (TALE-L, TALE-R), the nickase (FokI nickase) and the deaminase (i.e., a schematic diagram of a vector containing a nickase, an exonuclease, a deaminase and a uracil glycosylase inhibitor as parts of the vector, taking the TALE targeting OsBADH2 as an example; the corresponding coding sequence of TALE may be designed depending on the target sequence) and two additional constructs, i.e., UGI-NLS and exonuclease-NLS. Each of the recombinant expression constructs (UGI-NLS and exonuclease-NLS) has a UBI promoter and a CaMV terminator, which drive the expression of UGI and the exonuclease. UGI (a uracil-DNA glycosylase inhibitor derived from Bacillus subtilis bacteriophage) protects the uracil(s) in DNA by irreversibly inhibiting uracil-DNA glycosylase which is the key DNA repair enzyme. Other components shown in the Figure include a CaMV 35S promoter (a Cauliflower Mosaic Virus-derived promoter), the hygromycin resistance gene Hyg, the nopaline synthase terminator Nos of Agrobacterium tumefaciens, and a CaMV poly(A) signal terminator.

FIG. 16D is a schematic representation of a recombinant expression construct comprising the fusion protein of the sequence-specific DNA binding proteins (TALE-L, TALE-R), the nickase (FokI nickase), the deaminase and UGI (i.e., a schematic diagram of a vector containing NLS-deaminase-TALE-L-FokI-_{nickase}-TALEN-R-UGI and exonuclease-NLS as parts of the vector, taking the TALE targeting OsBADH2 as an example; the corresponding coding sequence of TALE may be designed depending on the target sequence) and an additional construct, i.e., exonuclease-NLS. The recombinant expression construct (exonuclease-NLS) has a UBI promoter and a CaMV terminator to drive the expression of exonuclease. UGI (a uracil-DNA glycosylase inhibitor derived from a Bacillus subtilis bacteriophage) protects the uracil(s) in DNA by irreversibly inhibiting uracil-DNA glycosylase which is the key DNA repair enzyme. Other components shown in the Figure include a CaMV 35S promoter (a Cauliflower Mosaic Virus-derived promoter), the hygromycin resistance gene Hyg, the nopaline synthase terminator Nos of Agrobacterium tumefaciens, and a CaMV poly(A) signal terminator.

FIG. 16E is a schematic representation of a recombinant expression construct comprising the fusion protein of the sequence-specific DNA binding proteins (TALE-L, TALE-R), the nickase (FokI nickase), the deaminase, the exonuclease and UGI (a schematic diagram of NLS-deaminase-TALE-L-FokI-_{nickase}-TALEN-R-UGI-exonuclease vector, taking the TALE targeting OsBADH2 as an example, the corresponding coding sequence of TALE may be designed depending on the target sequence), having the additional feature that UGI and exonuclease are encoded in the construct rather than being introduced into the cell in separate constructs.

FIG. 17A to FIG. 17H are representative illustrations of the recombinant expression constructs encoding the base editors used in the Examples set forth herein for mitochondrial editing in human cells.

FIG. 17A is a representation of the recombinant expression construct MTS-TALE-L-FokI-L for mitochondria (a schematic diagram of the MTS-TALE-L-FokI-L vector targeting mitochondrial ND6), wherein the TALE sequence could be replaced correspondingly depending on targets. The expression vector MTS-TALE-L-FokI-L has a CMV promoter and a bGH poly(A) signal terminator to drive the expression of the MTS-TALE-L-FokI-L fusion protein, wherein the N- and C-terminal regions of TALE comprise the corresponding truncations (ΔN152/C63), flanking the DNA-binding domain of TALE (see Mok et al., 2020, Nature 583: 631-637). MTS is a mitochondrial targeting sequence of Homo sapiens superoxide dismutase 2 that facilitates the translocation of proteins into mitochondria. The CMV promoter is a human herpesvirus 5-derived promoter, which has been demonstrated to be highly active in animal cells. The CMV enhancer is a cytomegalovirus promoter region-containing fragment capable of enhancing the transcriptional efficiency of the CMV promoter. The bGH poly(A) signal is a somatotropin poly-adenylylation signal-derived terminator.

FIG. 17B is a representation of the recombinant expression construct MTS-TALE-R-FokI-R for mitochondria (a schematic diagram of the MTS-TALE-R-FokI-R vector targeting mitochondrial ND6), wherein the TALE sequence could be replaced correspondingly depending on targets. The expression vector MTS-TALE-R-FokI-R has a CMV promoter and a bGH poly(A) signal terminator to drive the expression of the MTS-TALE-R-FokI-R fusion protein, wherein the N- and C-terminal regions of TALE comprise the corresponding truncations (ΔN152/C63), flanking the DNA-binding domain of TALE (see Mok et al., 2020, Nature 583: 631-637). In this vector, MTS is a mitochondrial targeting sequence of Cytochrome c oxidase subunit 8 that facilitates the translocation of proteins into mitochondria. The CMV promoter is a human herpesvirus 5-derived promoter, which has been demonstrated to be highly active in animal cells. The CMV enhancer is a cytomegalovirus promoter region-containing fragment capable of enhancing the transcriptional efficiency of the CMV promoter. The bGH poly(A) signal is a somatotropin poly-adenylylation signal-derived terminator.

FIG. 17C is a schematic diagram of the recombinant expression construct MTS-deaminase for mitochondria (a schematic diagram of the MTS-deaminase vector). This recombinant expression construct has a CMV promoter and a bGH poly(A) signal terminator to drive the expression of MTS-deaminase in human mitochondria. The MTS, the CMV promoter, the CMV enhancer and the bGH poly(A) signal terminator are as described in FIG. 17A.

FIG. 17D is a representation of the recombinant expression construct MTS-exonuclease for mitochondria (a schematic diagram of the MTS-exonuclease vector). This recombinant expression construct has a CMV promoter and a bGH poly(A) signal terminator to drive the expression of MTS-exonuclease in human mitochondria. The MTS, the CMV promoter, the CMV enhancer and the bGH poly(A) signal terminator are as described in FIG. 17A.

FIG. 17E is a representation of the recombinant expression construct MTS-UGI for mitochondria (a schematic diagram of the MTS-UGI vector). This recombinant expression construct has a CMV promoter and a bGH poly(A) signal terminator to drive the expression of MTS-UGI (a uracil glycosylase inhibitor derived from a Bacillus subtilis bacteriophage) in human mitochondria. The MTS, the CMV promoter, the CMV enhancer and the bGH poly(A) signal terminator are as described in FIG. 17A.

FIG. 17F is a schematic diagram of the recombinant expression construct MTS-deaminase-TALE-L-FokI-L for mitochondria (a schematic diagram the MTS-deaminase-TALE-L-FokI-L vector). The recombinant expression construct MTS-deaminase-TALE-L-FokI-L has a CMV promoter and a bGH poly(A) signal terminator to drive the expression of the MTS-deaminase-TALE-L fusion protein. Components such as the MTS, the CMV promoter, the CMV enhancer and the bGH poly(A) signal terminator are as described in FIG. 17A.

FIG. 17G is a schematic diagram of the recombinant expression construct MTS-exonuclease-TALE-R-FokI-R for mitochondria (a schematic diagram of the MTS-exonuclease-TALE-R-FokI-R vector). The recombinant expression construct MTS-exonuclease-TALE-R-FokI-R has a CMV promoter and a bGH poly(A) signal terminator to drive the expression of the MTS-exonuclease-TALE-R fusion protein. Components such as the MTS, the CMV promoter, the CMV enhancer and the bGH poly(A) signal terminator are as described in FIG. 17B.

FIG. 17H is a schematic diagram of the recombinant expression construct MTS-UGI-exonuclease-TALE-R-FokI-R for mitochondria (a schematic diagram of the MTS-UGI-exonuclease-TALE-R-FokI-R vector). The recombinant expression construct MTS-UGI-exonuclease-TALE-R-FokI-R has a CMV promoter and a bGH poly(A) signal terminator to drive the expression of the MTS-exonuclease-TALE-R fusion protein. Components such as the MTS, the CMV promoter, the CMV enhancer and the bGH poly(A) signal terminator are as described in FIG. 17B.

FIG. 18 is a schematic structural diagram of CyDENT for nuclear genome editing.

FIG. 19A shows the C-to-T conversion frequency and indel frequency achieved by nuCyDENT-R and TALEN at the OsDEP1, OsSD1, OsCKX2 and OsBADH2 sites in rice protoplast.

FIG. 19B shows the base editing windows of CyDENT at the OsDEP1, OsSD1, OsCKX2 and OsBADH2 sites in rice protoplast. In the figure, the gray regions represent the TALE binding sites, and the middle region is the spacer region.

FIG. 20 shows the base editing of CyDENT at the OsCKX2 and OsSD1 sites in rice protoplast. The gray regions are the TALE binding sites.

FIG. 21 shows the base editing of CyDENT at human SIRT6 site. The gray regions are the TALE binding sites.

FIG. 22A is a schematic overview of the modular CyDENT construct used in chloroplast genome editing, and cpCyDENT-R is taken as an example.

FIG. 22B shows the base editing window of CyDENT at the OsrbcL site in rice protoplast. The gray regions are the TALE binding sites.

FIG. 23A is a schematic diagram of the structure of the modular CyDENT used in mitochondria. mtCyDENT-R is taken as an example.

FIG. 23B shows the base editing at the mitochondrial ND6 site in HEK293T cells by mtCyDENT-L or mtCyDENT-R in various fusion states with γb.

FIG. 24 shows the editing frequencies of DdCBE, mtCyDENT-R, mtCyDENT1b-R, mtCyDENT-L and mtCyDENT1b-L at the ND1.2, ND1.3, ND3 and ND6.2 sites in the mitochondria in HEK293T cells.

FIG. 25 shows the indel frequencies of DdCBE, mtCyDENT1b-R and mtCyDENT1b-L at different sites in the mitochondria of HEK293T cells.

FIG. 26 shows the base editing sites of mtCyDENT at different sites in the mitochondria of HEK293T cells. The gray regions are the TALE binding sites.

FIG. 27 shows the editing frequencies achieved by using Sdd7 deaminase mtCyDENT1b-L and mtCyDENT1b-R at the ND5.1, ND6 and ND1.3 sites in HEK293T cells.

FIG. 28A is a schematic diagram of the mtCyDENT2 construct in the mitochondrial genome.

FIG. 28B shows the base editing efficiency of DdCBE as well as mtCyDENT2-L and mtCyDENT2-R comprising different deaminases at the ND6 site in HEK293T cells and the ratio of various editing events.

FIG. 29 shows the editing frequencies and the strand preferences in editing of DdCBE and mtCyDENT2-L comprising Sdd3 deaminase at the ND1.2 and ND6.2 sites in HEK293T cells, wherein the gray regions represent the TALE binding sites.

FIG. 30 shows the strand preferences in editing of mtCyDENT2-L (Sdd3 deaminase+TALE-L1+TALE-R1) (designed for the pathogenic mutation of Leigh's syndrome at the ND6.2 site) at the ND6.2 site in HEK293T cells.

FIG. 31A shows the Whole-Genome Sequencing (WGS) analysis and Next-Generation Sequencing (NGS) analysis of the editing frequencies at the target sites ND3 and ND6.2.

FIG. 31B shows the Logo diagram of the off-target C:G to T:A base conversion and G:C to A:T base conversion of each editor.

FIG. 31C shows the SNV frequency distribution and indel frequency distribution in potential TALE-dependent off-target sites.

### DETAILED DESCRIPTION

### Terms

Unless otherwise defined, all technical terms used herein have the same meaning as those commonly understood by a person skilled in the art.

A numerical range includes the number(s) defining the range, and explicitly includes each integer and non-integer fraction within the defined range. Unless otherwise indicated, all technical and scientific terms used herein have the same meaning as those commonly understood by one of ordinary skill in the art.

The terms “structure”, “recombinant expression structure” or “recombinant expression construct” used in the present disclosure refers to an artificially designed DNA fragment that may be used to introduce the genetic material into a target cell (for example, a recombinant expression structure is used to produce a base editor or the components thereof). The term “express” refers to the transcription and translation of a nucleic acid encoding sequence, resulting in the production of an encoded polypeptide.

The term “genetically engineered” used in the present disclosure refers to change the genetic makeup of the cells by biotechnology, including the transfer of genes within and across species boundaries, to produce improved or non-naturally occurring cells. In particular uses of this term, the construct encodes the base editor or the components thereof, and the base editor is produced by the genetically engineered cells. A cell that contains an exogenous, recombinant, synthetic and/or otherwise modified polynucleotide is considered to be a genetically engineered cell, and thus non-naturally occurring relative to any naturally occurring counterpart. In some cases, a genetically engineered cell comprises one or more recombinant nucleic acids. In other cases, a genetically engineered cell comprises one or more synthetic or genetically engineered nucleic acids (for example, a nucleic acid containing at least one artificially created insertion, deletion, inversion or substitution relative to the sequence of its naturally occurring counterpart). Methods for producing genetically engineered cells are known in the art, for example, as described in Sambrook et al., Molecular Cloning, A Laboratory Manual (Fourth Edition), Cold Spring Harbor Press, Cold Spring Harbor, N.Y. (2012).

The term “genetically engineered cell” or “genetically engineered host cell” or “recombinant expression host cell” used in the present disclosure may be a cell that has been modified using a gene editing technique. Gene editing refers to a type of genetic engineering in which DNA is inserted, deleted, modified or replaced in the genome of a living cell. Compared with other genetic engineering techniques that may randomly insert the genetic material into a host genome, gene editing is capable of targeting an insertions to a specific location (e.g., AAVS1 alleles). Examples of gene editing techniques include but are not limited to restriction enzymes, zinc finger nucleases, TALENs and CRISPR-Cas9. The base editor disclosed herein is a specific example of gene editing that permits changes in one or more single nucleotides to result in, inter alia, the alteration of phenotype of cell.

The term “deaminase”, “base-specific deaminase” or “deaminase domain” as used in the present disclosure refers to a protein or an enzyme that catalyzes a deamination reaction. In the present disclosure, “deaminase” and “base-specific deaminase” may be used interchangeably. In some embodiments, the deaminase or deaminase domain is a cytidine deaminase, which catalyzes the hydrolytic deamination of cytidine or deoxycytidine respectively to generate uridine, which is finally converted to thymidine (T) during cell modification and DNA replication. In some embodiments, the deaminase or deaminase domain is an adenosine deaminase domain, which catalyzes the hydrolytic deamination of adenosine or deoxyadenosine to generate inosine or deoxyinosine (I), which is finally converted to guanosine or deoxyguanosine (G) during cell modification and DNA replication. In some embodiments, the deaminase or deaminase domain is a naturally occurring deaminase derived from an organism, such as a microorganism, a plant, an animal, such as a human, a chimpanzee, a gorilla, a monkey, a cattle, a dog, a rat, or a mouse. In some embodiments, the deaminase or deaminase domain is a variant of a naturally occurring deaminase derived from an organism, which does not exist in nature. For example, in some embodiments, the deaminase or deaminase domain is at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% identical to a naturally occurring deaminase derived from an organism.

The term “linker peptide” or “Linker” as used in the present disclosure refers to an element linking two molecules or moieties, for example, two domains of a fusion protein. In some embodiments, the linker peptide is an organic molecule, a group, a polymer or a chemical moiety. In some embodiments, the linker peptide is a linker peptide that is 5 to 100 amino acids in length, for example, 4, 5, 6, 7, 8, 9, 10, 11, 12, 13, 14, 15, 16, 17, 18, 19, 20, 21, 22, 23, 24, 25, 26, 27, 28, 29, 30, 30 to 35, 35 to 40, 40 to 45, 45 to 50, 50 to 60, 60 to 70, 70 to 80, 80 to 90, 90 to 100, 100 to 150, or 150 to 200 amino acids in length. Longer or shorter linker peptides have also been considered.

The term “mutation” as used in the present disclosure refers to the substitution of a residue in a sequence (for example, nucleic acid sequence or amino acid sequence) with another residue or the deletion or insertion of one or more residues in the sequence. In the present disclosure, mutations are generally described by the identification of the initial residue, followed by the identification of the position of the residue in the sequence and the identity of the newly substituted residue. Various methods for generating the amino acid substitutions (mutations) provided herein are well known in the art, and are provided in, for example, Green and Sambrook, Molecular Cloning: A Laboratory Manual (4^{th} ed., Cold Spring Harbor Laboratory Press, Cold Spring Harbor, N.Y. (2012)).

The term “uracil glycosylase inhibitor” or “UGI” as used in the present disclosure refers to a protein capable of inhibiting uracil-DNA glycosylase as a base excision repair enzyme.

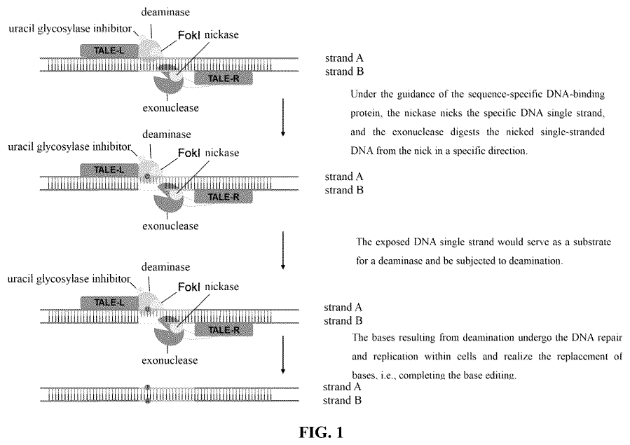

The terms “top strand” or “strand A” and “bottom strand” or “strand B” as used in the present disclosure are merely intended to distinguish the relative positions of the two strands at the target site of DNA in a certain example for ease of the exemplary description of the editing effect of the base editor of the present disclosure on a single-stranded DNA, and have no specific limitation on a specific double-stranded DNA structure. Among them, “top strand” and “strand A” is interchangeable, and “bottom strand” and “strand B” is interchangeable. Unless otherwise specified, the “top strand” or the “strand A” that conforms to the schematic diagram of the present application (FIG. 1) is a DNA single strand that interacts with TALE-L, and correspondingly, the “bottom strand” or the “strand B” is a DNA single strand that interacts with TALE-R.

Various examples according to the composition and the method of the present disclosure are now described in the following non-limiting examples. This example is merely for the purpose of illustration and does not limit the scope of the present disclosure in any way.

### Nucleic Acid Base Editor

The base editing function of the nucleic acid base editor of the present disclosure is as shown in FIG. 1. Its components include a sequence-specific DNA binding protein (SSDBP), a nickase, an exonuclease (having 5′ or 3′ exonuclease activity), a cytidine deaminase or an adenosine deaminase, optionally a uracil glycosylase inhibitor (UGI), and optionally a localization sequence. These components may be expressed by separate constructs or fused in one or more constructs using appropriate linker peptides.

### Sequence-Specific DNA Binding Protein

In the base editor disclosed herein, SSDBP may be a TALE protein, a zinc-finger protein (ZFA protein), a CRISPR-Cas endonuclease (Cas protein) or a meganuclease, wherein a TALE protein is selected in some specific embodiments. A transcription activator-like effector (TALE) protein is derived from the transcription activator-like effector of Xanthomonas spp., and is artificially modified into a sequence-specific DNA binding protein. A TALE protein comprises 1 to 33 repeating units with a length of 33˜35 amino acid residues, wherein each repeating unit and the half-repeating unit at the terminus are capable of specifically recognizing and binding to a specific nucleotide target site. In each repeat sequence, the type of the DNA base capable of being recognized and bound to by TALE is determined by two hypervariable residues (referred to as repeat-variable di-residues (RVDs)) at positions 12 and 13 that target a specific base pair. The code or type of DNA recognition by RVDs has been deciphered: RVDs His/Asp (HD), Asn/Gly (NG), Asn/Asn (NN) and Asn/Ile (NI) recognize cytosine (C), thymine (T), guanine (G) and adenine (A), respectively (see, Boch & Bonas, 2010, Annu. Rev. Phytopathol. 48: 419-436; Deng et al., 2012, Cell Res. 22: 1502-1504). TALE repeating units are modular, and RVDs may be artificially designed for the target binding of DNA. As disclosed in the present disclosure, a pair of TALE proteins (respectively referred to as TALE-L or TALE-L protein and TALE-R or TALE-R protein) are used to bind DNA at two adjacent sites on DNA, wherein the DNA sequence between the adjacent sites is a spacer sequence, also referred to as a target sequence, wherein the binding sites of TALE-L and TALE-R are defined as Left Binding Site and Right Binding Site. The sequence specificity of the TALE protein is used to determine the target site in the base editor disclosed in the present disclosure. In addition, in some cases, only one TALE (rather than a pair) is needed for binding and targeting the dsDNA, and the base editing function of the present disclosure may also be realized.

The structures of exemplary TALE proteins that may be used as the component of the base editor disclosed in the present disclosure are provided below, including but not limited to the N-terminal as set forth in SEQ ID NO. 1, the C-terminal as set forth in SEQ ID NO. 2 and repeating units as set forth in SEQ ID NO. 3-35.

TALE-NTD (Δ152):
(SEQ ID NO. 1)
MVDLRTLGYSQQQQEKIKPKVRSTVAQHHEALVGHGFTHAHIVALSQHP
AALGTVAVTYQHIITALPEATHEDIVGVGKQWSGARALEALLTDAGELR
GPPLQLDTGQLVKIAKRGGVTAMEAVHASRNALTGAPLN
TALE-CTD (C63):
(SEQ ID NO. 2)
SIVAQLSRPDPALAALTNDHLVALACLGGRPAMDAVKKGLPHAPELIRR
VNRRIGERTSHRVA
OsBADH2-TALE-Left repeat:
(SEQ ID NO. 3)
LTPDQVVAIASNNGGKQALETVQRLLPVLCQDHG
(SEQ ID NO. 4)
LTPDQVVAIASHDGGKQALETVQRLLPVLCQDHG
(SEQ ID NO. 5)
LTPDQVVAIASNGGGKQALETVQRLLPVLCQDHG
(SEQ ID NO. 6)
LTPDQVVAIASNNGGKQALETVQRLLPVLCQDHG
(SEQ ID NO. 7)
LTPDQVVAIASNNGGKQALETVQRLLPVLCQDHG
(SEQ ID NO. 8)
LTPDQVVAIASNIGGKQALETVQRLLPVLCQDHG
(SEQ ID NO. 9)
LTPDQVVAIASNGGGKQALETVQRLLPVLCQDHG
(SEQ ID NO. 10)
LTPDQVVAIASNNGGKQALETVQRLLPVLCQDHG
(SEQ ID NO. 11)
LTPDQVVAIASHDGGKQALETVQRLLPVLCQDHG
(SEQ ID NO. 12)
LTPDQVVAIASNGGGKQALETVQRLLPVLCQDHG
(SEQ ID NO. 13)
LTPDQVVAIASNGGGKQALETVQRLLPVLCQDHG
(SEQ ID NO. 14)
LTPDQVVAIASNGGGKQALETVQRLLPVLCQDHG
(SEQ ID NO. 15)
LTPDQVVAIASNNGGKQALETVQRLLPVLCQDHG
(SEQ ID NO. 16)
LTPDQVVAIASNIGGKQALETVQRLLPVLCQDHG
(SEQ ID NO. 17)
LTPDQVVAIASNNGGKQALETVQRLLPVLCQDHG
(SEQ ID NO. 18)
LTPDQVVAIASNGGGKQALETVQRLLPVLCQDHG
(SEQ ID NO. 19)
LTPDQVVAIASNIGGKQALE
OsBADH2-TALE-Right repeat:
(SEQ ID NO. 20)
LTPDQVVAIASNNGGKQALETVQRLLPVLCQDHG
(SEQ ID NO. 21)
LTPDQVVAIASHDGGKQALETVQRLLPVLCQDHG
(SEQ ID NO. 22)
LTPDQVVAIASHDGGKQALETVQRLLPVLCQDHG
(SEQ ID NO. 23)
LTPDQVVAIASNGGGKQALETVQRLLPVLCQDHG
(SEQ ID NO. 24)
LTPDQVVAIASNGGGKQALETVQRLLPVLCQDHG
(SEQ ID NO. 25)
LTPDQVVAIASNGGGKQALETVQRLLPVLCQDHG
(SEQ ID NO. 26)
LTPDQVVAIASNGGGKQALETVQRLLPVLCQDHG
(SEQ ID NO. 27)
LTPDQVVAIASNNGGKQALETVQRLLPVLCQDHG
(SEQ ID NO. 28)
LTPDQVVAIASNGGGKQALETVQRLLPVLCQDHG
(SEQ ID NO. 29)
LTPDQVVAIASHDGGKQALETVQRLLPVLCQDHG
(SEQ ID NO. 30)
LTPDQVVAIASHDGGKQALETVQRLLPVLCQDHG
(SEQ ID NO. 31)
LTPDQVVAIASNIGGKQALETVQRLLPVLCQDHG
(SEQ ID NO. 32)
LTPDQVVAIASNIGGKQALETVQRLLPVLCQDHG
(SEQ ID NO. 33)
LTPDQVVAIASNNGGKQALETVQRLLPVLCQDHG
(SEQ ID NO. 34)
LTPDQVVAIASNNGGKQALETVQRLLPVLCQDHG
(SEQ ID NO. 35)
LTPDQVVAIASNIGGKQALETVQRLLPVLCQDHG

### Nickase

Nickase used as the component of the base editor disclosed herein is capable of cleaving one of the double strands of a target DNA. In the base editor disclosed herein, an exemplary nickase is FokI (or referred to as FokI protein) derived from Flavobacterium okeanokoites and in particular amino acid sequence variants wherein the dsDNA cleavage activity is converted into a nick produced in only one strand of a target DNA, including but not limited to D450A/D467A mutant. In addition, alternative nickases comprising bacterium type IIS restriction enzymes may also be used as the component of the base editor disclosed herein.

Wild-type FokI consists of two functional domains, which are a recognition domain and a cleavage domain, respectively. The recognition domain is removed artificially so as to obtain an FokICD merely retaining the cleavage domain. When two FokICD monomers interact with each other to form a dimer, the cleavage activity of FokICD would be activated, thus being capable of cleaving both strands of a double-stranded DNA. Exemplary FokICD monomers that may be used in the present disclosure are provided below, including but not limited to those as set forth in SEQ ID NO.87-88.

FokI-L:
(SEQ ID NO. 87)
QLVKSELEEKKSELRHKLKYVPHEYIELIEIARNSTQDRILEMKVMEFF
MKVYGYRGKHLGGSRKPDGAIYTVGSPIDYGVIVDTKAYSGGYNLPIGQ
ADEMQRYVKENQTRNKHINPNEWWKVYPSSVTEFKFLFVSGHFKGNYKA
QLTRLNHKTNCNGAVLSVEELLIGGEMIKAGTLTLEEVRRKFNNGEINF
FokI-R:
(SEQ ID NO. 88)
QLVKSELEEKKSELRHKLKYVPHEYIELIEIARNSTQDRILEMKVMEFF
MKVYGYRGKHLGGSRKPDGAIYTVGSPIDYGVIVDTKAYSGGYNLPIGQ
ADEMERYVEENQTRNKHLNPNEWWKVYPSSVTEFKFLFVSGHFKGNYKA
QLTRLNHITNCNGAVLSVEELLIGGEMIKAGTLTLEEVRRKFNNGEINF

When the aspartic acid at position 450 (the first amino acid of the wild-type FokI comprising the recognition domain is designated as the 1st amino acid; if the first amino acid of the FokICD merely comprising the cleavage domain is designated as the 1st amino acid, then the position is position 67) and/or position 467 (the first amino acid of the wild-type FokI comprising the recognition domain is designated as the 1st amino acid; if the first amino acid of the FokICD merely comprising the cleavage domain is designated as the 1st amino acid, then the position is position 84) in an FokICD monomer of the dimer is mutated to alanine (D450A or D467A), this FokICD monomer would lose the cleavage activity, while another FokICD monomer without amino acid mutation in the dimer still retains the cleavage activity.

The FokICD dimer thus obtained could and could only cleave one strand of a double-stranded DNA and could not cleave the other strand. Such dimer of FokICD is referred to as FokI_{nickase}, i.e., FokI nickase. For the convenience of description, an FokICD monomer fused to TALE-L is referred to as FokI-L (for example, as set forth in SEQ ID NO.87) by the inventors, and an FokICD monomer fused to TALE-R is referred to as FokI-R (for example, as set forth in SEQ ID NO.88). Further, FokICD mutant monomers that comprise FokI D450A and/or D467A mutation and thus lose the cleavage activity are referred to as FokI-L_{D450A/D467A} and FokI-R_{D450A/D467A}, respectively. In the present disclosure, the FokICD dimer formed by the interaction between FokI-L and FokI-R_{D450A/D467A} merely retains the cleavage activity of FokI-L, and this dimer is referred to as FokI-L_{nickase} (or referred to as FokI-L nickase); correspondingly, the FokICD dimer formed by the interaction between FokI-L_{D450A/D467A} and FokI-R merely retains the cleavage activity of FokI-R and is referred to as FokI-R_{nickase} (or referred to as FokI-R nickase).

It should be pointed out that FokI-L_{nickase} and FokI-R_{nickase} tend to nick different single strands in a double-stranded DNA, that is, FokI-L_{nickase} and FokI-R_{nickase} have single-strand specificity or preference upon nicking DNA. As shown in FIG. 1, at this target site, if FokI-R_{nickase} is used, then strand B tends to be nicked, correspondingly, if FokI-L_{nickase} is used, then strand A tends to be nicked (as shown in FIG. 1). The strand specificities exhibited by FokI-L_{nickase} and FokI-R_{nickase} are advantageous for the selection of the desired DNA single strand for the subsequent deamination step. Accompanied by the sequence-specific binding to the left binding site and the right binding site by TALE-L and TALE-R, FokI-L_{nickase} or FokI-R_{nickase} nicks the target sequence, leaving a nick in strand A or strand B, respectively. The strand specificity of the nickase determines the further deamination of the DNA single strand under the action of the base editor of the present disclosure.

Nickase protein monomers that may be used as the components of exemplary nucleic acid base editors of the present disclosure are provided below, including but not limited to those as set forth in SEQ ID NO.60-63.

FokI-L_{D450A}:
(SEQ ID NO. 60)
QLVKSELEEKKSELRHKLKYVPHEYIELIEIARNSTQDRILEMKVMEFF
MKVYGYRGKHLGGSRKPAGAIYTVGSPIDYGVIVDTKAYSGGYNLPIGQ
ADEMQRYVKENQTRNKHINPNEWWKVYPSSVTEFKFLFVSGHFKGNYKA
QLTRLNHKTNCNGAVLSVEELLIGGEMIKAGTLTLEEVRRKFNNGEINF
FokI-L_{D467A}:
(SEQ ID NO. 61)
QLVKSELEEKKSELRHKLKYVPHEYIELIEIARNSTQDRILEMKVMEFF
MKVYGYRGKHLGGSRKPDGAIYTVGSPIDYGVIVATKAYSGGYNLPIGQ
ADEMQRYVKENQTRNKHINPNEWWKVYPSSVTEFKFLFVSGHFKGNYKA
QLTRLNHKTNCNGAVLSVEELLIGGEMIKAGTLTLEEVRRKENNGEINF
FokI-R_{D450A}:
(SEQ ID NO. 62)
QLVKSELEEKKSELRHKLKYVPHEYIELIEIARNSTQDRILEMKVMEFF
MKVYGYRGKHLGGSRKPAGAIYTVGSPIDYGVIVDTKAYSGGYNLPIGQ
ADEMERYVEENQTRNKHLNPNEWWKVYPSSVTEFKFLFVSGHFKGNYKA
QLTRLNHITNCNGAVLSVEELLIGGEMIKAGTLTLEEVRRKFNNGEINF
FokI-R_{D467A}:
(SEQ ID NO. 63)
QLVKSELEEKKSELRHKLKYVPHEYIELIEIARNSTQDRILEMKVMEFF
MKVYGYRGKHLGGSRKPDGAIYTVGSPIDYGVIVATKAYSGGYNLPIGQ
ADEMERYVEENQTRNKHLNPNEWWKVYPSSVTEFKFLFVSGHFKGNYKA
QLTRLNHITNCNGAVLSVEELLIGGEMIKAGTLTLEEVRRKFNNGEINF

### Exonuclease

Depending on the type of the exonuclease used, the exonuclease component of the nucleic acid base editor of the present disclosure digests the nicked DNA strand from the nick site in 5′→3′ direction or in 3′→5′ direction. After exonuclease digestion, a short ssDNA fragment is exposed at the complementary DNA strand. The type of exonuclease determines the ssDNA region (or editing window) to be deaminated. Exonucleases that may be used as the component of the nucleic acid base editor disclosed herein include but are not limited to DNA Polymerases I and III (E. coli), mammalian p53 protein, exonucleases I-VII (E. coli) (such as exonucleases I and V (having 3′→5′ exonuclease activity)), bacteriophage-derived polymerases (such as T4 DNA polymerase (having 3′→5′ exonuclease activity)), Thermus aquaticus polymerase (having 5′->3′ exonuclease activity), and 3′→5′ exonuclease as reported by Shevelev and Hübscher (Shevelev & Hübscher, 2002, Nat. Rev. Molec. Cell Biol. 3: 364-376).

Exonuclease proteins that may be used as the components of exemplary base editors of the present disclosure are provided below, including but not limited to the proteins as set forth in sequences SEQ ID NO.64-67 and 153.

Exonuclease V (ExoV):
(SEQ ID NO. 153)
MAETGEEETASAEASGFSDLSDSELVEFLDLEEAKESAVSLSKPGPSAE
LPGKDDKPVSLQNWKGGLDVLSPMERFHLKYLYVTDLCTQNWCELQMVY
GKELPGSLTPEKAAVLDTGASIHLAKELELHDLVTVPIATKEDAWAVKF
LNILAMIPALQSEGRVREFPVFGEVEGIFLVGVIDELHYTSKGELELAE
LKTRRRPVLPLPAQKKKDYFQVSLYKYIFDAMVQGKVTPASLIHHTKLC
LDKPLGPSVLRHARQGGVSVKSLGDLMELVFLSLTLSDLPAIDTLKLEY
IHQETATILGTEIVAFEEKEVKSKVQHYVAYWMGHRDPQGVDVEEAWKC
RTCDYVDICEWRRGSGVLSSSWEPKAKKFK
mExoI:
(SEQ ID NO. 64)
MGIQGLLQFIQEASEPVNVKKYKGQAVAVDTYCWLHKGAIACAEKLAKG
EPTDRYVGFCMKFVNMLLSYGVKPILIFDGCTLPSKKEVERSRRERRQS
NLLKGKQLLREGKVSEARDCFARSINITHAMAHKVIKAARALGVDCLVA
PYEADAQLAYLNKAGIVQAVITEDSDLLAFGCKKVILKMDQFGNGLEVD
QARLGMCKQLGDVFTEEKFRYMCILSGCDYLASLRGIGLAKACKVLRLA
NNPDIVKVIKKIGHYLRMNITVPEDYITGFIRANNTFLYQLVFDPIQRK
LVPLNAYGDDVNPETLTYAGQYVGDSVALQIALGNRDVNTFEQIDDYSP
DTMPAHSRSHSWNEKAGQKPPGTNSIWHKNYCPRLEVNSVSHAPQLKEK
PSTLGLKQVISTKGLNLPRKSCVLKRPRNEALAEDDLLSQYSSVSKKIK
ENGCGDGTSPNSSKMSKSCPDSGTAHKTDAHTPSKMRNKFATFLQRRNE
ESGAVVVPGTRSRFFCSSQDFDNFIPKKESGQPLNETVATGKATTSLLG
ALDCPDTEGHKPVDANGTHNLSSQIPGNAAVSPEDEAQSSETSKLLGAM
SPPSLGTLRSCFSWSGTLREFSRTPSPSASTTLQQFRRKSDPPACLPEA
SAVVTDRCDSKSEMLGETSQPLHELGCSSRSQESMDSSCGLNTSSLSQP
SSRDSGSEESDCNNKSLDNQGEQNSKQHLPHFSKKDGLRRNKVPGLCRS
SSMDSFSTTKIKPLVPARVSGLSKKSGSMQTRKHHDVENKPGLQTKISE
LWKNFGFKKDSEKLPSCKKPLSPVKDNIQLTPETEDEIFNKPECVRAQR
AIFH
mTrex2:
(SEQ ID NO. 65)
MSEPPRAETFVFLDLEATGLPNMDPEIAEISLFAVHRSSLENPERDDSG
SLVLPRVLDKLTLCMCPERPFTAKASEITGLSSESLMHCGKAGFNGAVV
RTLQGFLSRQEGPICLVAHNGFDYDFPLLCTELQRLGAHLPQDTVCLDT
LPALRGLDRAHSHGTRAQGRKSYSLASLFHRYFQAEPSAAHSAEGDVHT
LLLIFLHRAPELLAWADEQARSWAHIEPMYVPPDGPSLEA
mArtimes:
(SEQ ID NO. 66)
MSSGMAYTSDRDRNKARAYSHCHKDHMKGRASKRRCSKVYCSVTKTSKY
RWNRTTTSVDASGKVVVTAGHCGSVMGSNGTVYTGDRAKGASRMHSGGR
VKDSVYDTTCDRYSRCRGVRSWVTRSHHVVWNCKAAYGYYTNSGVVHVD
KDMKNMDHHTTDRNTHACRHKACWNKCGTSNKTAHTSKSTMWGRTRKTN
VVRTGSSYRACSHSSSKDSYCVNVYNVVGTVDKVMDVKCRSSVKYKGKK
RARTHDSDDDDDTRHKVYTSMKADRSGGCKASVWSSANDCSNSDSGTSG
GGSTVNADDVDWVKRRDTGCHSSTGGSSKCSDSKCSDSKCSDSDGDSTH
SSNSSSTHTDGSGWDSCDTVSSKSGGDSTSNKGAYKKKSSASDACDTHC
DKSRAVNGACVDTSGRKSKTSSTRADSSSSDSTATHCYRKATGSVVKRK
CSDS
T5 exo:
(SEQ ID NO. 67)
MSKSWGKFIEEEEAEMASRRNLMIVDGTNLGFRFKHNNSKKPFASSYVS
TIQSLAKSYSARTTIVLGDKGKSVFRLEHLPEYKGNRDEKYAQRTEEEK
ALDEQFFEYLKDAFELCKTTFPTFTIRGVEADDMAAYIVKLIGHLYDHV
WLISTDGDWDTLLTDKVSRFSFTTRREYHLRDMYEHHNVDDVEQFISLK
AIMGDLGDNIRGVEGIGAKRGYNIIREFGNVLDIIDQLPLPGKQKYIQN
LNASEELLFRNLILVDLPTYCVDAIAAVGQDVLDKFTKDILEIAEQ

### Deaminase

Deaminases that may be used as the component of the base editor of the present disclosure include cytidine deaminases and adenosine deaminases. Cytidine deaminases include but are not limited to hAPOBEC3A (Zong et al., 2018, Nat. Biotechnol. October 1. doi: 10.1038/nbt.4261), rAPOBEC1, C57 and Sdd (Huang J et al., 2023, Cell, doi: 10.1101/2023.05.21.541555), which produce a C-to-T conversion at the base site. Alternative adenosine deaminases include TadA-8e (Richter et al., 2020, Nat. Biotechnol. 38: 883-891), which produce an A-to-G conversion at the base site.

Deaminases that may be used as the components of exemplary base editors of the present disclosure are provided below, including but not limited to the deaminases set forth in Table 1 (the proteins as set forth in SEQ ID NO. 36-59 and 80-86).

TABLE 1
Type of deaminases

| Name of cytidine | | |
|---|---|---|
| deaminases and | | |
| adenosine | | |
| deaminases | SEQ ID NO. | Reference/doi |
| rAPOBEC1 | SEQ ID NO. 36 | 10.1038/nature17946 |
| hAPOBEC3A | SEQ ID NO. 37 | 10.1038/nbt.4198/10.1038/nbt.4261 |
| hAPOBEC3G-CTD | SEQ ID NO. 38 | 10.1101/658351 |
| PmCDA1 | SEQ ID NO. 39 | 10.1126/science.aaf8729 |
| tCDAIEQ | SEQ ID NO. 40 | 10.1038/s41467-022-32157-8 |
| hAID | SEQ ID NO. 41 | 10.1038/ncomms13330 |
| PpAPOBEC1 | SEQ ID NO. 42 | 10.1038/s41467-020-15887-5 |
| RrA3F | SEQ ID NO. 43 | 10.1038/s41467-020-15887-5 |
| AmAPOBEC1 | SEQ ID NO. 44 | 10.1038/s41467-020-15887-5 |
| SsAPOBEC3B | SEQ ID NO. 45 | 10.1038/s41467-020-15887-5 |
| hA3B | SEQ ID NO. 46 | 10.1016/j.molcel.2020.07.005 |
| hA3C | SEQ ID NO. 47 | 10.1016/j.molcel.2020.07.005 |
| hA3D | SEQ ID NO. 48 | 10.1016/j.molcel.2020.07.005 |
| hA3F | SEQ ID NO. 49 | 10.1016/j.molcel.2020.07.005 |
| hA3G | SEQ ID NO. 50 | 10.1016/j.molcel.2020.07.005 |
| hA3H | SEQ ID NO. 51 | 10.1016/j.molcel.2020.07.005 |
| hA3Bctd | SEQ ID NO. 52 | 10.1016/j.molcel.2020.07.005 |
| FERNY | SEQ ID NO. 53 | 10.1038/s41587-019-0193-0 |
| ecTadA | SEQ ID NO. 54 | 10.1038/nature24644 |
| mADA | SEQ ID NO. 55 | 10.1038/nature24644 |
| hADAR2 | SEQ ID NO. 56 | 10.1038/nature24644 |
| hADAT2 | SEQ ID NO. 57 | 10.1038/nature24644 |
| ecTadA*(7.10) | SEQ ID NO. 58 | 10.1038/nature24644 |
| TadA-8e | SEQ ID NO. 59 | 10.1038/s41587-020-0453-z |
| Sdd2 | SEQ ID NO. 80 | 10.1101/2023.05.21.541555 |
| Sdd3 | SEQ ID NO. 81 | 10.1101/2023.05.21.541555 |
| Sdd4 | SEQ ID NO. 82 | 10.1101/2023.05.21.541555 |
| Sdd6 | SEQ ID NO. 83 | 10.1101/2023.05.21.541555 |
| Sdd7/C57 | SEQ ID NO. 84 | 10.1101/2023.05.21.541555 |
| Sdd10 | SEQ ID NO. 85 | 10.1101/2023.05.21.541555 |
| Sdd59 | SEQ ID NO. 86 | 10.1101/2023.05.21.541555 |

rAPOBEC1:
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIARLY
HHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLK (SEQ ID NO.
36)
hAPOBEC3A:
MEASPASGPRHLMDPHIFTSNFNNGIGRHKTYLCYEVERLDNGTSVKMDQHRGFLHN
QAKNLLCGFYGRHAELRFLDLVPSLQLDPAQIYRVTWFISWSPCFSWGCAGEVRAFLQ
ENTHVRLRIFAARIYDYDPLYKEALQMLRDAGAQVSIMTYDEFKHCWDTFVDHQGCP
FQPWDGLDEHSQALSGRLRAILQNQGN (SEQ ID NO. 37)
hAPOBEC3G-CTD:
MDPPTFTFNFNNEPWVRGRHETYLCYEVERMHNDTWVLLNQRRGFLCNQAPHKHGF
LEGRHAELCFLDVIPFWKLDLDQDYRVTCFTSWSPCFSCAQEMAKFISKNKHVSLCIFT
ARIYDDQGRCQEGLRTLAEAGAKISIMTYSEFKHCWDTFVDHQGCPFQPWDGLDEHS
QDLSGRLRAILQNQEN (SEQ ID NO. 38)
PmCDA1:
MTDAEYVRIHEKLDIYTFKKQFFNNKKSVSHRCYVLFELKRRGERRACFWGYAVNKP
QSGTERGIHAEIFSIRKVEEYLRDNPGQFTINWYSSWSPCADCAEKILEWYNQELRGNG
HTLKIWACKLYYEKNARNQIGLWNLRDNGVGLNVMVSEHYQCCRKIFIQSSHNQLNE
NRWLEKTLKRAEKRRSELSIMIQVKILHTTKSPAVSRGSG (SEQ ID NO. 39)
tCDAIEQ:
SHRCYVLFELKRRGERRACFWGYAVNKPQSGTERGIHAEIFSIRKVEEYLRDNPGQFTI
NWYSSWSPCADCAEKILEWYNQELRGNGHTLKIEACKLYYEKNARNQIGLQNLRDNG
VGLNV (SEQ ID NO. 40)
hAID:
MDSLLMNRRKFLYQFKNVRWAKGRRETYLCYVVKRRDSATSFSLDFGYLRNKNGCH
VELLFLRYISDWDLDPGRCYRVTWFTSWSPCYDCARHVADFLRGNPNLSLRIFTARLYF
CEDRKAEPEGLRRLHRAGVQIAIMTFKDYFYCWNTFVENHERTFKAWEGLHENSVRL
SRQLRRILLPLYEVDDLRDAFRTLGL (SEQ ID NO. 41)
PpAPOBEC1:
MTSEKGPSTGDPTLRRRIESWEFDVFYDPRELRKETCLLYEIKWGMSRKIWRSSGKNT
TNHVEVNFIKKFTSERRFHSSISCSITWFLSWSPCWECSQAIREFLSQHPGVTLVIYVARL
FWHMDQRNRQGLRDLVNSGVTIQIMRASEYYHCWRNFVNYPPGDEAHWPQYPPLW
MMLYALELHCIILSLPPCLKISRRWQNHLAFFRLHLQNCHYQTIPPHILLATGLIHPSVT
WR (SEQ ID NO. 42)
RrA3F:
MKPQIRDHRPNPMEAMYPHIFYFHFENLEKAYGRNETWLCFTVEIIKQYLPVPWKKGV
FRNQVDPETHCHAEKCFLSWFCNNTLSPKKNYQVTWYTSWSPCPECAGEVAEFLAEH
SNVKLTIYTARLYYFWDTDYQEGLRSLSEEGASVEIMDYEDFQYCWENFVYDDGEPFK
RWKGLKYNFQSLTRRLREILQ (SEQ ID NO. 43)
AmAPOBEC1:
MADSSEKMRGQYISRDTFEKNYKPIDGTKEAHLLCEIKWGKYGKPWLHWCQNQRMN
IHAEDYFMNNIFKAKKHPVHCYVTWYLSWSPCADCASKIVKFLEERPYLKLTIYVAQL
YYHTEEENRKGLRLLRSKKVIIRVMDISDYNYCWKVFVSNQNGNEDYWPLQFDPWV
KENYSRLLDIFWESKCRSPNPW (SEQ ID NO. 44)
SsAPOBEC3B:
MDPQRLRQWPGPGPASRGGYGQRPRIRNPEEWFHELSPRTFSFHFRNLRFASGRNRSYI
CCQVEGKNCFFQGIFQNQVPPDPPCHAELCFLSWFQSWGLSPDEHYYVTWFISWSPCC
ECAAKVAQFLEENRNVSLSLSAARLYYFWKSESREGLRRLSDLGAQVGIMSFQDFQHC
WNNFVHNLGMPFQPWKKLHKNYQRLVTELKQILREEPATYGSPQAQGKVRIGSTAAG
LRHSHSHTRSEAHLRPNHSSRQHRILNPPREARARTCVLVDASWICYR (SEQ ID NO.
45)
hA3B:
MNPQIRNPMERMYRDTFYDNFENEPILYGRSYTWLCYEVKIKRGRSNLLWDTGVFRG
QVYFKPQYHAEMCFLSWFCGNQLPAYKCFQITWFVSWTPCPDCVAKLAEFLSEHPNV
TLTISAARLYYYWERDYRRALCRLSQAGARVKIMDYEEFAYCWENFVYNEGQQFMP
WYKFDENYAFLHRTLKEILRYLMDPDTFTFNFNNDPLVLRRRQTYLCYEVERLDNGT
WVLMDQHMGFLCNEAKNLLCGFYGRHAELRFLDLVPSLQLDPAQIYRVTWFISWSPC
FSWGCAGEVRAFLQENTHVRLRIFAARIYDYDPLYKEALQMLRDAGAQVSIMTYDEF
EYCWDTFVYRQGCPFQPWDGLEEHSQALSGRLRAILQNQGN (SEQ ID NO. 46)
hA3C:
MNPQIRNPMKAMYPGTFYFQFKNLWEANDRNETWLCFTVEGIKRRSVVSWKTGVFR
NQVDSETHCHAERCFLSWFCDDILSPNTKYQVTWYTSWSPCPDCAGEVAEFLARHSN
VNLTIFTARLYYFQYPCYQEGLRSLSQEGVAVEIMDYEDFKYCWENFVYNDNEPFKPW
KGLKTNFRLLKRRLRESLQ (SEQ ID NO. 47)
hA3D:
MNPQIRNPMERMYRDTFYDNFENEPILYGRSYTWLCYEVKIKRGRSNLLWDTGVFRG
PVLPKRQSNHRQEVYFRFENHAEMCFLSWFCGNRLPANRRFQITWFVSWNPCLPCVV
KVTKFLAEHPNVTLTISAARLYYYRDRDWRWVLLRLHKAGARVKIMDYEDFAYCWE
NFVCNEGQPFMPWYKFDDNYASLHRTLKEILRNPMEAMYPHIFYFHFKNLLKACGRN
ESWLCFTMEVTKHHSAVFRKRGVFRNQVDPETHCHAERCFLSWFCDDILSPNTNYEVT
WYTSWSPCPECAGEVAEFLARHSNVNLTIFTARLCYFWDTDYQEGLCSLSQEGASVKI
MGYKDFVSCWKNFVYSDDEPFKPWKGLQTNFRLLKRRLREILQ (SEQ ID NO. 48)
hA3F:
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKLAEFLAEHPNVTL
TISAARLYYYWERDYRRALCRLSQAGARVKIMDDEEFAYCWENFVYSEGQPFMPWYK
FDDNYAFLHRTLKEILRNPMEAMYPHIFYFHFKNLRKAYGRNESWLCFTMEVVKHHSP
VSWKRGVFRNQVDPETHCHAERCFLSWFCDDILSPNTNYEVTWYTSWSPCPECAGEV
AEFLARHSNVNLTIFTARLYYFWDTDYQEGLRSLSQEGASVEIMGYKDFKYCWENFV
YNDDEPFKPWKGLKYNFLFLDSKLQEILE (SEQ ID NO. 49)
hA3G:
MKPHFRNTVERMYRDTFSYNFYNRPILSRRNTVWLCYEVKTKGPSRPPLDAKIFRGQV
YSELKYHPEMRFFHWFSKWRKLHRDQEYEVTWYISWSPCTKCTRDMATFLAEDPKV
TLTIFVARLYYFWDPDYQEALRSLCQKRDGPRATMKIMNYDEFQHCWSKFVYSQRELF
EPWNNLPKYYILLHIMLGEILRHSMDPPTFTFNFNNEPWVRGRHETYLCYEVERMHND
TWVLLNQRRGFLCNQAPHKHGFLEGRHAELCFLDVIPFWKLDLDQDYRVTCFTSWSP
CFSCAQEMAKFISKNKHVSLCIFTARIYDDQGRCQEGLRTLAEAGAKISIMTYSEFKHC
WDTFVDHQGCPFQPWDGLDEHSQDLSGRLRAILQNQEN (SEQ ID NO. 50)
hA3H:
MALLTAETFRLQFNNKRRLRRPYYPRKALLCYQLTPQNGSTPTRGYFENKKKCHAEIC
FINEIKSMGLDETQCYQVTCYLTWSPCSSCAWELVDFIKAHDHLNLRIFASRLYYHWCK
PQQDGLRLLCGSQVPVEVMGFPEFADCWENFVDHEKPLSFNPYKMLEELDKNSRAIK
RRLDRIKS (SEQ ID NO. 51)
hA3Bctd:
MEILRYLMDPDTFTFNFNNDPLVLRRRQTYLCYEVERLDNGTWVLMDQHMGFLCNE
AKNLLCGFYGRHAELRFLDLVPSLQLDPAQIYRVTWFISWSPCFSWGCAGEVRAFLQE
NTHVRLRIFAARIYDYDPLYKEALQMLRDAGAQVSIMTYDEFEYCWDTFVYRQGCPF
QPWDGLEEHSQALSGRLRAILQNQGN (SEQ ID NO. 52)
FERNY:
FERNYDPRELRKETYLLYEIKWGKSGKLWRHWCQNNRTQHAEVYFLENIFNARRFNP
STHCSITWYLSWSPCAECSQKIVDFLKEHPNVNLEIYVARLYYHEDERNRQGLRDLVNS
GVTIRIMDLPDYNYCWKTFVSDQGGDEDYWPGHFAPWIKQYSLKL (SEQ ID NO. 53)
ecTadA:
MSEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTGA
AGSLMDVLHHPGMNHRVEITEGILADECAALLSDFFRMRRQEIKAQKKAQSSTD (SEQ
ID NO. 54)
mADA:
MAQTPAFNKPKVELHVHLDGAIKPETILYFGKKRGIALPADTVEELRNIIGMDKPLSLP
GFLAKFDYYMPVIAGCREAIKRIAYEFVEMKAKEGVVYVEVRYSPHLLANSKVDPMP
WNQTEGDVTPDDVVDLVNQGLQEGEQAFGIKVRSILCCMRHQPSWSLEVLELCKKYN
QKTVVAMDLAGDETIEGSSLFPGHVEAYEGAVKNGIHRTVHAGEVGSPEVVREAVDIL
KTERVGHGYHTIEDEALYNRLLKENMHFEVCPWSSYLTGAWDPKTTHAVVRFKNDKA
NYSLNTDDPLIFKSTLDTDYQMTKKDMGFTEEEFKRLNINAAKSSFLPEEEKKELLERL
YREYQ (SEQ ID NO. 55)
hADAR2:
MHLDQTPSRQPIPSEGLQLHLPQVLADAVSRLVLGKFGDLTDNFSSPHARRKVLAGVV
MTTGTDVKDAKVISVSTGTKCINGEYMSDRGLALNDCHAEIISRRSLLRFLYTQLELYL
NNKDDQKRSIFQKSERGGFRLKENVQFHLYISTSPCGDARIFSPHEPILEEPADRHPNRK
ARGQLRTKIESGEGTIPVRSNASIQTWDGVLQGERLLTMSCSDKIARWNVVGIQGSLLS
IFVEPIYFSSIILGSLYHGDHLSRAMYQRISNIEDLPPLYTLNKPLLSGISNAEARQPGKAP
NFSVNWTVGDSAIEVINATTGKDELGRASRLCKHALYCRWMRVHGKVPSHLLRSKITK
PNVYHESKLAAKEYQAAKARLFTAFIKAGLGAWVEKPTEQDQFSLTP (SEQ ID NO. 56)
hADAT2:
MEAKAAPKPAASGACSVSAEETEKWMEEAMHMAKEALENTEVPVGCLMVYNNEVV
GKGRNEVNQTKNATRHAEMVAIDQVLDWCRQSGKSPSEVFEHTVLYVTVEPCIMCAA
ALRLMKIPLVVYGCQNERFGGCGSVLNIASADLPNTGRPFQCIPGYRAEEAVEMLKTF
YKQENPNAPKSKVRKKECQKS (SEQ ID NO. 57)
ecTadA*(7.10):
MSEVEFSHEYWMRHALTLAKRARDEREVPVGAVLVLNNRVIGEGWNRAIGLHDPTAH
AEIMALRQGGLVMQNYRLIDATLYVTFEPCVMCAGAMIHSRIGRVVFGVRNAKTGAA
GSLMDVLHYPGMNHRVEITEGILADECAALLCYFFRMPRQVFNAQKKAQSSTD (SEQ
ID NO. 58)
TadA*ABE8e (TadA-8e):
MSEVEFSHEYWMRHALTLAKRARDEREVPVGAVLVLNNRVIGEGWNRAIGLHDPTAH
AEIMALRQGGLVMQNYRLIDATLYVTFEPCVMCAGAMIHSRIGRVVFGVRNSKRGAA
GSLMNVLNYPGMNHRVEITEGILADECAALLCDFYRMPRQVFNAQKKAQSSIN (SEQ
ID NO. 59)
Sdd2
MAPDSLVWFDPLGLIVLQQVPYNDHPLFGAVSEFIQGKSRSDLRGRNVAAVLLDDGTVI
VRASEGGGNHAERVLMGLSEVDPAKVVAVYTERSPCTGRINCHDLLDSSLGADVPVY
YTHEMIRGQEGKTAQQIEADRNQFCRGG (SEQ ID NO. 80)
Sdd3
MSASAQLNTYLAAIGNSTTTVEAQPEAAPPPAAAESLDSTPRLPDGGIDFHALAKRLGL
LEARPTEQPPFDPRRFNPACWQGLKPYDQAGTAEGNLFIAPGKRWNTRPMQASKLEV
GPQSDLHPQWRSRKAPWHIEGKIAAYMRQKGFTDGCVYLNARPCSGPDGCARNLPDL
LPVGSTLHVHARYIDRTGETRFYYREYRGTGKALT (SEQ ID NO. 81)
Sdd4
MLDAMDAYLSEIAGGNAPARAGPKAPEPKQPGGSSSPRARDGRIDFRALLERLKAQGV
VGLEGRSDDPIPDFDPKKQNPACYQGLAPRQKGKPVRGNLFFPDGRRWNDVALESSRG
EPAFDLNIIKPEYRSLSPARGHLEGNVAAWMRSTFHQEMVLYINESPCRKHGKGCLYTL
EHFLPRGYVLHVWSRNDRGEWRGNTFRGSGEAFTEGA (SEQ ID NO. 82)
Sdd6
MVETRDKIIAAKSRSDAGLLAFQQATNGSIDSRPAEAIANLQRAKTHLDEAQRLVANSD
AAVDNYINAILGGASAATAQPSAVIPASKPSRFKPMRTDPAKADEIRPHVGKDRAVATL
WDADGNRVLGLHSADDDGPAATAAWKPPWRDYVRLRRHVEAHAAARMHQDGHKT
MVMYINLPPCKYFDGCKLNLEDILPKGSTLWMHRVFQNGGTKIYQFNGTGRAYV
(SEQ ID NO. 83)
Sdd7 (also represented as C57 in the present specification)
MLEAVRARLIGEGGGPGAVPEGGDGPPAVPAEEVERLRGELPPPVVPGTGQKTHGRWI
GPDGRVRAIVSGRDEDAALVHAQLAAKGIPDEPTRNSDVEQKLAAHMVANGIRHVTL
VINHRPCRGFDDSCDTLVPIILPEGCTLTVHGQTDKGMRVRVRYTGGARPWWS (SEQ
ID NO. 84)
Sdd10
MLDAALGAVRRIIAALGTSGAERASPGANGSERVDELAERLPPTVVPNTSAKTHGWW
FTGQGAAQELISGEGPDARAAYEALREEGYPRPGMPFVAMHVEIKLAAHMRRNDIEHA
TVVINNIPCPLVWGCENLIGVVLPEGSSLTVHGSNGYERTFTGGRKPPWPR (SEQ ID NO.
85)
Sdd59
MLLTPPPRPAAPPTTRPKPLVARTGDAYPPGTEWALPLIVQPHPPVGGTVPVEGHVRAL
RPESQISHVFHPGGGHWTEQARARLRVLPGFGWAVNLGHHVELQIAAWMTACGIHHA
ELVLNRPPCGERYGLGCHQALPVLLPRGYRLTVSSTRGGPQPYQHHYEGKA (SEQ ID
NO. 86)

### Uracil Glycosylase Inhibitor (UGI)

In some embodiments, when a cytidine deaminase is used, a uracil glycosylase inhibitor (UGI) is fused to the N-terminal of the deaminase, whereas UGI is not required when an adenosine deaminase is used.

Exemplary UGI proteins that may be used as the component of the base editor of the present disclosure are disclosed below, including but not limited to the protein as set forth in SEQ ID NO.68.

TNLSDIIEKETGKQLVIQESILMLPEEVEEVIGNKPESDILVHTAYDESTDENVMLLT SDAPEYKPWALVIQDSNGENKIKML (SEQ ID NO.68)

### Nuclear Localization Sequence (NLS)

In some embodiments of the present disclosure, the NLS of the fusion protein of the present disclosure may be located at N-terminal and/or C-terminal. In some embodiments of the present disclosure, the NLS of the fusion protein of the present disclosure may be located between the adenine deamination domain, the cytosine deamination domain, the nucleic acid-targeting domain and/or UGI. In some embodiments, the fusion protein comprises approximately 1, 2, 3, 4, 5, 6, 7, 8, 9, 10 or more NLS. In some embodiments, the fusion protein comprises approximately 1, 2, 3, 4, 5, 6, 7, 8, 9, 10 or more NLS at or near N-terminal. In some embodiments, the fusion protein comprises approximately 1, 2, 3, 4, 5, 6, 7, 8, 9, 10 or more NLS at or near C-terminal. In some embodiments, the polypeptide comprises a combination thereof, for example, comprising one or more NLS at N-terminal and one or more NLS at C-terminal. When more than one NLS are present, each NLS may be selected to be independent of other NLS.

Generally, NLS consists of one or more short sequences that are derived from positively charged lysine or arginine exposed on the surface of the protein, however, other types of NLS are also known. Non-limiting examples of NLS include KKRKV (SEQ ID NO. 150), PKKKRKV (SEQ ID NO. 151) or KRPAATKKAGQAKKKK (SEQ ID NO. 152).

### Recombinant Expression Construct

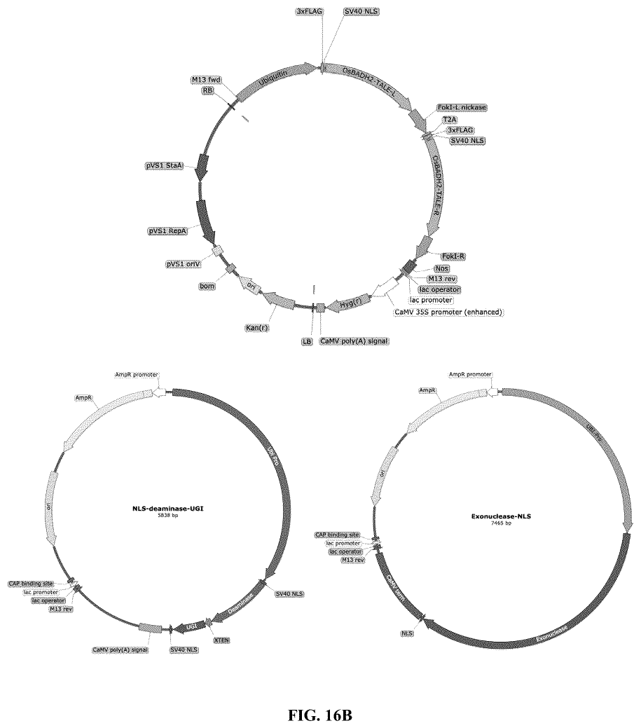

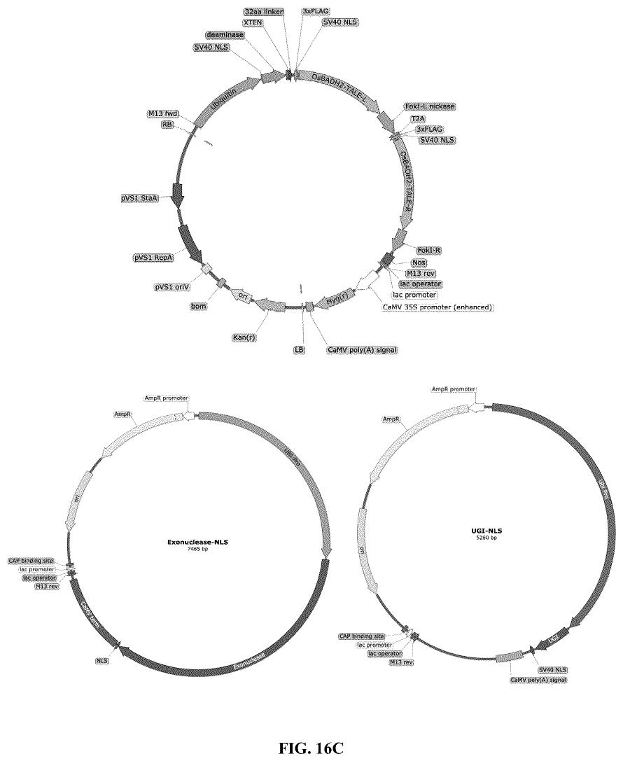

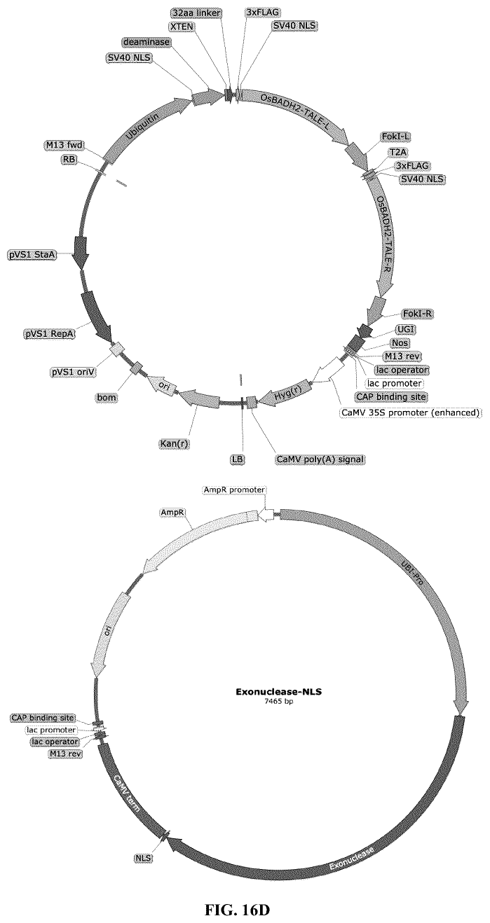

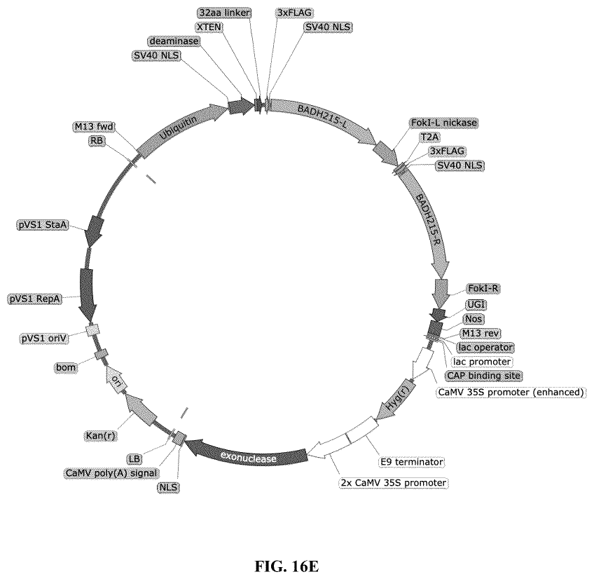

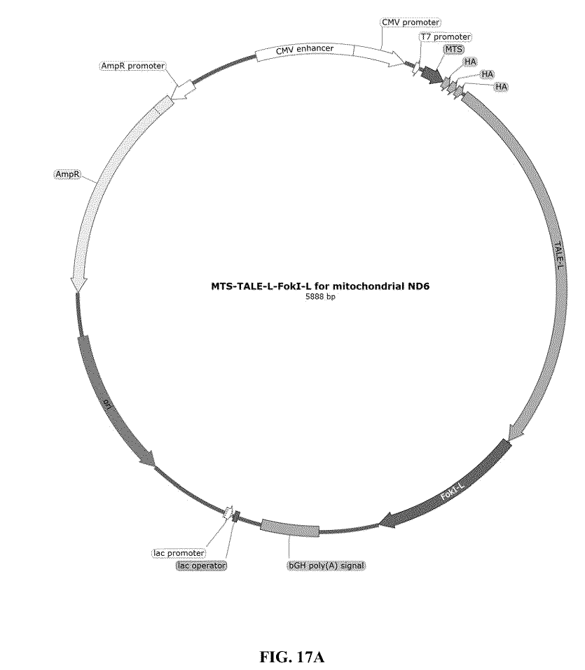

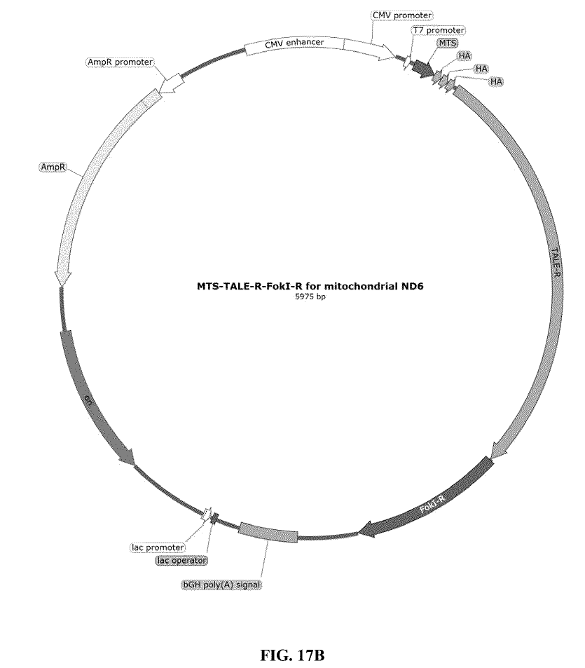

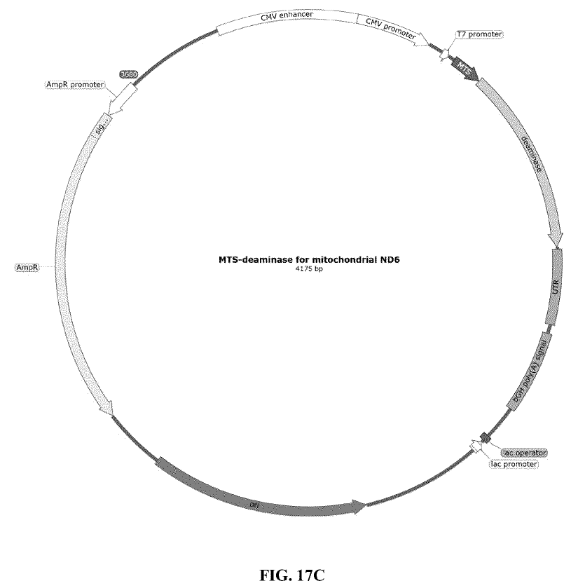

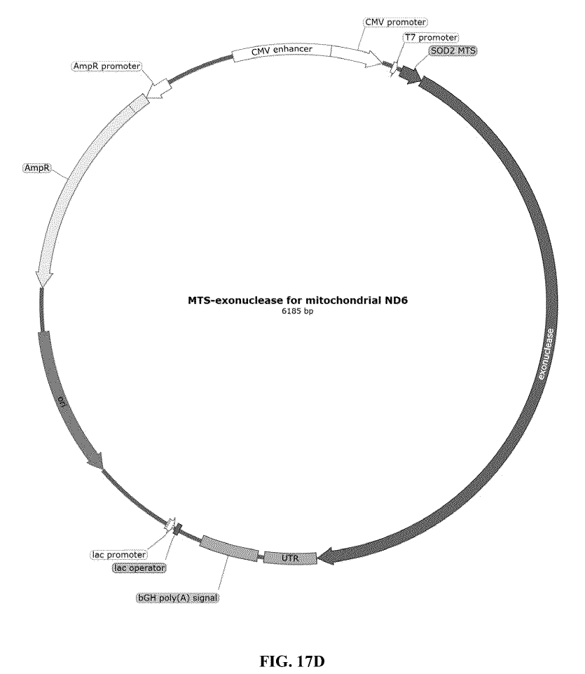

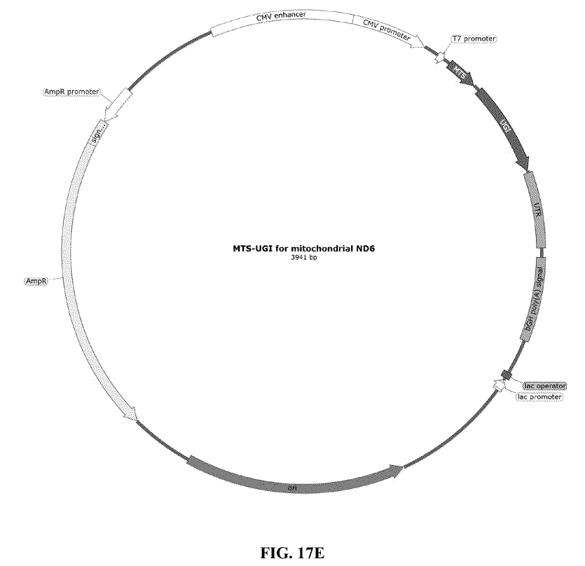

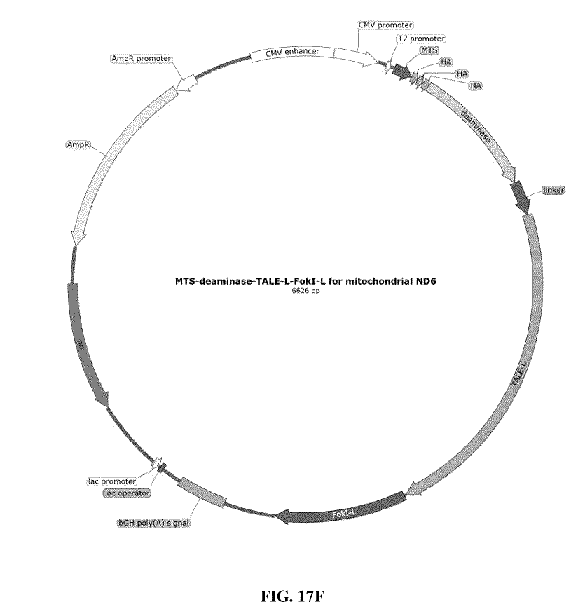

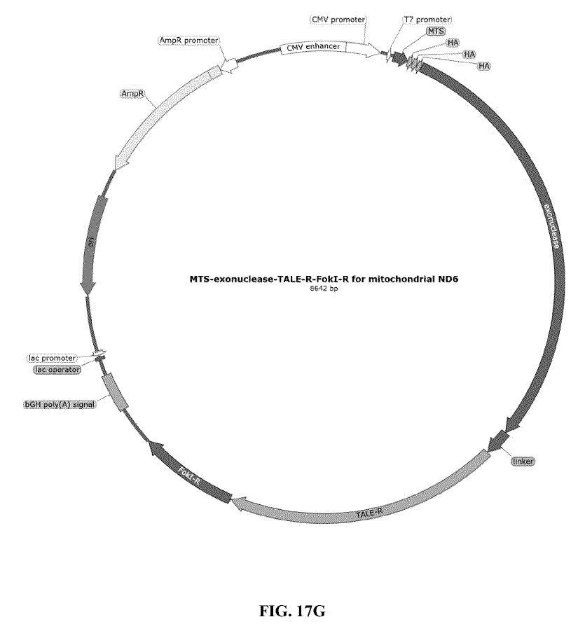

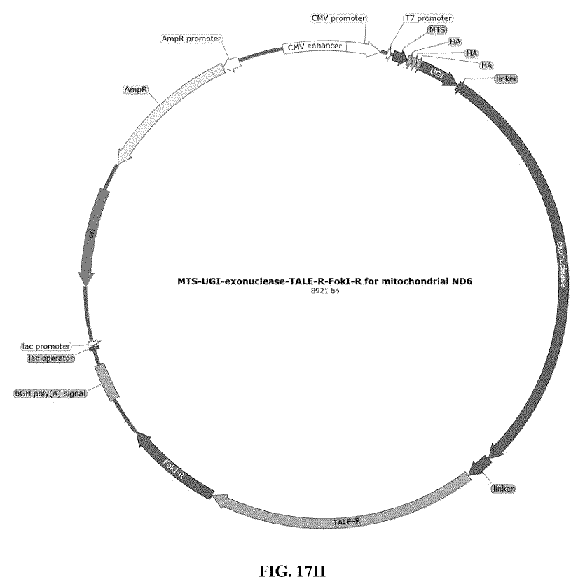

Each component in the base editor of the present disclosure may be expressed separately, and may also be expressed as one or more fusion proteins. Alternatively, the above-mentioned elements or components are expressed separately or together by using the recombinant expression constructs used in recombinant genetic engineering technology. Exemplary recombinant expression constructs of the present disclosure are as set forth in for example, FIG. 16A to FIG. 16E and FIG. 17A to FIG. 17H.

The types, functions and references of the genes and the regulatory elements in the above-mentioned exemplary recombinant expression constructs (FIG. 16A to FIG. 16E and FIGS. 17A to 17H) are explained and exemplified below, as set forth in Table 2 below.

TABLE 2
Examples of the genes and the regulatory element in constructs

| Vector element | Function | Reference |
|---|---|---|
| MTS | mitochondrial targeting peptide | Lei et al. Mitochondrial base editor |
| (Mitochondrial | of Homo sapiens superoxide | induces substantial nuclear off-target |
| Targeting | dismutase 2 that helps the | mutations. Nature Vol. 606, 7915 (2022): |
| Sequence) | translocation of proteins or | 804-811. doi |
| | fusion proteins including | 10.1038/s41586-022-04836-5 |
| | TALE, exonuclease, | |
| | deaminase, UGI and the like to | |
| | mitochondria. | |
| CTP | chloroplast translocation | Kang et al. Chloroplast and |
| (Chloroplast | peptide that helps the | mitochondrial DNA editing in plants. |
| Transit Peptide) | translocation of proteins or | Nature Plants Vol. 7, 2021: 899-905. |
| | fusion proteins including | doi: 10.1038/s41477-021-00943-9). |
| | TALE, exonuclease, | |
| | deaminase, UGI and the like to | |
| | chloroplasts. | |
| HA | human influenza | Lei et al. Mitochondrial base editor |
| | hemagglutinin epitope tag, | induces substantial nuclear off-target |
| | which is used for protein | mutations. Nature Vol. 606, 7915 (2022): |
| | detection and purification. | 804-811. doi: |
| | | 10.1038/s41586-022-04836-5 |
| CMV enhancer | fragment that enhances the | Boshart et al. A very strong enhancer is |
| | expression of CMV promoter. | located upstream of an immediate early |
| | | gene of human cytomegalovirus. Cell |
| | | Vol. 41, 2 (1985): 521-30. doi: |
| | | 10.1016/s0092-8674(85)80025-8 |
| CMV promoter | human cytomegalovirus 5′ | Thomsen et al. Promoter-regulatory |
| | promoter region fragment that | region of the major immediate early gene |
| | drives the expression of the | of human cytomegalovirus. PNAS Vol. |
| | downstream genes of interest | 81, 3 (1984): 659-63. doi: |
| | (such as TALE, nickase and | 10.1073/pnas.81.3.659 |
| | UGI). | |
| bGH poly(A) | bovine somatotropin | Pfarr et al. Differential Effects of |
| signal | polyadenylylation signal, | Polyadenylation Regions on Gene |
| | which is used for the | Expression in Mammalian Cells. DNA |
| | termination of transcription. | Vol. 5, 2 (1986): 115-122. doi: |
| | | 10.1089/dna.1986.5.115 |
| UTR | untranslated region. | Lei et al. Mitochondrial base editor |
| | | induces substantial nuclear off-target |
| | | mutations. Nature Vol. 606, 7915 (2022): |
| | | 804-811. doi: |
| | | 10.1038/s41586-022-04836-5 |
| Amp^{R} | gene encoding β-lactamase, | Lei et al. Mitochondrial base editor |
| | which confers resistance to | induces substantial nuclear off-target |
| | ampicillin, carbenicillin, and | mutations. Nature Vol. 606, 7915 (2022): |
| | related antibiotics. | 804-811. doi |
| | | 10.1038/s41586-022-04836-5 |
| Amp^{R} promoter | promoter that drives the | Lei et al. Mitochondrial base editor |
| | expression of AmpR gene. | induces substantial nuclear off-target |
| | | mutations. Nature Vol. 606, 7915 (2022): |
| | | 804-811. doi |
| | | 10.1038/s41586-022-04836-5 |
| T7 promoter | A promoter synthesized by | Lei et al. Mitochondrial base editor |
| | bacteriophage that could be | induces substantial nuclear off-target |
| | recognized by T7 RNA | mutations. Nature Vol. 606, 7915 (2022): |
| | polymerase. | 804-811. doi: |
| | | 10.1038/s41586-022-04836-5 |
| UGI | inhibitor of uracil-DNA | Mo et al. Crystal structure of human |
| | glycosylase derived from a | uracil-DNA glycosylase in complex with |
| | Bacillus subtilis bacteriophage | a protein inhibitor: protein mimicry of |
| | that protects the uracil in DNA | DNA. Cell Vol. 82, 5 (1995): 701-8. |
| | by irreversibly inhibiting | doi: 10.1016/0092-8674(95)90467-0 |
| | uracil-DNA glycosylase which | |
| | is the key DNA repair enzyme | |
| | (UDG). | |
| deaminase | including cytidine deaminases | Komor et al. Programmable editing of a |
| | that convert C to U and | target base in genomic DNA without |
| | adenosine deaminases that | double-stranded DNA cleavage. Nature |
| | convert A to I | Vol. 533, 7603 (2016): 420-4. doi: |
| | | 10.1038/nature17946; Gaudelli et al. |
| | | Programmable base editing of A•T to |
| | | G•C in genomic DNA without DNA |
| | | cleavage. Nature Vol. 551, 7681 (2017): |
| | | 464-471. doi: 10.1038/nature24644 |
| exonuclease | including 5′ exonuclease such | Lee et al. Expression specificity of the |
| | as mExol and 3′ exonuclease | mouse exonuclease 1 (mExo1) gene. |
| | such as Trex2, for the digestion | Nucleic Acids Research. Vol. 27, 20 |
| | of the nicked DNA strand. | (2022): 4114-20. doi: |
| | | 10.1093/nar/27.20.4114 |
| linker | linker peptide, sequence | Komor et al. Programmable editing of a |
| | between two protein domains | target base in genomic DNA without |
| | of a fusion protein, for flexible | double-stranded DNA cleavage. Nature |
| | linkage, wherein an XTEN | Vol. 533, 7603 (2016): 420-4. doi: |
| | linker peptide may be selected. | 10.1038/nature17946 |
| CaMV 35S | a constitutive promoter, which | Odell, J. T., Nagy, F. & Chua, N. H. |
| promoter | is used to drive high-level gene | Identification of DNA sequences required |
| | expression in dicotyledon. | for activity of the cauliflower mosaic |
| | | virus 35S promoter. Nature 313, 810-812, |
| | | doi: 10.1038/313810a0 (1985). |
| Enhanced | a CaMV 35S promoter | Shan, Q. et al. Rapid and efficient gene |
| CaMV 35S | derivative, which is used to | modification in rice and Brachypodium |
| promoter | drive the expression of the | using TALENs. Mol Plant 6, 1365-1368, |
| | downstream genes (Hyg, etc). | doi: 10.1093/mp/sss162 (2013) |
| 2 × CaMV 35S | a CaMV 35S promoter | Shan, Q. et al. Rapid and efficient gene |
| promoter | derivative that drive the | modification in rice and Brachypodium |
| | expression of the downstream | using TALENs. Mol Plant 6, 1365-1368, |
| | genes. | doi: 10.1093/mp/sss162 (2013) |
| Ubi-promoter | a Zea mays-derived promoter | Shan, Q. et al. Rapid and efficient gene |
| | that has high expression | modification in rice and Brachypodium |
| | activity in monocotyledon and | using TALENs. Mol Plant 6, 1365-1368, |
| | is used to drive the expression | doi: 10.1093/mp/sss162 (2013) |
| | of the downstream genes | |
| | (TALE-L, TALE-R, etc). | |
| CaMV poly(A) | poly(A) signal from CaMV, | Shan, Q. et al. Rapid and efficient gene |
| signal | which is used for the | modification in rice and Brachypodium |
| | termination of gene | using TALENs. Mol Plant 6, 1365-1368, |
| | transcription. | doi: 10.1093/mp/sss162 (2013) |
| Nos terminator | NOS terminator of | Shan, Q. et al. Rapid and efficient gene |
| | Agrobacterium tumefaciens, | modification in rice and Brachypodium |
| | which is used for the | using TALENs. Mol Plant 6, 1365-1368, |
| | termination of gene | doi: 10.1093/mp/sss162 (2013) |
| | transcription. | |
| E9 terminator | terminator of pea rbcS E9 | Xing, H. L. et al. A CRISPR/Cas9 toolkit |
| | gene, which is used for the | for multiplex genome editing in plants. |
| | termination of gene | BMC Plant Biol 14, 327, |
| | transcription. | doi: 10.1186/s12870-014-0327-y (2014) |
| pUC ori | Origin of replication of a | Shan, Q. et al. Rapid and efficient gene |
| | high-copy expression plasmid | modification in rice and Brachypodium |
| | in E. coli. | using TALENs. Mol Plant 6, 1365-1368, |
| | | doi: 10.1093/mp/sss162 (2013) |
| CAP binding | binding site of Catabolite | Shan, Q. et al. Rapid and efficient gene |
| site | activator protein, which | modification in rice and Brachypodium |
| | activates transcription of the | using TALENs. Mol Plant 6, 1365-1368, |
| | α-subunit of RNA Polymerase | doi: 10.1093/mp/sss162 (2013) |
| | through the protein-protein | |
| | interaction. | |
| bom | a structure required for plasmid | Hajdukiewicz, P., Svab, Z. & Maliga, P. |
| | transfer during bacterial | The small, versatile pPZP family of |
| | conjugation. | Agrobacterium binary vectors for plant |
| | | transformation. Plant Mol Biol 25, |
| | | 989-994, doi: 10.1007/bf00014672 (1994) |
| HygR | Hygromycin B-resistance gene, | Gritz, L. & Davies, J. Plasmid-encoded |
| | as a selection marker for | hygromycin B resistance: the sequence of |
| | Agrobacterium-mediated | hygromycin B phosphotransferase gene |
| | transformation. | and its expression in Escherichia coli and |
| | | Saccharomyces cerevisiae. Gene 25, |
| | | 179-188, |
| | | doi: 10.1016/0378-1119(83)90223-8 |
| | | (1983) |
| Kan^{R} | gene encoding neomycin | Hajdukiewicz, P., Svab, Z. & Maliga, P. |
| | phosphotransferase, which | The small, versatile pPZP family of |
| | confers resistance to | Agrobacterium binary vectors for plant |
| | kanamycin. | transformation. Plant Mol Biol 25, |
| | | 989-994, doi: 10.1007/bf00014672 (1994) |
| pVS1 oriV | for replication/plasmid stability | Hajdukiewicz, P., Svab, Z. & Maliga, P. |
| | in Agrobacterium, for | The small, versatile pPZP family of |
| | Agrobacterium-mediated | Agrobacterium binary vectors for plant |
| | transformation. | transformation. Plant Mol Biol 25, |
| | | 989-994, doi: 10.1007/bf00014672 (1994) |
| pVS1 repA | for replication/plasmid stability | Hajdukiewicz, P., Svab, Z. & Maliga, P. |
| | in Agrobacterium, for | The small, versatile pPZP family of |
| | Agrobacterium-mediated | Agrobacterium binary vectors for plant |
| | transformation. | transformation. Plant Mol Biol 25, |
| | | 989-994, doi: 10.1007/bf00014672 (1994) |
| pVS1 staA | for replication/plasmid stability | Hajdukiewicz, P., Svab, Z. & Maliga, P. |
| | in Agrobacterium, for | The small, versatile pPZP family of |
| | Agrobacterium-mediated | Agrobacterium binary vectors for plant |
| | transformation. | transformation. Plant Mol Biol 25, |
| | | 989-994, doi: 10.1007/bf00014672 (1994) |
| LB | T-DNA left border repeat | Zambryski, P., Depicker, A., Kruger, K. |
| | sequence, which is used for the | & Goodman, H. M. Tumor induction by |
| | definition and delimitation of | Agrobacterium tumefaciens: analysis of |
| | T-DNA region. | the boundaries of T-DNA. J Mol Appl |
| | | Genet 1, 361-370 (1982) |
| RB | T-DNA right border repeat | Zambryski, P., Depicker, A., Kruger, K. |
| | sequence, which is used for the | & Goodman, H. M. Tumor induction by |
| | definition and delimitation of | Agrobacterium tumefaciens: analysis of |
| | T-DNA region. | the boundaries of T-DNA. J Mol Appl |
| | | Genet 1, 361-370 (1982) |
| SV40 NLS | composed of 7-amino acid | Zhang et al. Transcription activator-like |
| | PKKKRKV, SV (simian virus) | effector nucleases enable efficient plant |
| | 40 nuclear localization signal | genome engineering. Plant physiology |
| | as a signal fragment, mediating | vol. 161, 1 (2013): 20-7. |
| | the transport of proteins of | doi: 10.1104/pp.112.205179 |
| | interest into the nucleus | |
| TALEN scaffold | a modified TALEN scaffold | Zhang et al. Transcription activator-like |
| | with truncations in N-terminal | effector nucleases enable efficient plant |
| | region and C-terminal region | genome engineering. Plant physiology |
| | respectively (ΔN152/C63) | vol. 161, 1 (2013): 20-7. |
| | | doi: 10.1104/pp.112.205179 |
| TALE-L and | synthetic repeat sequences | Shan, Q. et al. Rapid and efficient gene |
| TALE-R | encoding TALE-L and | modification in rice and Brachypodium |
| | TALE-R protein, which are | using TALENs. Mol Plant 6, 1365-1368, |
| | used for the targeted binding of | doi: 10.1093/mp/sss162 (2013) |
| | DNA sequences of interest. | |
| FokI-L and | encoding the cleavage domains | Miller, JC et al. An improved zinc-finger |
| FokI-R | of FokI enzyme, for realizing | nuclease architecture for highly specific |
| | the nick of DNA strands of | genome editing. Nature biotechnology |
| | interest when fused to TALE, | vol. 25, 7 (2007): 778-85. |
| | working as heterodimer. | doi: 10.1038/nbt1319 |
| T2A | Thosea asigna virus 2A | Szymczak, A. L. & Vignali, D. A. |
| | peptide separates polypeptides | Development of 2A peptide-based |
| | during the translation process | strategies in the design of multicistronic |
| | in eukaryotic cells so as to | vectors. Expert Opin Biol Ther 5, |
| | express a plurality of proteins | 627-638, doi: 10.1517/14712598.5.5.627 |
| | in a single ORF. | (2005). |

Specifically, the genes and the regulatory elements in exemplary recombinant constructs used in the present disclosure include but are not limited to the following sequences: promoter sequences as set forth in SEQ ID NO. 69-72; terminator sequences as set forth in SEQ ID NO. 73-76; mitochondrial targeting sequences (MTS) as set forth in SEQ ID NO. 77-78; and chloroplast translocation peptide (CTP) sequence as set forth in SEQ ID NO. 79.

UBI promoter:
(SEQ ID NO. 69)
TGACCCGGTCGTGCCCCTCTCTAGAGATAATGAGCATTGCATGTCTAAGTTATAAAA
AATTACCACATATTTTTTTTGTCACACTTGTTTGAAGTGCAGTTTATCTATCTTTATAC
ATATATTTAAACTTTACTCTACGAATAATATAATCTATAGTACTACAATAATATCAGTGT
TTTAGAGAATCATATAAATGAACAGTTAGACATGGTCTAAAGGACAATTGAGTATTTT
GACAACAGGACTCTACAGTTTTATCTTTTTAGTGTGCATGTGTTCTCCTTTTTTTTTG
CAAATAGCTTCACCTATATAATACTTCATCCATTTTATTAGTACATCCATTTAGGGTTTA
GGGTTAATGGTTTTTATAGACTAATTTTTTTAGTACATCTATTTTATTCTATTTTAGCCT
CTAAATTAAGAAAACTAAAACTCTATTTTAGTTTTTTTATTTAATAATTTAGATATAAA
ATAGAATAAAATAAAGTGACTAAAAATTAAACAAATACCCTTTAAGAAATTAAAAAA
ACTAAGGAAACATTTTTCTTGTTTCGAGTAGATAATGCCAGCCTGTTAAACGCCGTC
GACGAGTCTAACGGACACCAACCAGCGAACCAGCAGCGTCGCGTCGGGCCAAGCG
AAGCAGACGGCACGGCATCTCTGTCGCTGCCTCTGGACCCCTCTCGATCGAGAGTT
CCGCTCCACCGTTGGACTTGCTCCGCTGTCGGCATCCAGAAATTGCGTGGCGGAGC
GGCAGACGTGAGCCGGCACGGCAGGCGGCCTCCTCCTCCTCTCACGGCACCGGCA
GCTACGGGGGATTCCTTTCCCACCGCTCCTTCGCTTTCCCTTCCTCGCCCGCCGTAAT
AAATAGACACCCCCTCCACACCCTCTTTCCCCAACCTCGTGTTGTTCGGAGCGCAC
ACACACACAACCAGATCTCCCCCAAATCCACCCGTCGGCACCTCCGCTTCAAGGTA
CGCCGCTCGTCCTCCCCCCCCCCCCCTCTCTACCTTCTCTAGATCGGCGTTCCGGTC
CATGGTTAGGGCCCGGTAGTTCTACTTCTGTTCATGTTTGTGTTAGATCCGTGTTTGT
GTTAGATCCGTGCTGCTAGCGTTCGTACACGGATGCGACCTGTACGTCAGACACGTT
CTGATTGCTAACTTGCCAGTGTTTCTCTTTGGGGAATCCTGGGATGGCTCTAGCCGT
TCCGCAGACGGGATCGATTTCATGATTTTTTTTGTTTCGTTGCATAGGGTTTGGTTTG
CCCTTTTCCTTTATTTCAATATATGCCGTGCACTTGTTTGTCGGGTCATCTTTTCATGC
TTTTTTTTGTCTTGGTTGTGATGATGTGGTCTGGTTGGGCGGTCGTTCTAGATCGGAG
TAGAATTAATTCTGTTTCAAACTACCTGGTGGATTTATTAATTTTGGATCTGTATGTGT
GTGCCATACATATTCATAGTTACGAATTGAAGATGATGGATGGAAATATCGATCTAGG
ATAGGTATACATGTTGATGCGGGTTTTACTGATGCATATACAGAGATGCTTTTTGTTC
GCTTGGTTGTGATGATGTGGTGTGGTTGGGCGGTCGTTCATTCGTTCTAGATCGGAG
TAGAATACTGTTTCAAACTACCTGGTGTATTTATTAATTTTGGAACTGTATGTGTGTG
TCATACATCTTCATAGTTACGAGTTTAAGATGGATGGAAATATCGATCTAGGATAGGT
ATACATGTTGATGTGGGTTTTACTGATGCATATACATGATGGCATATGCAGCATCTATT
CATATGCTCTAACCTTGAGTACCTATCTATTATAATAAACAAGTATGTTTTATAATTATT
TTGATCTTGATATACTTGGATGATGGCATATGCAGCAGCTATATGTGGATTTTTTTAGC
CCTGCCTTCATACGCTATTTATTTGCTTGGTACTGTTTCTTTTGTCGATGCTCACCCTG
TTGTTTGGTGTTACTTCTGCA
CaMV 35S promoter (enhanced):
(SEQ ID NO. 70)
TGAGACTTTTCAACAAAGGGTAATATCGGGAAACCTCCTCGGATTCCATTGCCCAGC
TATCTGTCACTTCATCAAAAGGACAGTAGAAAAGGAAGGTGGCACCTACAAATGCC
ATCATTGCGATAAAGGAAAGGCTATCGTTCAAGATGCCTCTGCCGACAGTGGTCCCA
AAGATGGACCCCCACCCACGAGGAGCATCGTGGAAAAAGAAGACGTTCCAACCAC
GTCTTCAAAGCAAGTGGATTGATGTGATAACATGGTGGAGCACGACACTCTCGTCT
ACTCCAAGAATATCAAAGATACAGTCTCAGAAGACCAAAGGGCTATTGAGACTTTT
CAACAAAGGGTAATATCGGGAAACCTCCTCGGATTCCATTGCCCAGCTATCTGTCAC
TTCATCAAAAGGACAGTAGAAAAGGAAGGTGGCACCTACAAATGCCATCATTGCGA
TAAAGGAAAGGCTATCGTTCAAGATGCCTCTGCCGACAGTGGTCCCAAAGATGGAC
CCCCACCCACGAGGAGCATCGTGGAAAAAGAAGACGTTCCAACCACGTCTTCAAA
GCAAGTGGATTGATGTGATATCTCCACTGACGTAAGGGATGACGCACAATCCCACTA
TCCTTCGCAAGACCTTCCTCTATATAAGGAAGTTCATTTCATTTGGAGAGGACACGC
TGA
CaMV 2 x 35S promoter
(SEQ ID NO. 71)
CCTGCAGGTCAACATGGTGGAGCACGACACACTTGTCTACTCCAAAAATATCAAAG
ATACAGTCTCAGAAGACCAAAGGGCAATTGAGACTTTTCAACAAAGGGTAATATCC
GGAAACCTCCTCGGATTCCATTGCCCAGCTATCTGTCACTTTATTGTGAAGATAGTG
GAAAAGGAAGGTGGCTCCTACAAATGCCATCATTGCGATAAAGGAAAGGCCATCGT
TGAAGATGCCTCTGCCGACAGTGGTCCCAAAGATGGACCCCCACCCACGAGGAGC
ATCGTGGAAAAAGAAGACGTTCCAACCACGTCTTCAAAGCAAGTGGATTGATGTGA
TAACATGGTGGAGCACGACACACTTGTCTACTCCAAAAATATCAAAGATACAGTCTC
AGAAGACCAAAGGGCAATTGAGACTTTTCAACAAAGGGTAATATCCGGAAACCTCC
TCGGATTCCATTGCCCAGCTATCTGTCACTTTATTGTGAAGATAGTGGAAAAGGAAG
GTGGCTCCTACAAATGCCATCATTGCGATAAAGGAAAGGCCATCGTTGAAGATGCCT
CTGCCGACAGTGGTCCCAAAGATGGACCCCCACCCACGAGGAGCATCGTGGAAAA
AGAAGACGTTCCAACCACGTCTTCAAAGCAAGTGGATTGATGTGATATCTCCACTG
ACGTAAGGGATGACGCACAATCCCACTATCCTTCGCAAGACCCTTCCTCTATATAAG
GAAGTTCATTTCATTTGGAGAGGACCTCGACCTCAACACAACATATACAAAACAAA
CGAATCTCAAGCAATCAAGCATTCTACTTCTATTGCAGCAATTTAAATCATTTCTTTT
AAAGCAAAAGCAATTTTCTGAAAATTTTCACCATTTACGAACGATA
CMV promoter:
(SEQ ID NO. 72)
GTGATGCGGTTTTGGCAGTACATCAATGGGCGTGGATAGCGGTTTGACTCACGGGG
ATTTCCAAGTCTCCACCCCATTGACGTCAATGGGAGTTTGTTTTGGCACCAAAATCA
ACGGGACTTTCCAAAATGTCGTAACAACTCCGCCCCATTGACGCAAATGGGCGGTA
GGCGTGTACGGTGGGAGGTCTATATAAGCAGAGCT
Nos terminator:
(SEQ ID NO. 73)
GAATTTCCCCGATCGTTCAAACATTTGGCAATAAAGTTTCTTAAGATTGAATCCTGTT
GCCGGTCTTGCGATGATTATCATATAATTTCTGTTGAATTACGTTAAGCATGTAATAAT
TAACATGTAATGCATGACGTTATTTATGAGATGGGTTTTTATGATTAGAGTCCCGCAA
TTATACATTTAATACGCGATAGAAAACAAAATATAGCGCGCAAACTAGGATAAATTAT
CGCGCGCGGTGTCATCTATGTTACT
E9 terminator:
(SEQ ID NO. 74)
AGAGCTTTCGTTCGTATCATCGGTTTCGACAACGTTCGTCAAGTTCAATGCATCAGT
TTCATTGCGCACACACCAGAATCCTACTGAGTTTGAGTATTATGGCATTGGGAAAAC
TGTTTTTCTTGTACCATTTGTTGTGCTTGTAATTTACTGTGTTTTTTATTCGGTTTTCG
CTATCGAACTGTGAAATGGAAATGGATGGAGAAGAGTTAATGAATGATATGGTCCTT
TTGTTCATTCTCAAATTAATATTATTTGTTTTTTCTCTTATTTGTTGTGTGTTGAATTTG
AAATTATAAGAGATATGCAAACATTTTGTTTTGAGTAAAAATGTGTCAAATCGTGGC
CTCTAATGACCGAAGTTAATATGAGGAGTAAAACACTTGTAGTTGTACCATTATGCTT
ATTCACTAGGCAACAAATATATTTTCAGACCTAGAAAAGCTGCAAATGTTACTGAAT
ACAAGTATGTCCTCTTGTGTTTTAGACATTTATGAACTTTCCTTTATGTAATTTTCCAG
AATCCTTGTCAGATTCTAATCATTGCTTTATAATTATAGTTATACTCATGGATTTGTAGT
TGAGTATGAAAATATTTTTTAATGCATTTTATGACTTGCCAATTGATTGACAAC
CaMV poly(A) signal:
(SEQ ID NO. 75)
TTTCTCCATAATAATGTGTGAGTAGTTCCCAGATAAGGGAATTAGGGTTCCTATAGGG
TTTCGCTCATGTGTTGAGCATATAAGAAACCCTTAGTATGTATTTGTATTTGTAAAATA
CTTCTATCAATAAAATTTCTAATTCCTAAAACCAAAATCCAGTACTAAAATCCAGATC
bGH poly(A) signal:
(SEQ ID NO. 76)
CTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGG.
SOD2 MTS:
(SEQ ID NO. 77)
MLSRAVCGTSRQLAPVLGYLGSRQKHSLPD
COX8 MTS:
(SEQ ID NO. 78)
MSVLTPLLLRGLTGSARRLPVPRAK
CTP:
(SEQ ID NO. 79)
MAPTVMMASSATAVAPFQGLKSAASLPVARRSTRSLGNVSNGGRIRCMQ

### Target Cells of Interest

The recombinant expression construct provided by the present disclosure may be produced according to the genetic engineering methods known in the art. In some embodiments, a base editor or a recombinant expression construct thereof is introduced into a cell to edit a target gene and enable its expression, thereby forming an edited genetically engineered cell.

Any cell derived from any organism may be used with the nucleic acids, polypeptides, compositions and methods of the present disclosure. Cells include but are not limited to a human cell, a non-human cell, an animal cell, a mammalian cell, a bacterium, a protist, a fungus, an insect cell, a yeast, a non-conventional yeast and a plant cell, and include a monocotyledon, a dicotyledon and a plant element, as well as a plant and a seed produced by the method of the present disclosure. In some aspects, the cell of the organism is a germ cell, a somatic cell, a meiotic cell, a mitotic cell, a stem cell or a pluripotent stem cell.

In some embodiments, animal cells may include but are not limited to cells derived from the organisms of phylums including phylum Chordata, phylum Arthropoda, phylum Mollusca, phylum Annelida, phylum Coelenterata or phylum Echinodermata and the organisms of classes including mammal, insect, bird, amphibian, reptile or fish. In some aspects, the animal is a human, a mouse, a Caenorhabditis elegans, a rat, a fruit fly, a zebrafish, a chicken, a dog, a cattle, a sheep, a pig, a guinea pig, a hamster, a chicken, a Japanese rice fish, a sea lamprey, a puffer, a tree frog, a monkey or a chimpanzee.

Specific types of animal cell include a haploid cell, a diploid cell, a germ cell, a neuron, a muscle cell, an endocrine cell or an exocrine cell, an epithelial cell, a muscle cell, a tumor cell, an embryonic cell, a hematopoietic cell, an osteocyte, a germplasm cell, a somatic cell, a stem cell, a pluripotent stem cell, an induced pluripotent stem cell, a progenitor cell, a meiotic cell, and a mitotic cell. In some aspects, multiple cells derived from an organism may be used.

In some embodiments, plant cells include cells derived from monocotyledons and dicotyledons. Examples of monocotyledons that may be used include but are not limited to corn (Zea mays), rice (Oryza sativa), rye (Secale cereale), sorghum (Sorghum bicolor, Sorghum vulgare), millet (for example, pearl millet, Pennisetum glaucum), maiden cane (Panicum miliaceum), unhusked rice (Setaria italica), finger millet (Eleusine coracana), wheat (Triticum spp., for example, Triticum aestivum, Triticum monococcum), sugarcane (Saccharum spp.), oat (Avena), barley (Hordeum), switchgrass (Panicum virgatum), pineapple (Ananas comosus), banana (Musa spp.), palm, an ornamental plant, turfgrass, and other grasses. Examples of dicotyledons that may be used include but are not limited to soybean (Glycine max), Brassica species (such as, but not limited to oilseed rape or canola), Brassica napus, B. campestris, Brassica rapa, Brassica. juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), peanut (Arachis hypogaea), tomato (Solanum lycopersicum), potato (Solanum tuberosum). Additional plants that may be used include safflower (Carthamus tinctorius), sweet potato (Ipomoea batatas), cassava (Manihot esculenta), coffee (Coffea spp.), coconut (Cocos nucifera), citrus tree (Citrus spp.), cocoa (Theobroma cacao), tea tree (tea, Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugarbeet (Beta vulgaris), vegetable, an ornamental plant, and a conifer. Vegetables that may be used include tomato (Lycopersicon esculentum), lettuce (for example, Lactuca sativa), green bean (Phaseolus vulgaris), lima bean (Phaseolus limensis), pea (Lathyrus spp.) and members of genus Cucumis such as cucumber (C. sativus), cantaloupe (C. cantalupensis), musk melon (C. melo). Ornamental plants include rhododendrons (Rhododendron spp.), hydrangea (Macrophylla hydrangea), Hibiscus rosasanensis, rose (Rosa spp.), tulip (Tulipa spp.), narcissus (Narcissus spp.), Petunia hybrida, Dianthus caryophyllus, Euphorbia pulcherrima and chrysanthemums. Conifers that may be used include pine trees such as loblolly pine (Pinus taeda), slash pine (Pinus elliotii), ponderosa pine (Pinus ponderosa), lodgepole pine (Pinus contorta), and Monterey pine (Pinus radiata); Douglasfir (Pseudotsuga menziesii); Western hemlock (Tsuga canadensis); Sitka spruce (Picea glauca); redwood (Sequoia sempervirens); true firs, such as silver fir (Abies amabilis) and balsam fir (Abies balsamea); and cedars, such as Thuja plicata and Chamaecyparis nootkatensis.

Specific types of plant cell include but are not limited to cells derived from a whole plant, a seedling, a meristem, a ground tissue, a vascular tissue, a dermal tissue, a seed, a leaf, a root, a bud, a stem, a flower, a fruit, a stolon, a bulb, a tuber, a corm, an asexual terminal branch, a bud, a budlet, a tumor tissue, and various forms of cells and cultures (for example, a single cell, a protoplast, an embryo, a callus). They may exist in a plant or a plant organ, a tissue culture, or a cell culture.

### Therapeutic Use

The present disclosure also encompasses the use of the base editor of the present disclosure in the treatment of diseases.

The up-regulation, down-regulation, inactivation, activation or mutation correction of disease-related genes, the introduction of disease-related genes to disease-related sites or the like may be achieved by modifying disease-related genes with the base editor of the present disclosure, thereby realizing the prevention and/or treatment of diseases and/or the establishment of disease-related models. For example, the target nucleic acid region as described in the present disclosure may be located in the protein coding region of a disease-related gene, or, for example, may be located in a regulatory region of gene expression such as a promoter region or an enhancer region, thereby capable of achieving the functional modification of the disease-related gene or the modification of the expression of the disease-related gene. Therefore, the modifications of a disease-related gene as described herein include the modifications of the disease-related gene itself (for example, the protein coding region), as well as the modifications of its expression regulatory regions (such as a promoter, an enhancer, an intron, etc.).

A “disease-related” gene refers to any gene that produces a transcription product or translation product at an abnormal level or in an abnormal form in cells derived from a disease-affected tissue as compared with the non-disease control tissue or cell. In a case where the modified expression is associated with the occurrence and/or progression of a disease, it may be a gene that is expressed at an abnormally high level, and it may be a gene that is expressed at an abnormally low level. A disease-related gene also refers to a genetically mutated gene that has one or more mutations, or is directly responsible for the etiology of the disease or in linkage disequilibrium with one or more genes responsible for the etiology of the disease. The mutation or genetic variation is, for example, a single nucleotide variation (SNV). The products of transcription or translation may be known or unknown, and may be at a normal level or an abnormal level.

Accordingly, the present disclosure also provides a method for treating a disease in a subject in need thereof, comprising delivering an effective amount of the base editor of the present disclosure to the subject so as to modify a gene related to the disease (for example, subjecting the mitochondrial DNA to deamination via one or more fusion proteins). The present disclosure also provides the use of the base editor in the preparation of a pharmaceutical composition for treating a disease in a subject in need thereof, wherein the base editor is used to modify a gene related to the disease. The present disclosure also provides a pharmaceutical composition for treating a disease in a subject in need thereof, comprising the base editor of the present disclosure and optionally a pharmaceutically acceptable carrier, wherein the base editor is used to modify a gene related to the disease.

In some embodiments, the fusion protein or the base editor described in the present disclosure is used to introduce a point mutation into a nucleic acid by subjecting the target nucleobase (for example, C residue) to deamination. In some embodiments, the deamination of the target nucleobase results in the correction of a genetic defect, for example, upon correcting a point mutation that leads to the loss of function in the genetic product. In some embodiments, the genetic defect is associated with a disease or condition (for example, lysosomal storage disease or a metabolic disease such as Type I diabetes). In some embodiments, the method provided herein may be used to introduce an inactivating point mutation into a gene or an allele encoding a genetic product associated with the disease or disorder.

In some embodiments, the embodiments described in the present disclosure are intended to restore the function of a dysfunctional gene via genome editing. The nucleobase editing protein provided herein may be used for in-vitro gene editing of human cells, such as the correction of a disease-related mutation in a human cell culture.

In some embodiments, the embodiments described in the present disclosure are intended for the treatment of a disease associated with or caused by a point mutation, and the point mutation may be corrected by the DNA base editing fusion protein provided herein. In some embodiments, the disease is a proliferative disease. In some embodiments, the disease is a genetic disease. In some embodiments, the disease is a de novo disease. In some embodiments, the disease is a metabolic disease. In some embodiments, the disease is lysosomal storage disease.

In some embodiments, the embodiments described in the present disclosure are intended for the treatment of mitochondrial diseases or disorders. As used herein, a “mitochondrial disease” refers to a disease caused by abnormal mitochondria, for example, a mitochondrial gene mutation, a gene mutation in enzymatic pathway, etc. Examples of the disease include but are not limited to neurological diseases, loss of motion control, muscle weakness and pain, gastrointestinal diseases and difficulty in swallowing, poor growth, heart diseases, liver diseases, diabetes, respiratory complications, epilepsy, vision/hearing problems, lactic acidosis, developmental retardation and susceptibility to infection.

Examples of the diseases described in the present disclosure include but are not limited to genetic diseases, circulatory system diseases, muscle diseases, diseases in brain, nervous centralis and immune system, Alzheimer's disease, secretase disorders, amyotrophic lateral sclerosis (ALS), autism, trinucleotide repeat expansion disorder, hearing diseases, gene-targeted therapy of non-dividing cells (neurons, muscle cells), liver and kidney diseases, diseases in epithelial cells and lung, cancer, Usher syndrome or retinitis pigmentosa-39, cystic fibrosis, HIV and AIDS, β-mediterranean anemia, sickle cell disease, herpes simplex virus, autism, drug addiction, age-related macular degeneration, and schizophrenia. Other diseases treated by correcting point mutations or introducing inactivating mutations into disease-related genes are known to a person skilled in the art, and therefore, the present disclosure is not limited in this regard. In addition to the diseases illustratively described in the present disclosure, the strategy and the fusion protein provided by the present disclosure may also be used to treat other related diseases, and this application is apparent to a person skilled in the art. For diseases or targets applicable to the present disclosure, please refer to the related diseases for which base editors are applicable as listed in WO2015089465A1 (PCT/US2014/070135), WO2016205711A1 (PCT/US2016/038181), WO2018141835A1 (PCT/EP2018/052491), WO2020191234A1 (PCT/US2020/023713), WO2020191233A1 (PCT/US2020/023712), WO2019079347A1 (PCT/US2018/056146), and WO2021155065A1 (PCT/US2021/015580).

### Use in Plants

The base editing fusion protein, the base editor and the method for producing genetically modified cells of the present disclosure are particularly suitable for the genetic modification of plants. Preferably, the plant is a crop plant, including but not limited to wheat, rice, corn, soybean, sunflower, sorghum, oilseed rape, alfalfa, cotton, barley, millet, sugar cane, tomato, tobacco, cassava and potato. More preferably, the plant is rice.

In another aspect, the present disclosure provides a method for producing a genetically modified plant, comprising introducing the base editor of the present disclosure into at least one plant, thereby resulting in one or more nucleotide substitutions within the target nucleic acid region in the genome of said at least one plant.

In some embodiments, the method further comprises screening a plant having one or more nucleotide substitutions as desired from said at least one plant.

In the method of the present disclosure, the base editing composition may be introduced into a plant via various methods well known to a person skilled in the art. Methods that may be used to introduce the base editor of the present disclosure into a plant include but are not limited to biolistic method, PEG-mediated protoplast transformation, Agrobacterium-mediated transformation, plant virus-mediated transformation, pollen tube channel method and ovary injection method. Preferably, the base editing composition is introduced into a plant via transient transformation.

In the method of the present disclosure, the modification of the target sequence may be achieved by simply introducing the base editing fusion protein into a plant cell or producing the base editing fusion protein in a plant cell, and said modification may be stably inherited without the need of the stable transformation of the exogenous polynucleotide encoding the components of the base editor into the plant. This avoids the potential off-target effects of the stably existing (continuously produced) base editing composition, and avoids the integration of the exogenous nucleotide sequence(s) in the plant genome as well, thereby having higher biological safety.

In some preferred embodiments, said introduction is performed in the absence of selection pressure, thereby avoiding the integration of the exogenous nucleotide sequence(s) in the plant genome.

In some embodiments, said introduction include transforming the base editor of the present disclosure into an isolated plant cell or tissue and enabling the regeneration of the transformed plant cell or tissue into an intact plant. Preferably, said regeneration is performed in the absence of selection pressure, that is, any selection agent for the selection gene carried on the expression vector is not used during tissue culture. The regeneration efficiency of plant may be enhanced without the use of a selection agent, and a modified plant that does not comprise an exogenous nucleotide sequence is obtained.

In some other embodiments, the base editor of the present disclosure may be transformed into a specific part of an intact plant, such as leaf, stem tip, pollen tube, young ear, or hypocotyl. This is particularly suitable for the transformation of the plants that are difficult to regenerate by tissue culture.

Therefore, in some embodiments, a plant whose genome is free of the integration of exogenous polynucleotide, i.e., a transgene-free modified plant, may be obtained by conducting the genetic modification and breeding of plant using the method of the present disclosure.

In some embodiments of the present disclosure, the modified target nucleic acid region is associated with plant traits such as an agronomic trait. As a result, said one or more nucleotide substitutions result in the plant having altered (preferably, improved) traits such as an agronomic trait, as compared with the wild-type plant.

In some embodiments, the method further comprises a step of screening a plant having one or more nucleotide substitutions as desired and/or a desired trait such as an agronomic trait.

In some embodiments of the present disclosure, the method further comprises obtaining the progeny of the genetically modified plant. Preferably, the genetically modified plant or the progeny thereof has one or more nucleotide substitutions as desired and/or a desired trait such as an agronomic trait.

In another aspect, the present disclosure also provides a genetically modified plant, a progeny thereof or a part thereof, wherein the plant is obtained by the above-mentioned method of the present disclosure. In some embodiments, the genetically modified plant, the progeny thereof or the part thereof is non-transgenic. Preferably, the genetically modified plant or the progeny thereof has a desired genetic modification and/or a desired trait such as an agronomic trait.

In another aspect, the present disclosure also provides a method for plant breeding, comprising the hybridization of a first genetically modified plant that comprises one or more nucleotide substitutions in the target nucleic acid region and is obtained by the above-mentioned method of the present disclosure and a second plant free of said one or more nucleotide substitutions, thereby introducing said one or more nucleotide substitutions into the second plant. Preferably, the first genetically modified plant has a desired trait such as an agronomic trait.

### EXAMPLES

A further understanding of the present disclosure may be obtained by referring to some specific examples given herein. These examples are merely for the illustration of the present disclosure and are not intended to impose any limitation to the scope of the present disclosure. Apparently, a variety of modifications and changes may be made to the present disclosure without departing from the essence of the present disclosure. Accordingly, these modifications and changes are also within the scope as claimed by the present application.

Partial element sequences used in subsequent examples are as set forth below.

OsBADH2 Left TALE repeat
(SEQ ID NO. 89)
LTPDQVVAIASNNGGKQALETVQRLLPVLCQDHGLTPDQVVAIASHDGGKQALETVQR
LLPVLCQDHGLTPDQVVAIASNGGGKQALETVQRLLPVLCQDHGLTPDQVVAIASNNG
GKQALETVQRLLPVLCQDHGLTPDQVVAIASNNGGKQALETVQRLLPVLCQDHGLTP
DQVVAIASNIGGKQALETVQRLLPVLCQDHGLTPDQVVAIASNGGGKQALETVQRLLP
VLCQDHGLTPDQVVAIASNNGGKQALETVQRLLPVLCQDHGLTPDQVVAIASHDGGK
QALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNGGGKQALETVQRLLPVL
CQDHGLTPDQVVAIASNNGGKQALETVQRLLPVLCQDHGLTPDQVVAIASNIGGKQAL
ETVQRLLPVLCQDHGLTPDQVVAIASNNGGKQALETVQRLLPVLCQDHGLTPDQVVAI
ASNGGGKQALETVQRLLPVLCQDHGLTPDQVVAIASNIGGKQALE
OsBADH2 Right TALE repeat
(SEQ ID NO. 90)
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASNGGGKQALETVQRLLPVLCQDHGLTP
DQVVAIASNGGGKQALETVQRLLPVLCQDHGLTPDQVVAIASNGGGKQALETVQRLL
PVLCQDHGLTPDQVVAIASNNGGKQALETVQRLLPVLCQDHGLTPDQVVAIASNGGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNIGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNGGGKQALE
OsDEP1 Left TALE repeat
(SEQ ID NO. 91)
LTPDQVVAIASNNGGKQALETVQRLLPVLCQDHGLTPDQVVAIASHDGGKQALETVQR
LLPVLCQDHGLTPDQVVAIASNIGGKQALETVQRLLPVLCQDHGLTPDQVVAIASNIGG
KQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNNGGKQALETVQRLLPVL
CQDHGLTPDQVVAIASNIGGKQALETVQRLLPVLCQDHGLTPDQVVAIASHDGGKQAL
ETVQRLLPVLCQDHGLTPDQVVAIASHDGGKQALETVQRLLPVLCQDHGLTPDQVVAI
ASNIGGKQALETVQRLLPVLCQDHGLTPDQVVAIASNIGGKQALETVQRLLPVLCQDH
GLTPDQVVAIASNNGGKQALETVQRLLPVLCQDHGLTPDQVVAIASNNGGKQALETV
QRLLPVLCQDHGLTPDQVVAIASNGGGKQALETVQRLLPVLCQDHGLTPDQVVAIASN
NGGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNGGGKQALE
OsDEP1 Right TALE repeat
(SEQ ID NO. 92)
LTPDQVVAIASNNGGKQALETVQRLLPVLCQDHGLTPDQVVAIASNNGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASHDGGKQALETVQRLLPVLCQDHGLTP
DQVVAIASNNGGKQALETVQRLLPVLCQDHGLTPDQVVAIASNGGGKQALETVQRLL
PVLCQDHGLTPDQVVAIASHDGGKQALETVQRLLPVLCQDHGLTPDQVVAIASNNGG
KQALETVQRLLPVLCQDHGLTPDQVVAIASHDGGKQALETVQRLLPVLCQDHGLTPD
QVVAIASNIGGKQALETVQRLLPVLCQDHGLTPDQVVAIASNNGGKQALETVQRLLPV
LCQDHGLTPDQVVAIASHDGGKQALETVQRLLPVLCQDHGLTPDQVVAIASNIGGKQA
LETVQRLLPVLCQDHGLTPDQVVAIASNGGGKQALE
OsCKX2 Left TALE repeat
(SEQ ID NO. 93)
LTPDQVVAIASHDGGKQALETVQRLLPVLCQDHGLTPDQVVAIASHDGGKQALETVQR
LLPVLCQDHGLTPDQVVAIASNGGGKQALETVQRLLPVLCQDHGLTPDQVVAIASNNG
GKQALETVQRLLPVLCQDHGLTPDQVVAIASNNGGKQALETVQRLLPVLCQDHGLTP
DQVVAIASHDGGKQALETVQRLLPVLCQDHGLTPDQVVAIASHDGGKQALETVQRLL
PVLCQDHGLTPDQVVAIASNNGGKQALETVQRLLPVLCQDHGLTPDQVVAIASHDGG
KQALETVQRLLPVLCQDHGLTPDQVVAIASNNGGKQALETVQRLLPVLCQDHGLTPD
QVVAIASNGGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAIASNIGGKQ
ALETVQRLLPVLCQDHGLTPDQVVAIASHDGGKQALETVQRLLPVLCQDHGLTPDQVV
AIASNNGGKQALE
OsCKX2 Right TALE repeat
(SEQ ID NO. 94)
LTPDQVVAIASHDGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNIG
GKQALETVQRLLPVLCQDHGLTPDQVVAIASNNGGKQALETVQRLLPVLCQDHGLTP
DQVVAIASNIGGKQALETVQRLLPVLCQDHGLTPDQVVAIASNNGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNNGGKQALETVQRLLPVL
CQDHGLTPDQVVAIASHDGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASHDGGKQALETVQRLLPVLCQDHGLTPDQVVAIASNNGGKQALETVQRLLPVLCQ
DHGLTPDQVVAIASHDGGKQALE
OsSD1 Left TALE repeat
(SEQ ID NO. 95)
LTPDQVVAIASHDGGKQALETVQRLLPVLCQDHGLTPDQVVAIASHDGGKQALETVQR
LLPVLCQDHGLTPDQVVAIASNIGGKQALETVQRLLPVLCQDHGLTPDQVVAIASNNG
GKQALETVQRLLPVLCQDHGLTPDQVVAIASNNGGKQALETVQRLLPVLCQDHGLTP
DQVVAIASNIGGKQALETVQRLLPVLCQDHGLTPDQVVAIASHDGGKQALETVQRLLP
VLCQDHGLTPDQVVAIASNNGGKQALETVQRLLPVLCQDHGLTPDQVVAIASNIGGKQ
ALETVQRLLPVLCQDHGLTPDQVVAIASHDGGKQALETVQRLLPVLCQDHGLTPDQVV
AIASNNGGKQALETVQRLLPVLCQDHGLTPDQVVAIASNGGGKQALETVQRLLPVLC
QDHGLTPDQVVAIASHDGGKQALETVQRLLPVLCQDHGLTPDQVVAIASNNGGKQAL
ETVQRLLPVLCQDHGLTPDQVVAIASNNGGKQALETVQRLLPVLCQDHGLTPDQVVAI
ASHDGGKQALETVQRLLPVLCQDHGLTPDQVVAIASNNGGKQALETVQRLLPVLCQD
HGLTPDQVVAIASNNGGKQALE
OsSD1 Right TALE repeat
(SEQ ID NO. 96)
LTPDQVVAIASNNGGKQALETVQRLLPVLCQDHGLTPDQVVAIASNIGGKQALETVQR
LLPVLCQDHGLTPDQVVAIASHDGGKQALETVQRLLPVLCQDHGLTPDQVVAIASNNG
GKQALETVQRLLPVLCQDHGLTPDQVVAIASNNGGKQALETVQRLLPVLCQDHGLTP
DQVVAIASNNGGKQALETVQRLLPVLCQDHGLTPDQVVAIASNNGGKQALETVQRLL
PVLCQDHGLTPDQVVAIASHDGGKQALETVQRLLPVLCQDHGLTPDQVVAIASNNGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNGGGKQALETVQRLLPVLCQDHGLTPDQVVAIASNGGGKQ
ALETVQRLLPVLCQDHGLTPDQVVAIASHDGGKQALETVQRLLPVLCQDHGLTPDQVV
AIASNNGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNNGGKQAL
E
SIRT6 Left TALE repeat
(SEQ ID NO. 97)
LTPDQVVAIASNGGGKQALETVQRLLPVLCQDHGLTPEQVVAIASNIGGKQALETVQR
LLPVLCQAHGLTPDQVVAIASHDGGKQALETVQRLLPVLCQAHGLTPAQVVAIANNNG
GKQALETVQRLLPVLCQDHGLTPDQVVAIASHDGGKQALETVQRLLPVLCQDHGLTPE
QVVAIANNNGGKQALETVQRLLPVLCQAHGLTPDQVVAIANNNGGKQALETVQRLLP
VLCQAHGLTPAQVVAIASHDGGKQALETVQRLLPVLCQDHGLTPDQVVAIANNNGGK
QALETVQRLLPVLCQDHGLTPEQVVAIANNNGGKQALETVQRLLPVLCQAHGLTPDQ
VVAIANNNGGKQALETVQRLLPVLCQAHGLTPAQVVAIANNNGGKQALETVQRLLPV
LCQDHGLTPDQVVAIASHDGGKQALETVQRLLPVLCQDHGLTPEQVVAIASNGGGKQ
ALETVQRLLPVLCQAHGLTPDQVVAIANNNGGKQALETVQRLLPVLCQAHGLTPAQV
VAIASNGGGKQALETVQRLLPVLCQDHGLTPEQVVAIASHDGGRPALE
SIRT6 Right TALE repeat
(SEQ ID NO. 98)
LTPDQVVAIASHDGGKQALETVQRLLPVLCQDHGLTPEQVVAIASHDGGKQALETVQR
LLPVLCQAHGLTPDQVVAIANNNGGKQALETVQRLLPVLCQAHGLTPAQVVAIANNN
GGKQALETVQRLLPVLCQDHGLTPDQVVAIANNNGGKQALETVQRLLPVLCQDHGLT
PEQVVAIASNIGGKQALETVQRLLPVLCQAHGLTPDQVVAIANNNGGKQALETVQRLL
PVLCQAHGLTPAQVVAIANNNGGKQALETVQRLLPVLCQDHGLTPDQVVAIASHDGG
KQALETVQRLLPVLCQDHGLTPEQVVAIASHDGGKQALETVQRLLPVLCQAHGLTPDQ
VVAIANNNGGKQALETVQRLLPVLCQAHGLTPAQVVAIASHDGGKQALETVQRLLPV
LCQDHGLTPDQVVAIASNIGGKQALETVQRLLPVLCQDHGLTPEQVVAIASHDGGKQA
LETVQRLLPVLCQAHGLTPDQVVAIASNGGGKQALETVQRLLPVLCQAHGLTPAQVVA
IASNGGGKQALETVQRLLPVLCQDHGLTPEQVVAIASNNGGRPALE
OsRbcL Left TALE repeat
(SEQ ID NO. 99)
LTPDQVVAIASNGGGKQALETVQRLLPVLCQDHGLTPDQVVAIASNGGGKQALETVQR
LLPVLCQDHGLTPDQVVAIASNIGGKQAVETVQRLLPVLCQAHGLTPAQVVAIASHDG
GKQAVETVQRLLPVLCQDHGLTPDQVVAIASHDGGKQAVETVQRLLPVLCQDHGLTP
DQVVAIASNIGGKQALETLQRLLPVLCQAHGLTPAQVVAIASNIGGKQALETVQRLLPV
LCQDHGLTPDQVVAIASNIGGKQALETVQRLLPVLCQDHGLTPDQVVAIASNNGGKQA
LETVQRLLPVLCQAHGLTPAQVVAIASNIGGKQALETVQRLLPVLCQAHGLTPDQVVAI
ASNGGGKQAVETVQRLLPVLCQAHGLTPAQVVAIASNNGGKQAVETVQRLLPVLCQA
HGLTPAQVVAIASNIGGKQAVETVQRLLPVLCQDHGLTPEQVVAIASNGGGKQALE
OsRbcL Right TALE repeat
(SEQ ID NO. 100)
LTPAQVVAIASHDGGKQALETVQRLLPVLCQAHGLTPDQVVAIASHDGGKQAVETVQR
LLPVLCQDHGLTPDQVVAIASNIGGKQAVETVQRLLPVLCQAHGLTPAQVVAIASNIGG
KQAVETVQRLLPVLCQDHGLTPDQVVAIASHDGGKQALETLQRLLPVLCQDHGLTPDQ
VVAIASNNGGKQALETVQRLLPVLCQAHGLTPAQVVAIASHDGGKQALETVQRLLPVL
CQDHGLTPDQVVAIASNIGGKQALETVQRLLPVLCQDHGLTPDQVVAIASNGGGKQAL
ETVQRLLPVLCQDHGLTPDQVVAIASNIGGKQAVETVQRLLPVLCQDHGLTPDQVVAIA
SNIGGKQAVETVQRLLPVLCQDHGLTPDQVVAIASNIGGKQAVETVQRLLPVLCQDHG
LTPDQVVAIASNGGGKQALETVQRLLPVLCQDHGLTPDQVVAIASNNGGKQALETVQR
LLPVLCQDHGLTPDQVVAIASNNGGKQALETVQRLLPVLCQDHGLTPAQVVAIASNGG
GKQALETVQRLLPVLCQDHGLTPEQVVAIASNGGGKQALE
ND6 Left TALE repeat
(SEQ ID NO. 101)
LTPEQVVAIASNNGGKQALETVQRLLPVLCQAHGLTPEQVVAIASNIGGKQALETVQRL
LPVLCQAHGLTPEQVVAIASHDGGKQALETVQRLLPVLCQAHGLTPEQVVAIASHDGG
KQALETVQRLLPVLCQAHGLTPEQVVAIASHDGGKQALETVQRLLPVLCQAHGLTPEQ
VVAIASHDGGKQALETVQRLLPVLCQAHGLTPEQVVAIASHDGGKQALETVQRLLPVL
CQAHGLTPEQVVAIASNIGGKQALETVQRLLPVLCQAHGLTPEQVVAIASNGGGKQAL
ETVQRLLPVLCQAHGLTPEQVVAIASNGGGRPALE
ND6 Right TALE repeat
(SEQ ID NO. 102)
LTPEQVVAIASNNGGKQALETVQRLLPVLCQAHGLTPEQVVAIASNIGGKQALETVQRL
LPVLCQAHGLTPEQVVAIASNGGGKQALETVQRLLPVLCQAHGLTPEQVVAIASNNGG
KQALETVQRLLPVLCQAHGLTPEQVVAIASNNGGKQALETVQRLLPVLCQAHGLTPEQ
VVAIASHDGGKQALETVQRLLPVLCQAHGLTPEQVVAIASNGGGKQALETVQRLLPVL
CQAHGLTPEQVVAIASNIGGKQALETVQRLLPVLCQAHGLTPEQVVAIASNGGGKQAL
ETVQRLLPVLCQAHGLTPEQVVAIASNGGGKQALETVQRLLPVLCQAHGLTPEQVVAI
ASNGGGRPALE
ND5.1 Left TALE repeat
(SEQ ID NO. 103)
LTPEQVVAIASNIGGKQALETVQRLLPVLCQAHGLTPEQVVAIASNNGGKQALETVQRL
LPVLCQAHGLTPEQVVAIASHDGGKQALETVQRLLPVLCQAHGLTPEQVVAIASNIGG
KQALETVQRLLPVLCQAHGLTPEQVVAIASNGGGKQALETVQRLLPVLCQAHGLTPEQ
VVAIASNGGGKQALETVQRLLPVLCQAHGLTPEQVVAIASNIGGKQALETVQRLLPVL
CQAHGLTPEQVVAIASNNGGKQALETVQRLLPVLCQAHGLTPEQVVAIASHDGGKQAL
ETVQRLLPVLCQAHGLTPEQVVAIASNIGGKQALETVQRLLPVLCQAHGLTPEQVVAIA
SNNGGKQALETVQRLLPVLCQAHGLTPEQVVAIASNNGGKQALETVQRLLPVLCQAH
GLTPEQVVAIASNIGGKQALETVQRLLPVLCQAHGLTPEQVVAIASNIGGKQALETVQR
LLPVLCQAHGLTPEQVVAIASNGGGRPALE
ND5.1 Right TALE repeat
(SEQ ID NO. 104)
LTPEQVVAIASHDGGKQALETVQRLLPVLCQAHGLTPEQVVAIASNGGGKQALETVQR
LLPVLCQAHGLTPEQVVAIASNGGGKQALETVQRLLPVLCQAHGLTPEQVVAIASNGG
GKQALETVQRLLPVLCQAHGLTPEQVVAIASNNGGKQALETVQRLLPVLCQAHGLTPE
QVVAIASNNGGKQALETVQRLLPVLCQAHGLTPEQVVAIASNIGGKQALETVQRLLPV
LCQAHGLTPEQVVAIASNNGGKQALETVQRLLPVLCQAHGLTPEQVVAIASNGGGKQA
LETVQRLLPVLCQAHGLTPEQVVAIASNIGGKQALETVQRLLPVLCQAHGLTPEQVVAI
ASNNGGKQALETVQRLLPVLCQAHGLTPEQVVAIASNIGGKQALETVQRLLPVLCQAH
GLTPEQVVAIASNIGGRPALE
ND3 Left TALE repeat
(SEQ ID NO. 105)
LTPEQVVAIASNIGGKQALETVQALLPVLCQAHGLTPQQVVAIASHDGGKQALETVQR
LLPVLCQAHGLTPQQVVAIASNNGGKQALETVQRLLPVLCQAHGLTPEQVVAIASNIG
GKQALETVQALLPVLCQAHGLTPEQVVAIASNNGGKQALETVQALLPVLCQAHGLTPE
QVVAIASNGGGKQALETVQALLPVLCQAHGLTPEQVVAIASNNGGKQALETVQRLLPV
LCQAHGLTPEQVVAIASHDGGKQALETVQALLPVLCQAHGLTPEQVVAIASNNGGKQ
ALETVQRLLPVLCQAHGLTPEQVVAIASNNGGKQALETVQALLPVLCQAHGLTPEQVV
AIASHDGGKQALETVQALLPVLCQAHGLTPEQVVAIASNGGGKQALETVQALLPVLC
QAHGLTPEQVVAIASNGGGKQALETVQRLLPVLCQAHGLTPQQVVAIASHDGGKQAL
ETVQRLLPVLCQAHGLTPEQVVAIASNNGGKQALETVQALLPVLCQAHGLTPQQVVAI
ASNIGGRPALE
ND3 Right TALE repeat
(SEQ ID NO. 106)
LTPEQVVAIASNIGGKQALETVQALLPVLCQAHGLTPQQVVAIASNGGGKQALETVQR
LLPVLCQAHGLTPQQVVAIASNNGGKQALETVQRLLPVLCQAHGLTPEQVVAIASNNG
GKQALETVQALLPVLCQAHGLTPEQVVAIASNIGGKQALETVQALLPVLCQAHGLTPE
QVVAIASNNGGKQALETVQALLPVLCQAHGLTPEQVVAIASNIGGKQALETVQRLLPV
LCQAHGLTPEQVVAIASNIGGKQALETVQALLPVLCQAHGLTPEQVVAIASNIGGKQAL
ETVQRLLPVLCQAHGLTPEQVVAIASNNGGKQALETVQALLPVLCQAHGLTPEQVVAI
ASNNGGKQALETVQALLPVLCQAHGLTPEQVVAIASNNGGKQALETVQALLPVLCQA
HGLTPEQVVAIASNIGGKQLETVQRLLPVLCQAHGLTPQQVVAIASHDGGKQALETVQ
RLLPVLCQAHGLTPEQVVAIASNNGGKQALETVQALLPVLCQAHGLTPQQVVAIASHD
GGRPALE
ND1.3 Left TALE repeat
(SEQ ID NO. 107)
LTPEQVVAIASHDGGKQALETVQALLPVLCQAHGLTPQQVVAIASNGGGKQALETVQR
LLPVLCQAHGLTPQQVVAIASHDGGKQALETVQRLLPVLCQAHGLTPEQVVAIASHDG
GKQALETVQALLPVLCQAHGLTPEQVVAIASNIGGKQALETVQALLPVLCQAHGLTPE
QVVAIASHDGGKQALETVQALLPVLCQAHGLTPEQVVAIASNIGGKQALETVQRLLPV
LCQAHGLTPEQVVAIASHDGGKQALETVQALLPVLCQAHGLTPEQVVAIASNGGGKQ
ALETVQRLLPVLCQAHGLTPEQVVAIASNIGGKQALETVQALLPVLCQAHGLTPEQVV
AIASNNGGKQALETVQALLPVLCQAHGLTPEQVVAIASHDGGKQALETVQALLPVLC
QAHGLTPEQVVAIASNIGGKQALETVQRLLPVLCQAHGLTPQQVVAIASNNGGKQALE
TVQRLLPVLCQAHGLTPEQVVAIASNIGGKQALETVQALLPVLCQAHGLTPQQVVAIAS
NNGGRPALE
ND1.3 Right TALE repeat
(SEQ ID NO. 108)
LTPEQVVAIASHDGGKQALETVQALLPVLCQAHGLTPQQVVAIASHDGGKQALETVQR
LLPVLCQAHGLTPQQVVAIASHDGGKQALETVQRLLPVLCQAHGLTPEQVVAIASHDG
GKQALETVQALLPVLCQAHGLTPEQVVAIASNGGGKQALETVQALLPVLCQAHGLTPE
QVVAIASNGGGKQALETVQALLPVLCQAHGLTPEQVVAIASHDGGKQALETVQRLLPV
LCQAHGLTPEQVVAIASNNGGKQALETVQALLPVLCQAHGLTPEQVVAIASNNGGKQ
ALETVQRLLPVLCQAHGLTPEQVVAIASHDGGKQALETVQALLPVLCQAHGLTPEQVV
AIASNIGGKQALETVQALLPVLCQAHGLTPEQVVAIASNIGGKQALETVQALLPVLCQA
HGLTPEQVVAIASNNGGKQALETVQRLLPVLCQAHGLTPQQVVAIASNNGGKQALETV
QRLLPVLCQAHGLTPEQVVAIASNGGGKQALETVQALLPVLCQAHGLTPEQVVAIASN
GGGKQALETVQALLPVLCQAHGLTPQQVVAIASHDGGRPALE
ND1.2 Left TALE repeat
(SEQ ID NO. 109)
LTPEQVVAIASHDGGKQALETVQALLPVLCQAHGLTPQQVVAIASHDGGKQALETVQR
LLPVLCQAHGLTPQQVVAIASNGGGKQALETVQRLLPVLCQAHGLTPEQVVAIASNIG
GKQALETVQALLPVLCQAHGLTPEQVVAIASNGGGKQALETVQALLPVLCQAHGLTPE
QVVAIASNGGGKQALETVQALLPVLCQAHGLTPEQVVAIASNGGGKQALETVQRLLPV
LCQAHGLTPEQVVAIASNIGGKQALETVQALLPVLCQAHGLTPEQVVAIASNGGGKQA
LETVQRLLPVLCQAHGLTPEQVVAIASNGGGKQALETVQALLPVLCQAHGLTPEQVVA
IASHDGGKQALETVQALLPVLCQAHGLTPEQVVAIASNGGGKQALETVQALLPVLCQ
AHGLTPEQVVAIASNIGGKQALETVQALLPVLCQAHGLTPQQVVAIASNNGGRPALE
ND1.2 Right TALE repeat
(SEQ ID NO. 110)
LTPEQVVAIASNGGGKQALETVQALLPVLCQAHGLTPQQVVAIASNNGGKQALETVQR
LLPVLCQAHGLTPQQVVAIASNIGGKQALETVQRLLPVLCQAHGLTPEQVVAIASNNG
GKQALETVQALLPVLCQAHGLTPEQVVAIASNGGGKQALETVQALLPVLCQAHGLTPE
QVVAIASNIGGKQALETVQALLPVLCQAHGLTPEQVVAIASNIGGKQALETVQRLLPVL
CQAHGLTPEQVVAIASNIGGKQALETVQALLPVLCQAHGLTPEQVVAIASHDGGKQAL
ETVQRLLPVLCQAHGLTPEQVVAIASNNGGKQALETVQALLPVLCQAHGLTPEQVVAI
ASNNGGKQALETVQALLPVLCQAHGLTPQQVVAIASHDGGRPALE
ND6.2 Left TALE repeat (TALE-L2)
(SEQ ID NO. 111)
LTPEQVVAIASHDGGKQALETVQALLPVLCQAHGLTPQQVVAIASHDGGKQALETVQR
LLPVLCQAHGLTPQQVVAIASHDGGKQALETVQRLLPVLCQAHGLTPEQVVAIASNIG
GKQALETVQALLPVLCQAHGLTPEQVVAIASNGGGKQALETVQALLPVLCQAHGLTPE
QVVAIASNNGGKQALETVQALLPVLCQAHGLTPEQVVAIASHDGGKQALETVQRLLPV
LCQAHGLTPEQVVAIASHDGGKQALETVQALLPVLCQAHGLTPEQVVAIASNGGGKQ
ALETVQRLLPVLCQAHGLTPEQVVAIASHDGGKQALETVQALLPVLCQAHGLTPEQVV
AIASNIGGKQALETVQALLPVLCQAHGLTPEQVVAIASNNGGKQALETVQALLPVLCQ
AHGLTPEQVVAIASNNGGKQALETVQRLLPVLCQAHGLTPQQVVAIASNIGGKQALET
VQRLLPVLCQAHGLTPEQVVAIASNGGGKQALETVQALLPVLCQAHGLTPQQVVAIAS
NIGGRPALE
ND6.2 Right TALE repeat (TALE-R2)
(SEQ ID NO. 112)
LTPEQVVAIASNGGGKQALETVQALLPVLCQAHGLTPQQVVAIASNGGGKQALETVQR
LLPVLCQAHGLTPQQVVAIASNNGGKQALETVQRLLPVLCQAHGLTPEQVVAIASNNG
GKQALETVQALLPVLCQAHGLTPEQVVAIASNIGGKQALETVQALLPVLCQAHGLTPE
QVVAIASNGGGKQALETVQALLPVLCQAHGLTPEQVVAIASNIGGKQALETVQRLLPV
LCQAHGLTPEQVVAIASNGGGKQALETVQALLPVLCQAHGLTPEQVVAIASNIGGKQA
LETVQRLLPVLCQAHGLTPEQVVAIASHDGGKQALETVQALLPVLCQAHGLTPEQVVA
IASNGGGKQALETVQALLPVLCQAHGLTPEQVVAIASNIGGKQALETVQALLPVLCQA
HGLTPEQVVAIASHDGGKQALETVQRLLPVLCQAHGLTPQQVVAIASNIGGKQALETV
QRLLPVLCQAHGLTPEQVVAIASNNGGKQALETVQALLPVLCQAHGLTPQQVVAIASH
DGGRPALE
ND6.2 Left TALE repeat (TALE-L1)
(SEQ ID NO. 185)
LTPDQVVAIASNNGGKQALETVQRLLPVLCQDHGLTPDQVVAIASNIGGKQALETVQR
LLPVLCQDHGLTPDQVVAIASHDGGKQALETVQRLLPVLCQDHGLTPDQVVAIASHDG
GKQALETVQRLLPVLCQDHGLTPDQVVAIASHDGGKQALETVQRLLPVLCQDHGLTP
DQVVAIASHDGGKQALETVQRLLPVLCQDHGLTPDQVVAIASHDGGKQALETVQRLL
PVLCQDHGLTPDQVVAIASNIGGKQALETVQRLLPVLCQDHGLTPDQVVAIASNGGGK
QALETVQRLLPVLCQDHGLTPDQVVAIASNNGGKQALETVQRLLPVLCQDHG
ND6.2 Left TALE repeat (TALE-L3)
(SEQ ID NO. 186)
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASHDGGKQALETVQRLLPVLCQDHGLTP
DQVVAIASNIGGKQALETVQRLLPVLCQDHGLTPDQVVAIASNNGGKQALETVQRLLP
VLCQDHGLTPDQVVAIASNNGGKQALETVQRLLPVLCQDHGLTPDQVVAIASNIGGKQ
ALETVQRLLPVLCQDHGLTPDQVVAIASNGGGKQALETVQRLLPVLCQDHGLTPDQVV
AIASNIGGKQALETVQRLLPVLCQDHGLTPDQVVAIASHDGGKQALETVQRLLPVLCQ
DHGLTPDQVVAIASNGGGKQALETVQRLLPVLCQDHGLTPDQVVAIASHDGGKQALE
TVQRLLPVLCQDHGLTPDQVVAIASHDGGKQALETVQRLLPVLCQDHGLTPDQVVAIA
SNGGGKQALETVQRLLPVLCQDHGLTPDQVVAIASHDGGKQALETVQRLLPVLCQDH
G
ND6.2 Right TALE repeat (TALE-R1)
(SEQ ID NO. 187)
LTPDQVVAIASHDGGKQALETVQRLLPVLCQDHGLTPDQVVAIASNNGGKQALETVQR
LLPVLCQDHGLTPDQVVAIASNIGGKQALETVQRLLPVLCQDHGLTPDQVVAIASNGG
GKQALETVQRLLPVLCQDHGLTPDQVVAIASNNGGKQALETVQRLLPVLCQDHGLTP
DQVVAIASNNGGKQALETVQRLLPVLCQDHGLTPDQVVAIASHDGGKQALETVQRLL
PVLCQDHGLTPDQVVAIASNGGGKQALETVQRLLPVLCQDHGLTPDQVVAIASNIGGK
QALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNNGGKQALETVQRLLPVL
CQDHG
XTEN linker peptide
(SEQ ID NO. 113)
NSGSETPGTSESATPES
48-amino acid linker peptide
(SEQ ID NO. 114)
SGSETPGTSESATPESSGGSSGGSSGSETPGTSESATPESSGGSSGGS
16-amino acid linker peptide
(SEQ ID NO. 115)
SGSETPGTSESATPES
14-amino acid linker peptide
(SEQ ID NO. 116)
SGGGSGGSGGSGGS
11-amino acid linker peptide
(SEQ ID NO. 117)
SGGSGGSGGSS
4-amino acid linker peptide
(SEQ ID NO. 118)
SGGS
yb
(SEQ ID NO. 119)
MMATFSCVCCGTLTTSTYCGKRCERKHVYSETRNKRLELYKKYLLEPQKCALNGIVG
HSCGMPCSIAEEACDQLPIVSRFCGQKHADLYDSLLKRSEQELLLEFLQKKMQELKLS
HIVKMAKLESEVNAIRKSVASSFEDSVGCDDSSSVSK

The amino acid sequences of the vectors or elements involved in FIG. 16A to 16E and FIG. 17A to 17H are as set forth below. Unless otherwise specified in subsequent examples, corresponding fusion proteins may be constructed based on the schematic diagrams of constructs as shown in FIG. 16 A to 16E and FIG. 17A to 17H and the sequences disclosed in the present specification.

OsBADH2-NLS-TALEN_{WT} (FIG. 16A)
(SEQ ID NO. 120)
MDYKDHDGDYKDHDIDYKDDDDKMAPKKKRKVGIHGVPSRMVDLRTLGYSQQQQE
KIKPKVRSTVAQHHEALVGHGFTHAHIVALSQHPAALGTVAVTYQHIITALPEATHEDIV
GVGKQWSGARALEALLTDAGELRGPPLQLDTGQLVKIAKRGGVTAMEAVHASRNALT
GAPLNLTPDQVVAIASNNGGKQALETVQRLLPVLCQDHGLTPDQVVAIASHDGGKQAL
ETVQRLLPVLCQDHGLTPDQVVAIASNGGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASNIGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIASH
DGGKQALETVQRLLPVLCQDHGLTPDQVVAIASNGGGKQALETVQRLLPVLCQDHGL
TPDQVVAIASNGGGKQALETVQRLLPVLCQDHGLTPDQVVAIASNGGGKQALETVQR
LLPVLCQDHGLTPDQVVAIASNNGGKQALETVQRLLPVLCQDHGLTPDQVVAIASNIG
GKQALETVQRLLPVLCQDHGLTPDQVVAIASNNGGKQALETVQRLLPVLCQDHGLTP
DQVVAIASNGGGKQALETVQRLLPVLCQDHGLTPDQVVAIASNIGGKQALESIVAQLSR
PDPALAALTNDHLVALACLGGRPAMDAVKKGLPHAPELIRRVNRRIGERTSHRVAGSQL
VKSELEEKKSELRHKLKYVPHEYIELIEIARNSTQDRILEMKVMEFFMKVYGYRGKHL
GGSRKPDGAIYTVGSPIDYGVIVDTKAYSGGYNLPIGQADEMQRYVKENQTRNKHINP
NEWWKVYPSSVTEFKFLFVSGHFKGNYKAQLTRLNHKTNCNGAVLSVEELLIGGEMI
KAGTLTLEEVRRKFNNGEINFEGRGSLLTCGDVEENPGPRMDYKDHDGDYKDHDIDY
KDDDDKMAPKKKRKVGIHGVPARMVDLRTLGYSQQQQEKIKPKVRSTVAQHHEALV
GHGFTHAHIVALSQHPAALGTVAVTYQHIITALPEATHEDIVGVGKQWSGARALEALLT
DAGELRGPPLQLDTGQLVKIAKRGGVTAMEAVHASRNALTGAPLNLTPDQVVAIASNN
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNGGGKQALETVQRL
LPVLCQDHGLTPDQVVAIASNGGGKQALETVQRLLPVLCQDHGLTPDQVVAIASNGGG
KQALETVQRLLPVLCQDHGLTPDQVVAIASNGGGKQALETVQRLLPVLCQDHGLTPD
QVVAIASNNGGKQALETVQRLLPVLCQDHGLTPDQVVAIASNGGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNIGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALESIVAQLSRPDPALAALTNDHLVALA
CLGGRPAMDAVKKGLPHAPELIRRVNRRIGERTSHRVAGSQLVKSELEEKKSELRHKLK
YVPHEYIELIEIARNSTQDRILEMKVMEFFMKVYGYRGKHLGGSRKPDGAIYTVGSPID
YGVIVDTKAYSGGYNLPIGQADEMERYVEENQTRNKHLNPNEWWKVYPSSVTEFKFL
FVSGHFKGNYKAQLTRLNHITNCNGAVLSVEELLIGGEMIKAGTLTLEEVRRKENNGEI
NF
OsBADH2-NLS-TALE-L-FokI-L-T2A-TALE-R-FokI-RD_{450A} (FIG. 16B)
(SEQ ID NO. 121)
MDYKDHDGDYKDHDIDYKDDDDKMAPKKKRKVGIHGVPSRMVDLRTLGYSQQQQE
KIKPKVRSTVAQHHEALVGHGFTHAHIVALSQHPAALGTVAVTYQHIITALPEATHEDIV
GVGKQWSGARALEALLTDAGELRGPPLQLDTGQLVKIAKRGGVTAMEAVHASRNALT
GAPLNLTPDQVVAIASNNGGKQALETVQRLLPVLCQDHGLTPDQVVAIASHDGGKQAL
ETVQRLLPVLCQDHGLTPDQVVAIASNGGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASNIGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIASH
DGGKQALETVQRLLPVLCQDHGLTPDQVVAIASNGGGKQALETVQRLLPVLCQDHGL
TPDQVVAIASNGGGKQALETVQRLLPVLCQDHGLTPDQVVAIASNGGGKQALETVQR
LLPVLCQDHGLTPDQVVAIASNNGGKQALETVQRLLPVLCQDHGLTPDQVVAIASNIG
GKQALETVQRLLPVLCQDHGLTPDQVVAIASNNGGKQALETVQRLLPVLCQDHGLTP
DQVVAIASNGGGKQALETVQRLLPVLCQDHGLTPDQVVAIASNIGGKQALESIVAQLSR
PDPALAALTNDHLVALACLGGRPAMDAVKKGLPHAPELIRRVNRRIGERTSHRVAGSQL
VKSELEEKKSELRHKLKYVPHEYIELIEIARNSTQDRILEMKVMEFFMKVYGYRGKHL
GGSRKPDGAIYTVGSPIDYGVIVDTKAYSGGYNLPIGQADEMQRYVKENQTRNKHINP
NEWWKVYPSSVTEFKFLFVSGHFKGNYKAQLTRLNHKTNCNGAVLSVEELLIGGEMI
KAGTLTLEEVRRKFNNGEINFEGRGSLLTCGDVEENPGPRMDYKDHDGDYKDHDIDY
KDDDDKMAPKKKRKVGIHGVPARMVDLRTLGYSQQQQEKIKPKVRSTVAQHHEALV
GHGFTHAHIVALSQHPAALGTVAVTYQHIITALPEATHEDIVGVGKQWSGARALEALLT
DAGELRGPPLQLDTGQLVKIAKRGGVTAMEAVHASRNALTGAPLNLTPDQVVAIASNN
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNGGGKQALETVQRL
LPVLCQDHGLTPDQVVAIASNGGGKQALETVQRLLPVLCQDHGLTPDQVVAIASNGGG
KQALETVQRLLPVLCQDHGLTPDQVVAIASNGGGKQALETVQRLLPVLCQDHGLTPD
QVVAIASNNGGKQALETVQRLLPVLCQDHGLTPDQVVAIASNGGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNIGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALESIVAQLSRPDPALAALTNDHLVALA
CLGGRPAMDAVKKGLPHAPELIRRVNRRIGERTSHRVAGSQLVKSELEEKKSELRHKLK
YVPHEYIELIEIARNSTQDRILEMKVMEFFMKVYGYRGKHLGGSRKPAGAIYTVGSPID
YGVIVDTKAYSGGYNLPIGQADEMERYVEENQTRNKHLNPNEWWKVYPSSVTEFKFL
FVSGHFKGNYKAQLTRLNHITNCNGAVLSVEELLIGGEMIKAGTLTLEEVRRKFNNGEI
NF
OsBADH2-NLS-TALE-L-FokI-L_{D450A}-T2A-TALE-R-FokI-R (FIG. 16B)
(SEQ ID NO. 122)
MDYKDHDGDYKDHDIDYKDDDDKMAPKKKRKVGIHGVPSRMVDLRTLGYSQQQQE
KIKPKVRSTVAQHHEALVGHGFTHAHIVALSQHPAALGTVAVTYQHIITALPEATHEDIV
GVGKQWSGARALEALLTDAGELRGPPLQLDTGQLVKIAKRGGVTAMEAVHASRNALT
GAPLNLTPDQVVAIASNNGGKQALETVQRLLPVLCQDHGLTPDQVVAIASHDGGKQAL
ETVQRLLPVLCQDHGLTPDQVVAIASNGGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASNIGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIASH
DGGKQALETVQRLLPVLCQDHGLTPDQVVAIASNGGGKQALETVQRLLPVLCQDHGL
TPDQVVAIASNGGGKQALETVQRLLPVLCQDHGLTPDQVVAIASNGGGKQALETVQR
LLPVLCQDHGLTPDQVVAIASNNGGKQALETVQRLLPVLCQDHGLTPDQVVAIASNIG
GKQALETVQRLLPVLCQDHGLTPDQVVAIASNNGGKQALETVQRLLPVLCQDHGLTP
DQVVAIASNGGGKQALETVQRLLPVLCQDHGLTPDQVVAIASNIGGKQALESIVAQLSR
PDPALAALTNDHLVALACLGGRPAMDAVKKGLPHAPELIRRVNRRIGERTSHRVAGSQL
VKSELEEKKSELRHKLKYVPHEYIELIEIARNSTQDRILEMKVMEFFMKVYGYRGKHL
GGSRKPAGAIYTVGSPIDYGVIVDTKAYSGGYNLPIGQADEMQRYVKENQTRNKHINP
NEWWKVYPSSVTEFKFLFVSGHFKGNYKAQLTRLNHKTNCNGAVLSVEELLIGGEMI
KAGTLTLEEVRRKFNNGEINFEGRGSLLTCGDVEENPGPRMDYKDHDGDYKDHDIDY
KDDDDKMAPKKKRKVGIHGVPARMVDLRTLGYSQQQQEKIKPKVRSTVAQHHEALV
GHGFTHAHIVALSQHPAALGTVAVTYQHIITALPEATHEDIVGVGKQWSGARALEALLT
DAGELRGPPLQLDTGQLVKIAKRGGVTAMEAVHASRNALTGAPLNLTPDQVVAIASNN
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNGGGKQALETVQRL
LPVLCQDHGLTPDQVVAIASNGGGKQALETVQRLLPVLCQDHGLTPDQVVAIASNGGG
KQALETVQRLLPVLCQDHGLTPDQVVAIASNGGGKQALETVQRLLPVLCQDHGLTPD
QVVAIASNNGGKQALETVQRLLPVLCQDHGLTPDQVVAIASNGGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNIGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALESIVAQLSRPDPALAALTNDHLVALA
CLGGRPAMDAVKKGLPHAPELIRRVNRRIGERTSHRVAGSQLVKSELEEKKSELRHKLK
YVPHEYIELIEIARNSTQDRILEMKVMEFFMKVYGYRGKHLGGSRKPDGAIYTVGSPID
YGVIVDTKAYSGGYNLPIGQADEMERYVEENQTRNKHLNPNEWWKVYPSSVTEFKFL
FVSGHFKGNYKAQLTRLNHITNCNGAVLSVEELLIGGEMIKAGTLTLEEVRRKENNGEI
NF
NLS-A3A-XTEN-UGI (FIG. 16B)
(SEQ ID NO. 123)
MKRTADGSEFESPKKKRKVMEASPASGPRHLMDPHIFTSNFNNGIGRHKTYLCYEVER
LDNGTSVKMDQHRGFLHNQAKNLLCGFYGRHAELRFLDLVPSLQLDPAQIYRVTWFIS
WSPCFSWGCAGEVRAFLQENTHVRLRIFAARIYDYDPLYKEALQMLRDAGAQVSIMT
YDEFKHCWDTFVDHQGCPFQPWDGLDEHSQALSGRLRAILQNQGNSGSETPGTSESA
TPESTNLSDIIEKETGKQLVIQESILMLPEEVEEVIGNKPESDILVHTAYDESTDENVMLL
TSDAPEYKPWALVIQDSNGENKIKML
NLS-UGI (FIG. 16B)
(SEQ ID NO. 163)
MTNLSDIIEKETGKQLVIQESILMLPEEVEEVIGNKPESDILVHTAYDESTDENVMLLTSD
APEYKPWALVIQDSNGENKIKMLMKRTADGSEFESPKKKRKV
NLS-C57-XTEN-UGI (FIG. 16B)
(SEQ ID NO. 124)
MKRTADGSEFESPKKKRKVLEAVRARLIGEGGGPGAVPEGGDGPPAVPAEEVERLRGE
LPPPVVPGTGQKTHGRWIGPDGRVRAIVSGRDEDAALVHAQLAAKGIPDEPTRNSDVE
QKLAAHMVANGIRHVTLVINHRPCRGFDDSCDTLVPIILPEGCTLTVHGQTDKGMRVR
VRYTGGARPWWSNSGSETPGTSESATPESTNLSDIIEKETGKQLVIQESILMLPEEVEEVI
GNKPESDILVHTAYDESTDENVMLLTSDAPEYKPWALVIQDSNGENKIKML
NLS-rAPOBEC1-XTEN-UGI (FIG. 16B)
(SEQ ID NO. 164)
MKRTADGSEFESPKKKRKVSSETGPVAVDPTLRRRIEPHEFEVFFDPRELRKETCLLYEI
NWGGRHSIWRHTSQNTNKHVEVNFIEKFTTERYFCPNTRCSITWFLSWSPCGECSRAIT
EFLSRYPHVTLFIYIARLYHHADPRNRQGLRDLISSGVTIQIMTEQESGYCWRNFVNYSP
SNEAHWPRYPHLWVRLYVLELYCIILGLPPCLNILRRKQPQLTFFTIALQSCHYQRLPPHI
LWATGLKNSGSETPGTSESATPESTNLSDIIEKETGKQLVIQESILMLPEEVEEVIGNKPES
DILVHTAYDESTDENVMLLTSDAPEYKPWALVIQDSNGENKIKML
TadA8e-NLS (FIG. 16B)
(SEQ ID NO. 166)
MSEVEFSHEYWMRHALTLAKRARDEREVPVGAVLVLNNRVIGEGWNRAIGLHDPTAH
AEIMALRQGGLVMQNYRLIDATLYVTFEPCVMCAGAMIHSRIGRVVFGVRNSKRGAA
GSLMNVLNYPGMNHRVEITEGILADECAALLCDFYRMPRQVFNAQKKAQSSINSGGS
MKRTADGSEFESPKKKRKV
mExoI-NLS (FIG. 16B)
(SEQ ID NO. 125)
MGIQGLLQFIQEASEPVNVKKYKGQAVAVDTYCWLHKGAIACAEKLAKGEPTDRYVG
FCMKFVNMLLSYGVKPILIFDGCTLPSKKEVERSRRERRQSNLLKGKQLLREGKVSEA
RDCFARSINITHAMAHKVIKAARALGVDCLVAPYEADAQLAYLNKAGIVQAVITEDSD
LLAFGCKKVILKMDQFGNGLEVDQARLGMCKQLGDVFTEEKFRYMCILSGCDYLASL
RGIGLAKACKVLRLANNPDIVKVIKKIGHYLRMNITVPEDYITGFIRANNTFLYQLVFDP
IQRKLVPLNAYGDDVNPETLTYAGQYVGDSVALQIALGNRDVNTFEQIDDYSPDTMPA
HSRSHSWNEKAGQKPPGTNSIWHKNYCPRLEVNSVSHAPQLKEKPSTLGLKQVISTKG
LNLPRKSCVLKRPRNEALAEDDLLSQYSSVSKKIKENGCGDGTSPNSSKMSKSCPDSGT
AHKTDAHTPSKMRNKFATFLQRRNEESGAVVVPGTRSRFFCSSQDFDNFIPKKESGQPL
NETVATGKATTSLLGALDCPDTEGHKPVDANGTHNLSSQIPGNAAVSPEDEAQSSETSK
LLGAMSPPSLGTLRSCFSWSGTLREFSRTPSPSASTTLQQFRRKSDPPACLPEASAVVTD
RCDSKSEMLGETSQPLHELGCSSRSQESMDSSCGLNTSSLSQPSSRDSGSEESDCNNKS
LDNQGEQNSKQHLPHFSKKDGLRRNKVPGLCRSSSMDSFSTTKIKPLVPARVSGLSKKS
GSMQTRKHHDVENKPGLQTKISELWKNFGFKKDSEKLPSCKKPLSPVKDNIQLTPETE
DEIFNKPECVRAQRAIFHMKRTADGSEFESPKKKRKV
Trex2-NLS (FIG. 16B)
(SEQ ID NO. 126)
MSEPPRAETFVFLDLEATGLPNMDPEIAEISLFAVHRSSLENPERDDSGSLVLPRVLDKLT
LCMCPERPFTAKASEITGLSSESLMHCGKAGFNGAVVRTLQGFLSRQEGPICLVAHNGF
DYDFPLLCTELQRLGAHLPQDTVCLDTLPALRGLDRAHSHGTRAQGRKSYSLASLFHR
YFQAEPSAAHSAEGDVHTLLLIFLHRAPELLAWADEQARSWAHIEPMYVPPDGPSLEA
MKRTADGSEFESPKKKRKV
OsBADH2-NLS-A3A-TALE-L-FokI-L-T2A-TALE-R-FokI-RD450A (FIG. 16C)
(SEQ ID NO. 127)
MDYKDHDGDYKDHDIDYKDDDDKMAPKKKRKVEASPASGPRHLMDPHIFTSNFNNG
IGRHKTYLCYEVERLDNGTSVKMDQHRGFLHNQAKNLLCGFYGRHAELRFLDLVPSL
QLDPAQIYRVTWFISWSPCFSWGCAGEVRAFLQENTHVRLRIFAARIYDYDPLYKEALQ
MLRDAGAQVSIMTYDEFKHCWDTFVDHQGCPFQPWDGLDEHSQALSGRLRAILQNQ
GNSGSETPGTSESATPESSGGSSGGSSGSETPGTSESATPESSGGSSGGSGIHGVPSRMVD
LRTLGYSQQQQEKIKPKVRSTVAQHHEALVGHGFTHAHIVALSQHPAALGTVAVTYQHI
ITALPEATHEDIVGVGKQWSGARALEALLTDAGELRGPPLQLDTGQLVKIAKRGGVTA
MEAVHASRNALTGAPLNLTPDQVVAIASNNGGKQALETVQRLLPVLCQDHGLTPDQV
VAIASHDGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASNNGGKQAL
ETVQRLLPVLCQDHGLTPDQVVAIASNIGGKQALETVQRLLPVLCQDHGLTPDQVVAI
ASNGGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNGGGKQALET
VQRLLPVLCQDHGLTPDQVVAIASNGGGKQALETVQRLLPVLCQDHGLTPDQVVAIAS
NGGGKQALETVQRLLPVLCQDHGLTPDQVVAIASNNGGKQALETVQRLLPVLCQDHG
LTPDQVVAIASNIGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASNIG
GKQALESIVAQLSRPDPALAALTNDHLVALACLGGRPAMDAVKKGLPHAPELIRRVNR
RIGERTSHRVAGSQLVKSELEEKKSELRHKLKYVPHEYIELIEIARNSTQDRILEMKVME
FFMKVYGYRGKHLGGSRKPDGAIYTVGSPIDYGVIVDTKAYSGGYNLPIGQADEMQR
YVKENQTRNKHINPNEWWKVYPSSVTEFKFLFVSGHFKGNYKAQLTRLNHKTNCNGA
VLSVEELLIGGEMIKAGTLTLEEVRRKFNNGEINFEGRGSLLTCGDVEENPGPRMDYKD
HDGDYKDHDIDYKDDDDKMAPKKKRKVGIHGVPARMVDLRTLGYSQQQQEKIKPKV
RSTVAQHHEALVGHGFTHAHIVALSQHPAALGTVAVTYQHIITALPEATHEDIVGVGKQ
WSGARALEALLTDAGELRGPPLQLDTGQLVKIAKRGGVTAMEAVHASRNALTGAPLN
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASNGGGKQALETVQRLLPVLCQDHGLTP
DQVVAIASNGGGKQALETVQRLLPVLCQDHGLTPDQVVAIASNGGGKQALETVQRLL
PVLCQDHGLTPDQVVAIASNNGGKQALETVQRLLPVLCQDHGLTPDQVVAIASNGGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNIGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNGGGKQALESIVAQLSRPDPAL
AALTNDHLVALACLGGRPAMDAVKKGLPHAPELIRRVNRRIGERTSHRVAGSQLVKSEL
EEKKSELRHKLKYVPHEYIELIEIARNSTQDRILEMKVMEFFMKVYGYRGKHLGGSRK
PAGAIYTVGSPIDYGVIVDTKAYSGGYNLPIGQADEMERYVEENQTRNKHLNPNEWW
KVYPSSVTEFKFLFVSGHFKGNYKAQLTRLNHITNCNGAVLSVEELLIGGEMIKAGTLT
LEEVRRKFNNGEINF
OsBADH2-NLS-A3A-TALE-L-FokI-L_{D450A}-T2A-TALE-R-FokI-R (FIG. 16C)
(SEQ ID NO. 128)
MDYKDHDGDYKDHDIDYKDDDDKMAPKKKRKVEASPASGPRHLMDPHIFTSNFNNG
IGRHKTYLCYEVERLDNGTSVKMDQHRGFLHNQAKNLLCGFYGRHAELRFLDLVPSL
QLDPAQIYRVTWFISWSPCFSWGCAGEVRAFLQENTHVRLRIFAARIYDYDPLYKEALQ
MLRDAGAQVSIMTYDEFKHCWDTFVDHQGCPFQPWDGLDEHSQALSGRLRAILQNQ
GNSGSETPGTSESATPESSGGSSGGSSGSETPGTSESATPESSGGSSGGSGIHGVPSRMVD
LRTLGYSQQQQEKIKPKVRSTVAQHHEALVGHGFTHAHIVALSQHPAALGTVAVTYQHI
ITALPEATHEDIVGVGKQWSGARALEALLTDAGELRGPPLQLDTGQLVKIAKRGGVTA
MEAVHASRNALTGAPLNLTPDQVVAIASNNGGKQALETVQRLLPVLCQDHGLTPDQV
VAIASHDGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASNNGGKQAL
ETVQRLLPVLCQDHGLTPDQVVAIASNIGGKQALETVQRLLPVLCQDHGLTPDQVVAI
ASNGGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNGGGKQALET
VQRLLPVLCQDHGLTPDQVVAIASNGGGKQALETVQRLLPVLCQDHGLTPDQVVAIAS
NGGGKQALETVQRLLPVLCQDHGLTPDQVVAIASNNGGKQALETVQRLLPVLCQDHG
LTPDQVVAIASNIGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASNIG
GKQALESIVAQLSRPDPALAALTNDHLVALACLGGRPAMDAVKKGLPHAPELIRRVNR
RIGERTSHRVAGSQLVKSELEEKKSELRHKLKYVPHEYIELIEIARNSTQDRILEMKVME
FFMKVYGYRGKHLGGSRKPAGAIYTVGSPIDYGVIVDTKAYSGGYNLPIGQADEMQR
YVKENQTRNKHINPNEWWKVYPSSVTEFKFLFVSGHFKGNYKAQLTRLNHKTNCNGA
VLSVEELLIGGEMIKAGTLTLEEVRRKFNNGEINFEGRGSLLTCGDVEENPGPRMDYKD
HDGDYKDHDIDYKDDDDKMAPKKKRKVGIHGVPARMVDLRTLGYSQQQQEKIKPKV
RSTVAQHHEALVGHGFTHAHIVALSQHPAALGTVAVTYQHIITALPEATHEDIVGVGKQ
WSGARALEALLTDAGELRGPPLQLDTGQLVKIAKRGGVTAMEAVHASRNALTGAPLN
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASNGGGKQALETVQRLLPVLCQDHGLTP
DQVVAIASNGGGKQALETVQRLLPVLCQDHGLTPDQVVAIASNGGGKQALETVQRLL
PVLCQDHGLTPDQVVAIASNNGGKQALETVQRLLPVLCQDHGLTPDQVVAIASNGGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNIGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNGGGKQALESIVAQLSRPDPAL
AALTNDHLVALACLGGRPAMDAVKKGLPHAPELIRRVNRRIGERTSHRVAGSQLVKSEL
EEKKSELRHKLKYVPHEYIELIEIARNSTQDRILEMKVMEFFMKVYGYRGKHLGGSRK
PDGAIYTVGSPIDYGVIVDTKAYSGGYNLPIGQADEMERYVEENQTRNKHLNPNEWW
KVYPSSVTEFKFLFVSGHFKGNYKAQLTRLNHITNCNGAVLSVEELLIGGEMIKAGTLT
LEEVRRKFNNGEINF
mExoI-NLS (FIG. 16C)
(SEQ ID NO. 129)
MGIQGLLQFIQEASEPVNVKKYKGQAVAVDTYCWLHKGAIACAEKLAKGEPTDRYVG
FCMKFVNMLLSYGVKPILIFDGCTLPSKKEVERSRRERRQSNLLKGKQLLREGKVSEA
RDCFARSINITHAMAHKVIKAARALGVDCLVAPYEADAQLAYLNKAGIVQAVITEDSD
LLAFGCKKVILKMDQFGNGLEVDQARLGMCKQLGDVFTEEKFRYMCILSGCDYLASL
RGIGLAKACKVLRLANNPDIVKVIKKIGHYLRMNITVPEDYITGFIRANNTFLYQLVFDP
IQRKLVPLNAYGDDVNPETLTYAGQYVGDSVALQIALGNRDVNTFEQIDDYSPDTMPA
HSRSHSWNEKAGQKPPGTNSIWHKNYCPRLEVNSVSHAPQLKEKPSTLGLKQVISTKG
LNLPRKSCVLKRPRNEALAEDDLLSQYSSVSKKIKENGCGDGTSPNSSKMSKSCPDSGT
AHKTDAHTPSKMRNKFATFLQRRNEESGAVVVPGTRSRFFCSSQDFDNFIPKKESGQPL
NETVATGKATTSLLGALDCPDTEGHKPVDANGTHNLSSQIPGNAAVSPEDEAQSSETSK
LLGAMSPPSLGTLRSCFSWSGTLREFSRTPSPSASTTLQQFRRKSDPPACLPEASAVVTD
RCDSKSEMLGETSQPLHELGCSSRSQESMDSSCGLNTSSLSQPSSRDSGSEESDCNNKS
LDNQGEQNSKQHLPHFSKKDGLRRNKVPGLCRSSSMDSFSTTKIKPLVPARVSGLSKKS
GSMQTRKHHDVENKPGLQTKISELWKNFGFKKDSEKLPSCKKPLSPVKDNIQLTPETE
DEIFNKPECVRAQRAIFHMKRTADGSEFESPKKKRKV
Trex2-NLS (FIG. 16C)
(SEQ ID NO. 130)
MSEPPRAETFVFLDLEATGLPNMDPEIAEISLFAVHRSSLENPERDDSGSLVLPRVLDKLT
LCMCPERPFTAKASEITGLSSESLMHCGKAGFNGAVVRTLQGFLSRQEGPICLVAHNGF
DYDFPLLCTELQRLGAHLPQDTVCLDTLPALRGLDRAHSHGTRAQGRKSYSLASLFHR
YFQAEPSAAHSAEGDVHTLLLIFLHRAPELLAWADEQARSWAHIEPMYVPPDGPSLEA
MKRTADGSEFESPKKKRKV
UGI-NLS (FIG. 16C)
(SEQ ID NO. 131)
MKRTADGSEFESPKKKRKVTNLSDIIEKETGKQLVIQESILMLPEEVEEVIGNKPESDILV
HTAYDESTDENVMLLTSDAPEYKPWALVIQDSNGENKIKML
OsBADH2-NLS-A3A-TALE-L-FokI-L-T2A-TALE-R-FokI-R_{D450A}-UGI (FIG. 16D)
(SEQ ID NO. 132)
MDYKDHDGDYKDHDIDYKDDDDKMAPKKKRKVEASPASGPRHLMDPHIFTSNFNNG
IGRHKTYLCYEVERLDNGTSVKMDQHRGFLHNQAKNLLCGFYGRHAELRFLDLVPSL
QLDPAQIYRVTWFISWSPCFSWGCAGEVRAFLQENTHVRLRIFAARIYDYDPLYKEALQ
MLRDAGAQVSIMTYDEFKHCWDTFVDHQGCPFQPWDGLDEHSQALSGRLRAILQNQ
GNSGSETPGTSESATPESSGGSSGGSSGSETPGTSESATPESSGGSSGGSGIHGVPSRMVD
LRTLGYSQQQQEKIKPKVRSTVAQHHEALVGHGFTHAHIVALSQHPAALGTVAVTYQHI
ITALPEATHEDIVGVGKQWSGARALEALLTDAGELRGPPLQLDTGQLVKIAKRGGVTA
MEAVHASRNALTGAPLNLTPDQVVAIASNNGGKQALETVQRLLPVLCQDHGLTPDQV
VAIASHDGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASNNGGKQAL
ETVQRLLPVLCQDHGLTPDQVVAIASNIGGKQALETVQRLLPVLCQDHGLTPDQVVAI
ASNGGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNGGGKQALET
VQRLLPVLCQDHGLTPDQVVAIASNGGGKQALETVQRLLPVLCQDHGLTPDQVVAIAS
NGGGKQALETVQRLLPVLCQDHGLTPDQVVAIASNNGGKQALETVQRLLPVLCQDHG
LTPDQVVAIASNIGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASNIG
GKQALESIVAQLSRPDPALAALTNDHLVALACLGGRPAMDAVKKGLPHAPELIRRVNR
RIGERTSHRVAGSQLVKSELEEKKSELRHKLKYVPHEYIELIEIARNSTQDRILEMKVME
FFMKVYGYRGKHLGGSRKPDGAIYTVGSPIDYGVIVDTKAYSGGYNLPIGQADEMQR
YVKENQTRNKHINPNEWWKVYPSSVTEFKFLFVSGHFKGNYKAQLTRLNHKTNCNGA
VLSVEELLIGGEMIKAGTLTLEEVRRKFNNGEINFEGRGSLLTCGDVEENPGPRMDYKD
HDGDYKDHDIDYKDDDDKMAPKKKRKVGIHGVPARMVDLRTLGYSQQQQEKIKPKV
RSTVAQHHEALVGHGFTHAHIVALSQHPAALGTVAVTYQHIITALPEATHEDIVGVGKQ
WSGARALEALLTDAGELRGPPLQLDTGQLVKIAKRGGVTAMEAVHASRNALTGAPLN
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASNGGGKQALETVQRLLPVLCQDHGLTP
DQVVAIASNGGGKQALETVQRLLPVLCQDHGLTPDQVVAIASNGGGKQALETVQRLL
PVLCQDHGLTPDQVVAIASNNGGKQALETVQRLLPVLCQDHGLTPDQVVAIASNGGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNIGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNGGGKQALESIVAQLSRPDPAL
AALTNDHLVALACLGGRPAMDAVKKGLPHAPELIRRVNRRIGERTSHRVAGSQLVKSEL
EEKKSELRHKLKYVPHEYIELIEIARNSTQDRILEMKVMEFFMKVYGYRGKHLGGSRK
PAGAIYTVGSPIDYGVIVDTKAYSGGYNLPIGQADEMERYVEENQTRNKHLNPNEWW
KVYPSSVTEFKFLFVSGHFKGNYKAQLTRLNHITNCNGAVLSVEELLIGGEMIKAGTLT
LEEVRRKFNNGEINFSGGSGGSGGSTNLSDIIEKETGKQLVIQESILMLPEEVEEVIGNKP
ESDILVHTAYDESTDENVMLLTSDAPEYKPWALVIQDSNGENKIKML
OsBADH2-NLS-A3A-TALE-L-FokI-L_{D450A}-T2A-TALE-R-FokI-R-UGI (FIG. 16D)
(SEQ ID NO. 133)
MDYKDHDGDYKDHDIDYKDDDDKMAPKKKRKVEASPASGPRHLMDPHIFTSNFNNG
IGRHKTYLCYEVERLDNGTSVKMDQHRGFLHNQAKNLLCGFYGRHAELRFLDLVPSL
QLDPAQIYRVTWFISWSPCFSWGCAGEVRAFLQENTHVRLRIFAARIYDYDPLYKEALQ
MLRDAGAQVSIMTYDEFKHCWDTFVDHQGCPFQPWDGLDEHSQALSGRLRAILQNQ
GNSGSETPGTSESATPESSGGSSGGSSGSETPGTSESATPESSGGSSGGSGIHGVPSRMVD
LRTLGYSQQQQEKIKPKVRSTVAQHHEALVGHGFTHAHIVALSQHPAALGTVAVTYQHI
ITALPEATHEDIVGVGKQWSGARALEALLTDAGELRGPPLQLDTGQLVKIAKRGGVTA
MEAVHASRNALTGAPLNLTPDQVVAIASNNGGKQALETVQRLLPVLCQDHGLTPDQV
VAIASHDGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASNNGGKQAL
ETVQRLLPVLCQDHGLTPDQVVAIASNIGGKQALETVQRLLPVLCQDHGLTPDQVVAI
ASNGGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNGGGKQALET
VQRLLPVLCQDHGLTPDQVVAIASNGGGKQALETVQRLLPVLCQDHGLTPDQVVAIAS
NGGGKQALETVQRLLPVLCQDHGLTPDQVVAIASNNGGKQALETVQRLLPVLCQDHG
LTPDQVVAIASNIGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASNIG
GKQALESIVAQLSRPDPALAALTNDHLVALACLGGRPAMDAVKKGLPHAPELIRRVNR
RIGERTSHRVAGSQLVKSELEEKKSELRHKLKYVPHEYIELIEIARNSTQDRILEMKVME
FFMKVYGYRGKHLGGSRKPAGAIYTVGSPIDYGVIVDTKAYSGGYNLPIGQADEMQR
YVKENQTRNKHINPNEWWKVYPSSVTEFKFLFVSGHFKGNYKAQLTRLNHKTNCNGA
VLSVEELLIGGEMIKAGTLTLEEVRRKFNNGEINFEGRGSLLTCGDVEENPGPRMDYKD
HDGDYKDHDIDYKDDDDKMAPKKKRKVGIHGVPARMVDLRTLGYSQQQQEKIKPKV
RSTVAQHHEALVGHGFTHAHIVALSQHPAALGTVAVTYQHIITALPEATHEDIVGVGKQ
WSGARALEALLTDAGELRGPPLQLDTGQLVKIAKRGGVTAMEAVHASRNALTGAPLN
LTPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASNGG
GKQALETVQRLLPVLCQDHGLTPDQVVAIASNGGGKQALETVQRLLPVLCQDHGLTP
DQVVAIASNGGGKQALETVQRLLPVLCQDHGLTPDQVVAIASNGGGKQALETVQRLL
PVLCQDHGLTPDQVVAIASNNGGKQALETVQRLLPVLCQDHGLTPDQVVAIASNGGG
KQALETVQRLLPVLCQDHGLTPDQVVAIASHDGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNIGGKQALETVQRLLPVLCQDHGLTPDQVVAIASNNGGKQA
LETVQRLLPVLCQDHGLTPDQVVAIASNNGGKQALETVQRLLPVLCQDHGLTPDQVVA
IASNIGGKQALETVQRLLPVLCQDHGLTPDQVVAIASNGGGKQALESIVAQLSRPDPAL
AALTNDHLVALACLGGRPAMDAVKKGLPHAPELIRRVNRRIGERTSHRVAGSQLVKSEL
EEKKSELRHKLKYVPHEYIELIEIARNSTQDRILEMKVMEFFMKVYGYRGKHLGGSRK
PDGAIYTVGSPIDYGVIVDTKAYSGGYNLPIGQADEMERYVEENQTRNKHLNPNEWW
KVYPSSVTEFKFLFVSGHFKGNYKAQLTRLNHITNCNGAVLSVEELLIGGEMIKAGTLT
LEEVRRKFNNGEINFSGGSGGSGGSTNLSDIIEKETGKQLVIQESILMLPEEVEEVIGNKP
ESDILVHTAYDESTDENVMLLTSDAPEYKPWALVIQDSNGENKIKML
mExoI-NLS (FIG. 16D)
(SEQ ID NO. 134)
MGIQGLLQFIQEASEPVNVKKYKGQAVAVDTYCWLHKGAIACAEKLAKGEPTDRYVG
FCMKFVNMLLSYGVKPILIFDGCTLPSKKEVERSRRERRQSNLLKGKQLLREGKVSEA
RDCFARSINITHAMAHKVIKAARALGVDCLVAPYEADAQLAYLNKAGIVQAVITEDSD
LLAFGCKKVILKMDQFGNGLEVDQARLGMCKQLGDVFTEEKFRYMCILSGCDYLASL
RGIGLAKACKVLRLANNPDIVKVIKKIGHYLRMNITVPEDYITGFIRANNTFLYQLVFDP
IQRKLVPLNAYGDDVNPETLTYAGQYVGDSVALQIALGNRDVNTFEQIDDYSPDTMPA
HSRSHSWNEKAGQKPPGTNSIWHKNYCPRLEVNSVSHAPQLKEKPSTLGLKQVISTKG
LNLPRKSCVLKRPRNEALAEDDLLSQYSSVSKKIKENGCGDGTSPNSSKMSKSCPDSGT
AHKTDAHTPSKMRNKFATFLQRRNEESGAVVVPGTRSRFFCSSQDFDNFIPKKESGQPL
NETVATGKATTSLLGALDCPDTEGHKPVDANGTHNLSSQIPGNAAVSPEDEAQSSETSK
LLGAMSPPSLGTLRSCFSWSGTLREFSRTPSPSASTTLQQFRRKSDPPACLPEASAVVTD
RCDSKSEMLGETSQPLHELGCSSRSQESMDSSCGLNTSSLSQPSSRDSGSEESDCNNKS
LDNQGEQNSKQHLPHFSKKDGLRRNKVPGLCRSSSMDSFSTTKIKPLVPARVSGLSKKS
GSMQTRKHHDVENKPGLQTKISELWKNFGFKKDSEKLPSCKKPLSPVKDNIQLTPETE
DEIFNKPECVRAQRAIFHMKRTADGSEFESPKKKRKV
Trex2-NLS (FIG. 16D)
(SEQ ID NO. 135)
MSEPPRAETFVFLDLEATGLPNMDPEIAEISLFAVHRSSLENPERDDSGSLVLPRVLDKLT
LCMCPERPFTAKASEITGLSSESLMHCGKAGFNGAVVRTLQGFLSRQEGPICLVAHNGF
DYDFPLLCTELQRLGAHLPQDTVCLDTLPALRGLDRAHSHGTRAQGRKSYSLASLFHR
YFQAEPSAAHSAEGDVHTLLLIFLHRAPELLAWADEQARSWAHIEPMYVPPDGPSLEA
MKRTADGSEFESPKKKRKV
OsBADH2-NLS-A3A-TALE-L-FokI-L-T2A-TALE-R-FokI-R_{D450A}-UGI--mExoI-NLS
(FIG. 16E)
(SEQ ID NO. 136)
cassette1-[MDYKDHDGDYKDHDIDYKDDDDKMAPKKKRKVEASPASGPRHLMDPHIF
TSNFNNGIGRHKTYLCYEVERLDNGTSVKMDQHRGFLHNQAKNLLCGFYGRHAELRF
LDLVPSLQLDPAQIYRVTWFISWSPCFSWGCAGEVRAFLQENTHVRLRIFAARIYDYDP
LYKEALQMLRDAGAQVSIMTYDEFKHCWDTFVDHQGCPFQPWDGLDEHSQALSGRL
RAILQNQGNSGSETPGTSESATPESSGGSSGGSSGSETPGTSESATPESSGGSSGGSGIHG
VPSRMVDLRTLGYSQQQQEKIKPKVRSTVAQHHEALVGHGFTHAHIVALSQHPAALGT
VAVTYQHIITALPEATHEDIVGVGKQWSGARALEALLTDAGELRGPPLQLDTGQLVKIA
KRGGVTAMEAVHASRNALTGAPLNLTPDQVVAIASNNGGKQALETVQRLLPVLCQDH
GLTPDQVVAIASHDGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIASN
NGGKQALETVQRLLPVLCQDHGLTPDQVVAIASNIGGKQALETVQRLLPVLCQDHGLT
PDQVVAIASNGGGKQALETVQRLLPVLCQDHGLTPDQVVAIASNNGGKQALETVQRL
LPVLCQDHGLTPDQVVAIASHDGGKQALETVQRLLPVLCQDHGLTPDQVVAIASNGGG
KQALETVQRLLPVLCQDHGLTPDQVVAIASNGGGKQALETVQRLLPVLCQDHGLTPD
QVVAIASNGGGKQALETVQRLLPVLCQDHGLTPDQVVAIASNNGGKQALETVQRLLP
VLCQDHGLTPDQVVAIASNIGGKQALETVQRLLPVLCQDHGLTPDQVVAIASNNGGKQ
ALETVQRLLPVLCQDHGLTPDQVVAIASNGGGKQALETVQRLLPVLCQDHGLTPDQVV
AIASNIGGKQALESIVAQLSRPDPALAALTNDHLVALACLGGRPAMDAVKKGLPHAPEL
IRRVNRRIGERTSHRVAGSQLVKSELEEKKSELRHKLKYVPHEYIELIEIARNSTQDRILE
MKVMEFFMKVYGYRGKHLGGSRKPDGAIYTVGSPIDYGVIVDTKAYSGGYNLPIGQA
DEMQRYVKENQTRNKHINPNEWWKVYPSSVTEFKFLFVSGHFKGNYKAQLTRLNHK
TNCNGAVLSVEELLIGGEMIKAGTLTLEEVRRKFNNGEINFEGRGSLLTCGDVEENPGP
RMDYKDHDGDYKDHDIDYKDDDDKMAPKKKRKVGIHGVPARMVDLRTLGYSQQQQ
EKIKPKVRSTVAQHHEALVGHGFTHAHIVALSQHPAALGTVAVTYQHIITALPEATHEDI
VGVGKQWSGARALEALLTDAGELRGPPLQLDTGQLVKIAKRGGVTAMEAVHASRNAL
TGAPLNLTPDQVVAIASNNGGKQALETVQRLLPVLCQDHGLTPDQVVAIASHDGGKQA
LETVQRLLPVLCQDHGLTPDQVVAIASHDGGKQALETVQRLLPVLCQDHGLTPDQVVA
IASNGGGKQALETVQRLLPVLCQDHGLTPDQVVAIASNGGGKQALETVQRLLPVLCQ
DHGLTPDQVVAIASNGGGKQALETVQRLLPVLCQDHGLTPDQVVAIASNGGGKQALE
TVQRLLPVLCQDHGLTPDQVVAIASNNGGKQALETVQRLLPVLCQDHGLTPDQVVAIA
SNGGGKQALETVQRLLPVLCQDHGLTPDQVVAIASHDGGKQALETVQRLLPVLCQDH
GLTPDQVVAIASHDGGKQALETVQRLLPVLCQDHGLTPDQVVAIASNIGGKQALETVQ
RLLPVLCQDHGLTPDQVVAIASNIGGKQALETVQRLLPVLCQDHGLTPDQVVAIASNN
GGKQALETVQRLLPVLCQDHGLTPDQVVAIASNNGGKQALETVQRLLPVLCQDHGLT
PDQVVAIASNIGGKQALETVQRLLPVLCQDHGLTPDQVVAIASNGGGKQALESIVAQLS
RPDPALAALTNDHLVALACLGGRPAMDAVKKGLPHAPELIRRVNRRIGERTSHRVAGSQ
LVKSELEEKKSELRHKLKYVPHEYIELIEIARNSTQDRILEMKVMEFFMKVYGYRGKH
LGGSRKPAGAIYTVGSPIDYGVIVDTKAYSGGYNLPIGQADEMERYVEENQTRNKHLN
PNEWWKVYPSSVTEFKFLFVSGHFKGNYKAQLTRLNHITNCNGAVLSVEELLIGGEMI
KAGTLTLEEVRRKFNNGEINFSGGSGGSGGSTNLSDIIEKETGKQLVIQESILMLPEEVEE
VIGNKPESDILVHTAYDESTDENVMLLTSDAPEYKPWALVIQDSNGENKIKML]-cassette
2-[MGIQGLLQFIQEASEPVNVKKYKGQAVAVDTYCWLHKGAIACAEKLAKGEPTDRY
VGFCMKFVNMLLSYGVKPILIFDGCTLPSKKEVERSRRERRQSNLLKGKQLLREGKVS
EARDCFARSINITHAMAHKVIKAARALGVDCLVAPYEADAQLAYLNKAGIVQAVITED
SDLLAFGCKKVILKMDQFGNGLEVDQARLGMCKQLGDVFTEEKFRYMCILSGCDYLA
SLRGIGLAKACKVLRLANNPDIVKVIKKIGHYLRMNITVPEDYITGFIRANNTFLYQLVF
DPIQRKLVPLNAYGDDVNPETLTYAGQYVGDSVALQIALGNRDVNTFEQIDDYSPDTM
PAHSRSHSWNEKAGQKPPGTNSIWHKNYCPRLEVNSVSHAPQLKEKPSTLGLKQVIST
KGLNLPRKSCVLKRPRNEALAEDDLLSQYSSVSKKIKENGCGDGTSPNSSKMSKSCPD
SGTAHKTDAHTPSKMRNKFATFLQRRNEESGAVVVPGTRSRFFCSSQDFDNFIPKKESG
QPLNETVATGKATTSLLGALDCPDTEGHKPVDANGTHNLSSQIPGNAAVSPEDEAQSSE
TSKLLGAMSPPSLGTLRSCFSWSGTLREFSRTPSPSASTTLQQFRRKSDPPACLPEASAV
VTDRCDSKSEMLGETSQPLHELGCSSRSQESMDSSCGLNTSSLSQPSSRDSGSEESDCN
NKSLDNQGEQNSKQHLPHFSKKDGLRRNKVPGLCRSSSMDSFSTTKIKPLVPARVSGLS
KKSGSMQTRKHHDVENKPGLQTKISELWKNFGFKKDSEKLPSCKKPLSPVKDNIQLTP
ETEDEIFNKPECVRAQRAIFHMKRTADGSEFESPKKKRKV]
OsBADH2-NLS-A3A-TALE-L-FokI-L_{D450A}-T2A-TALE-R-FokI-R-UGI--mExoI-NLS
(FIG. 16E)
(SEQ ID NO. 137)
cassette1-[MDYKDHDGDYKDHDIDYKDDDDKMAPKKKRKVEASPASGPRHLMDPHIF
TSNFNNGIGRHKTYLCYEVERLDNGTSVKMDQHRGFLHNQAKNLLCGFYGRHAELRF
LDLVPSLQLDPAQIYRVTWFISWSPCFSWGCAGEVRAFLQENTHVRLRIFAARIYDYDP
LYKEALQMLRDAGAQVSIMTYDEFKHCWDTFVDHQGCPFQPWDGLDEHSQALSGRL
RAILQNQGNSGSETPGTSESATPESSGGSSGGSSGSETPGTSESATPESSGGSSGGSGIHG
VPSRMVDLRTLGYSQQQQEKIKPKVRSTVAQHHEALVGHGFTHAHIVALSQHPAALGT
VAVTYQHIITALPEATHEDIVGVGKQWSGARALEALLTDAGELRGPPLQLDTGQLVKIA
KRGGVTAMEAVHASRNALTGAPLNLTPDQVVAIASNNGGKQALETVQRLLPVLCQDH
GLTPDQVVAIASHDGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIASN
NGGKQALETVQRLLPVLCQDHGLTPDQVVAIASNIGGKQALETVQRLLPVLCQDHGLT
PDQVVAIASNGGGKQALETVQRLLPVLCQDHGLTPDQVVAIASNNGGKQALETVQRL
LPVLCQDHGLTPDQVVAIASHDGGKQALETVQRLLPVLCQDHGLTPDQVVAIASNGGG
KQALETVQRLLPVLCQDHGLTPDQVVAIASNGGGKQALETVQRLLPVLCQDHGLTPD
QVVAIASNGGGKQALETVQRLLPVLCQDHGLTPDQVVAIASNNGGKQALETVQRLLP
VLCQDHGLTPDQVVAIASNIGGKQALETVQRLLPVLCQDHGLTPDQVVAIASNNGGKQ
ALETVQRLLPVLCQDHGLTPDQVVAIASNGGGKQALETVQRLLPVLCQDHGLTPDQVV
AIASNIGGKQALESIVAQLSRPDPALAALTNDHLVALACLGGRPAMDAVKKGLPHAPEL
IRRVNRRIGERTSHRVAGSQLVKSELEEKKSELRHKLKYVPHEYIELIEIARNSTQDRILE
MKVMEFFMKVYGYRGKHLGGSRKPAGAIYTVGSPIDYGVIVDTKAYSGGYNLPIGQA
DEMQRYVKENQTRNKHINPNEWWKVYPSSVTEFKFLFVSGHFKGNYKAQLTRLNHK
TNCNGAVLSVEELLIGGEMIKAGTLTLEEVRRKFNNGEINFEGRGSLLTCGDVEENPGP
RMDYKDHDGDYKDHDIDYKDDDDKMAPKKKRKVGIHGVPARMVDLRTLGYSQQQQ
EKIKPKVRSTVAQHHEALVGHGFTHAHIVALSQHPAALGTVAVTYQHIITALPEATHEDI
VGVGKQWSGARALEALLTDAGELRGPPLQLDTGQLVKIAKRGGVTAMEAVHASRNAL
TGAPLNLTPDQVVAIASNNGGKQALETVQRLLPVLCQDHGLTPDQVVAIASHDGGKQA
LETVQRLLPVLCQDHGLTPDQVVAIASHDGGKQALETVQRLLPVLCQDHGLTPDQVVA
IASNGGGKQALETVQRLLPVLCQDHGLTPDQVVAIASNGGGKQALETVQRLLPVLCQ
DHGLTPDQVVAIASNGGGKQALETVQRLLPVLCQDHGLTPDQVVAIASNGGGKQALE
TVQRLLPVLCQDHGLTPDQVVAIASNNGGKQALETVQRLLPVLCQDHGLTPDQVVAIA
SNGGGKQALETVQRLLPVLCQDHGLTPDQVVAIASHDGGKQALETVQRLLPVLCQDH
GLTPDQVVAIASHDGGKQALETVQRLLPVLCQDHGLTPDQVVAIASNIGGKQALETVQ
RLLPVLCQDHGLTPDQVVAIASNIGGKQALETVQRLLPVLCQDHGLTPDQVVAIASNN
GGKQALETVQRLLPVLCQDHGLTPDQVVAIASNNGGKQALETVQRLLPVLCQDHGLT
PDQVVAIASNIGGKQALETVQRLLPVLCQDHGLTPDQVVAIASNGGGKQALESIVAQLS
RPDPALAALTNDHLVALACLGGRPAMDAVKKGLPHAPELIRRVNRRIGERTSHRVAGSQ
LVKSELEEKKSELRHKLKYVPHEYIELIEIARNSTQDRILEMKVMEFFMKVYGYRGKH
LGGSRKPDGAIYTVGSPIDYGVIVDTKAYSGGYNLPIGQADEMERYVEENQTRNKHLN
PNEWWKVYPSSVTEFKFLFVSGHFKGNYKAQLTRLNHITNCNGAVLSVEELLIGGEMI
KAGTLTLEEVRRKFNNGEINFSGGSGGSGGSTNLSDIIEKETGKQLVIQESILMLPEEVEE
VIGNKPESDILVHTAYDESTDENVMLLTSDAPEYKPWALVIQDSNGENKIKML]-cassette
2-[MGIQGLLQFIQEASEPVNVKKYKGQAVAVDTYCWLHKGAIACAEKLAKGEPTDRY
VGFCMKFVNMLLSYGVKPILIFDGCTLPSKKEVERSRRERRQSNLLKGKQLLREGKVS
EARDCFARSINITHAMAHKVIKAARALGVDCLVAPYEADAQLAYLNKAGIVQAVITED
SDLLAFGCKKVILKMDQFGNGLEVDQARLGMCKQLGDVFTEEKFRYMCILSGCDYLA
SLRGIGLAKACKVLRLANNPDIVKVIKKIGHYLRMNITVPEDYITGFIRANNTFLYQLVF
DPIQRKLVPLNAYGDDVNPETLTYAGQYVGDSVALQIALGNRDVNTFEQIDDYSPDTM
PAHSRSHSWNEKAGQKPPGTNSIWHKNYCPRLEVNSVSHAPQLKEKPSTLGLKQVIST
KGLNLPRKSCVLKRPRNEALAEDDLLSQYSSVSKKIKENGCGDGTSPNSSKMSKSCPD
SGTAHKTDAHTPSKMRNKFATFLQRRNEESGAVVVPGTRSRFFCSSQDFDNFIPKKESG
QPLNETVATGKATTSLLGALDCPDTEGHKPVDANGTHNLSSQIPGNAAVSPEDEAQSSE
TSKLLGAMSPPSLGTLRSCFSWSGTLREFSRTPSPSASTTLQQFRRKSDPPACLPEASAV
VTDRCDSKSEMLGETSQPLHELGCSSRSQESMDSSCGLNTSSLSQPSSRDSGSEESDCN
NKSLDNQGEQNSKQHLPHFSKKDGLRRNKVPGLCRSSSMDSFSTTKIKPLVPARVSGLS
KKSGSMQTRKHHDVENKPGLQTKISELWKNFGFKKDSEKLPSCKKPLSPVKDNIQLTP
ETEDEIFNKPECVRAQRAIFHMKRTADGSEFESPKKKRKV]
ND6-MTS-TALE-L-FokI-L (FIG. 17A)
(SEQ ID NO. 138)
MALSRAVCGTSRQLAPVLGYLGSRQKHSLPDYPYDVPDYAGYPYDVPDYAGYPYDVP
DYAMDIADLRTLGYSQQQQEKIKPKVRSTVAQHHEALVGHGFTHAHIVALSQHPAALG
TVAVKYQDMIAALPEATHEAIVGVGKQWSGARALEALLTVAGELRGPPLQLDTGQLLK
IAKRGGVTAVEAVHAWRNALTGAPLNLTPEQVVAIASNNGGKQALETVQRLLPVLCQA
HGLTPEQVVAIASNIGGKQALETVQRLLPVLCQAHGLTPEQVVAIASHDGGKQALETV
QRLLPVLCQAHGLTPEQVVAIASHDGGKQALETVQRLLPVLCQAHGLTPEQVVAIASH
DGGKQALETVQRLLPVLCQAHGLTPEQVVAIASHDGGKQALETVQRLLPVLCQAHGL
TPEQVVAIASHDGGKQALETVQRLLPVLCQAHGLTPEQVVAIASNIGGKQALETVQRL
LPVLCQAHGLTPEQVVAIASNGGGKQALETVQRLLPVLCQAHGLTPEQVVAIASNGGG
RPALESIVAQLSRPDPALAALTNDHLVALACLGGRPALDAVKKGLGGSQLVKSELEEKK
SELRHKLKYVPHEYIELIEIARNSTQDRILEMKVMEFFMKVYGYRGKHLGGSRKPDGA
IYTVGSPIDYGVIVDTKAYSGGYNLPIGQADEMQRYVKENQTRNKHINPNEWWKVYP
SSVTEFKFLFVSGHFKGNYKAQLTRLNHKTNCNGAVLSVEELLIGGEMIKAGTLTLEEV
RRKFNNGEINF
ND6-MTS-TALE-R-FokI-R_{D450A} (FIG. 17B)
(SEQ ID NO. 139)
MASVLTPLLLRGLTGSARRLPVPRAKIHSLDYKDHDGDYKDHDIDYKDDDDKMDIAD
LRTLGYSQQQQEKIKPKVRSTVAQHHEALVGHGFTHAHIVALSQHPAALGTVAVKYQD
MIAALPEATHEAIVGVGKRGAGARALEALLTVAGELRGPPLQLDTGQLLKIAKRGGVT
AVEAVHAWRNALTGAPLNLTPEQVVAIASNNGGKQALETVQRLLPVLCQAHGLTPEQV
VAIASNIGGKQALETVQRLLPVLCQAHGLTPEQVVAIASNGGGKQALETVQRLLPVLC
QAHGLTPEQVVAIASNNGGKQALETVQRLLPVLCQAHGLTPEQVVAIASNNGGKQALE
TVQRLLPVLCQAHGLTPEQVVAIASHDGGKQALETVQRLLPVLCQAHGLTPEQVVAIA
SNGGGKQALETVQRLLPVLCQAHGLTPEQVVAIASNIGGKQALETVQRLLPVLCQAHG
LTPEQVVAIASNGGGKQALETVQRLLPVLCQAHGLTPEQVVAIASNGGGKQALETVQR
LLPVLCQAHGLTPEQVVAIASNGGGRPALESIVAQLSRPDPALAALTNDHLVALACLGG
RPALDAVKKGLGGSQLVKSELEEKKSELRHKLKYVPHEYIELIEIARNSTQDRILEMKV
MEFFMKVYGYRGKHLGGSRKPAGAIYTVGSPIDYGVIVDTKAYSGGYNLPIGQADEM
ERYVEENQTRNKHLNPNEWWKVYPSSVTEFKFLFVSGHFKGNYKAQLTRLNHITNCN
GAVLSVEELLIGGEMIKAGTLTLEEVRRKFNNGEINF
ND6-MTS-TALE-L-FokI-L_{D450A} (FIG. 17A)
(SEQ ID NO. 140)
MALSRAVCGTSRQLAPVLGYLGSRQKHSLPDYPYDVPDYAGYPYDVPDYAGYPYDVP
DYAMDIADLRTLGYSQQQQEKIKPKVRSTVAQHHEALVGHGFTHAHIVALSQHPAALG
TVAVKYQDMIAALPEATHEAIVGVGKQWSGARALEALLTVAGELRGPPLQLDTGQLLK
IAKRGGVTAVEAVHAWRNALTGAPLNLTPEQVVAIASNNGGKQALETVQRLLPVLCQA
HGLTPEQVVAIASNIGGKQALETVQRLLPVLCQAHGLTPEQVVAIASHDGGKQALETV
QRLLPVLCQAHGLTPEQVVAIASHDGGKQALETVQRLLPVLCQAHGLTPEQVVAIASH
DGGKQALETVQRLLPVLCQAHGLTPEQVVAIASHDGGKQALETVQRLLPVLCQAHGL
TPEQVVAIASHDGGKQALETVQRLLPVLCQAHGLTPEQVVAIASNIGGKQALETVQRL
LPVLCQAHGLTPEQVVAIASNGGGKQALETVQRLLPVLCQAHGLTPEQVVAIASNGGG
RPALESIVAQLSRPDPALAALTNDHLVALACLGGRPALDAVKKGLGGSQLVKSELEEKK
SELRHKLKYVPHEYIELIEIARNSTQDRILEMKVMEFFMKVYGYRGKHLGGSRKPAGA
IYTVGSPIDYGVIVDTKAYSGGYNLPIGQADEMQRYVKENQTRNKHINPNEWWKVYP
SSVTEFKFLFVSGHFKGNYKAQLTRLNHKTNCNGAVLSVEELLIGGEMIKAGTLTLEEV
RRKFNNGEINF
ND6-MTS-TALE-R-FokI-R (FIG. 17B)
(SEQ ID NO. 141)
MASVLTPLLLRGLTGSARRLPVPRAKIHSLDYKDHDGDYKDHDIDYKDDDDKMDIAD
LRTLGYSQQQQEKIKPKVRSTVAQHHEALVGHGFTHAHIVALSQHPAALGTVAVKYQD
MIAALPEATHEAIVGVGKRGAGARALEALLTVAGELRGPPLQLDTGQLLKIAKRGGVT
AVEAVHAWRNALTGAPLNLTPEQVVAIASNNGGKQALETVQRLLPVLCQAHGLTPEQV
VAIASNIGGKQALETVQRLLPVLCQAHGLTPEQVVAIASNGGGKQALETVQRLLPVLC
QAHGLTPEQVVAIASNNGGKQALETVQRLLPVLCQAHGLTPEQVVAIASNNGGKQALE
TVQRLLPVLCQAHGLTPEQVVAIASHDGGKQALETVQRLLPVLCQAHGLTPEQVVAIA
SNGGGKQALETVQRLLPVLCQAHGLTPEQVVAIASNIGGKQALETVQRLLPVLCQAHG
LTPEQVVAIASNGGGKQALETVQRLLPVLCQAHGLTPEQVVAIASNGGGKQALETVQR
LLPVLCQAHGLTPEQVVAIASNGGGRPALESIVAQLSRPDPALAALTNDHLVALACLGG
RPALDAVKKGLGGSQLVKSELEEKKSELRHKLKYVPHEYIELIEIARNSTQDRILEMKV
MEFFMKVYGYRGKHLGGSRKPDGAIYTVGSPIDYGVIVDTKAYSGGYNLPIGQADEM
ERYVEENQTRNKHLNPNEWWKVYPSSVTEFKFLFVSGHFKGNYKAQLTRLNHITNCN
GAVLSVEELLIGGEMIKAGTLTLEEVRRKFNNGEINF
MTS-mExoI (FIG. 17D)
(SEQ ID NO. 142)
MALSRAVCGTSRQLAPVLGYLGSRQKHSLPDMGIQGLLQFIQEASEPVNVKKYKGQAV
AVDTYCWLHKGAIACAEKLAKGEPTDRYVGFCMKFVNMLLSYGVKPILIFDGCTLPSK
KEVERSRRERRQSNLLKGKQLLREGKVSEARDCFARSINITHAMAHKVIKAARALGVD
CLVAPYEADAQLAYLNKAGIVQAVITEDSDLLAFGCKKVILKMDQFGNGLEVDQARL
GMCKQLGDVFTEEKFRYMCILSGCDYLASLRGIGLAKACKVLRLANNPDIVKVIKKIG
HYLRMNITVPEDYITGFIRANNTFLYQLVFDPIQRKLVPLNAYGDDVNPETLTYAGQYV
GDSVALQIALGNRDVNTFEQIDDYSPDTMPAHSRSHSWNEKAGQKPPGTNSIWHKNY
CPRLEVNSVSHAPQLKEKPSTLGLKQVISTKGLNLPRKSCVLKRPRNEALAEDDLLSQ
YSSVSKKIKENGCGDGTSPNSSKMSKSCPDSGTAHKTDAHTPSKMRNKFATFLQRRNE
ESGAVVVPGTRSRFFCSSQDFDNFIPKKESGQPLNETVATGKATTSLLGALDCPDTEGH
KPVDANGTHNLSSQIPGNAAVSPEDEAQSSETSKLLGAMSPPSLGTLRSCFSWSGTLRE
FSRTPSPSASTTLQQFRRKSDPPACLPEASAVVTDRCDSKSEMLGETSQPLHELGCSSRS
QESMDSSCGLNTSSLSQPSSRDSGSEESDCNNKSLDNQGEQNSKQHLPHFSKKDGLRR
NKVPGLCRSSSMDSFSTTKIKPLVPARVSGLSKKSGSMQTRKHHDVENKPGLQTKISEL
WKNFGFKKDSEKLPSCKKPLSPVKDNIQLTPETEDEIFNKPECVRAQRAIFH
MTS-Trex2 (FIG. 17D)
(SEQ ID NO. 143)
MALSRAVCGTSRQLAPVLGYLGSRQKHSLPDMSEPPRAETFVFLDLEATGLPNMDPEI
AEISLFAVHRSSLENPERDDSGSLVLPRVLDKLTLCMCPERPFTAKASEITGLSSESLMHC
GKAGFNGAVVRTLQGFLSRQEGPICLVAHNGFDYDFPLLCTELQRLGAHLPQDTVCLD
TLPALRGLDRAHSHGTRAQGRKSYSLASLFHRYFQAEPSAAHSAEGDVHTLLLIFLHR
APELLAWADEQARSWAHIEPMYVPPDGPSLEA
MTS-A3A (FIG. 17C)
(SEQ ID NO. 144)
MALSRAVCGTSRQLAPVLGYLGSRQKHSLPDEASPASGPRHLMDPHIFTSNFNNGIGR
HKTYLCYEVERLDNGTSVKMDQHRGFLHNQAKNLLCGFYGRHAELRFLDLVPSLQLD
PAQIYRVTWFISWSPCFSWGCAGEVRAFLQENTHVRLRIFAARIYDYDPLYKEALQML
RDAGAQVSIMTYDEFKHCWDTFVDHQGCPFQPWDGLDEHSQALSGRLRAILQNQGN
MTS-C57/Sdd7 (FIG. 17C)
(SEQ ID NO. 145)
MALSRAVCGTSRQLAPVLGYLGSRQKHSLPDLEAVRARLIGEGGGPGAVPEGGDGPPA
VPAEEVERLRGELPPPVVPGTGQKTHGRWIGPDGRVRAIVSGRDEDAALVHAQLAAK
GIPDEPTRNSDVEQKLAAHMVANGIRHVTLVINHRPCRGFDDSCDTLVPIILPEGCTLTV
HGQTDKGMRVRVRYTGGARPWWS
MTS-UGI (FIG. 17E)
(SEQ ID NO. 146)
MALSRAVCGTSRQLAPVLGYLGSRQKHSLPDGSSGGSTNLSDIIEKETGKQLVIQESIL
MLPEEVEEVIGNKPESDILVHTAYDESTDENVMLLTSDAPEYKPWALVIQDSNGENKIK
ML
ND6-MTS-A3A-TALE-L-FokI-L (FIG. 17F)
(SEQ ID NO. 147)
MALSRAVCGTSRQLAPVLGYLGSRQKHSLPDYPYDVPDYAGYPYDVPDYAGYPYDVP
DYAEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAFL
QENTHVRLRIFAARIYDYDPLYKEALQMLRDAGAQVSIMTYDEFKHCWDTFVDHQGC
PFQPWDGLDEHSQALSGRLRAILQNQGNSGSETPGTSESATPESSGGSSGGSSGSETPGT
SESATPESSGGSSGGSMDIADLRTLGYSQQQQEKIKPKVRSTVAQHHEALVGHGFTHA
HIVALSQHPAALGTVAVKYQDMIAALPEATHEAIVGVGKQWSGARALEALLTVAGELR
GPPLQLDTGQLLKIAKRGGVTAVEAVHAWRNALTGAPLNLTPEQVVAIASNNGGKQAL
ETVQRLLPVLCQAHGLTPEQVVAIASNIGGKQALETVQRLLPVLCQAHGLTPEQVVAIA
SHDGGKQALETVQRLLPVLCQAHGLTPEQVVAIASHDGGKQALETVQRLLPVLCQAH
GLTPEQVVAIASHDGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIG
GKQALETVQRLLPVLCQAHGLTPEQVVAIASNGGGKQALETVQRLLPVLCQAHGLTPE
QVVAIASNGGGRPALESIVAQLSRPDPALAALTNDHLVALACLGGRPALDAVKKGLGGS
QLVKSELEEKKSELRHKLKYVPHEYIELIEIARNSTQDRILEMKVMEFFMKVYGYRGK
HLGGSRKPDGAIYTVGSPIDYGVIVDTKAYSGGYNLPIGQADEMQRYVKENQTRNKHI
NPNEWWKVYPSSVTEFKFLFVSGHFKGNYKAQLTRLNHKTNCNGAVLSVEELLIGGE
MIKAGTLTLEEVRRKFNNGEINF
ND6-MTS-Trex2-TALE-R-FokI-R_{D450A} (FIG. 17G)
(SEQ ID NO. 148)
MASVLTPLLLRGLTGSARRLPVPRAKIHSLDYKDHDGDYKDHDIDYKDDDDKMSEPP
RAETFVFLDLEATGLPNMDPEIAEISLFAVHRSSLENPERDDSGSLVLPRVLDKLTLCMC
PERPFTAKASEITGLSSESLMHCGKAGFNGAVVRTLQGFLSRQEGPICLVAHNGFDYDF
PLLCTELQRLGAHLPQDTVCLDTLPALRGLDRAHSHGTRAQGRKSYSLASLFHRYFQA
EPSAAHSAEGDVHTLLLIFLHRAPELLAWADEQARSWAHIEPMYVPPDGPSLEASGSET
PGTSESATPESSGGSSGGSSGSETPGTSESATPESSGGSSGGSMDIADLRTLGYSQQQQE
KIKPKVRSTVAQHHEALVGHGFTHAHIVALSQHPAALGTVAVKYQDMIAALPEATHEAI
VGVGKRGAGARALEALLTVAGELRGPPLQLDTGQLLKIAKRGGVTAVEAVHAWRNAL
TGAPLNLTPEQVVAIASNNGGKQALETVQRLLPVLCQAHGLTPEQVVAIASNIGGKQAL
ETVQRLLPVLCQAHGLTPEQVVAIASNGGGKQALETVQRLLPVLCQAHGLTPEQVVAI
ASNNGGKQALETVQRLLPVLCQAHGLTPEQVVAIASNNGGKQALETVQRLLPVLCQA
HGLTPEQVVAIASHDGGKQALETVQRLLPVLCQAHGLTPEQVVAIASNGGGKQALETV
QRLLPVLCQAHGLTPEQVVAIASNIGGKQALETVQRLLPVLCQAHGLTPEQVVAIASNG
GGKQALETVQRLLPVLCQAHGLTPEQVVAIASNGGGKQALETVQRLLPVLCQAHGLTP
EQVVAIASNGGGRPALESIVAQLSRPDPALAALTNDHLVALACLGGRPALDAVKKGLGG
SQLVKSELEEKKSELRHKLKYVPHEYIELIEIARNSTQDRILEMKVMEFFMKVYGYRG
KHLGGSRKPAGAIYTVGSPIDYGVIVDTKAYSGGYNLPIGQADEMERYVEENQTRNKH
LNPNEWWKVYPSSVTEFKFLFVSGHFKGNYKAQLTRLNHITNCNGAVLSVEELLIGGE
MIKAGTLTLEEVRRKFNNGEINF
ND6-MTS-UGI-Trex2-TALE-R-FokI-R_{D450A} (FIG. 17H)
(SEQ ID NO. 149)
MASVLTPLLLRGLTGSARRLPVPRAKIHSLDYKDHDGDYKDHDIDYKDDDDKTNLSDI
IEKETGKQLVIQESILMLPEEVEEVIGNKPESDILVHTAYDESTDENVMLLTSDAPEYKP
WALVIQDSNGENKIKMLSGGSGGSGGSMSEPPRAETFVFLDLEATGLPNMDPEIAEISLF
AVHRSSLENPERDDSGSLVLPRVLDKLTLCMCPERPFTAKASEITGLSSESLMHCGKAG
FNGAVVRTLQGFLSRQEGPICLVAHNGFDYDFPLLCTELQRLGAHLPQDTVCLDTLPAL
RGLDRAHSHGTRAQGRKSYSLASLFHRYFQAEPSAAHSAEGDVHTLLLIFLHRAPELL
AWADEQARSWAHIEPMYVPPDGPSLEASGSETPGTSESATPESSGGSSGGSSGSETPGTS
ESATPESSGGSSGGSMDIADLRTLGYSQQQQEKIKPKVRSTVAQHHEALVGHGFTHAHI
VALSQHPAALGTVAVKYQDMIAALPEATHEAIVGVGKRGAGARALEALLTVAGELRGP
PLQLDTGQLLKIAKRGGVTAVEAVHAWRNALTGAPLNLTPEQVVAIASNNGGKQALET
VQRLLPVLCQAHGLTPEQVVAIASNIGGKQALETVQRLLPVLCQAHGLTPEQVVAIASN
GGGKQALETVQRLLPVLCQAHGLTPEQVVAIASNNGGKQALETVQRLLPVLCQAHGL
TPEQVVAIASNNGGKQALETVQRLLPVLCQAHGLTPEQVVAIASHDGGKQALETVQRL
LPVLCQAHGLTPEQVVAIASNGGGKQALETVQRLLPVLCQAHGLTPEQVVAIASNIGG
KQALETVQRLLPVLCQAHGLTPEQVVAIASNGGGKQALETVQRLLPVLCQAHGLTPEQ
VVAIASNGGGKQALETVQRLLPVLCQAHGLTPEQVVAIASNGGGRPALESIVAQLSRPD
PALAALTNDHLVALACLGGRPALDAVKKGLGGSQLVKSELEEKKSELRHKLKYVPHEY
IELIEIARNSTQDRILEMKVMEFFMKVYGYRGKHLGGSRKPAGAIYTVGSPIDYGVIVD
TKAYSGGYNLPIGQADEMERYVEENQTRNKHLNPNEWWKVYPSSVTEFKFLFVSGHF
KGNYKAQLTRLNHITNCNGAVLSVEELLIGGEMIKAGTLTLEEVRRKENNGEINF

In Examples, the exemplary amino acid sequences of the elements or fusion proteins are as set forth below. Unless otherwise specified in the subsequent Examples, corresponding fusion proteins may be constructed in accordance with the schematic diagrams of the constructs shown in FIG. 16 A to 16E and FIG. 17A to 17H, based on the exemplary sequences as set forth below and the sequence disclosed in the present specification.

In subsequent Examples, the nickases used in the experiments for editing OsBADH2 were set forth below.

TALEN_{WT}
(SEQ ID NO. 154)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALETVQRLLPVLCQDHGLTPDQVVAIA
SNNGGKQALETVQRLLPVLCQDHGLTPDQVVAIASHDGGKQALETVQRLLPVLCQDH
GLTPDQVVAIASNGGGKQALETVQRLLPVLCQDHGLTPDQVVAIASNGGGKQALETV
QRLLPVLCQDHGLTPDQVVAIASNGGGKQALETVQRLLPVLCQDHGLTPDQVVAIASN
NGGKQALETVQRLLPVLCQDHGLTPDQVVAIASNIGGKQALETVQRLLPVLCQDHGLT
PDQVVAIASNNGGKQALETVQRLLPVLCQDHGLTPDQVVAIASNGGGKQALETVQRL
LPVLCQDHGLTPDQVVAIASNIGGKQALESIVAQLSRPDPALAALTNDHLVALACLGGR
PAMDAVKKGLPHAPELIRRVNRRIGERTSHRVAGSQLVKSELEEKKSELRHKLKYVPHE
YIELIEIARNSTQDRILEMKVMEFFMKVYGYRGKHLGGSRKPDGAIYTVGSPIDYGVIV
DTKAYSGGYNLPIGQADEMQRYVKENQTRNKHINPNEWWKVYPSSVTEFKFLFVSGH
FKGNYKAQLTRLNHKTNCNGAVLSVEELLIGGEMIKAGTLTLEEVRRKENNGEINFRS
GGGEGRGSLLTCGDVEENPGPRMDYKDHDGDYKDHDIDYKDDDDKMAPKKKRKVG
IHGVPARMVDLRTLGYSQQQQEKIKPKVRSTVAQHHEALVGHGFTHAHIVALSQHPAA
LGTVAVTYQHIITALPEATHEDIVGVGKQWSGARALEALLTDAGELRGPPLQLDTGQLV
KIAKRGGVTAMEAVHASRNALTGAPLNLTPDQVVAIASNNGGKQALETVQRLLPVLCQ
DHGLTPDQVVAIASHDGGKQALETVQRLLPVLCQDHGLTPDQVVAIASHDGGKQALE
TVQRLLPVLCQDHGLTPDQVVAIASNGGGKQALETVQRLLPVLCQDHGLTPDQVVAIA
SNGGGKQALETVQRLLPVLCQDHGLTPDQVVAIASNGGGKQALETVQRLLPVLCQDH
GLTPDQVVAIASNGGGKQALETVQRLLPVLCQDHGLTPDQVVAIASNNGGKQALETV
QRLLPVLCQDHGLTPDQVVAIASNGGGKQALETVQRLLPVLCQDHGLTPDQVVAIASH
DGGKQALETVQRLLPVLCQDHGLTPDQVVAIASHDGGKQALETVQRLLPVLCQDHGL
TPDQVVAIASNIGGKQALETVQRLLPVLCQDHGLTPDQVVAIASNIGGKQALETVQRLL
PVLCQDHGLTPDQVVAIASNNGGKQALETVQRLLPVLCQDHGLTPDQVVAIASNNGG
KQALETVQRLLPVLCQDHGLTPDQVVAIASNIGGKQALETVQRLLPVLCQDHGLTPDQ
VVAIASNGGGKQALESIVAQLSRPDPALAALTNDHLVALACLGGRPAMDAVKKGLPHA
PELIRRVNRRIGERTSHRVAGSQLVKSELEEKKSELRHKLKYVPHEYIELIEIARNSTQDR
ILEMKVMEFFMKVYGYRGKHLGGSRKPDGAIYTVGSPIDYGVIVDTKAYSGGYNLPIG
QADEMERYVEENQTRNKHLNPNEWWKVYPSSVTEFKFLFVSGHFKGNYKAQLTRLN
HITNCNGAVLSVEELLIGGEMIKAGTLTLEEVRRKFNNGEINF
TALE-FokI-R_{nickase(D450A)} or referred to as TALE-FokI-R_{nickase}
(SEQ ID NO. 155)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALETVQRLLPVLCQDHGLTPDQVVAIA
SNNGGKQALETVQRLLPVLCQDHGLTPDQVVAIASHDGGKQALETVQRLLPVLCQDH
GLTPDQVVAIASNGGGKQALETVQRLLPVLCQDHGLTPDQVVAIASNGGGKQALETV
QRLLPVLCQDHGLTPDQVVAIASNGGGKQALETVQRLLPVLCQDHGLTPDQVVAIASN
NGGKQALETVQRLLPVLCQDHGLTPDQVVAIASNIGGKQALETVQRLLPVLCQDHGLT
PDQVVAIASNNGGKQALETVQRLLPVLCQDHGLTPDQVVAIASNGGGKQALETVQRL
LPVLCQDHGLTPDQVVAIASNIGGKQALESIVAQLSRPDPALAALTNDHLVALACLGGR
PAMDAVKKGLPHAPELIRRVNRRIGERTSHRVAGSQLVKSELEEKKSELRHKLKYVPHE
YIELIEIARNSTQDRILEMKVMEFFMKVYGYRGKHLGGSRKPAGAIYTVGSPIDYGVIV
DTKAYSGGYNLPIGQADEMQRYVKENQTRNKHINPNEWWKVYPSSVTEFKFLFVSGH
FKGNYKAQLTRLNHKTNCNGAVLSVEELLIGGEMIKAGTLTLEEVRRKFNNGEINFRS
GGGEGRGSLLTCGDVEENPGPRMDYKDHDGDYKDHDIDYKDDDDKMAPKKKRKVG
IHGVPARMVDLRTLGYSQQQQEKIKPKVRSTVAQHHEALVGHGFTHAHIVALSQHPAA
LGTVAVTYQHIITALPEATHEDIVGVGKQWSGARALEALLTDAGELRGPPLQLDTGQLV
KIAKRGGVTAMEAVHASRNALTGAPLNLTPDQVVAIASNNGGKQALETVQRLLPVLCQ
DHGLTPDQVVAIASHDGGKQALETVQRLLPVLCQDHGLTPDQVVAIASHDGGKQALE
TVQRLLPVLCQDHGLTPDQVVAIASNGGGKQALETVQRLLPVLCQDHGLTPDQVVAIA
SNGGGKQALETVQRLLPVLCQDHGLTPDQVVAIASNGGGKQALETVQRLLPVLCQDH
GLTPDQVVAIASNGGGKQALETVQRLLPVLCQDHGLTPDQVVAIASNNGGKQALETV
QRLLPVLCQDHGLTPDQVVAIASNGGGKQALETVQRLLPVLCQDHGLTPDQVVAIASH
DGGKQALETVQRLLPVLCQDHGLTPDQVVAIASHDGGKQALETVQRLLPVLCQDHGL
TPDQVVAIASNIGGKQALETVQRLLPVLCQDHGLTPDQVVAIASNIGGKQALETVQRLL
PVLCQDHGLTPDQVVAIASNNGGKQALETVQRLLPVLCQDHGLTPDQVVAIASNNGG
KQALETVQRLLPVLCQDHGLTPDQVVAIASNIGGKQALETVQRLLPVLCQDHGLTPDQ
VVAIASNGGGKQALESIVAQLSRPDPALAALTNDHLVALACLGGRPAMDAVKKGLPHA
PELIRRVNRRIGERTSHRVAGSQLVKSELEEKKSELRHKLKYVPHEYIELIEIARNSTQDR
ILEMKVMEFFMKVYGYRGKHLGGSRKPDGAIYTVGSPIDYGVIVDTKAYSGGYNLPIG
QADEMERYVEENQTRNKHLNPNEWWKVYPSSVTEFKFLFVSGHFKGNYKAQLTRLN
HITNCNGAVLSVEELLIGGEMIKAGTLTLEEVRRKFNNGEINF
TALE-FokI-R_{nickase(D467A)}
(SEQ ID NO. 156)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASNGGGKQALETVQRLLPVLCQDHGLTPDQVVAIA
SNNGGKQALETVQRLLPVLCQDHGLTPDQVVAIASHDGGKQALETVQRLLPVLCQDH
GLTPDQVVAIASNGGGKQALETVQRLLPVLCQDHGLTPDQVVAIASNGGGKQALETV
QRLLPVLCQDHGLTPDQVVAIASNGGGKQALETVQRLLPVLCQDHGLTPDQVVAIASN
NGGKQALETVQRLLPVLCQDHGLTPDQVVAIASNIGGKQALETVQRLLPVLCQDHGLT
PDQVVAIASNNGGKQALETVQRLLPVLCQDHGLTPDQVVAIASNGGGKQALETVQRL
LPVLCQDHGLTPDQVVAIASNIGGKQALESIVAQLSRPDPALAALTNDHLVALACLGGR
PAMDAVKKGLPHAPELIRRVNRRIGERTSHRVAGSQLVKSELEEKKSELRHKLKYVPHE
YIELIEIARNSTQDRILEMKVMEFFMKVYGYRGKHLGGSRKPDGAIYTVGSPIDYGVIV
ATKAYSGGYNLPIGQADEMQRYVKENQTRNKHINPNEWWKVYPSSVTEFKFLFVSGH
FKGNYKAQLTRLNHKTNCNGAVLSVEELLIGGEMIKAGTLTLEEVRRKFNNGEINFRS
GGGEGRGSLLTCGDVEENPGPRMDYKDHDGDYKDHDIDYKDDDDKMAPKKKRKVG
IHGVPARMVDLRTLGYSQQQQEKIKPKVRSTVAQHHEALVGHGFTHAHIVALSQHPAA
LGTVAVTYQHIITALPEATHEDIVGVGKQWSGARALEALLTDAGELRGPPLQLDTGQLV
KIAKRGGVTAMEAVHASRNALTGAPLNLTPDQVVAIASNNGGKQALETVQRLLPVLCQ
DHGLTPDQVVAIASHDGGKQALETVQRLLPVLCQDHGLTPDQVVAIASHDGGKQALE
TVQRLLPVLCQDHGLTPDQVVAIASNGGGKQALETVQRLLPVLCQDHGLTPDQVVAIA
SNGGGKQALETVQRLLPVLCQDHGLTPDQVVAIASNGGGKQALETVQRLLPVLCQDH
GLTPDQVVAIASNGGGKQALETVQRLLPVLCQDHGLTPDQVVAIASNNGGKQALETV
QRLLPVLCQDHGLTPDQVVAIASNGGGKQALETVQRLLPVLCQDHGLTPDQVVAIASH
DGGKQALETVQRLLPVLCQDHGLTPDQVVAIASHDGGKQALETVQRLLPVLCQDHGL
TPDQVVAIASNIGGKQALETVQRLLPVLCQDHGLTPDQVVAIASNIGGKQALETVQRLL
PVLCQDHGLTPDQVVAIASNNGGKQALETVQRLLPVLCQDHGLTPDQVVAIASNNGG
KQALETVQRLLPVLCQDHGLTPDQVVAIASNIGGKQALETVQRLLPVLCQDHGLTPDQ
VVAIASNGGGKQALESIVAQLSRPDPALAALTNDHLVALACLGGRPAMDAVKKGLPHA
PELIRRVNRRIGERTSHRVAGSQLVKSELEEKKSELRHKLKYVPHEYIELIEIARNSTQDR
ILEMKVMEFFMKVYGYRGKHLGGSRKPDGAIYTVGSPIDYGVIVDTKAYSGGYNLPIG
QADEMERYVEENQTRNKHLNPNEWWKVYPSSVTEFKFLFVSGHFKGNYKAQLTRLN
HITNCNGAVLSVEELLIGGEMIKAGTLTLEEVRRKFNNGEINF

Nickases used in the experiments for editing OsDEP1:

TALEN_{WT}
(SEQ ID NO. 157)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNIGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALETVQ
RLLPVLCQDHGLTPDQVVAIASNNGGKQALETVQRLLPVLCQDHGLTPDQVVAIASNI
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNIGGKQALETVQRLL
PVLCQDHGLTPDQVVAIASNIGGKQALETVQRLLPVLCQDHGLTPDQVVAIASNNGGK
QALETVQRLLPVLCQDHGLTPDQVVAIASNNGGKQALETVQRLLPVLCQDHGLTPDQ
VVAIASNGGGKQALETVQRLLPVLCQDHGLTPDQVVAIASNNGGKQALETVQRLLPVL
CQDHGLTPDQVVAIASHDGGKQALETVQRLLPVLCQDHGLTPDQVVAIASHDGGKQA
LETVQRLLPVLCQDHGLTPDQVVAIASNGGGKQALESIVAQLSRPDPALAALTNDHLVA
LACLGGRPAMDAVKKGLPHAPELIRRVNRRIGERTSHRVAGSQLVKSELEEKKSELRHK
LKYVPHEYIELIEIARNSTQDRILEMKVMEFFMKVYGYRGKHLGGSRKPDGAIYTVGS
PIDYGVIVDTKAYSGGYNLPIGQADEMQRYVKENQTRNKHINPNEWWKVYPSSVTEF
KFLFVSGHFKGNYKAQLTRLNHKTNCNGAVLSVEELLIGGEMIKAGTLTLEEVRRKFN
NGEINFRSGGGEGRGSLLTCGDVEENPGPRMDYKDHDGDYKDHDIDYKDDDDKMAP
KKKRKVGIHGVPARMVDLRTLGYSQQQQEKIKPKVRSTVAQHHEALVGHGFTHAHIV
ALSQHPAALGTVAVTYQHIITALPEATHEDIVGVGKQWSGARALEALLTDAGELRGPPL
QLDTGQLVKIAKRGGVTAMEAVHASRNALTGAPLNLTPDQVVAIASNNGGKQALETV
QRLLPVLCQDHGLTPDQVVAIASNNGGKQALETVQRLLPVLCQDHGLTPDQVVAIASH
DGGKQALETVQRLLPVLCQDHGLTPDQVVAIASNGGGKQALETVQRLLPVLCQDHGL
TPDQVVAIASHDGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASHDG
GKQALETVQRLLPVLCQDHGLTPDQVVAIASNNGGKQALETVQRLLPVLCQDHGLTP
DQVVAIASHDGGKQALETVQRLLPVLCQDHGLTPDQVVAIASNIGGKQALETVQRLLP
VLCQDHGLTPDQVVAIASNNGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNGGGKQALESIVAQLSRPDPALAALTNDHLVALACLGGRPAMDAVKKGLPHAP
ELIRRVNRRIGERTSHRVAGSQLVKSELEEKKSELRHKLKYVPHEYIELIEIARNSTQDRI
LEMKVMEFFMKVYGYRGKHLGGSRKPDGAIYTVGSPIDYGVIVDTKAYSGGYNLPIG
QADEMERYVEENQTRNKHLNPNEWWKVYPSSVTEFKFLFVSGHFKGNYKAQLTRLN
HITNCNGAVLSVEELLIGGEMIKAGTLTLEEVRRKFNNGEINF
TALE-FokI-R_{nickase(D450A)} or referred to as TALE-FokI-R_{nickase}
(SEQ ID NO. 158)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNIGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALETVQ
RLLPVLCQDHGLTPDQVVAIASNNGGKQALETVQRLLPVLCQDHGLTPDQVVAIASNI
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNIGGKQALETVQRLL
PVLCQDHGLTPDQVVAIASNIGGKQALETVQRLLPVLCQDHGLTPDQVVAIASNNGGK
QALETVQRLLPVLCQDHGLTPDQVVAIASNNGGKQALETVQRLLPVLCQDHGLTPDQ
VVAIASNGGGKQALETVQRLLPVLCQDHGLTPDQVVAIASNNGGKQALETVQRLLPVL
CQDHGLTPDQVVAIASHDGGKQALETVQRLLPVLCQDHGLTPDQVVAIASHDGGKQA
LETVQRLLPVLCQDHGLTPDQVVAIASNGGGKQALESIVAQLSRPDPALAALTNDHLVA
LACLGGRPAMDAVKKGLPHAPELIRRVNRRIGERTSHRVAGSQLVKSELEEKKSELRHK
LKYVPHEYIELIEIARNSTQDRILEMKVMEFFMKVYGYRGKHLGGSRKPAGAIYTVGS
PIDYGVIVDTKAYSGGYNLPIGQADEMQRYVKENQTRNKHINPNEWWKVYPSSVTEF
KFLFVSGHFKGNYKAQLTRLNHKTNCNGAVLSVEELLIGGEMIKAGTLTLEEVRRKFN
NGEINFRSGGGEGRGSLLTCGDVEENPGPRMDYKDHDGDYKDHDIDYKDDDDKMAP
KKKRKVGIHGVPARMVDLRTLGYSQQQQEKIKPKVRSTVAQHHEALVGHGFTHAHIV
ALSQHPAALGTVAVTYQHIITALPEATHEDIVGVGKQWSGARALEALLTDAGELRGPPL
QLDTGQLVKIAKRGGVTAMEAVHASRNALTGAPLNLTPDQVVAIASNNGGKQALETV
QRLLPVLCQDHGLTPDQVVAIASNNGGKQALETVQRLLPVLCQDHGLTPDQVVAIASH
DGGKQALETVQRLLPVLCQDHGLTPDQVVAIASNGGGKQALETVQRLLPVLCQDHGL
TPDQVVAIASHDGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASHDG
GKQALETVQRLLPVLCQDHGLTPDQVVAIASNNGGKQALETVQRLLPVLCQDHGLTP
DQVVAIASHDGGKQALETVQRLLPVLCQDHGLTPDQVVAIASNIGGKQALETVQRLLP
VLCQDHGLTPDQVVAIASNNGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNGGGKQALESIVAQLSRPDPALAALTNDHLVALACLGGRPAMDAVKKGLPHAP
ELIRRVNRRIGERTSHRVAGSQLVKSELEEKKSELRHKLKYVPHEYIELIEIARNSTQDRI
LEMKVMEFFMKVYGYRGKHLGGSRKPDGAIYTVGSPIDYGVIVDTKAYSGGYNLPIG
QADEMERYVEENQTRNKHLNPNEWWKVYPSSVTEFKFLFVSGHFKGNYKAQLTRLN
HITNCNGAVLSVEELLIGGEMIKAGTLTLEEVRRKFNNGEINF
TALE-FokI-R_{nickase(D467A)}
(SEQ ID NO. 159)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASNNGGKQ
ALETVQRLLPVLCQDHGLTPDQVVAIASHDGGKQALETVQRLLPVLCQDHGLTPDQVV
AIASNIGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALETVQ
RLLPVLCQDHGLTPDQVVAIASNNGGKQALETVQRLLPVLCQDHGLTPDQVVAIASNI
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNIGGKQALETVQRLL
PVLCQDHGLTPDQVVAIASNIGGKQALETVQRLLPVLCQDHGLTPDQVVAIASNNGGK
QALETVQRLLPVLCQDHGLTPDQVVAIASNNGGKQALETVQRLLPVLCQDHGLTPDQ
VVAIASNGGGKQALETVQRLLPVLCQDHGLTPDQVVAIASNNGGKQALETVQRLLPVL
CQDHGLTPDQVVAIASHDGGKQALETVQRLLPVLCQDHGLTPDQVVAIASHDGGKQA
LETVQRLLPVLCQDHGLTPDQVVAIASNGGGKQALESIVAQLSRPDPALAALTNDHLVA
LACLGGRPAMDAVKKGLPHAPELIRRVNRRIGERTSHRVAGSQLVKSELEEKKSELRHK
LKYVPHEYIELIEIARNSTQDRILEMKVMEFFMKVYGYRGKHLGGSRKPDGAIYTVGS
PIDYGVIVATKAYSGGYNLPIGQADEMQRYVKENQTRNKHINPNEWWKVYPSSVTEFK
FLFVSGHFKGNYKAQLTRLNHKTNCNGAVLSVEELLIGGEMIKAGTLTLEEVRRKENN
GEINFRSGGGEGRGSLLTCGDVEENPGPRMDYKDHDGDYKDHDIDYKDDDDKMAPK
KKRKVGIHGVPARMVDLRTLGYSQQQQEKIKPKVRSTVAQHHEALVGHGFTHAHIVA
LSQHPAALGTVAVTYQHIITALPEATHEDIVGVGKQWSGARALEALLTDAGELRGPPLQ
LDTGQLVKIAKRGGVTAMEAVHASRNALTGAPLNLTPDQVVAIASNNGGKQALETVQ
RLLPVLCQDHGLTPDQVVAIASNNGGKQALETVQRLLPVLCQDHGLTPDQVVAIASHD
GGKQALETVQRLLPVLCQDHGLTPDQVVAIASNGGGKQALETVQRLLPVLCQDHGLT
PDQVVAIASHDGGKQALETVQRLLPVLCQDHGLTPDQVVAIASNNGGKQALETVQRL
LPVLCQDHGLTPDQVVAIASNGGGKQALETVQRLLPVLCQDHGLTPDQVVAIASHDGG
KQALETVQRLLPVLCQDHGLTPDQVVAIASNNGGKQALETVQRLLPVLCQDHGLTPD
QVVAIASHDGGKQALETVQRLLPVLCQDHGLTPDQVVAIASNIGGKQALETVQRLLPV
LCQDHGLTPDQVVAIASNNGGKQALETVQRLLPVLCQDHGLTPDQVVAIASHDGGKQ
ALETVQRLLPVLCQDHGLTPDQVVAIASNIGGKQALETVQRLLPVLCQDHGLTPDQVV
AIASNGGGKQALESIVAQLSRPDPALAALTNDHLVALACLGGRPAMDAVKKGLPHAPE
LIRRVNRRIGERTSHRVAGSQLVKSELEEKKSELRHKLKYVPHEYIELIEIARNSTQDRIL
EMKVMEFFMKVYGYRGKHLGGSRKPDGAIYTVGSPIDYGVIVDTKAYSGGYNLPIGQ
ADEMERYVEENQTRNKHLNPNEWWKVYPSSVTEFKFLFVSGHFKGNYKAQLTRLNHI
TNCNGAVLSVEELLIGGEMIKAGTLTLEEVRRKFNNGEINF

Nickases used in the experiments for editing OsCKX2:

TALEN_{WT}
(SEQ ID NO. 160)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASHDGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SHDGGKQALETVQRLLPVLCQDHGLTPDQVVAIASNNGGKQALETVQRLLPVLCQDH
GLTPDQVVAIASHDGGKQALETVQRLLPVLCQDHGLTPDQVVAIASNNGGKQALETV
QRLLPVLCQDHGLTPDQVVAIASNIGGKQALETVQRLLPVLCQDHGLTPDQVVAIASNI
GGKQALETVQRLLPVLCQDHGLTPDQVVAIASNIGGKQALETVQRLLPVLCQDHGLTP
DQVVAIASNNGGKQALETVQRLLPVLCQDHGLTPDQVVAIASNIGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNGGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SNNGGKQALESIVAQLSRPDPALAALTNDHLVALACLGGRPAMDAVKKGLPHAPELIRR
VNRRIGERTSHRVAGSQLVKSELEEKKSELRHKLKYVPHEYIELIEIARNSTQDRILEMK
VMEFFMKVYGYRGKHLGGSRKPDGAIYTVGSPIDYGVIVDTKAYSGGYNLPIGQADE
MQRYVKENQTRNKHINPNEWWKVYPSSVTEFKFLFVSGHFKGNYKAQLTRLNHKTN
CNGAVLSVEELLIGGEMIKAGTLTLEEVRRKFNNGEINFRSGGGEGRGSLLTCGDVEEN
PGPRMDYKDHDGDYKDHDIDYKDDDDKMAPKKKRKVGIHGVPARMVDLRTLGYSQ
QQQEKIKPKVRSTVAQHHEALVGHGFTHAHIVALSQHPAALGTVAVTYQHIITALPEAT
HEDIVGVGKQWSGARALEALLTDAGELRGPPLQLDTGQLVKIAKRGGVTAMEAVHAS
RNALTGAPLNLTPDQVVAIASHDGGKQALETVQRLLPVLCQDHGLTPDQVVAIASHDG
GKQALETVQRLLPVLCQDHGLTPDQVVAIASHDGGKQALETVQRLLPVLCQDHGLTP
DQVVAIASNIGGKQALETVQRLLPVLCQDHGLTPDQVVAIASNNGGKQALETVQRLLP
VLCQDHGLTPDQVVAIASNIGGKQALETVQRLLPVLCQDHGLTPDQVVAIASNNGGKQ
ALETVQRLLPVLCQDHGLTPDQVVAIASHDGGKQALETVQRLLPVLCQDHGLTPDQVV
AIASHDGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNNGGKQAL
ETVQRLLPVLCQDHGLTPDQVVAIASHDGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNNGGKQALET
VQRLLPVLCQDHGLTPDQVVAIASHDGGKQALESIVAQLSRPDPALAALTNDHLVALAC
LGGRPAMDAVKKGLPHAPELIRRVNRRIGERTSHRVAGSQLVKSELEEKKSELRHKLKY
VPHEYIELIEIARNSTQDRILEMKVMEFFMKVYGYRGKHLGGSRKPDGAIYTVGSPIDY
GVIVDTKAYSGGYNLPIGQADEMERYVEENQTRNKHLNPNEWWKVYPSSVTEFKFLF
VSGHFKGNYKAQLTRLNHITNCNGAVLSVEELLIGGEMIKAGTLTLEEVRRKENNGEIN
F
TALE-FokI-R_{nickase}
(SEQ ID NO. 161)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASHDGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SHDGGKQALETVQRLLPVLCQDHGLTPDQVVAIASNNGGKQALETVQRLLPVLCQDH
GLTPDQVVAIASHDGGKQALETVQRLLPVLCQDHGLTPDQVVAIASNNGGKQALETV
QRLLPVLCQDHGLTPDQVVAIASNIGGKQALETVQRLLPVLCQDHGLTPDQVVAIASNI
GGKQALETVQRLLPVLCQDHGLTPDQVVAIASNIGGKQALETVQRLLPVLCQDHGLTP
DQVVAIASNNGGKQALETVQRLLPVLCQDHGLTPDQVVAIASNIGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNGGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SNNGGKQALESIVAQLSRPDPALAALTNDHLVALACLGGRPAMDAVKKGLPHAPELIRR
VNRRIGERTSHRVAGSQLVKSELEEKKSELRHKLKYVPHEYIELIEIARNSTQDRILEMK
VMEFFMKVYGYRGKHLGGSRKPAGAIYTVGSPIDYGVIVDTKAYSGGYNLPIGQADE
MQRYVKENQTRNKHINPNEWWKVYPSSVTEFKFLFVSGHFKGNYKAQLTRLNHKTN
CNGAVLSVEELLIGGEMIKAGTLTLEEVRRKFNNGEINFRSGGGEGRGSLLTCGDVEEN
PGPRMDYKDHDGDYKDHDIDYKDDDDKMAPKKKRKVGIHGVPARMVDLRTLGYSQ
QQQEKIKPKVRSTVAQHHEALVGHGFTHAHIVALSQHPAALGTVAVTYQHIITALPEAT
HEDIVGVGKQWSGARALEALLTDAGELRGPPLQLDTGQLVKIAKRGGVTAMEAVHAS
RNALTGAPLNLTPDQVVAIASHDGGKQALETVQRLLPVLCQDHGLTPDQVVAIASHDG
GKQALETVQRLLPVLCQDHGLTPDQVVAIASHDGGKQALETVQRLLPVLCQDHGLTP
DQVVAIASNIGGKQALETVQRLLPVLCQDHGLTPDQVVAIASNNGGKQALETVQRLLP
VLCQDHGLTPDQVVAIASNIGGKQALETVQRLLPVLCQDHGLTPDQVVAIASNNGGKQ
ALETVQRLLPVLCQDHGLTPDQVVAIASHDGGKQALETVQRLLPVLCQDHGLTPDQVV
AIASHDGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNNGGKQAL
ETVQRLLPVLCQDHGLTPDQVVAIASHDGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNNGGKQALET
VQRLLPVLCQDHGLTPDQVVAIASHDGGKQALESIVAQLSRPDPALAALTNDHLVALAC
LGGRPAMDAVKKGLPHAPELIRRVNRRIGERTSHRVAGSQLVKSELEEKKSELRHKLKY
VPHEYIELIEIARNSTQDRILEMKVMEFFMKVYGYRGKHLGGSRKPDGAIYTVGSPIDY
GVIVDTKAYSGGYNLPIGQADEMERYVEENQTRNKHLNPNEWWKVYPSSVTEFKFLF
VSGHFKGNYKAQLTRLNHITNCNGAVLSVEELLIGGEMIKAGTLTLEEVRRKFNNGEIN
F
TALE-FokI-L_{nickase}
(SEQ ID NO. 162)
MAPKKKRKVGIHGVPSRMVDLRTLGYSQQQQEKIKPKVRSTVAQHHEALVGHGF
THAHIVALSQHPAALGTVAVTYQHIITALPEATHEDIVGVGKQWSGARALEALLTDAGE
LRGPPLQLDTGQLVKIAKRGGVTAMEAVHASRNALTGAPLNLTPDQVVAIASHDGGKQ
ALETVQRLLPVLCQDHGLTPDQVVAIASHDGGKQALETVQRLLPVLCQDHGLTPDQVV
AIASNGGGKQALETVQRLLPVLCQDHGLTPDQVVAIASNNGGKQALETVQRLLPVLC
QDHGLTPDQVVAIASNNGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SHDGGKQALETVQRLLPVLCQDHGLTPDQVVAIASNNGGKQALETVQRLLPVLCQDH
GLTPDQVVAIASHDGGKQALETVQRLLPVLCQDHGLTPDQVVAIASNNGGKQALETV
QRLLPVLCQDHGLTPDQVVAIASNIGGKQALETVQRLLPVLCQDHGLTPDQVVAIASNI
GGKQALETVQRLLPVLCQDHGLTPDQVVAIASNIGGKQALETVQRLLPVLCQDHGLTP
DQVVAIASNNGGKQALETVQRLLPVLCQDHGLTPDQVVAIASNIGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNIGGKQALETVQRLLPVLCQDHGLTPDQV
VAIASNGGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVAIA
SNNGGKQALESIVAQLSRPDPALAALTNDHLVALACLGGRPAMDAVKKGLPHAPELIRR
VNRRIGERTSHRVAGSQLVKSELEEKKSELRHKLKYVPHEYIELIEIARNSTQDRILEMK
VMEFFMKVYGYRGKHLGGSRKPDGAIYTVGSPIDYGVIVDTKAYSGGYNLPIGQADE
MQRYVKENQTRNKHINPNEWWKVYPSSVTEFKFLFVSGHFKGNYKAQLTRLNHKTN
CNGAVLSVEELLIGGEMIKAGTLTLEEVRRKFNNGEINFRSGGGEGRGSLLTCGDVEEN
PGPRMDYKDHDGDYKDHDIDYKDDDDKMAPKKKRKVGIHGVPARMVDLRTLGYSQ
QQQEKIKPKVRSTVAQHHEALVGHGFTHAHIVALSQHPAALGTVAVTYQHIITALPEAT
HEDIVGVGKQWSGARALEALLTDAGELRGPPLQLDTGQLVKIAKRGGVTAMEAVHAS
RNALTGAPLNLTPDQVVAIASHDGGKQALETVQRLLPVLCQDHGLTPDQVVAIASHDG
GKQALETVQRLLPVLCQDHGLTPDQVVAIASHDGGKQALETVQRLLPVLCQDHGLTP
DQVVAIASNIGGKQALETVQRLLPVLCQDHGLTPDQVVAIASNNGGKQALETVQRLLP
VLCQDHGLTPDQVVAIASNIGGKQALETVQRLLPVLCQDHGLTPDQVVAIASNNGGKQ
ALETVQRLLPVLCQDHGLTPDQVVAIASHDGGKQALETVQRLLPVLCQDHGLTPDQVV
AIASHDGGKQALETVQRLLPVLCQDHGLTPDQVVAIASHDGGKQALETVQRLLPVLC
QDHGLTPDQVVAIASHDGGKQALETVQRLLPVLCQDHGLTPDQVVAIASNNGGKQAL
ETVQRLLPVLCQDHGLTPDQVVAIASHDGGKQALETVQRLLPVLCQDHGLTPDQVVAI
ASNNGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNNGGKQALET
VQRLLPVLCQDHGLTPDQVVAIASHDGGKQALESIVAQLSRPDPALAALTNDHLVALAC
LGGRPAMDAVKKGLPHAPELIRRVNRRIGERTSHRVAGSQLVKSELEEKKSELRHKLKY
VPHEYIELIEIARNSTQDRILEMKVMEFFMKVYGYRGKHLGGSRKPAGAIYTVGSPIDY
GVIVDTKAYSGGYNLPIGQADEMERYVEENQTRNKHLNPNEWWKVYPSSVTEFKFLF
VSGHFKGNYKAQLTRLNHITNCNGAVLSVEELLIGGEMIKAGTLTLEEVRRKFNNGEIN
F

In Examples 1 to 6, mExoI is the aforementioned mExoI-NLS (FIG. 16B), SEQ ID NO. 125; A3A-UGI is the aforementioned NLS-A3A-XTEN-UGI (FIG. 16B), SEQ ID NO. 123; Trex2 is the aforementioned Trex2-NLS (FIG. 16B), SEQ ID NO. 126.

In Examples 1 to 6, the amino acid sequence of UGI is the aforementioned NLS-UGI (FIG. 16B) (SEQ ID NO. 163).

The amino acid sequence of APOBEC1-UGI in Example 4 is the aforementioned NLS-rAPOBEC1-XTEN-UGI (FIG. 16B) (SEQ ID NO. 164).

Amino acid sequence of ExoV (ExoV-NLS) in
Example 1 (SEQ ID NO. 165):
MAETGEEETASAEASGFSDLSDSELVEFLDLEEAKESAVSLSKPGPSAE
LPGKDDKPVSLQNWKGGLDVLSPMERFHLKYLYVTDLCTQNWCELQMVY
GKELPGSLTPEKAAVLDTGASIHLAKELELHDLVTVPIATKEDAWAVKF
LNILAMIPALQSEGRVREFPVFGEVEGIFLVGVIDELHYTSKGELELAE
LKTRRRPVLPLPAQKKKDYFQVSLYKYIFDAMVQGKVTPASLIHHTKLC
LDKPLGPSVLRHARQGGVSVKSLGDLMELVFLSLTLSDLPAIDTLKLEY
IHQETATILGTEIVAFEEKEVKSKVQHYVAYWMGHRDPQGVDVEEAWKC
RTCDYVDICEWRRGSGVLSSSWEPKAKKFKMKRTADGSEFESPKKKRKV

The amino acid sequence of TadA-8e in Example 5 is the aforementioned TadA8e-NLS (FIG. 16B) (SEQ ID NO. 166).

### In Example 6

Amino acid sequence of mExoI-16 aa-A3A-UGI (SEQ ID NO. 167):
MKRTADGSEFESPKKKRKVMGIQGLLQFIQEASEPVNVKKYKGQAVAVDTYCWL
HKGAIACAEKLAKGEPTDRYVGFCMKFVNMLLSYGVKPILIFDGCTLPSKKEVERSRR
ERRQSNLLKGKQLLREGKVSEARDCFARSINITHAMAHKVIKAARALGVDCLVAPYEA
DAQLAYLNKAGIVQAVITEDSDLLAFGCKKVILKMDQFGNGLEVDQARLGMCKQLGD
VFTEEKFRYMCILSGCDYLASLRGIGLAKACKVLRLANNPDIVKVIKKIGHYLRMNITV
PEDYITGFIRANNTFLYQLVFDPIQRKLVPLNAYGDDVNPETLTYAGQYVGDSVALQIAL
GNRDVNTFEQIDDYSPDTMPAHSRSHSWNEKAGQKPPGTNSIWHKNYCPRLEVNSVS
HAPQLKEKPSTLGLKQVISTKGLNLPRKSCVLKRPRNEALAEDDLLSQYSSVSKKIKEN
GCGDGTSPNSSKMSKSCPDSGTAHKTDAHTPSKMRNKFATFLQRRNEESGAVVVPGTR
SRFFCSSQDFDNFIPKKESGQPLNETVATGKATTSLLGALDCPDTEGHKPVDANGTHNL
SSQIPGNAAVSPEDEAQSSETSKLLGAMSPPSLGTLRSCFSWSGTLREFSRTPSPSASTTL
QQFRRKSDPPACLPEASAVVTDRCDSKSEMLGETSQPLHELGCSSRSQESMDSSCGLNT
SSLSQPSSRDSGSEESDCNNKSLDNQGEQNSKQHLPHFSKKDGLRRNKVPGLCRSSSM
DSFSTTKIKPLVPARVSGLSKKSGSMQTRKHHDVENKPGLQTKISELWKNFGFKKDSEK
LPSCKKPLSPVKDNIQLTPETEDEIFNKPECVRAQRAIFHSGSETPGTSESATPESMKRTA
DGSEFESPKKKRKVMEASPASGPRHLMDPHIFTSNFNNGIGRHKTYLCYEVERLDNGT
SVKMDQHRGFLHNQAKNLLCGFYGRHAELRFLDLVPSLQLDPAQIYRVTWFISWSPCF
SWGCAGEVRAFLQENTHVRLRIFAARIYDYDPLYKEALQMLRDAGAQVSIMTYDEFK
HCWDTFVDHQGCPFQPWDGLDEHSQALSGRLRAILQNQGNSGSETPGTSESATPESTN
LSDIIEKETGKQLVIQESILMLPEEVEEVIGNKPESDILVHTAYDESTDENVMLLTSDAPE
YKPWALVIQDSNGENKIKML
Amino acid sequence of mExoI-48 aa-A3A-UGI (SEQ ID NO. 168):
MKRTADGSEFESPKKKRKVMGIQGLLQFIQEASEPVNVKKYKGQAVAVDTYCWL
HKGAIACAEKLAKGEPTDRYVGFCMKFVNMLLSYGVKPILIFDGCTLPSKKEVERSRR
ERRQSNLLKGKQLLREGKVSEARDCFARSINITHAMAHKVIKAARALGVDCLVAPYEA
DAQLAYLNKAGIVQAVITEDSDLLAFGCKKVILKMDQFGNGLEVDQARLGMCKQLGD
VFTEEKFRYMCILSGCDYLASLRGIGLAKACKVLRLANNPDIVKVIKKIGHYLRMNITV
PEDYITGFIRANNTFLYQLVFDPIQRKLVPLNAYGDDVNPETLTYAGQYVGDSVALQIAL
GNRDVNTFEQIDDYSPDTMPAHSRSHSWNEKAGQKPPGTNSIWHKNYCPRLEVNSVS
HAPQLKEKPSTLGLKQVISTKGLNLPRKSCVLKRPRNEALAEDDLLSQYSSVSKKIKEN
GCGDGTSPNSSKMSKSCPDSGTAHKTDAHTPSKMRNKFATFLQRRNEESGAVVVPGTR
SRFFCSSQDFDNFIPKKESGQPLNETVATGKATTSLLGALDCPDTEGHKPVDANGTHNL
SSQIPGNAAVSPEDEAQSSETSKLLGAMSPPSLGTLRSCFSWSGTLREFSRTPSPSASTTL
QQFRRKSDPPACLPEASAVVTDRCDSKSEMLGETSQPLHELGCSSRSQESMDSSCGLNT
SSLSQPSSRDSGSEESDCNNKSLDNQGEQNSKQHLPHFSKKDGLRRNKVPGLCRSSSM
DSFSTTKIKPLVPARVSGLSKKSGSMQTRKHHDVENKPGLQTKISELWKNFGFKKDSEK
LPSCKKPLSPVKDNIQLTPETEDEIFNKPECVRAQRAIFHSGSETPGTSESATPESSGGSS
GGSSGSETPGTSESATPESSGGSSGGSMKRTADGSEFESPKKKRKVMEASPASGPRHLM
DPHIFTSNFNNGIGRHKTYLCYEVERLDNGTSVKMDQHRGFLHNQAKNLLCGFYGRH
AELRFLDLVPSLQLDPAQIYRVTWFISWSPCFSWGCAGEVRAFLQENTHVRLRIFAARI
YDYDPLYKEALQMLRDAGAQVSIMTYDEFKHCWDTFVDHQGCPFQPWDGLDEHSQA
LSGRLRAILQNQGNSGSETPGTSESATPESTNLSDIIEKETGKQLVIQESILMLPEEVEEVI
GNKPESDILVHTAYDESTDENVMLLTSDAPEYKPWALVIQDSNGENKIKML
A3A-TALE-FokI-R_{nickase}
(SEQ ID NO. 169)
MAPKKKRKVMEASPASGPRHLMDPHIFTSNFNNGIGRHKTYLCYEVERLDNGTSV
KMDQHRGFLHNQAKNLLCGFYGRHAELRFLDLVPSLQLDPAQIYRVTWFISWSPCFSW
GCAGEVRAFLQENTHVRLRIFAARIYDYDPLYKEALQMLRDAGAQVSIMTYDEFKHC
WDTFVDHQGCPFQPWDGLDEHSQALSGRLRAILQNQGSGSETPGTSESATPESSGGSS
GGSSGSETPGTSESATPESSGGSSGGSGIHGVPSRMVDLRTLGYSQQQQEKIKPKVRSTV
AQHHEALVGHGFTHAHIVALSQHPAALGTVAVTYQHIITALPEATHEDIVGVGKQWSG
ARALEALLTDAGELRGPPLQLDTGQLVKIAKRGGVTAMEAVHASRNALTGAPLNLTPD
QVVAIASNNGGKQALETVQRLLPVLCQDHGLTPDQVVAIASHDGGKQALETVQRLLP
VLCQDHGLTPDQVVAIASNIGGKQALETVQRLLPVLCQDHGLTPDQVVAIASNIGGKQ
ALETVQRLLPVLCQDHGLTPDQVVAIASNIGGKQALETVQRLLPVLCQDHGLTPDQVV
AIASNIGGKQALETVQRLLPVLCQDHGLTPDQVVAIASNNGGKQALETVQRLLPVLCQ
DHGLTPDQVVAIASNIGGKQALETVQRLLPVLCQDHGLTPDQVVAIASHDGGKQALET
VQRLLPVLCQDHGLTPDQVVAIASHDGGKQALETVQRLLPVLCQDHGLTPDQVVAIAS
NIGGKQALETVQRLLPVLCQDHGLTPDQVVAIASNIGGKQALETVQRLLPVLCQDHGL
TPDQVVAIASNNGGKQALETVQRLLPVLCQDHGLTPDQVVAIASNNGGKQALETVQR
LLPVLCQDHGLTPDQVVAIASNGGGKQALETVQRLLPVLCQDHGLTPDQVVAIASNNG
GKQALETVQRLLPVLCQDHGLTPDQVVAIASHDGGKQALETVQRLLPVLCQDHGLTP
DQVVAIASHDGGKQALETVQRLLPVLCQDHGLTPDQVVAIASNGGGKQALESIVAQLS
RPDPALAALTNDHLVALACLGGRPAMDAVKKGLPHAPELIRRVNRRIGERTSHRVAGSQ
LVKSELEEKKSELRHKLKYVPHEYIELIEIARNSTQDRILEMKVMEFFMKVYGYRGKH
LGGSRKPAGAIYTVGSPIDYGVIVDTKAYSGGYNLPIGQADEMQRYVKENQTRNKHIN
PNEWWKVYPSSVTEFKFLFVSGHFKGNYKAQLTRLNHKTNCNGAVLSVEELLIGGEMI
KAGTLTLEEVRRKFNNGEINFRSGGGEGRGSLLTCGDVEENPGPRMDYKDHDGDYKD
HDIDYKDDDDKMAPKKKRKVGIHGVPARMVDLRTLGYSQQQQEKIKPKVRSTVAQH
HEALVGHGFTHAHIVALSQHPAALGTVAVTYQHIITALPEATHEDIVGVGKQWSGARAL
EALLTDAGELRGPPLQLDTGQLVKIAKRGGVTAMEAVHASRNALTGAPLNLTPDQVVA
IASNNGGKQALETVQRLLPVLCQDHGLTPDQVVAIASNNGGKQALETVQRLLPVLCQ
DHGLTPDQVVAIASHDGGKQALETVQRLLPVLCQDHGLTPDQVVAIASNGGGKQALE
TVQRLLPVLCQDHGLTPDQVVAIASHDGGKQALETVQRLLPVLCQDHGLTPDQVVAIA
SNNGGKQALETVQRLLPVLCQDHGLTPDQVVAIASNGGGKQALETVQRLLPVLCQDH
GLTPDQVVAIASHDGGKQALETVQRLLPVLCQDHGLTPDQVVAIASNNGGKQALETV
QRLLPVLCQDHGLTPDQVVAIASHDGGKQALETVQRLLPVLCQDHGLTPDQVVAIASN
IGGKQALETVQRLLPVLCQDHGLTPDQVVAIASNNGGKQALETVQRLLPVLCQDHGLT
PDQVVAIASHDGGKQALETVQRLLPVLCQDHGLTPDQVVAIASNIGGKQALETVQRLL
PVLCQDHGLTPDQVVAIASNGGGKQALESIVAQLSRPDPALAALTNDHLVALACLGGRP
AMDAVKKGLPHAPELIRRVNRRIGERTSHRVAGSQLVKSELEEKKSELRHKLKYVPHE
YIELIEIARNSTQDRILEMKVMEFFMKVYGYRGKHLGGSRKPDGAIYTVGSPIDYGVIV
DTKAYSGGYNLPIGQADEMERYVEENQTRNKHLNPNEWWKVYPSSVTEFKFLFVSGH
FKGNYKAQLTRLNHITNCNGAVLSVEELLIGGEMIKAGTLTLEEVRRKENNGEINF
APOBEC1-TALE-FokI-R_{nickase}
(SEQ ID NO. 170)
MAPKKKRKVSSETGPVAVDPTLRRRIEPHEFEVFFDPRELRKETCLLYEINWGGRH
SIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHW
PRYPHLWVRLYVLELYCIILGLPPCLNILRRKQPQLTFFTIALQSCHYQRLPPHILWATGL
KSGSETPGTSESATPESSGGSSGGSSGSETPGTSESATPESSGGSSGGSGIHGVPSRMVDL
RTLGYSQQQQEKIKPKVRSTVAQHHEALVGHGFTHAHIVALSQHPAALGTVAVTYQHII
TALPEATHEDIVGVGKQWSGARALEALLTDAGELRGPPLQLDTGQLVKIAKRGGVTA
MEAVHASRNALTGAPLNLTPDQVVAIASNNGGKQALETVQRLLPVLCQDHGLTPDQV
VAIASHDGGKQALETVQRLLPVLCQDHGLTPDQVVAIASNIGGKQALETVQRLLPVLC
QDHGLTPDQVVAIASNIGGKQALETVQRLLPVLCQDHGLTPDQVVAIASNIGGKQALET
VQRLLPVLCQDHGLTPDQVVAIASNIGGKQALETVQRLLPVLCQDHGLTPDQVVAIAS
NNGGKQALETVQRLLPVLCQDHGLTPDQVVAIASNIGGKQALETVQRLLPVLCQDHGL
TPDQVVAIASHDGGKQALETVQRLLPVLCQDHGLTPDQVVAIASHDGGKQALETVQR
LLPVLCQDHGLTPDQVVAIASNIGGKQALETVQRLLPVLCQDHGLTPDQVVAIASNIGG
KQALETVQRLLPVLCQDHGLTPDQVVAIASNNGGKQALETVQRLLPVLCQDHGLTPD
QVVAIASNNGGKQALETVQRLLPVLCQDHGLTPDQVVAIASNGGGKQALETVQRLLP
VLCQDHGLTPDQVVAIASNNGGKQALETVQRLLPVLCQDHGLTPDQVVAIASHDGGK
QALETVQRLLPVLCQDHGLTPDQVVAIASHDGGKQALETVQRLLPVLCQDHGLTPDQ
VVAIASNGGGKQALESIVAQLSRPDPALAALTNDHLVALACLGGRPAMDAVKKGLPHA
PELIRRVNRRIGERTSHRVAGSQLVKSELEEKKSELRHKLKYVPHEYIELIEIARNSTQDR
ILEMKVMEFFMKVYGYRGKHLGGSRKPAGAIYTVGSPIDYGVIVDTKAYSGGYNLPIG
QADEMQRYVKENQTRNKHINPNEWWKVYPSSVTEFKFLFVSGHFKGNYKAQLTRLN
HKTNCNGAVLSVEELLIGGEMIKAGTLTLEEVRRKFNNGEINFRSGGGEGRGSLLTCGD
VEENPGPRMDYKDHDGDYKDHDIDYKDDDDKMAPKKKRKVGIHGVPARMVDLRTL
GYSQQQQEKIKPKVRSTVAQHHEALVGHGFTHAHIVALSQHPAALGTVAVTYQHIITAL
PEATHEDIVGVGKQWSGARALEALLTDAGELRGPPLQLDTGQLVKIAKRGGVTAMEA
VHASRNALTGAPLNLTPDQVVAIASNNGGKQALETVQRLLPVLCQDHGLTPDQVVAIA
SNNGGKQALETVQRLLPVLCQDHGLTPDQVVAIASHDGGKQALETVQRLLPVLCQDH
GLTPDQVVAIASNGGGKQALETVQRLLPVLCQDHGLTPDQVVAIASHDGGKQALETV
QRLLPVLCQDHGLTPDQVVAIASNNGGKQALETVQRLLPVLCQDHGLTPDQVVAIASN
GGGKQALETVQRLLPVLCQDHGLTPDQVVAIASHDGGKQALETVQRLLPVLCQDHGL
TPDQVVAIASNNGGKQALETVQRLLPVLCQDHGLTPDQVVAIASHDGGKQALETVQR
LLPVLCQDHGLTPDQVVAIASNIGGKQALETVQRLLPVLCQDHGLTPDQVVAIASNNG
GKQALETVQRLLPVLCQDHGLTPDQVVAIASHDGGKQALETVQRLLPVLCQDHGLTP
DQVVAIASNIGGKQALETVQRLLPVLCQDHGLTPDQVVAIASNGGGKQALESIVAQLSR
PDPALAALTNDHLVALACLGGRPAMDAVKKGLPHAPELIRRVNRRIGERTSHRVAGSQL
VKSELEEKKSELRHKLKYVPHEYIELIEIARNSTQDRILEMKVMEFFMKVYGYRGKHL
GGSRKPDGAIYTVGSPIDYGVIVDTKAYSGGYNLPIGQADEMERYVEENQTRNKHLNP
NEWWKVYPSSVTEFKFLFVSGHFKGNYKAQLTRLNHITNCNGAVLSVEELLIGGEMIK
AGTLTLEEVRRKFNNGEINF
A3A-TALE-FokI-L_{nickase}
(SEQ ID NO. 171)
MAPKKKRKVMEASPASGPRHLMDPHIFTSNFNNGIGRHKTYLCYEVERLDNGTSV
KMDQHRGFLHNQAKNLLCGFYGRHAELRFLDLVPSLQLDPAQIYRVTWFISWSPCFSW
GCAGEVRAFLQENTHVRLRIFAARIYDYDPLYKEALQMLRDAGAQVSIMTYDEFKHC
WDTFVDHQGCPFQPWDGLDEHSQALSGRLRAILQNQGSGSETPGTSESATPESSGGSS
GGSSGSETPGTSESATPESSGGSSGGSGIHGVPSRMVDLRTLGYSQQQQEKIKPKVRSTV
AQHHEALVGHGFTHAHIVALSQHPAALGTVAVTYQHIITALPEATHEDIVGVGKQWSG
ARALEALLTDAGELRGPPLQLDTGQLVKIAKRGGVTAMEAVHASRNALTGAPLNLTPD
QVVAIASHDGGKQALETVQRLLPVLCQDHGLTPDQVVAIASHDGGKQALETVQRLLP
VLCQDHGLTPDQVVAIASNGGGKQALETVQRLLPVLCQDHGLTPDQVVAIASNNGGK
QALETVQRLLPVLCQDHGLTPDQVVAIASNNGGKQALETVQRLLPVLCQDHGLTPDQ
VVAIASNIGGKQALETVQRLLPVLCQDHGLTPDQVVAIASHDGGKQALETVQRLLPVL
CQDHGLTPDQVVAIASHDGGKQALETVQRLLPVLCQDHGLTPDQVVAIASNNGGKQA
LETVQRLLPVLCQDHGLTPDQVVAIASHDGGKQALETVQRLLPVLCQDHGLTPDQVVA
IASNNGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALETVQ
RLLPVLCQDHGLTPDQVVAIASNNGGKQALETVQRLLPVLCQDHGLTPDQVVAIASNI
GGKQALETVQRLLPVLCQDHGLTPDQVVAIASHDGGKQALETVQRLLPVLCQDHGLT
PDQVVAIASHDGGKQALETVQRLLPVLCQDHGLTPDQVVAIASNIGGKQALETVQRLL
PVLCQDHGLTPDQVVAIASNGGGKQALETVQRLLPVLCQDHGLTPDQVVAIASHDGG
KQALETVQRLLPVLCQDHGLTPDQVVAIASHDGGKQALETVQRLLPVLCQDHGLTPD
QVVAIASNIGGKQALETVQRLLPVLCQDHGLTPDQVVAIASHDGGKQALETVQRLLPV
LCQDHGLTPDQVVAIASNNGGKQALESIVAQLSRPDPALAALTINDHLVALACLGGRPA
MDAVKKGLPHAPELIRRVNRRIGERTSHRVAGSQLVKSELEEKKSELRHKLKYVPHEYI
ELIEIARNSTQDRILEMKVMEFFMKVYGYRGKHLGGSRKPDGAIYTVGSPIDYGVIVD
TKAYSGGYNLPIGQADEMQRYVKENQTRNKHINPNEWWKVYPSSVTEFKFLFVSGHF
KGNYKAQLTRLNHKTNCNGAVLSVEELLIGGEMIKAGTLTLEEVRRKFNNGEINFRSG
GGEGRGSLLTCGDVEENPGPRMDYKDHDGDYKDHDIDYKDDDDKMAPKKKRKVGI
HGVPARMVDLRTLGYSQQQQEKIKPKVRSTVAQHHEALVGHGFTHAHIVALSQHPAAL
GTVAVTYQHIITALPEATHEDIVGVGKQWSGARALEALLTDAGELRGPPLQLDTGQLVK
IAKRGGVTAMEAVHASRNALTGAPLNLTPDQVVAIASHDGGKQALETVQRLLPVLCQD
HGLTPDQVVAIASHDGGKQALETVQRLLPVLCQDHGLTPDQVVAIASHDGGKQALET
VQRLLPVLCQDHGLTPDQVVAIASNIGGKQALETVQRLLPVLCQDHGLTPDQVVAIAS
NNGGKQALETVQRLLPVLCQDHGLTPDQVVAIASNIGGKQALETVQRLLPVLCQDHGL
TPDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASHDG
GKQALETVQRLLPVLCQDHGLTPDQVVAIASHDGGKQALETVQRLLPVLCQDHGLTP
DQVVAIASNNGGKQALETVQRLLPVLCQDHGLTPDQVVAIASHDGGKQALETVQRLL
PVLCQDHGLTPDQVVAIASNNGGKQALETVQRLLPVLCQDHGLTPDQVVAIASNNGG
KQALETVQRLLPVLCQDHGLTPDQVVAIASHDGGKQALETVQRLLPVLCQDHGLTPD
QVVAIASNNGGKQALETVQRLLPVLCQDHGLTPDQVVAIASHDGGKQALESIVAQLSR
PDPALAALTNDHLVALACLGGRPAMDAVKKGLPHAPELIRRVNRRIGERTSHRVAGSQL
VKSELEEKKSELRHKLKYVPHEYIELIEIARNSTQDRILEMKVMEFFMKVYGYRGKHL
GGSRKPAGAIYTVGSPIDYGVIVDTKAYSGGYNLPIGQADEMERYVEENQTRNKHLNP
NEWWKVYPSSVTEFKFLFVSGHFKGNYKAQLTRLNHITNCNGAVLSVEELLIGGEMIK
AGTLTLEEVRRKFNNGEINF
APOBEC1-TALE-FokI-L_{nickase}
(SEQ ID NO. 172)
MAPKKKRKVSSETGPVAVDPTLRRRIEPHEFEVFFDPRELRKETCLLYEINWGGRH
SIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHW
PRYPHLWVRLYVLELYCIILGLPPCLNILRRKQPQLTFFTIALQSCHYQRLPPHILWATGL
KSGSETPGTSESATPESSGGSSGGSSGSETPGTSESATPESSGGSSGGSGIHGVPSRMVDL
RTLGYSQQQQEKIKPKVRSTVAQHHEALVGHGFTHAHIVALSQHPAALGTVAVTYQHII
TALPEATHEDIVGVGKQWSGARALEALLTDAGELRGPPLQLDTGQLVKIAKRGGVTA
MEAVHASRNALTGAPLNLTPDQVVAIASHDGGKQALETVQRLLPVLCQDHGLTPDQV
VAIASHDGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASNNGGKQAL
ETVQRLLPVLCQDHGLTPDQVVAIASNIGGKQALETVQRLLPVLCQDHGLTPDQVVAI
ASHDGGKQALETVQRLLPVLCQDHGLTPDQVVAIASHDGGKQALETVQRLLPVLCQD
HGLTPDQVVAIASNNGGKQALETVQRLLPVLCQDHGLTPDQVVAIASHDGGKQALET
VQRLLPVLCQDHGLTPDQVVAIASNNGGKQALETVQRLLPVLCQDHGLTPDQVVAIAS
NIGGKQALETVQRLLPVLCQDHGLTPDQVVAIASNIGGKQALETVQRLLPVLCQDHGL
TPDQVVAIASNIGGKQALETVQRLLPVLCQDHGLTPDQVVAIASNNGGKQALETVQRL
LPVLCQDHGLTPDQVVAIASNIGGKQALETVQRLLPVLCQDHGLTPDQVVAIASHDGG
KQALETVQRLLPVLCQDHGLTPDQVVAIASHDGGKQALETVQRLLPVLCQDHGLTPD
QVVAIASNIGGKQALETVQRLLPVLCQDHGLTPDQVVAIASNGGGKQALETVQRLLPV
LCQDHGLTPDQVVAIASHDGGKQALETVQRLLPVLCQDHGLTPDQVVAIASHDGGKQ
ALETVQRLLPVLCQDHGLTPDQVVAIASNIGGKQALETVQRLLPVLCQDHGLTPDQVV
AIASHDGGKQALETVQRLLPVLCQDHGLTPDQVVAIASNNGGKQALESIVAQLSRPDP
ALAALTNDHLVALACLGGRPAMDAVKKGLPHAPELIRRVNRRIGERTSHRVAGSQLVKS
ELEEKKSELRHKLKYVPHEYIELIEIARNSTQDRILEMKVMEFFMKVYGYRGKHLGGS
RKPDGAIYTVGSPIDYGVIVDTKAYSGGYNLPIGQADEMQRYVKENQTRNKHINPNEW
WKVYPSSVTEFKFLFVSGHFKGNYKAQLTRLNHKTNCNGAVLSVEELLIGGEMIKAGT
LTLEEVRRKFNNGEINFRSGGGEGRGSLLTCGDVEENPGPRMDYKDHDGDYKDHDID
YKDDDDKMAPKKKRKVGIHGVPARMVDLRTLGYSQQQQEKIKPKVRSTVAQHHEAL
VGHGFTHAHIVALSQHPAALGTVAVTYQHIITALPEATHEDIVGVGKQWSGARALEALL
TDAGELRGPPLQLDTGQLVKIAKRGGVTAMEAVHASRNALTGAPLNLTPDQVVAIASH
DGGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNIGGKQALETVQRL
LPVLCQDHGLTPDQVVAIASNNGGKQALETVQRLLPVLCQDHGLTPDQVVAIASNIGG
KQALETVQRLLPVLCQDHGLTPDQVVAIASNNGGKQALETVQRLLPVLCQDHGLTPD
QVVAIASHDGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAIASHDGGK
QALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNNGGKQALETVQRLLPVL
CQDHGLTPDQVVAIASNNGGKQALETVQRLLPVLCQDHGLTPDQVVAIASHDGGKQA
LETVQRLLPVLCQDHGLTPDQVVAIASNNGGKQALETVQRLLPVLCQDHGLTPDQVVA
IASHDGGKQALESIVAQLSRPDPALAALTNDHLVALACLGGRPAMDAVKKGLPHAPELI
RRVNRRIGERTSHRVAGSQLVKSELEEKKSELRHKLKYVPHEYIELIEIARNSTQDRILE
MKVMEFFMKVYGYRGKHLGGSRKPAGAIYTVGSPIDYGVIVDTKAYSGGYNLPIGQA
DEMERYVEENQTRNKHLNPNEWWKVYPSSVTEFKFLFVSGHFKGNYKAQLTRLNHIT
NCNGAVLSVEELLIGGEMIKAGTLTLEEVRRKFNNGEINF
SIRT6-NLS-TALE-L-DddAN-UGI
(SEQ ID NO. 173)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPDQVVAIASNGGGKQALETVQRLLPV
LCQDHGLTPEQVVAIASNIGGKQALETVQRLLPVLCQAHGLTPDQVVAIASHDGGKQA
LETVQRLLPVLCQAHGLTPAQVVAIANNNGGKQALETVQRLLPVLCQDHGLTPDQVV
AIASHDGGKQALETVQRLLPVLCQDHGLTPEQVVAIANNNGGKQALETVQRLLPVLC
QAHGLTPDQVVAIANNNGGKQALETVQRLLPVLCQAHGLTPAQVVAIASHDGGKQAL
ETVQRLLPVLCQDHGLTPDQVVAIANNNGGKQALETVQRLLPVLCQDHGLTPEQVVAI
ANNNGGKQALETVQRLLPVLCQAHGLTPDQVVAIANNNGGKQALETVQRLLPVLCQ
AHGLTPAQVVAIANNNGGKQALETVQRLLPVLCQDHGLTPDQVVAIASHDGGKQALE
TVQRLLPVLCQDHGLTPEQVVAIASNGGGKQALETVQRLLPVLCQAHGLTPDQVVAIA
NNNGGKQALETVQRLLPVLCQAHGLTPAQVVAIASNGGGKQALETVQRLLPVLCQDH
GLTPEQVVAIASHDGGRPALESIVAQLSRPDPALAALTNDHLVALACLGGRPALDAVKK
GLGGSGSYALGPYQISAPQLPAYNGQTVGTFYYVNDAGGLESKVFSSGGPTPYPNYAN
AGHVEGQSALFMRDNGISEGLVFHNNPEGTCGFCVNMTETLLPENAKMTVVPPEGSG
GSTNLSDIIEKETGKQLVIQESILMLPEEVEEVIGNKPESDILVHTAYDESTDENVMLLTS
DAPEYKPWALVIQDSNGENKIKML
SIRT6-NLS-TALE-R-DddAc-UGI
(SEQ ID NO. 174)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPDQVVAIASHDGGKQALETVQRLLPVLCQDHGLTPE
QVVAIASHDGGKQALETVQRLLPVLCQAHGLTPDQVVAIANNNGGKQALETVQRLLP
VLCQAHGLTPAQVVAIANNNGGKQALETVQRLLPVLCQDHGLTPDQVVAIANNNGGK
QALETVQRLLPVLCQDHGLTPEQVVAIASNIGGKQALETVQRLLPVLCQAHGLTPDQV
VAIANNNGGKQALETVQRLLPVLCQAHGLTPAQVVAIANNNGGKQALETVQRLLPVL
CQDHGLTPDQVVAIASHDGGKQALETVQRLLPVLCQDHGLTPEQVVAIASHDGGKQA
LETVQRLLPVLCQAHGLTPDQVVAIANNNGGKQALETVQRLLPVLCQAHGLTPAQVV
AIASHDGGKQALETVQRLLPVLCQDHGLTPDQVVAIASNIGGKQALETVQRLLPVLCQ
DHGLTPEQVVAIASHDGGKQALETVQRLLPVLCQAHGLTPDQVVAIASNGGGKQALET
VQRLLPVLCQAHGLTPAQVVAIASNGGGKQALETVQRLLPVLCQDHGLTPEQVVAIAS
NNGGRPALESIVAQLSRPDPALAALTNDHLVALACLGGRPALDAVKKGLGGSAIPVKRG
ATGETKVFTGNSNSPKSPTKGGCSGGSTNLSDIIEKETGKQLVIQESILMLPEEVEEVIGN
KPESDILVHTAYDESTDENVMLLTSDAPEYKPWALVIQDSNGENKIKML

### In Examples 11, 14 and 15

ND6-MTS-TALE-L-DddA_{N}-UGI
(SEQ ID NO. 175)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPEQVVAIASNNGGKQALETVQRLLPV
LCQAHGLTPEQVVAIASNIGGKQALETVQRLLPVLCQAHGLTPEQVVAIASHDGGKQA
LETVQRLLPVLCQAHGLTPEQVVAIASHDGGKQALETVQRLLPVLCQAHGLTPEQVVA
IASHDGGKQALETVQRLLPVLCQAHGLTPEQVVAIASHDGGKQALETVQRLLPVLCQA
HGLTPEQVVAIASHDGGKQALETVQRLLPVLCQAHGLTPEQVVAIASNIGGKQALETV
QRLLPVLCQAHGLTPEQVVAIASNGGGKQALETVQRLLPVLCQAHGLTPEQVVAIASN
GGGRPALESIVAQLSRPDPALAALTNDHLVALACLGGRPALDAVKKGLGGSGSYALGPY
QISAPQLPAYNGQTVGTFYYVNDAGGLESKVFSSGGPTPYPNYANAGHVEGQSALFMR
DNGISEGLVFHNNPEGTCGFCVNMTETLLPENAKMTVVPPEGSGGSTNLSDIIEKETGK
QLVIQESILMLPEEVEEVIGNKPESDILVHTAYDESTDENVMLLTSDAPEYKPWALVIQD
SNGENKIKML
ND6-MTS-TALE-R-DddA_{C}-UGI
(SEQ ID NO. 176)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPEQVVAIASNNGGKQALETVQRLLPVLCQAHGLTPE
QVVAIASNIGGKQALETVQRLLPVLCQAHGLTPEQVVAIASNGGGKQALETVQRLLPV
LCQAHGLTPEQVVAIASNNGGKQALETVQRLLPVLCQAHGLTPEQVVAIASNNGGKQA
LETVQRLLPVLCQAHGLTPEQVVAIASHDGGKQALETVQRLLPVLCQAHGLTPEQVVA
IASNGGGKQALETVQRLLPVLCQAHGLTPEQVVAIASNIGGKQALETVQRLLPVLCQA
HGLTPEQVVAIASNGGGKQALETVQRLLPVLCQAHGLTPEQVVAIASNGGGKQALETV
QRLLPVLCQAHGLTPEQVVAIASNGGGRPALESIVAQLSRPDPALAALTNDHLVALACL
GGRPALDAVKKGLGGSAIPVKRGATGETKVFTGNSNSPKSPTKGGCSGGSTNLSDIIEK
ETGKQLVIQESILMLPEEVEEVIGNKPESDILVHTAYDESTDENVMLLTSDAPEYKPWAL
VIQDSNGENKIKML
ND1.2-MTS-TALE-L-DddA_{N}-UGI
(SEQ ID NO. 177)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPEQVVAIASHDGGKQALETVQALLPV
LCQAHGLTPQQVVAIASHDGGKQALETVQRLLPVLCQAHGLTPQQVVAIASNGGGKQ
ALETVQRLLPVLCQAHGLTPEQVVAIASNIGGKQALETVQALLPVLCQAHGLTPEQVV
AIASNGGGKQALETVQALLPVLCQAHGLTPEQVVAIASNGGGKQALETVQALLPVLC
QAHGLTPEQVVAIASNGGGKQALETVQRLLPVLCQAHGLTPEQVVAIASNIGGKQALE
TVQALLPVLCQAHGLTPEQVVAIASNGGGKQALETVQRLLPVLCQAHGLTPEQVVAIA
SNGGGKQALETVQALLPVLCQAHGLTPEQVVAIASHDGGKQALETVQALLPVLCQAH
GLTPEQVVAIASNGGGKQALETVQALLPVLCQAHGLTPEQVVAIASNIGGKQALETVQ
ALLPVLCQAHGLTPQQVVAIASNNGGRPALESIVAQLSRPDPALAALTNDHLVALACLG
GRPALDAVKKGLGGSGSYALGPYQISAPQLPAYNGQTVGTFYYVNDAGGLESKVFSSG
GPTPYPNYANAGHVEGQSALFMRDNGISEGLVFHNNPEGTCGFCVNMTETLLPENAK
MTVVPPEGSGGSTNLSDIIEKETGKQLVIQESILMLPEEVEEVIGNKPESDILVHTAYDES
TDENVMLLTSDAPEYKPWALVIQDSNGENKIKML
ND1.2-MTS-TALE-R-DddA_{C}-UGI
(SEQ ID NO. 178)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPEQVVAIASNGGGKQALETVQALLPVLCQAHGLTPQ
QVVAIASNNGGKQALETVQRLLPVLCQAHGLTPQQVVAIASNIGGKQALETVQRLLPV
LCQAHGLTPEQVVAIASNNGGKQALETVQALLPVLCQAHGLTPEQVVAIASNGGGKQ
ALETVQALLPVLCQAHGLTPEQVVAIASNIGGKQALETVQALLPVLCQAHGLTPEQVV
AIASNIGGKQALETVQRLLPVLCQAHGLTPEQVVAIASNIGGKQALETVQALLPVLCQA
HGLTPEQVVAIASHDGGKQALETVQRLLPVLCQAHGLTPEQVVAIASNNGGKQALETV
QALLPVLCQAHGLTPEQVVAIASNNGGKQALETVQALLPVLCQAHGSIVAQLSRPDPA
LAALTNDHLVALACLGGRPALDAVKKGLGGSAIPVKRGATGETKVFTGNSNSPKSPTK
GGCSGGSTNLSDIIEKETGKQLVIQESILMLPEEVEEVIGNKPESDILVHTAYDESTDENV
MLLTSDAPEYKPWALVIQDSNGENKIKML
ND1.3-MTS-TALE-L-DddA_{N}-UGI
(SEQ ID NO. 179)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPEQVVAIASHDGGKQALETVQALLPV
LCQAHGLTPQQVVAIASNGGGKQALETVQRLLPVLCQAHGLTPQQVVAIASHDGGKQ
ALETVQRLLPVLCQAHGLTPEQVVAIASHDGGKQALETVQALLPVLCQAHGLTPEQVV
AIASNIGGKQALETVQALLPVLCQAHGLTPEQVVAIASHDGGKQALETVQALLPVLCQ
AHGLTPEQVVAIASNIGGKQALETVQRLLPVLCQAHGLTPEQVVAIASHDGGKQALET
VQALLPVLCQAHGLTPEQVVAIASNGGGKQALETVQRLLPVLCQAHGLTPEQVVAIAS
NIGGKQALETVQALLPVLCQAHGLTPEQVVAIASNNGGKQALETVQALLPVLCQAHGL
TPEQVVAIASHDGGKQALETVQALLPVLCQAHGLTPEQVVAIASNIGGKQALETVQRL
LPVLCQAHGLTPQQVVAIASNNGGKQALETVQRLLPVLCQAHGLTPEQVVAIASNIGG
KQALETVQALLPVLCQAHGLTPQQVVAIASNNGGRPALESIVAQLSRPDPALAALTNDH
LVALACLGGRPALDAVKKGLGGSGSYALGPYQISAPQLPAYNGQTVGTFYYVNDAGGL
ESKVFSSGGPTPYPNYANAGHVEGQSALFMRDNGISEGLVFHNNPEGTCGFCVNMTET
LLPENAKMTVVPPEGSGGSTNLSDIIEKETGKQLVIQESILMLPEEVEEVIGNKPESDILV
HTAYDESTDENVMLLTSDAPEYKPWALVIQDSNGENKIKML
ND1.3-MTS-TALE-R-DddA_{C}-UGI
(SEQ ID NO. 180)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPEQVVAIASHDGGKQALETVQALLPVLCQAHGLTPQ
QVVAIASHDGGKQALETVQRLLPVLCQAHGLTPQQVVAIASHDGGKQALETVQRLLP
VLCQAHGLTPEQVVAIASHDGGKQALETVQALLPVLCQAHGLTPEQVVAIASNGGGK
QALETVQALLPVLCQAHGLTPEQVVAIASNGGGKQALETVQALLPVLCQAHGLTPEQV
VAIASHDGGKQALETVQRLLPVLCQAHGLTPEQVVAIASNNGGKQALETVQALLPVLC
QAHGLTPEQVVAIASNNGGKQALETVQRLLPVLCQAHGLTPEQVVAIASHDGGKQALE
TVQALLPVLCQAHGLTPEQVVAIASNIGGKQALETVQALLPVLCQAHGLTPEQVVAIAS
NIGGKQALETVQALLPVLCQAHGLTPEQVVAIASNNGGKQALETVQRLLPVLCQAHGL
TPQQVVAIASNNGGKQALETVQRLLPVLCQAHGLTPEQVVAIASNGGGKQALETVQAL
LPVLCQAHGLTPEQVVAIASNGGGKQALETVQALLPVLCQAHGLTPQQVVAIASHDGG
RPALESIVAQLSRPDPALAALTNDHLVALACLGGRPALDAVKKGLGGSAIPVKRGATGE
TKVFTGNSNSPKSPTKGGCSGGSTNLSDIIEKETGKQLVIQESILMLPEEVEEVIGNKPES
DILVHTAYDESTDENVMLLTSDAPEYKPWALVIQDSNGENKIKML
ND6.2-MTS-TALE-L-DddA_{N}-UGI
(SEQ ID NO. 181)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPEQVVAIASHDGGKQALETVQALLPV
LCQAHGLTPQQVVAIASHDGGKQALETVQRLLPVLCQAHGLTPQQVVAIASHDGGKQ
ALETVQRLLPVLCQAHGLTPEQVVAIASNIGGKQALETVQALLPVLCQAHGLTPEQVV
AIASNGGGKQALETVQALLPVLCQAHGLTPEQVVAIASNNGGKQALETVQALLPVLC
QAHGLTPEQVVAIASHDGGKQALETVQRLLPVLCQAHGLTPEQVVAIASHDGGKQALE
TVQALLPVLCQAHGLTPEQVVAIASNGGGKQALETVQRLLPVLCQAHGLTPEQVVAIA
SHDGGKQALETVQALLPVLCQAHGLTPEQVVAIASNIGGKQALETVQALLPVLCQAH
GLTPEQVVAIASNNGGKQALETVQALLPVLCQAHGLTPEQVVAIASNNGGKQALETVQ
RLLPVLCQAHGLTPQQVVAIASNIGGKQALETVQRLLPVLCQAHGLTPEQVVAIASNGG
GKQALETVQALLPVLCQAHGLTPQQVVAIASNIGGRPALESIVAQLSRPDPALAALTND
HLVALACLGGRPALDAVKKGLGGSGSYALGPYQISAPQLPAYNGQTVGTFYYVNDAG
GLESKVFSSGGPTPYPNYANAGHVEGQSALFMRDNGISEGLVFHNNPEGTCGFCVNMT
ETLLPENAKMTVVPPEGSGGSTNLSDIIEKETGKQLVIQESILMLPEEVEEVIGNKPESDI
LVHTAYDESTDENVMLLTSDAPEYKPWALVIQDSNGENKIKML
ND6.2-MTS-TALE-R-DddA_{C}-UGI
(SEQ ID NO. 182)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPEQVVAIASNGGGKQALETVQALLPVLCQAHGLTPQ
QVVAIASNGGGKQALETVQRLLPVLCQAHGLTPQQVVAIASNNGGKQALETVQRLLP
VLCQAHGLTPEQVVAIASNNGGKQALETVQALLPVLCQAHGLTPEQVVAIASNIGGKQ
ALETVQALLPVLCQAHGLTPEQVVAIASNGGGKQALETVQALLPVLCQAHGLTPEQVV
AIASNIGGKQALETVQRLLPVLCQAHGLTPEQVVAIASNGGGKQALETVQALLPVLCQ
AHGLTPEQVVAIASNIGGKQALETVQRLLPVLCQAHGLTPEQVVAIASHDGGKQALET
VQALLPVLCQAHGLTPEQVVAIASNGGGKQALETVQALLPVLCQAHGLTPEQVVAIAS
NIGGKQALETVQALLPVLCQAHGLTPEQVVAIASHDGGKQALETVQRLLPVLCQAHGL
TPQQVVAIASNIGGKQALETVQRLLPVLCQAHGLTPEQVVAIASNNGGKQALETVQAL
LPVLCQAHGLTPQQVVAIASHDGGRPALESIVAQLSRPDPALAALTNDHLVALACLGGR
PALDAVKKGLGGSAIPVKRGATGETKVFTGNSNSPKSPTKGGCSGGSTNLSDIIEKETG
KQLVIQESILMLPEEVEEVIGNKPESDILVHTAYDESTDENVMLLTSDAPEYKPWALVIQ
DSNGENKIKML
ND3-MTS-TALE-L-DddA_{N}-UGI
(SEQ ID NO. 183)
MALSRAVCGTSRQLAPVLGYLGSRQKHSLPDYPYDVPDYAGYPYDVPDYAGYPY
DVPDYAMDIADLRTLGYSQQQQEKIKPKVRSTVAQHHEALVGHGFTHAHIVALSQHPA
ALGTVAVKYQDMIAALPEATHEAIVGVGKQWSGARALEALLTVAGELRGPPLQLDTG
QLLKIAKRGGVTAVEAVHAWRNALTGAPLNLTPEQVVAIASNIGGKQALETVQALLPV
LCQAHGLTPQQVVAIASHDGGKQALETVQRLLPVLCQAHGLTPQQVVAIASNNGGKQ
ALETVQRLLPVLCQAHGLTPEQVVAIASNIGGKQALETVQALLPVLCQAHGLTPEQVV
AIASNNGGKQALETVQALLPVLCQAHGLTPEQVVAIASNGGGKQALETVQALLPVLC
QAHGLTPEQVVAIASNNGGKQALETVQRLLPVLCQAHGLTPEQVVAIASHDGGKQALE
TVQALLPVLCQAHGLTPEQVVAIASNNGGKQALETVQRLLPVLCQAHGLTPEQVVAIA
SNNGGKQALETVQALLPVLCQAHGLTPEQVVAIASHDGGKQALETVQALLPVLCQAH
GLTPEQVVAIASNGGGKQALETVQALLPVLCQAHGLTPEQVVAIASNGGGKQALETVQ
RLLPVLCQAHGLTPQQVVAIASHDGGKQALETVQRLLPVLCQAHGLTPEQVVAIASNN
GGKQALETVQALLPVLCQAHGLTPQQVVAIASNIGGRPALESIVAQLSRPDPALAALTN
DHLVALACLGGRPALDAVKKGLGGSGSYALGPYQISAPQLPAYNGQTVGTFYYVNDA
GGLESKVFSSGGPTPYPNYANAGHVEGQSALFMRDNGISEGLVFHNNPEGTCGFCVN
MTETLLPENAKMTVVPPEGSGGSTNLSDIIEKETGKQLVIQESILMLPEEVEEVIGNKPE
SDILVHTAYDESTDENVMLLTSDAPEYKPWALVIQDSNGENKIKML
ND3-MTS-TALE-R-DddA_{C}-UGI
(SEQ ID NO. 184)
MASVLTPLLLRGLTGSARRLPVPRAKIHSLDYKDHDGDYKDHDIDYKDDDDKMDI
ADLRTLGYSQQQQEKIKPKVRSTVAQHHEALVGHGFTHAHIVALSQHPAALGTVAVKY
QDMIAALPEATHEAIVGVGKRGAGARALEALLTVAGELRGPPLQLDTGQLLKIAKRGG
VTAVEAVHAWRNALTGAPLNLTPEQVVAIASNIGGKQALETVQALLPVLCQAHGLTPQ
QVVAIASNGGGKQALETVQRLLPVLCQAHGLTPQQVVAIASNNGGKQALETVQRLLP
VLCQAHGLTPEQVVAIASNNGGKQALETVQALLPVLCQAHGLTPEQVVAIASNIGGKQ
ALETVQALLPVLCQAHGLTPEQVVAIASNNGGKQALETVQALLPVLCQAHGLTPEQVV
AIASNIGGKQALETVQRLLPVLCQAHGLTPEQVVAIASNIGGKQALETVQALLPVLCQA
HGLTPEQVVAIASNIGGKQALETVQRLLPVLCQAHGLTPEQVVAIASNNGGKQALETV
QALLPVLCQAHGLTPEQVVAIASNNGGKQALETVQALLPVLCQAHGLTPEQVVAIASN
NGGKQALETVQALLPVLCQAHGLTPEQVVAIASNIGGKQLETVQRLLPVLCQAHGLTP
QQVVAIASHDGGKQALETVQRLLPVLCQAHGLTPEQVVAIASNNGGKQALETVQALLP
VLCQAHGLTPQQVVAIASHDGGRPALESIVAQLSRPDPALAALTNDHLVALACLGGRPA
LDAVKKGLGGSAIPVKRGATGETKVFTGNSNSPKSPTKGGCSGGSTNLSDIIEKETGKQ
LVIQESILMLPEEVEEVIGNKPESDILVHTAYDESTDENVMLLTSDAPEYKPWALVIQDS
NGENKIKML

The target sequences in the following Examples and the accompanying drawings involved therein are set forth below.

A strand of the OsBADH2 target site in the figure

SEQ ID NO. 188
GCTGGATGCTTTGAGTACTTTGCAGATCTTGCAGAATCCTTGGACAAAA
GGC

B strand of the OsBADH2 target site in the figure

SEQ ID NO. 189
CGACCTACGAAACTCATGAAACGTCTAGAACGTCTTAGGAACCTGTTTT
CCG

A strand of the OsDEP1 target site in the figure

SEQ ID NO. 190
GCAAAAGACCAAGGTGCCTCAATTGTTCTTGCAGCTCATGCTGCGACGA
GCC

B strand of the OsDEP1 target site in the figure

SEQ ID NO. 191
CGTTTTCTGGTTCCACGGAGTTAACAAGAACGTCGAGTACGACGCTGCT
CGG

A strand of the OsCKX2 target site in the figure

SEQ ID NO. 192
CCTGGACCGCGTCCACGACGGCGAGCTCAAGCTCCGCGCCGCGGGGCTC
TGGG

B strand of the OsCKX2 target site in the figure

SEQ ID NO. 193
GGACCTGGCGCAGGTGCTGCCGCTCGAGTTCGAGGCGCGGCGCCCCGAG
ACCC

A strand of the Human ND6 target site in the figure

SEQ ID NO. 194
CCCCTGACCCCCATGCCTCAGGATACTCCTCAATAGCCATCGCTGTA

B strand of the Human ND6 target site in the figure

SEQ ID NO. 195
GGGGACTGGGGGTACGGAGTCCTATGAGGAGTTATCGGTAGCGACAT

A strand of the OsSD1 target site in the figure

SEQ ID NO. 196
CCAGGACGACGTCGGCGGCCTCGAGGTCCTCGTCGACGGCGAATGGCGC
CCCGTC

B strand of the OsSD1 target site in the figure

SEQ ID NO. 197
GGTCCTGCTGCAGCCGCCGGAGCTCCAGGAGCAGCTGCCGCTTACCGCG
GGGCAG

A strand of the SIRT6 target site in the figure

SEQ ID NO. 198
TACGCGGGGGGCTGTCGCCGTACGCGGACAAGGGCAAGTGCGGCCTCCC
GG

B strand of the SIRT6 target site in the figure

SEQ ID NO. 199
ATGCGCCGCCCCGACAGCGGCATGCGCCTGTTCCCGTTCACGCCGGAGG
GCC

A strand of the OsRbcL target site in the figure

SEQ ID NO. 200
TTACCAAAGATGATGAAAACGTAAACTCACAACCATTTATGCGTTGG

B strand of the OsRbcL target site in the figure

SEQ ID NO. 201
AATGGTTTCTACTACTTTTGCATTTGAGTGTTGGTAAATACGCAACC

A strand of the ND6.2 target site in the figure

SEQ ID NO. 202
GACCCCCATGCCTCAGGATACTCCTCAATAGCCATCGCTGTAGTATAT
CCAA

B strand of the ND6.2 target site in the figure

SEQ ID NO. 203
CTGGGGGTACGGAGTCCTATGAGGAGTTATCGGTAGCGACATCATATA
GGTT

A strand of the ND1.2 target site in the figure

SEQ ID NO. 204
CCTATTTATTCTAGCCACCTCTAGCCTAGCCGTTTACTCA

B strand of the ND1.2 target site in the figure

SEQ ID NO. 205
GGATAAATAAGATCGGTGGAGATCGGATCGGCAAATGAGT

A strand of the ND1.3 target site in the figure

SEQ ID NO. 206
TCTCCACACTAGCAGAGACCAACCGAACCCCCTTCGACCTTGCCGAAG
GGG

B strand of the ND1.3 target site in the figure

SEQ ID NO. 207
AGAGGTGTGATCGTCTCTGGTTGGCTTGGGGGAAGCTGGAACGGCTTC
CCC

A strand of the ND3 target site in the figure

SEQ ID NO. 208
ACGAGTGCGGCTTCGACCCTATATCCCCCGCCCGCGTCCCTTTCTCCA
T

B strand of the ND3 target site in the figure

SEQ ID NO. 209
TGCTCACGCCGAAGCTGGGATATAGGGGGGGGCGCAGGGAAAGAGGT
A

A strand of the ND1 target site in the figure

SEQ ID NO. 210
CTAGCCTAGCCGTTTACTCAATCCTCTCATCAGGGTGAGCATCAAACT
C

B strand of the ND1 target site in the figure

SEQ ID NO. 211
GATCGGATCGGCAAATGAGTTAGGAGACTAGTCCCACTCGTAGTTTGA
G

A strand of the ND4 target site in the figure

SEQ ID NO. 212
GCTAGTAACCACGTTCTCCTGATCAAATATCACTCTCCTACTTACAG
G

B strand of the ND4 target site in the figure

SEQ ID NO. 213
CGATCATTGGTGCAAGAGGACTAGTTTATAGTGAGAGGATGAATGTC
C

A strand of the ND5.1 target site in the figure

SEQ ID NO. 214
AGCATTAGCAGGAATACCTTTCCTCACAGGTTTCTACTCCAAAG

B strand of the ND5.1 target site in the figure

SEQ ID NO. 215
TCGTAATCGTCCTTATGGAAAGGAGTGTCCAAAGATGAGGTTTC
SEQ ID NO. 216
GACCCCCATGCCTCAGGATACTCCTCAATAGCCATC
SEQ ID NO. 217
CTGGGGGTACGGAGTCCTATGAGGAGTTATCGGTAG
SEQ ID NO. 218
CCCCATGCCTCAGGATACTCCTCAATAGCCATCGCTGTAGTATATCCAA
SEQ ID NO. 219
GGGGTACGGAGTCCTATGAGGAGTTATCGGTAGCGACATCATATAGGTT

### Example 1: Synthesis and Determination of Base Editor

The synthesis strategy of the base editor of the present disclosure was as shown in FIG. 1.

In order to verify the above-mentioned strategy, a target site in OsBADH2 gene of rice was selected, two set of TALE encoding vectors modified to target the site were constructed, and the above-mentioned elements were listed in Table 3.

TABLE 3
Special examples of the combinations of base editors in Examples

| | Fusion protein of | | |
|---|---|---|---|
| | sequence-specific DNA | | Fusion protein of |
| | binding protein and | | deaminase and |
| Construct | nickase | Exonuclease | UGI |
| TALEN_{WT} | TALE-L-FokI-L and | Exonuclease I | hAPOBEC3A-UGI |
| | TALE-R-FokI-R | Exonuclease V | hAPOBEC3A-UGI |
| TALE-FokI-R_{nickase(D450A)} | TALE-L-FokI-L_{D450A} and | Exonuclease I | hAPOBEC3A-UGI |
| | TALE-R-FokI-R | Exonuclease V | hAPOBEC3A-UGI |
| TALE-FokI-R_{nickase(D467A)} | TALE-L-FokI-L_{D467A} and | Exonuclease I | hAPOBEC3A-UGI |
| | TALE-R-FokI-R | Exonuclease V | hAPOBEC3A-UGI |

An FokICD (or mutant) monomer was fused to the C-terminal of TALE-L and TALE-R, respectively, and wild-type FokI (without D450A or D467A mutation) was used as a control group (FIG. 16A). The application of two exonucleases (Exonuclease I (rat exonuclease I, simply referred to as mExoI) and Exonuclease V (simply referred to as ExoV)) and one deaminase (hAPOBEC3A, simply referred to as hA3A or A3A) in the novel base editor was evaluated, wherein UGI was fused to the carboxy terminal of the deaminase with an XTEN linker peptide in each group (FIG. 16B). The nuclear localization signal (NLS, i.e., SV40 NLS in Table 2) was fused to the terminal of the protein.

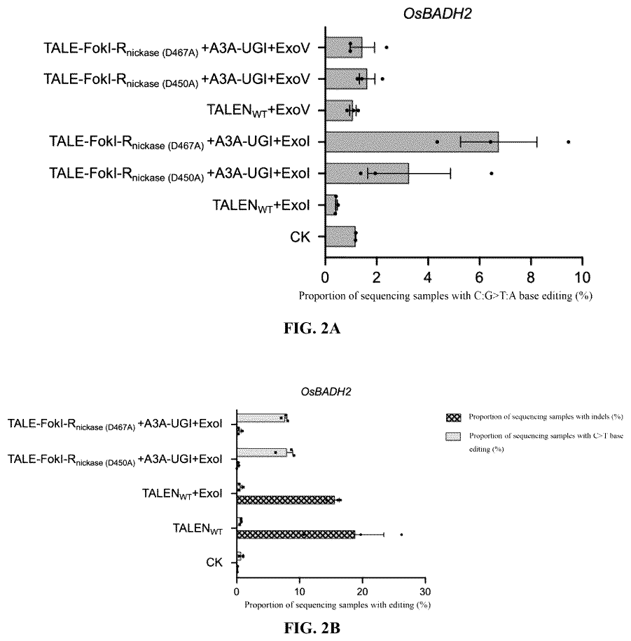

Recombinant expression constructs encoding these components were transformed into rice protoplasts via PEG-mediated transformation. Said constructs were as shown by FIGS. 16A-16B. Rice protoplasts were transformed with different construct combinations to target the OsBADH2 site, and next-generation sequencing (NGS) was used to determine C>T base editing frequency. Sequencing results (FIG. 2A) indicated that, for the combination comprising FokI nickase, deaminase, exonuclease and UGI, targeted cytosine base editing was achieved with a frequency up to about 10%. Importantly, the results of determination also indicated that the novel nucleic acid base editor merely resulted in indel byproducts at a very low level (as shown in FIG. 2B). The above-mentioned results indicated that the novel base editor had the characteristics of achieving high product purity, which was important for precise genome editing.

In FIG. 2A and FIG. 2B, the experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| TALEN_{WT} + mExoI | Exonuclease (mExoI) - nuclear localization |
| | signal fusion protein vector in FIG. 16A and Fig. |
| | 16C |
| TALE-FokI-R_{nickase (D450A)} + | FIG. 16B, wherein the corresponding nickase was |
| A3A-UGI + mExoI | FokI-R_{nickase (D450A)}, the corresponding deaminase |
| | was hAPOBEC3A, and the corresponding |
| | exonuclease was mExoI. |
| TALE-FokI-R_{nickase (D467A)} + | FIG. 16B, wherein the corresponding nickase was |
| A3A-UGI + mExoI | FokI-R_{nickase (D467A)}, the corresponding deaminase |
| | was hAPOBEC3A, and the corresponding |
| | exonuclease was mExoI. |
| TALEN_{WT} + ExoV | Exonuclease (ExoV) - nuclear localization |
| | signal fusion protein vector in FIG. 16A and Fig. |
| | 16C |
| TALE-FokI-R_{nickase (D450A)} + | FIG. 16B, wherein the corresponding nickase was |
| A3A-UGI + ExoV | FokI-R_{nickase (D450A)}, the corresponding deaminase |
| | was hAPOBEC3A, and the corresponding |
| | exonuclease was ExoV. |
| TALE-FokI-R_{nickase (D467A)} + | FIG. 16B, wherein the corresponding nickase was |
| A3A-UGI + ExoV | FokI-R_{nickase (D467A)}, the corresponding deaminase |
| | was hAPOBEC3A, and the corresponding |
| | exonuclease was ExoV. |

### Example 2: Characterization of Cleavage Performance of Base Editor on Single Strand

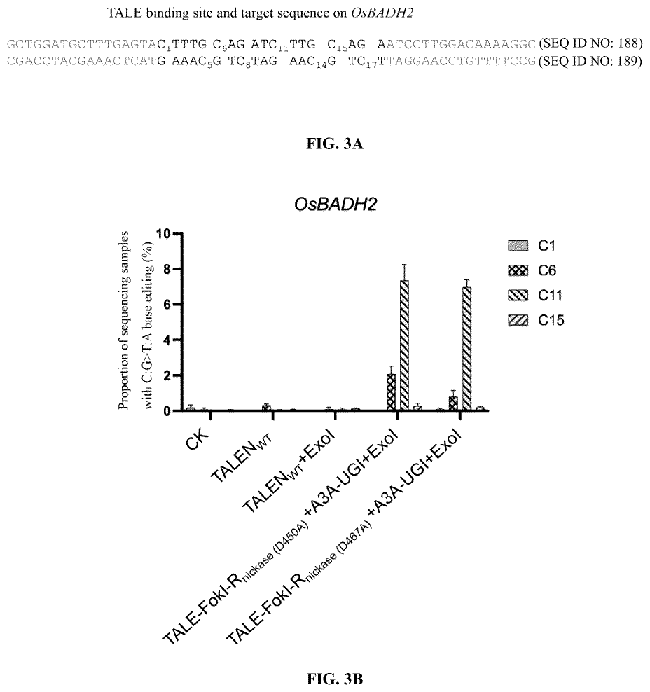

The base editing windows of the base editors tested in Example 1 were analyzed. Among the four C sites (C1, C6, C11 and C15, in the spacer sequence between two TALEs, the first base adjacent to TALE-L was counted as 1) present in strand A of the target gene (as shown in FIG. 3A), the C6 and C11 cytosines were efficiently edited (FIG. 3B).

In FIG. 3B, the experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALEN_{WT} + mExoI | Exonuclease (mExoI) - nuclear localization |
| | signal fusion protein vector in FIG. 16A and |
| | FIG. 16C |
| TALE-FokI-R_{nickase (D450A)} + | FIG. 16B, wherein the corresponding nickase |
| A3A-UGI + mExoI | was FokI-R_{nickase (D450A)}, the corresponding |
| | deaminase was hAPOBEC3A, and the |
| | corresponding exonuclease was mExoI. |
| TALE-FokI-R_{nickase (D467A)} + | FIG. 16B, wherein the corresponding nickase |
| A3A-UGI + mExoI | was FokI-R_{nickase (D467A)}, the corresponding |
| | deaminase was hAPOBEC3A, and the |
| | corresponding exonuclease was mExoI. |
| TALEN_{WT} | FIG. 16A |

These results indicated that the base editor comprising FokI-R_{nickase} (FokI-L in the dimeric nickase composed of FokI-L and FokI-R had a D450A or D467A mutation) tended to nick strand B by nickase, and the nicked single strand was subsequently digested by exonuclease, leaving a short fragment of ssDNA in strand A. The direction of digestion depended on the enzymatic direction (5′ to 3′ or 3 to 5′) of the exonuclease.

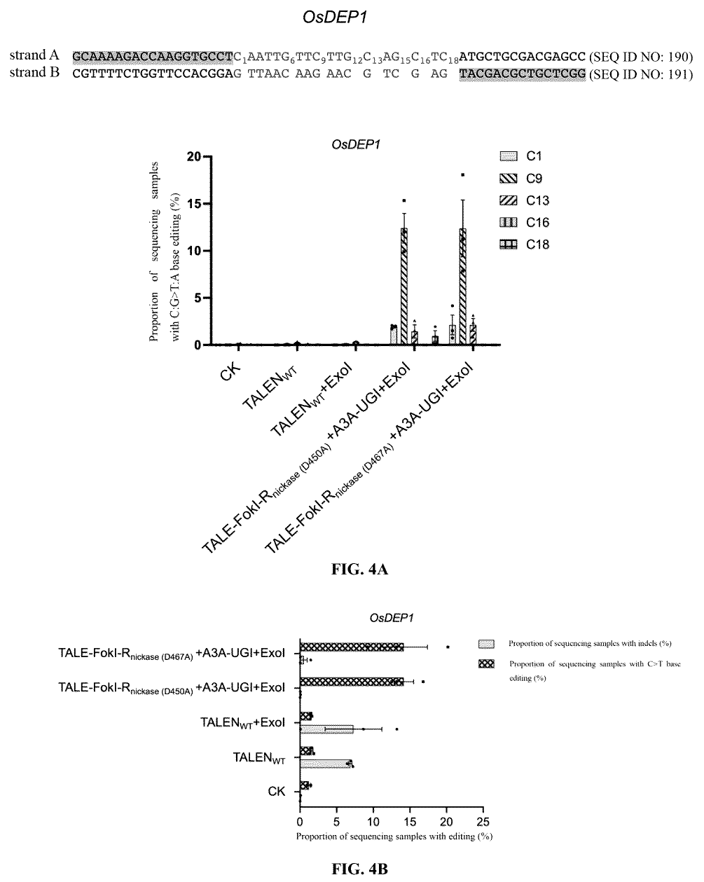

In order to verity the above-mentioned results, the inventors evaluated the nucleic acid base editor at another site (OsDEP1) of the present example, which comprised 5 C-bases (C1, C9, C13, C16 and C18) in strand A. Rice protoplasts were transformed with different construct combinations to target the OsDEP1 site, the NGS analysis results indicated that the base editing window was mainly located near the 5′ region (C9 and C1) in strand A, although C13 and C16 were also slightly edited (as shown in FIG. 4A), which was caused by the generation of a transient 3′ flap structure after nicking. Importantly, similar to the OsBADH2 site, indel byproducts merely appeared in the labeled products at the OsDEP1 site at an extremely low level (as shown in FIG. 4B). The above-mentioned results indicated that the novel base editor achieved the advantage of higher product purity.

In FIG. 4A and FIG. 4B, the experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALEN_{WT} + mExoI | Exonuclease (mExoI) - nuclear localization |
| | signal fusion protein vector in FIG. 16A and |
| | FIG. 16C |
| TALE-FokI-R_{nickase (D450A)} + | FIG. 16B, wherein the corresponding nickase was |
| A3A-UGI + mExoI | FokI-R_{nickase (D450A)}, the corresponding deaminase |
| | was hAPOBEC3A, and the corresponding exonuclease |
| | was mExoI. |
| TALE-FokI-R_{nickase (D467A)} + | FIG. 16B, wherein the corresponding nickase was |
| A3A-UGI + mExol | FokI-R_{nickase (D467A)}, the corresponding deaminase |
| | was hAPOBEC3A, and the corresponding exonuclease |
| | was mExoI. |
| TALEN_{WT} | FIG. 16A |

### Example 3: Effects of Exonuclease Digestion Direction and the Preference of Nickase for Single Strand on Editing Results

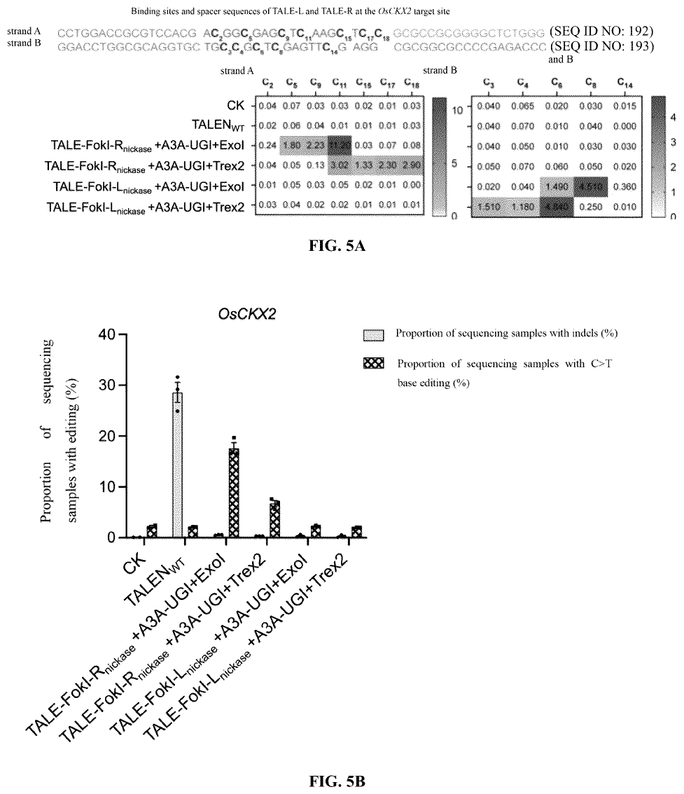

The exonuclease having 5′→3′ digestion directionality (for example, rat exonuclease I (mExoI)) resulted in the exposure of the cytosine residues located near the 5′ region of the target site in the complementary chain and the deamination of the cytosine residues by deaminase; while the 3′ exonuclease resulted in the exposure of the cytosine residues located near the 3′ region of the target site in the complementary chain and the deamination of the cytosine residues by deaminase. To verify the fact that the base editor disclosed in the present disclosure could achieve the expected effects for different exonuclease digestion directions, the inventors tested a 5′ exonuclease (mExoI) and a 3′ exonuclease (human-derived Trex2 exonuclease) at the OsCKX2 target simultaneously, and the editing window of the resulting base editor was analyzed by NGS. As shown by the experimental results, as for the FokI-R_{nickase}-mediated base editing, when the 5′ exonuclease mExoI was used, the editing window was mainly located in the 5′ region (C9 and C11) of strand A of the target site; on the contrary, when the 3′ exonuclease Trex2 was used, the editing window was shifted to 3′-adjacent region (C11 and C15) of strand A of the OsCKX2 target site, and cytosine residues in strand B were not edited (as shown in FIG. 5A and FIG. 5B). Further, the inventors evaluated the impacts of the preference of nickase used for single strand on a single strand where base editing might occur. FokI-R_{nickase} that preferred to nick strand B was replaced by FokI-L_{nickase} that preferred to nick strand A. As expected, the single strand where base editing occurred was switched from strand A to strand B (FIG. 5A). Meanwhile, as for the editing window, when the 5′ exonuclease mExoI was used, the editing window was the 5′-adjacent region (C6 and C8) of strand B of the OsCKX2 target site, correspondingly, when the 3′ exonuclease Trex2 was used, the editing window could be shifted to the 3′-adjacent region (C3 and C6) of strand B of the OsCKX2 target site, and the cytosine residues in strand A were not edited (FIG. 5A). It could be seen that the base editor of the present disclosure could use exonucleases with different digestion directions and exert the digestion effect of the corresponding exonuclease, thereby editing the target site selectively.

Rice protoplasts were transformed with different construct combinations to target the OsCKX2 site, and the C>T base editing efficiency and the frequency of indel byproducts were determined by NGS. In FIG. 5A and FIG. 5B, the experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALE-FokI-R_{nickase} + | FIG. 16B, wherein the corresponding nickase was |
| A3A-UGI + mExoI | FokI-R_{nickase}, the corresponding deaminase was |
| | hAPOBEC3A, and the corresponding exonuclease was |
| | mExoI. |
| TALE-FokI-R_{nickase} + | FIG. 16B, wherein the corresponding nickase was |
| A3A-UGI + Trex2 | FokI-R_{nickase}, the corresponding deaminase was |
| | hAPOBEC3A, and the corresponding exonuclease was |
| | Trex2. |
| TALE-FokI-L_{nickase} + | FIG. 16B, wherein the corresponding nickase was |
| A3A-UGI + mExoI | FokI-L_{nickase}, the corresponding deaminase was |
| | hAPOBEC3A, and the corresponding exonuclease was |
| | mExoI. |
| TALE-FokI-L_{nickase} + | FIG. 16B, wherein the corresponding nickase was |
| A3A-UGI + Trex2 | FokI-L_{nickase}, the corresponding deaminase was |
| | hAPOBEC3A, and the corresponding exonuclease was |
| | Trex2. |
| TALEN_{WT} | FIG. 16A |

### Example 4: Effects of Cytidine Deaminase Type

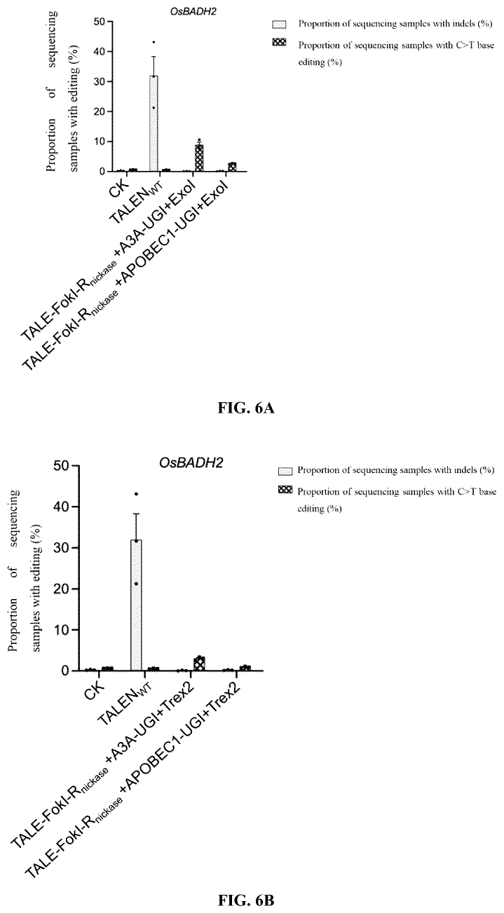

The novel base editor of the present disclosure had no dependence on the type of deaminase and was compatible with deaminases of different types. In order to exclude that the base editing ability of the novel base editor was deaminase hAPOBEC3A (A3A)-dependent, another cytidine deaminase rAPOBEC1 (APOBEC1) was tested by the inventor in this example. As indicated by NGS analysis results, in the presence of both an exonuclease, for example, mExoI (as shown in FIG. 6A) and Trex2 (as shown in FIG. 6B), targeted base editing was also achieved with high product purity after replacing hAPOBEC3A with rAPOBEC1 at the OsBADH2 site, indicating deaminases of different types were all suitable for the base editor of the present disclosure.

In FIG. 6A, rice protoplasts were transformed with different construct combinations to target the OsBADH2 site, and the C>T base editing efficiency and the frequency of indel byproducts were determined by NGS. The experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALE-FokI-R_{nickase} + | FIG. 16B, wherein the corresponding nickase was |
| A3A-UGI + mExoI | FokI-R_{nickase}, the corresponding deaminase was |
| | hAPOBEC3A, and the corresponding exonuclease was |
| | mExoI. |
| TALE-FokI-R_{nickase} + | FIG. 16B, wherein the corresponding nickase was |
| APOBEC1-UGI + mExoI | FokI-R_{nickase}, the corresponding deaminase was |
| | rAPOBEC1, and the corresponding exonuclease was |
| | mExoI. |
| TALEN_{WT} | FIG. 16A |

In FIG. 6B, rice protoplasts were transformed with different construct combinations to target the OsDEP1 site, and the C>T base editing efficiency and the frequency of indel byproducts were determined by NGS. The experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALE-FokI-R_{nickase} + | FIG. 16B, wherein the corresponding nickase was |
| A3A-UGI + Trex2 | FokI-R_{nickase}, the corresponding deaminase was |
| | hAPOBEC3A, and the corresponding exonuclease was |
| | Trex2. |
| TALE-FokI-R_{nickase} + | FIG. 16B, wherein the corresponding nickase was |
| APOBEC1-UGI + Trex2 | FokI-R_{nickase}, the corresponding deaminase was |
| | rAPOBEC1, and the corresponding exonuclease was |
| | Trex2. |
| TALEN_{WT} | FIG. 16A |

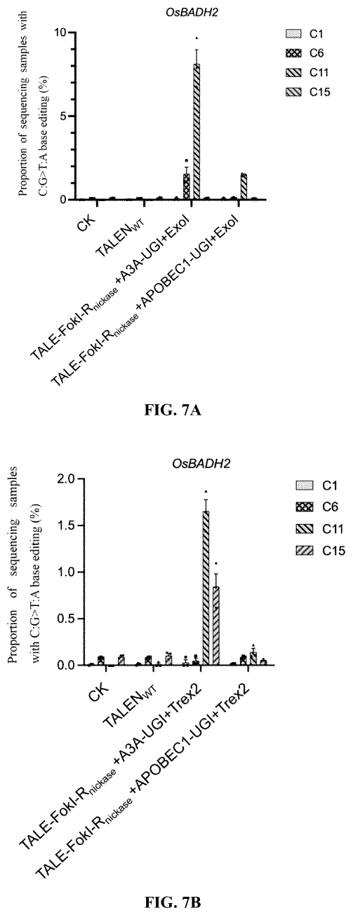

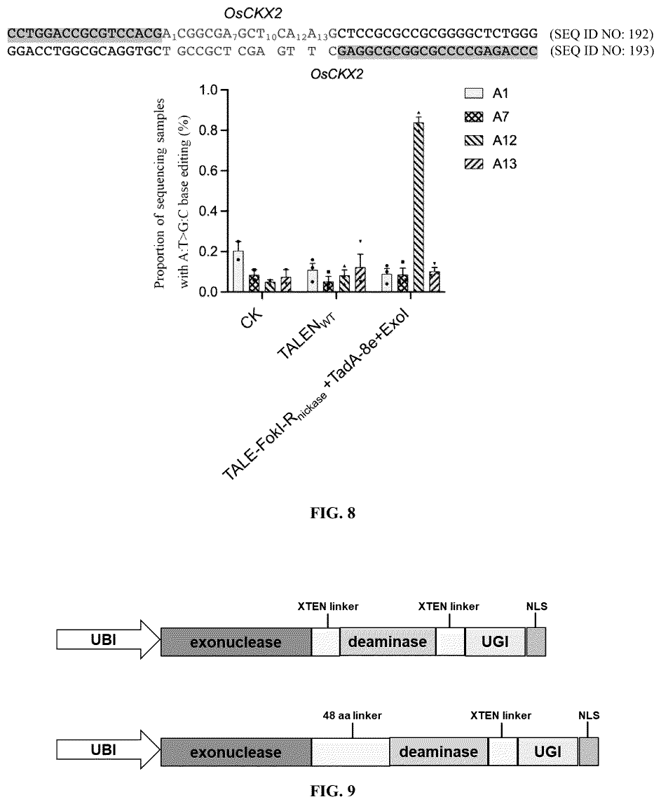

When the editing windows of these base editors were analyzed, cytosine residues located near the 5′ region of the target site in the complementary strand of the nicked single strand were efficiently edited in the groups containing mExoI (as shown in FIG. 7A), while the cytosine residues located near the 3′ region of the target site in the complementary chain were efficiently edited in the groups containing TREX2 (as shown in FIG. 7B), which were consistent with the results in the above-mentioned Example. These results indicated that the base editing method and the base editor disclosed in the present disclosure were compatible with different cytidine deaminases.

In FIG. 7A, the base editing window of the base editor was analyzed according to NGS results. The experimental treatments or construct combinations involved in figures and the schematic diagrams of related vectors were as shown below.

| Experimental treatments or | |
|---|---|
| construct combinations | |
| involved in figures | Schematic diagrams of related vectors |
| CK | None |
| TALE-FokI-R_{nickase} + | FIG. 16B, wherein the corresponding nickase was |
| A3A-UGI + mExoI | FokI-R_{nickase}, the corresponding deaminase was |
| | hAPOBEC3A, and the corresponding exonuclease was |
| | mExoI. |
| TALE-FokI-R_{nickase} + | FIG. 16B, wherein the corresponding nickase was |
| APOBEC1-UGI + mExoI | FokI-R_{nickase}, the corresponding deaminase was |
| | rAPOBEC1, and the corresponding exonuclease was |
| | mExoI. |
| TALEN_{WT} | FIG. 16A |

In FIG. 7B, the base editing window of the base editor was analyzed according to NGS results. The experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| TALE-FokI-R_{nickase} + | FIG. 16B, wherein the corresponding nickase was |
| A3A-UGI + | FokI-R_{nickase}, the corresponding deaminase was |
| Trex2 | hAPOBEC3A, and the corresponding exonuclease |
| | was Trex2. |
| TALE-FokI-R_{nickase} + | FIG. 16B, wherein the corresponding nickase was |
| APOBEC1-UGI + | FokI-R_{nickase}, the corresponding deaminase was Trex2, |
| Trex2 | and the corresponding exonuclease was Trex2. |
| TALEN_{WT} | FIG. 16A |

### Example 5: Base Editor Comprising Adenosine Deaminase

In order to expand the range of target sequences that could be edited by the base editor of the present disclosure, in this Example, an adenosine deaminase TadA-8e, which used deoxyadenosine (A) in single-stranded DNA as a substrate, was used as the deaminase to target A1, A7, A12 and A13 of the OsCKX2 site (as shown in FIG. 8). In this Example, UGI was not a necessary component of the base editor to be tested, since it was not essential for adenine base editing. The adenine base editing window of the base editor was analyzed according to NGS results. NGS analysis indicated that targeted A-to-G conversion occurred at the target site efficiently (FIG. 8), indicating that the base editor of the present disclosure was compatible with an adenosine deaminase for adenine base editing. Taken together, it could be seen from Examples 4 and 5 that the base editing method and the base editor disclosed in the present disclosure were compatible with different deaminases and were capable of exerting their corresponding editing effects.

In FIG. 8, the experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| TALE-FokI-R_{nickase} + | FIG. 16B, wherein the corresponding nickase was |
| TadA-8e + | FokI-R_{nickase}, the corresponding deaminase was TadA-8e, |
| mExoI | the corresponding exonuclease was mExol, and |
| | UGI was absent. |
| TALEN_{WT} | FIG. 16A |

### Example 6: Base Editors Comprising Fusion Proteins of Base Editing Components

After the function and effect of the base editor of the present disclosure were demonstrated by the above-mentioned Examples, whether the transformation efficiency (and thus the editing efficiency) could be improved by fusing modular elements into a single vector were verified in this Example. The structures of two examples of such base editor comprising fused elements were as shown in FIG. 9, wherein the exonuclease was fused to the amino terminal of the deaminase-UGI fusion protein via an XTEN linker peptide or a 48-amino acid linker peptide (48aa) so as to target the OsDEP1 gene, that is, the deaminase was fused to the exonuclease.

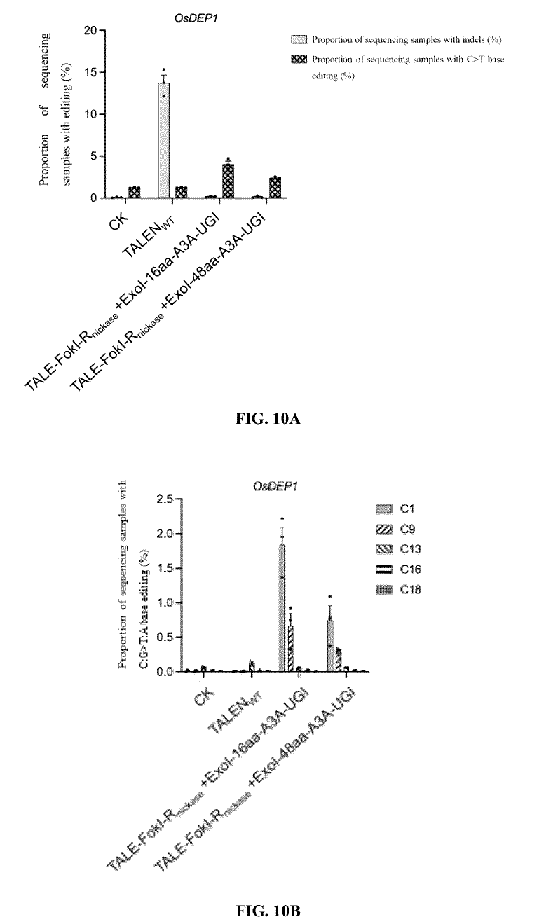

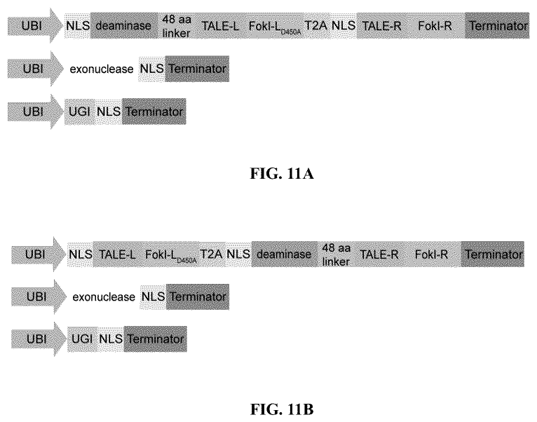

Rice protoplasts were transformed with different construct combinations to target the OsDEP1 site, and the C>T base editing efficiency and the frequency of indel byproducts were determined by NGS. The NGS analysis indicated that fusing an exonuclease to a deaminase could achieve targeted base editing while the efficiency achieved by such vector structure was similar to the efficiency achieved in a case where the exonuclease and the deaminase were expressed separately (as shown in FIG. 10A). When this base editor was used, C1 and C9 were preferred in the editing window (as shown in FIG. 10B), which was consistent with the catalytical direction of mExoI exonuclease.

In FIG. 10A and FIG. 10B, the experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| TALE-FokI-R_{nickase} + | FIG. 16B, wherein the corresponding nickase was |
| mExoI-16aa- | FokI-R_{nickase}; FIG. 9, wherein the corresponding |
| A3A-UGI | exonuclease was mExoI, the corresponding |
| | deaminase was hAPOBEC3A, and there was a |
| | 16-amino acid linker peptide (16aa) therebetween. |
| TALE-FokI-R_{nickase} + | FIG. 16B, wherein the corresponding nickase was |
| mExoI-48aa- | FokI-R_{nickase}; FIG. 9, wherein the corresponding |
| A3A-UGI | exonuclease was mExoI, the corresponding |
| | deaminase was hAPOBEC3A, and there was a |
| | 48-amino acid linker peptide therebetween. |
| TALEN_{WT} | FIG. 16A |

In addition, the inventors also tested other fusion protein structures. The structures of the above-mentioned base editors were shown in FIG. 11A and FIG. 11B, wherein the deaminase (hAPOBEC3A or rAPOBEC1) was fused to the amino terminal of TALE-L (FIG. 11A) or TALE-R (FIG. 11B) via a 48-amino acid linker peptide, UGI and the exonuclease were expressed by separate vectors, that is, the deaminase, the TALE protein and the nickase were fused.

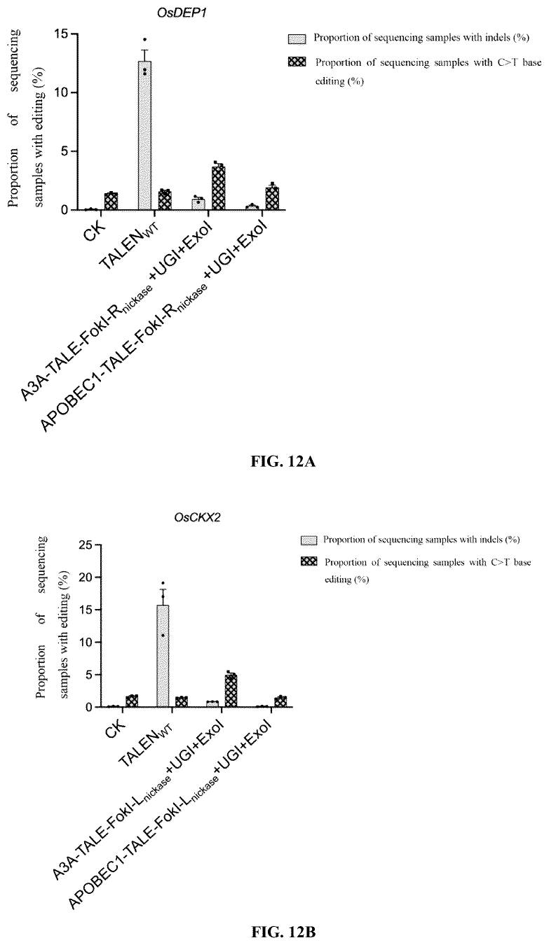

As for the deaminase-TALE-FokI-R_{nickase}, OsDEP1 was selected for characterization as the target gene to be tested (as shown in FIG. 12A), while for the deaminase-TALE-FokI-L_{nickase}, OsCKX2 was selected for characterization as the target gene to be tested (as shown in FIG. 123). The NGS analysis showed that both deaminase-TALE-FokI-L/R_{nickase} achieved C-to-T conversion at the target site, indicating that deaminase could form a fusion body with the TALE protein and the nickase without interfering with the exertion of their respective functions. In addition, the experimental results also further indicated that base editing could occur in a case where the deaminase hAPOBEC3A was used and in a case where the deaminase rAPOBEC1 was used (as shown in FIG. 12A and FIG. 12).

In FIG. 12A, the experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| A3A-TALE-FokI-R_{nickase} + | FIG. 16C or FIG. 11A, wherein the corresponding |
| UGI + mExoI | nickase was FokI-R_{nickase}, and the corresponding |
| | exonuclease was mExoI, the corresponding |
| | deaminase was hAPOBEC3A. |
| APOBEC1-TALE-FokI-R_{nickase} + | FIG. 16C or FIG. 11A, wherein the corresponding |
| UGI + mExoI | nickase was FokI-R_{nickase}, and the corresponding |
| | exonuclease was mExoI, the corresponding deaminase |
| | was APOBEC1. |
| TALEN_{WT} | FIG. 16A |

In FIG. 12B, the experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| A3A-TALE-FokI-L_{nickase} + | FIG. 16C, wherein the corresponding nickase |
| UGI + mExoI | was FokI-L_{nickase}, and the corresponding |
| | exonuclease was mExoI, the corresponding |
| | deaminase was hAPOBEC3A. |
| APOBEC1-TALE-FokI-L_{nickase} + | FIG. 16C, wherein the corresponding nickase |
| UGI + mExoI | was FokI-L_{nickase}, and the corresponding |
| | exonuclease was mExol, the corresponding |
| | deaminase was rAPOBEC1. |
| TALEN_{WT} | FIG. 16A |

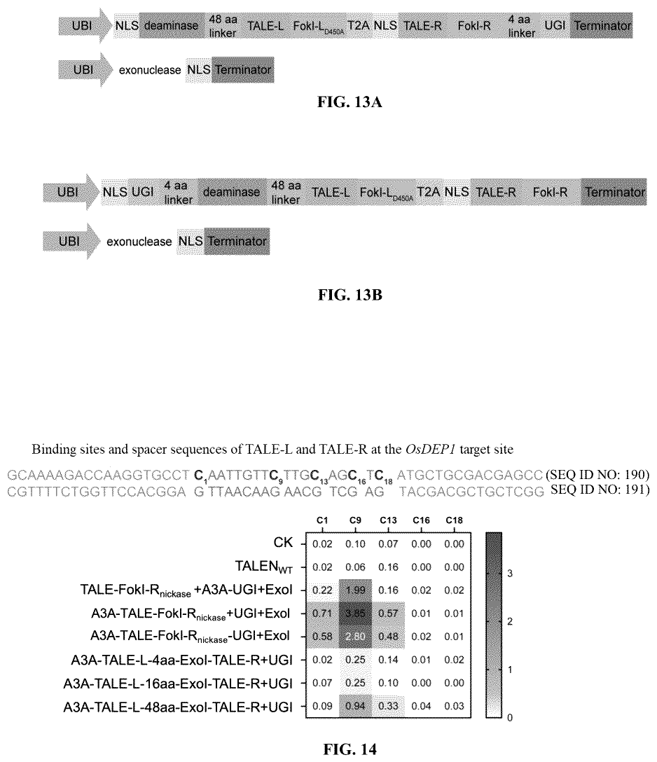

In order to investigate the influence of the fusion of UGI or exonuclease, in the deaminase-TALE-FokI-R_{nickase} construct having the same target specificity as that of the present disclosure, the base editor had a UGI linked to the carboxy terminal of FokI-L_{D450A} (as shown in FIG. 13A) or the amino terminal of the deaminase (as shown in FIG. 13B) via a 48-amino acid linker peptide or a 4-amino acid linker peptide. The NGS analysis indicated that the effect achieved by linking UGI to the fusion protein was similar to those of the embodiments in which UGI was separately expressed (FIG. 14). In addition, in the deaminase-TALE-FokI-R_{nickase} construct, the embodiments in which an exonuclease was fused to the carboxy terminal of FokI-R via a 4-amino acid linker peptide, a 16-amino acid linker peptide or a 48-amino acid linker peptide also achieved similar editing efficiency (FIG. 14). As a result, both expressing UGI/exonuclease separately and fusing UGI/exonuclease to the vector for co-expression were technical solutions that could be adopted in the present disclosure.

In FIG. 14, rice protoplasts were transformed with different construct combinations to target the OsDEP1 site, and the DNA strand and the editing window where base editing occurred were analyzed via the results of high-throughput sequencing. The experimental treatments or construct combinations involved in figures and the schematic diagrams of related vectors were as shown below.

| Experimental treatments or construct | Schematic diagrams of related vectors |
|---|---|
| combinations involved in figures | or the elements used |
| CK | None |
| TALEN_{WT} | FIG. 16A |
| TALE-FokI-R_{nickase} + | FIG. 16B, wherein the corresponding nickase |
| A3A-UGI + mExoI | was FokI-R_{nickase}, the corresponding deaminase |
| | was hAPOBEC3A, and the corresponding |
| | exonuclease was mExoI. |
| A3A-TALE-FokI-R_{nickase} + | FIG. 16C, wherein the corresponding nickase |
| UGI + mExoI | was FokI-R_{nickase}, the corresponding deaminase |
| | was hAPOBEC3A, and the corresponding |
| | exonuclease was mExoI. |
| A3A-TALE-FokI-R_{nickase}- | FIG. 16D, wherein the corresponding nickase was |
| UGI + mExoI | FokI-R_{nickase}, the corresponding deaminase was |
| | hAPOBEC3A fused to the N-terminal of TALE-L, and the |
| | corresponding exonuclease was mExoI. |
| A3A-TALE-L-mExoI-4aa- | the corresponding nickase was FokI-R_{nickase}, the |
| TALE-R + UGI | corresponding deaminase was hAPOBEC3A fused to the |
| | N-terminal of TALE-L, the corresponding exonuclease |
| | was ExoI fused to the N-terminal of TALE-R, and there |
| | was a 4-amino acid linker peptide therebetween. |
| A3A-TALE-L-mExoI-16aa- | the corresponding nickase was FokI-R_{nickase}, the |
| TALE-R + UGI | corresponding deaminase was hAPOBEC3A fused to the |
| | N-terminal of TALE-L, the corresponding exonuclease |
| | was mExoI fused to the N-terminal of TALE-R, and there |
| | was a 16-amino acid linker peptide therebetween. |
| A3A-TALE-L-mExoI-48aa- | the corresponding nickase was FokI-R_{nickase}, the |
| TALE-R + UGI | corresponding deaminase was hAPOBEC3A fused to the |
| | N-terminal of TALE-L, the corresponding exonuclease |
| | was mExoI fused to the N-terminal of TALE-R, and there |
| | was a 48-amino acid linker peptide therebetween. |

Taken the above results together, each modular element of the base editor of the present disclosure could be expressed individually, or each element could form one or more fusion proteins with each other.

### Example 7: Base Editing in Plant Nuclear Genome

In Examples above, the functions and characteristics of the base editor of the present disclosure were verified, that is, the composition of modular elements comprising a deaminase, an exonuclease, a nickase, a DNA-binding protein TALE could achieve efficient and precise DNA editing. For ease of description, the above-mentioned base editors were named DENT (Deaminase-Exonuclease-Nickase-TALE), and were respectively named CyDENT (Cytidine Deaminase-Exonuclease-Nickase-TALE) and AdDENT (Adenosine deaminase-Exonuclease-Nickase-TALE) according to the type of deaminase. In this Example, the applicable environments and scenarios of the base editor of the present disclosure were analyzed.

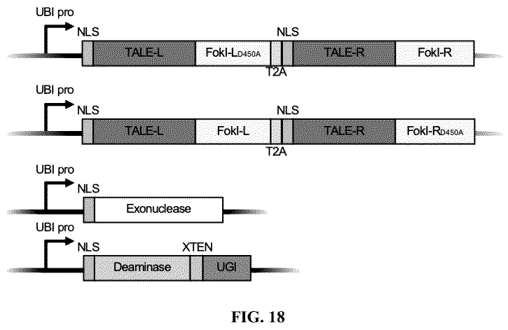

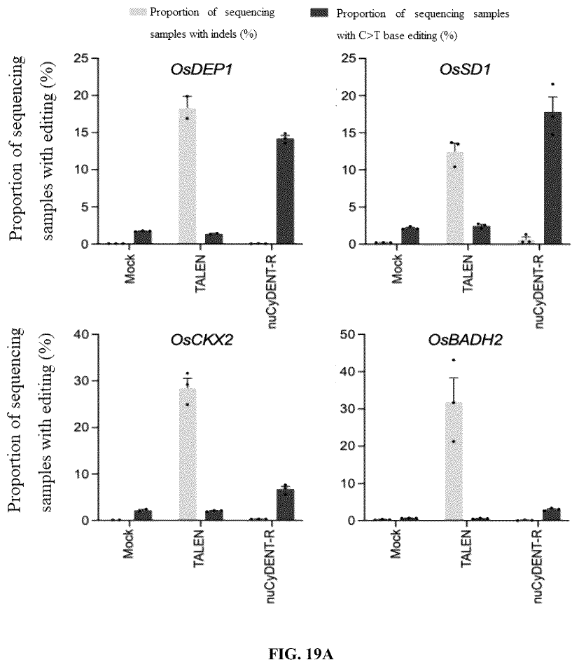

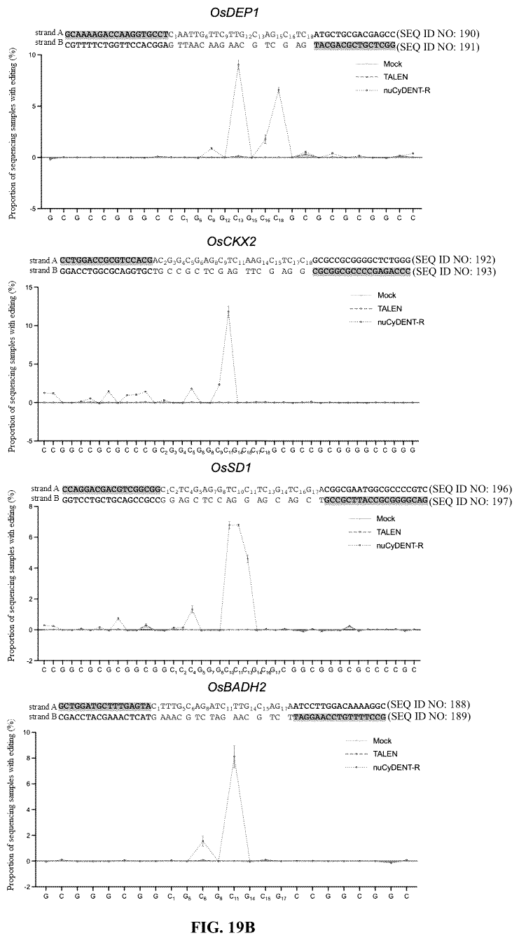

The inventors selected the nuclear genome of rice protoplast to evaluate the editing effect of the base editor of the present disclosure. In this Example, four pairs of TALE proteins were respectively designed for the endogenous gene loci of rice (i.e., OsDEP1, OsCKX2, OsBADH 2 and OsSD1). Exonucleases with 5′→3′ (mExol) cleavage preference or 3′→5′ (Trex2) cleavage preference were used to evaluate the effect of fusing the exonuclease and the nickase to form an ssDNA intermediate. In this Example, an efficient cytidine deaminase hAPOBEC3A (hA3A) was selected to deaminate cytosine(s) in the ssDNA intermediate, a uracil glycosylase inhibitor (UGI) peptide was fused to its C-terminal, and the editing efficiency was further improved by minimizing the influence of DNA base excision repair. Nuclear localization signals (NLS) were fused to the N-terminal of each component, thereby editing the nuclear genome directly. Such combination of the base editors targeting the nuclear genome was referred to as nuCyDENT herein, and the schematic diagram of the exemplary construct was as shown in FIG. 18. The nuCyDENT that targeted the OsDEP1, OsCKX2, OsBADH2 and OsSD1 sites in rice was introduced into the rice protoplast, and the editing efficiency was evaluated after 2 days. Targeted cytosine base editing was assessed within the 18 bp spacing regions between the TALE binding sites of all four nuclear genomic sites by utilizing NGS analysis. An editing efficiency of 3% to 18% and lower indel frequency (compared to that of the corresponding wild-type TALEN system) were observed (FIG. 19A and FIG. 19B). These results indicated that the base editor of the present disclosure could achieve efficient base editing in the nuclear genome while merely resulting in indel byproducts at a low level.

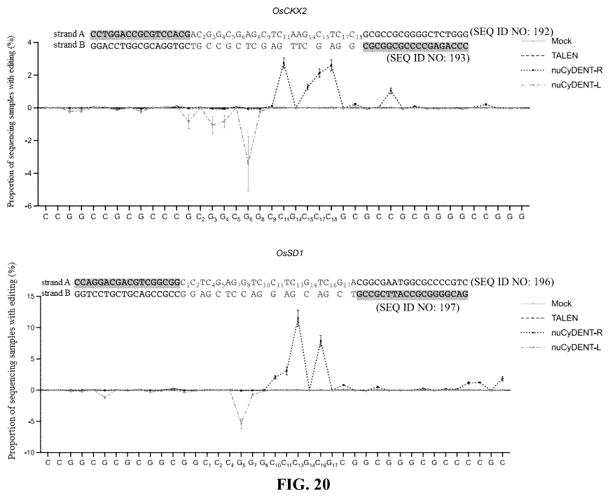

In terms of the single-strand editing performance, the inventors used nuCyDENT-L (nuCyDENT comprising an FokI-L_{nickase} structure) and nuCyDENT-R (nuCyDENT comprising an FokI-R_{nickase} structure) to perform respective base editing in rice genome loci OsCKX2 and OsSD1. As indicated by the results, the top strand of DNA was edited when using nuCyDENT-R for editing, and the bottom strand of DNA was edited when using nuCyDENT-L for editing (FIG. 20). This conclusion was the same as Example 2, which also showed the single-strand editing performance of CyDENT in the nuclear genome.

In FIG. 19A, FIG. 19B and FIG. 20, the experimental treatments or construct combinations involved in figures were as shown below.

| | Experimental treatments or construct combinations involved in figures |
|---|---|
| nuCyDENT for | OsDEP1-NLS-TALE-L-FokI-L_{D450A}-T2A-NLS-TALE-R-FokI-R + |
| OsDEP1 target site | NLS-A3A-UGI + NLS-mExoI |
| nuCyDENT-R for | OsSD1-NLS-TALE-L-FokI-L_{D450A}-T2A-NLS-TALE-R-FokI-R + |
| OsSD1 target site | NLS-A3A-UGI + NLS-mExoI |
| nuCyDENT-R for | OsCKX2-NLS-TALE-L-FokI-L_{D450A}-T2A-NLS-TALE-R-FokI-R + |
| OsCKX2 target site | NLS-A3A-UGI + NLS-mExoI |
| nuCyDENT for | OsBADH2-NLS-TALE-L-FokI-L_{D450A}-T2A-NLS-TALE-R-FokI-R + |
| OsBADH2 target site | NLS-A3A-UGI + NLS-mExoI |
| nuCyDENT-L for | OsCKX2-NLS-TALE-L-FokI-L-T2A-NLS-TALE-R-FokI-R_{D450A} + |
| OsCKX2 target site | NLS-A3A-UGI + NLS-mExoI |
| nuCyDENT-L for | OsSD1-NLS-TALE-L-FokI-L-T2A-NLS-TALE-R-FokI-R_{D450A} + |
| OsSD1 target site | NLS-A3A-UGI + NLS-Trex2 |
| TALEN | TALEN_{WT} |
| Mock | None, i.e., blank control, the same applied to |
| | the subsequent Examples. |

### Example 8: Base Editing in Animal Nuclear Genome

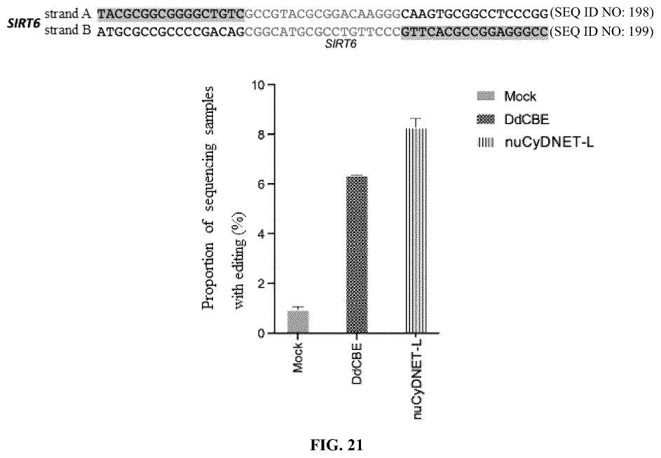

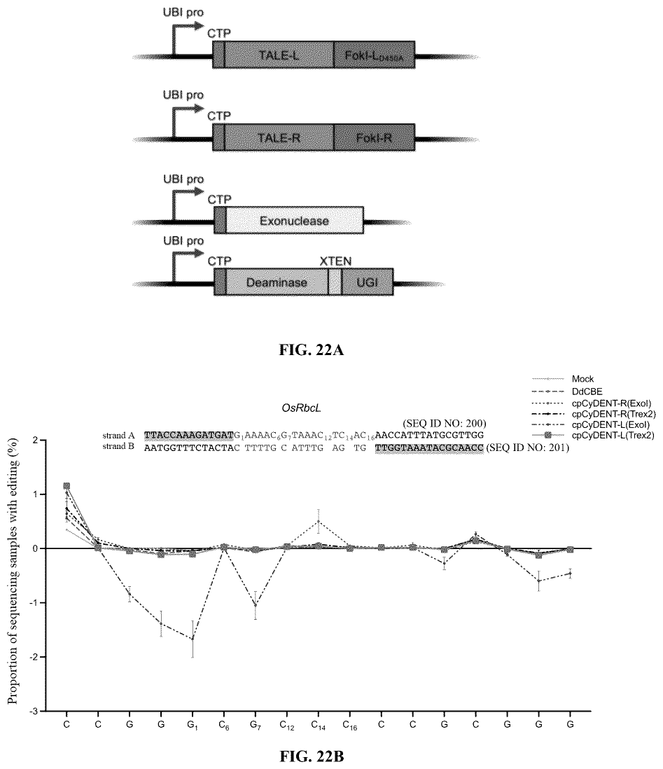

The effects of base editing of CyDENT and DdCBE at human SIRT6 gene (target site) were compared in this Example. The inventor designed a TALE protein for the SIRT6 target, designed and obtained nuCyDENT-L according to the method in Example 7, and designed and obtained a DddA-dependent DdCBE according to the method in the prior art (Nakazato, I. et al. Targeted base editing in the mitochondrial genome of Arabidopsis thaliana. Proc. Natl. Acad. Sci. USA. 119, e2121177119 (2022).). The experimental results showed that nuCyDENT-L had higher base editing efficiency than DdCBE at the target site (FIG. 21), indicating that the base editing system of the present disclosure had good base editing performance in the nuclear genome of animal cells.

In FIG. 21, the experimental treatments or construct combinations involved in figures were as shown below.

Experimental treatments or construct
combinations involved in figures

| | |
|---|---|
| nuCyDENT-L | SIRT6-NLS-TALE-L-FokI-L |
| | SIRT6-NLS-TALE-R-FokI-R_{D450A} |
| | NLS-A3A |
| | NLS-UGI |
| | NLS-mExoI |
| DdCBE | SIRT6-NLS-TALE-L-DddA_{N}-UGI |
| | SIRT6-NLS-TALE-R-DddA_{C}-UGI |

### Example 9: Base Editing of DNA in Organelle—Chloroplast

The base editor of the present disclosure could be used for mitochondrial DNA base editing and chloroplast DNA base editing, and had advantages over CRISPR base editors that needed to comprise nucleic acid components. The protein components in the base editor of the present disclosure could be translocated into mitochondria and chloroplasts via a mitochondrial targeting sequence (MTS) and a chloroplast translocation peptide (CTP) respectively. In these Examples, MTS or CTP could be selected to replace NLS according to the type of target organelle.

First, the inventors attempted to perform base editing on plant chloroplast DNA using the base editing strategy of CyDENT. Plant chloroplast DNA was an important organelle specific to plants, had its own genomic DNA (cpDNA), and could not be edited by using CRISPR-derived base editors. The inventor replaced NLS with chloroplast translocation peptide (CTP) in nuCyDENT that was designed with reference to the method in Example 7 (Kang, B. C. et al. Chloroplast and mitochondrial DNA editing in plants. Nat Plants 7, 899-905 (2021).) (FIG. 22A), and the resultant was named cpCyDENT. Rice protoplasts were transformed by the inventors with cpCyDENT-L (comprising FokI-L_{nickase}) and cpCyDENT-R (comprising FokI-R_{nickase}), which comprised a TALE protein targeting the endogenous ribulose-1,5-bisphosphate carboxylase/oxygenase (RuBisCO) large subunit gene (rbcL). Base editing at the rbcL target was detected in cpCyDENT-L treatment (FIG. 22B). It is worth noting that the precise editing of specific bases could be achieved by regulating the type and direction of the nickase and the exonuclease in cpCyDENT. For example, as for Gi base (the most 5′ nucleotide in the spacer region was designated as position 1, see FIG. 22B), this base could be edited efficiently with an editing efficiency of approximately 1.67% only when the cpCyDENT-L(mExol) tool comprising FokI-L_{nickase} and 5′→3′ mExol exonuclease was used.

This result conformed to the conclusion of the above-mentioned Examples. These results indicated that cpCyDENT was capable of performing base editing on the DNA strand in chloroplast genome selectively and precisely.

In FIG. 22B, the experimental treatments or construct combinations involved in figures were as shown below.

| | Experimental treatments or construct |
|---|---|
| | combinations involved in figures |
| cpCyDENT-R | OsRbcL-CTP-TALE-L-FokI-L_{D450}A + |
| (mExoI) | OsRbcL-CTP-TALE-R-FokI-R + |
| | CTP-A3A-UGI + CTP-mExoI |
| cpCyDENT-R | OsRbcL-CTP-TALE-L-FokI-L_{D450}A + |
| (Trex2) | OsRbcL-CTP-TALE-R-FokI-R + |
| | CTP-A3A-UGI + CTP-Trex2 |
| cpCyDENT-L | OsRbcL-CTP-TALE-L-FokI-L + |
| (mExoI) | OsRbcL-CTP-TALE-R-FokI-R_{D450A} + |
| | CTP-A3A-UGI + CTP-mExoI |
| cpCyDENT-L | OsRbcL-CTP-TALE-L-FokI-L + |
| (Trex2) | OsRbcL-CTP-TALE-R-FokI-R_{D450A} + |
| | CTP-A3A-UGI + CTP-Trex2 |

### Example 10: Base Editing of DNA in Organelle—Mitochondrion

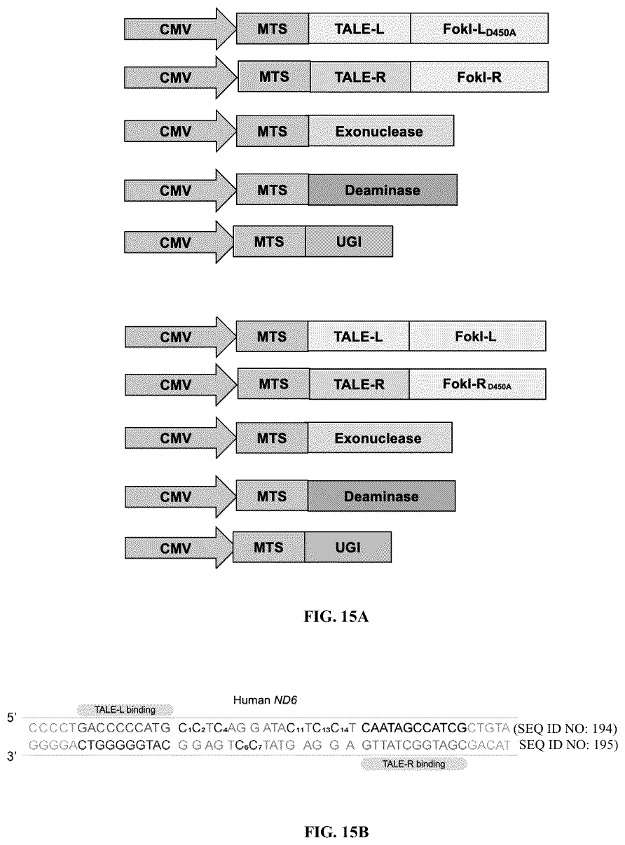

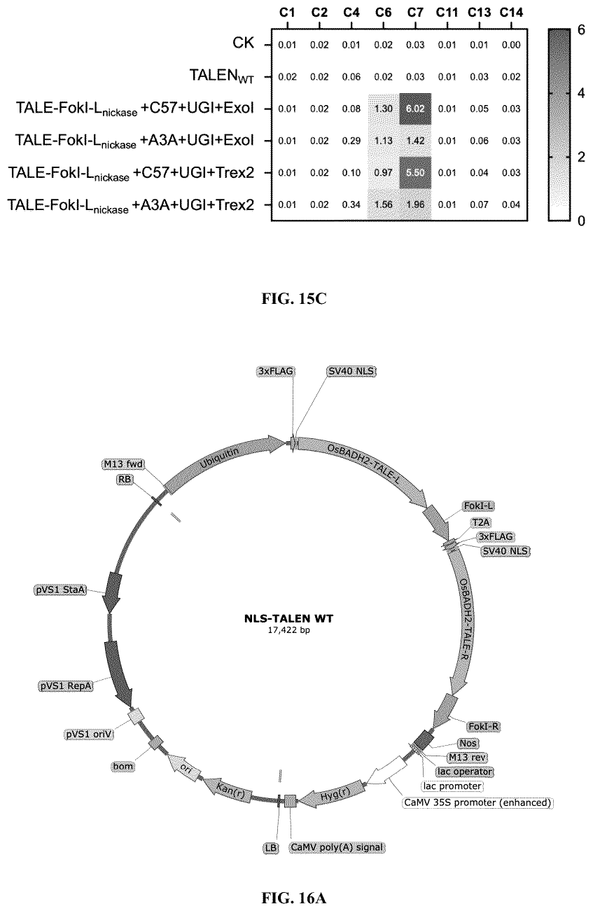

In this Example, the inventors assessed the influence of CyDENT base editing in mitochondrial DNA (mtDNA) base editing in human cells, replaced NLS with mitochondrial targeting sequence (MTS) and selected promoters and terminators suitable for expression in HEK293T cells, thereby obtaining a base editor for mtDNA, referred to as mtCyDENT. The mtCyDENT construct generated in this Example was as shown in FIG. 15A (TALE-FokI-R_{nickase} and TALE-FokI-L_{nickase}).

First, a target site in ND6 gene of human mitochondrial DNA was selected to construct TALE-FokI-R_{nickase} and TALE-FokI-L_{nickase} expression vectors in which the TALE proteins were modified to target the site, and said expression vectors were transfected into HEK293T cells together with the vectors expressing the deaminase (hAPOBEC3A or C57), the exonuclease (mExoI or Trex2) and UGI, wherein the mitochondrial targeting sequence (MTS) was fused to the terminal of the protein. NGS was used to determine the base editing frequency after the transfection by the base editor. The results indicated that targeted cytosine base editing was achieved with an efficiency of about 6.0% in the mitochondrial DNA target of human cells (FIG. 15C). The results indicated that the base editor of the present disclosure could be used for the base editing of organelle genome.

In FIG. 15C, HEK293T cells were transfected with different construct combinations to target the mitochondrial ND6 site, and the DNA strand and the editing window where base editing occurred were analyzed via the results of high-throughput sequencing. The experimental treatments or construct combinations involved in figures and the schematic diagrams of related vectors were as shown below.

| Experimental treatments or construct | |
|---|---|
| combinations involved in figures | Schematic diagrams of related vectors |
| CK | None |
| TALEN_{WT} | FIG. 17A and FIG. 17B |
| TALE-FokI-L_{nickase} + | FIGS. 17A, 17B, 17C, 17D and 17E, wherein the |
| C57 + UGI + mExoI | corresponding nickase was FokI-L_{nickase}, the |
| | corresponding exonuclease was mExoI, and the |
| | corresponding deaminase was C57. |
| TALE-FokI-L_{nickase} + | FIGS. 17A, 17B, 17C, 17D and 17E, wherein the |
| A3A + UGI + mExoI | corresponding nickase was FokI-L_{nickase}, the |
| | corresponding exonuclease was mExoI, and the |
| | corresponding deaminase was hAPOBEC3A. |
| TALE-FokI-L_{nickase} + | FIGS. 17A, 17B, 17C, 17D and 17E, wherein the |
| C57 + UGI + Trex2 | corresponding nickase was FokI-L_{nickase}, the |
| | corresponding exonuclease was Trex2, and the |
| | corresponding deaminase was C57. |
| TALE-FokI-L_{nickase} + | FIGS. 17A, 17B, 17C, 17D and 17E, wherein the |
| A3A + UGI + Trex2 | corresponding nickase was FokI-L_{nickase}, the |
| | corresponding exonuclease was Trex2, and the |
| | corresponding deaminase was hAPOBEC3A. |

### Example 11: Effects of the Fusion State of Base Editor in Mitochondrial DNA Editing

Next, the inventors verified the effects of the individually expressed deaminase, exonuclease, UGI and TALE-FokI nickase on mtDNA base editing efficiency.

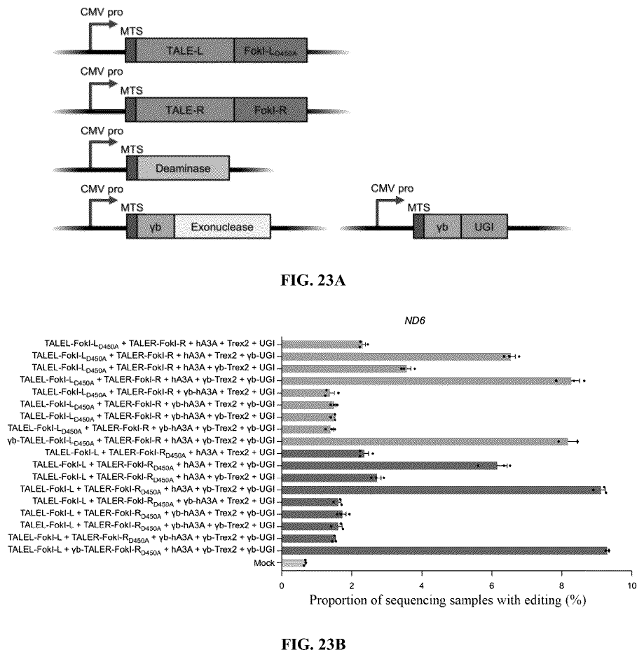

For this purpose, the inventors used a small peptide referred to as γb and γb was fused to the N-terminal of the domain of one or more modular components in mtCyDENT so as to drive the recruitment of each protein element (FIG. 23A). γb was an RNA silencing suppressor derived from barley stripe mosaic virus (BSMV) having self-interaction (Jiang, Z., Yang, M., Zhang, Y., Jackson, A. O. & Li, D. in Encyclopedia of Virology 420-429 (2021).). In this experiment, the exonuclease selected by the inventors was Trex2. The inventors designed a variety of schemes for the fusion between γb and each component, so as to screen out the base editor composition with optimal editing effect (FIG. 23B). Taking the size of the protein components entering mitochondria into consideration, a construct composition of five proteins/fusion proteins as shown in FIG. 23A was used for expression in this Example, and the proteins/fusion proteins were a fusion protein of TALE-L and FokI-L (simply referred to as TALE-L-FokI-L, TALEL-FL or TALEL-FokI-L), a fusion protein of TALE-R and FokI-R (simply referred to as TALE-R-FokI-R, TALEL-FR or TALER-FokI-R), hA3A deaminase protein, Trex2 exonuclease protein and UGI protein, respectively. Among them, the tail tag D450A represented a mutant, and WT represented “wild-type”_{∘}The experimental results indicated that higher editing effect could be achieved when γb was merely fused with UGI and Trex2. The base editor composition having a structure in which γb was fused to UGI and Trex2 was named mtCyDENT1b.

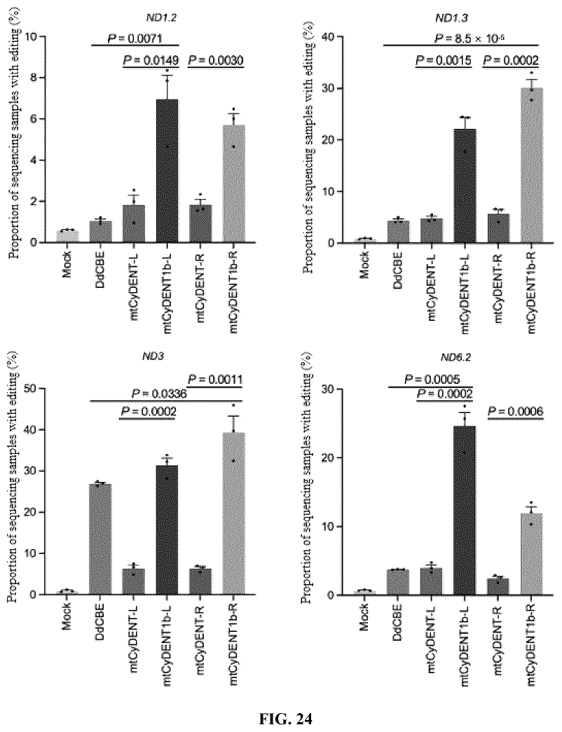

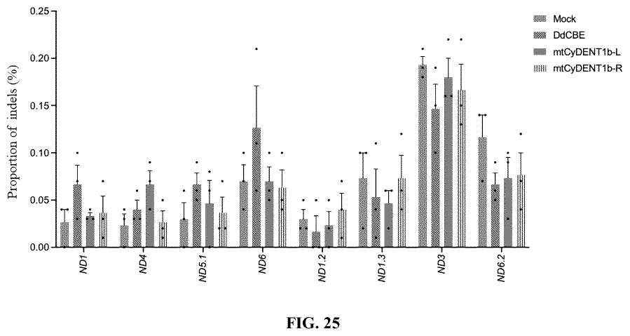

Next, mtCyDENT and mtCyDENT1b were assessed at seven additional endogenous mtDNA genomic loci by the inventors. It was observed by the inventors that the average editing frequency of mtCyDENT was 1.16% to 11.7%, while mtCyDENT1b could achieve an average editing efficiency that was further increased by 2.42-fold to 6.18-fold and was up to 4.55% to 39.3% (FIG. 24). Also, the editing efficiency of mtCyDENT1b was higher than that of DdCBE at ND1.2, ND1.3, ND3 and ND6.2 targets having the same TALE sequence. In addition, the inventors also noticed that using CyDENT for base editing at mtDNA target site resulted in lower indel frequency as compared with DdCBE (FIG. 25). In summary, both mtCyDENT and mtCyDENT1b were capable of achieving efficient base editing in human mitochondrial DNA.

In FIG. 23B, the experimental treatments or construct combinations involved in figures were as shown below (from top to bottom).

Experimental treatments or construct
combinations involved in figures

ND6-MTS-TALE-L-FokI-L_{D450A} + ND6-MTS-TALE-R-FokI-R +
MTS-A3A + MTS-Trex2 + MTS-UGI
ND6-MTS-TALE-L-FokI-L_{D450A} + ND6-MTS-TALE-R-FokI-R +
MTS-A3A + MTS-Trex2 + MTS-γb-UGI
ND6-MTS-TALE-L-FokI-L_{D450A} + ND6-MTS-TALE-R-FokI-R +
MTS-A3A + MTS-γb-Trex2 + MTS-UGI
ND6-MTS-TALE-L-FokI-L_{D450A} + ND6-MTS-TALE-R-FokI-R +
MTS-A3A + MTS-γb-Trex2 + MTS-γb-UGI
ND6-MTS-TALE-L-FokI-L_{D450A} + ND6-MTS-TALE-R-FokI-R +
MTS-γb-A3A + MTS-Trex2 + MTS-UGI
ND6-MTS-TALE-L-FokI-L_{D450A} + ND6-MTS-TALE-R-FokI-R +
MTS-γb-A3A + MTS-Trex2 + MTS-γb-UGI
ND6-MTS-TALE-L-FokI-L_{D450A} + ND6-MTS-TALE-R-FokI-R +
MTS-γb-A3A + MTS-γb-Trex2 + MTS-UGI
ND6-MTS-TALE-L-FokI-L_{D450A} + ND6-MTS-TALE-R-FokI-R +
MTS-γb-A3A + MTS-γb-Trex2 + MTS-γb-UGI
ND6-MTS-γb-TALE-L-FokI-L_{D450A} + ND6-MTS-TALE-R-FokI-R +
MTS-A3A + MTS-γb-Trex2 + MTS-γb-UGI
ND6-MTS-TALE-L-FokI-L + ND6-MTS-TALE-R-FokI-R_{D450A} +
MTS-A3A + MTS-Trex2 + MTS-UGI
ND6-MTS-TALE-L-FokI-L + ND6-MTS-TALE-R-FokI-R_{D450A} +
MTS-A3A + MTS-Trex2 + MTS-γb-UGI
ND6-MTS-TALE-L-FokI-L + ND6-MTS-TALE-R-FokI-R_{D450A} +
MTS-A3A + MTS-γb-Trex2 + MTS-UGI
ND6-MTS-TALE-L-FokI-L + ND6-MTS-TALE-R-FokI-R_{D450A} +
MTS-A3A + MTS-γb-Trex2 + MTS-γb-UGI
ND6-MTS-TALE-L-FokI-L + ND6-MTS-TALE-R-FokI-R_{D450A} +
MTS-γb-A3A + MTS-Trex2 + MTS-UGI
ND6-MTS-TALE-L-FokI-L + ND6-MTS-TALE-R-FokI-R_{D450A} +
MTS-γb-A3A + MTS-Trex2 + MTS-γb-UGI
ND6-MTS-TALE-L-FokI-L + ND6-MTS-TALE-R-FokI-R_{D450A} +
MTS-γb-A3A + MTS-γb-Trex2 + MTS-UGI
ND6-MTS-TALE-L-FokI-L + ND6-MTS-TALE-R-FokI-R_{D450A} +
MTS-γb-A3A + MTS-γb-Trex2 + MTS-γb-UGI
ND6-MTS-TALE-L-FokI-L + ND6-MTS-γb-TALE-R-FokI-R_{D450A} +
MTS-A3A + MTS-γb-Trex2 + MTS-γb-UGI
blank control Mock

In FIGS. 24 to 27, the experimental treatments or construct combinations involved in figures were as shown below.

| | Experimental treatments or construct combinations involved in figures |
|---|---|
| DdCBE for | ND1.2-MTS-TALE-L-DddA_{N}-UGI + ND1.2-MTS-TALE-R-DddA_{C}-UGI |
| ND1.2 target site | |
| DdCBE for | ND1.3-MTS-TALE-L-DddA_{N}-UGI + ND1.3-MTS-TALE-R-DddA_{C}-UGI |
| ND1.3 target site | |
| DdCBE for ND3 | ND3-MTS-TALE-L-DddA_{N}-UGI + ND3-MTS-TALE-R-DddA_{C}-UGI |
| target site | |
| DdCBE for ND6 | ND6-MTS-TALE-L-DddA_{N}-UGI + ND6-MTS-TALE-R-DddA_{C}-UGI |
| target site | |
| DdCBE for | ND6.2-MTS-TALE-L-DddA_{N}-UGI + ND6.2-MTS-TALE-R-DddA_{C}-UGI |
| ND6.2 target site | |
| mtCyDENT-L | ND1.2-MTS-TALE-L-FokI-L + ND1.2-MTS-TALE-R-FokI-R_{D450A} + |
| for ND1.2 | MTS-A3A + MTS-Trex2 + MTS-UGI |
| target site | |
| mtCyDENT1b-L | ND1.2-MTS-TALE-L-FokI-L + ND1.2-MTS-TALE-R-FokI-R_{D450A} + |
| for ND1.2 | MTS-A3A + MTS-γb-Trex2 + MTS-γb-UGI |
| target site | |
| mtCyDENT-R | ND1.2-MTS-TALE-L-FokI-L_{D450A} + ND1.2-MTS-TALE-R-FokI-R + |
| for ND1.2 | MTS-A3A + MTS-Trex2 + MTS-UGI |
| target site | |
| mtCyDENT1b-R | ND1.2-MTS-TALE-L-FokI-L_{D450A} + ND1.2-MTS-TALE-R-FokI-R + |
| for ND1.2 | MTS-A3A + MTS-γb-Trex2 + MTS-γb-UGI |
| target site | |
| mtCyDENT-L | ND1.3-MTS-TALE-L-FokI-L + ND1.3-MTS-TALE-R-FokI-R_{D450A} + |
| for ND1.3 | MTS-A3A + MTS-Trex2 + MTS-UGI |
| target site | |
| mtCyDENT1b-L | ND1.3-MTS-TALE-L-FokI-L + ND1.3-MTS-TALE-R-FokI-R_{D450A} + |
| for ND1.3 | MTS-A3A + MTS-γb-Trex2 + MTS-γb-UGI |
| target site | |
| mtCyDENT-R | ND1.3-MTS-TALE-L-FokI-L_{D450A} + ND1.3-MTS-TALE-R-FokI-R + |
| for ND1.3 | MTS-A3A + MTS-Trex2 + MTS-UGI |
| target site | |
| mtCyDENT1b-R | ND1.3-MTS-TALE-L-FokI-L_{D450A} + ND1.2-MTS-TALE-R-FokI-R + |
| for ND1.3 | MTS-A3A + MTS-γb-Trex2 + MTS-γb-UGI |
| target site | |
| mtCyDENT-L | ND3-MTS-TALE-L-FokI-L + ND3-MTS-TALE-R-FokI-R_{D450A} + |
| for ND3 | MTS-A3A + MTS-Trex2 + MTS-UGI |
| target site | |
| mtCyDENT1b-L | ND3-MTS-TALE-L-FokI-L + ND3-MTS-TALE-R-FokI-R_{D450A} + |
| for ND3 | MTS-A3A + MTS-γb-Trex2 + MTS-γb-UGI |
| target site | |
| mtCyDENT-R | ND3-MTS-TALE-L-FokI-L_{D450A} + ND3-MTS-TALE-R-FokI-R + |
| for ND3 | MTS-A3A + MTS-Trex2 + MTS-UGI |
| target site | |
| mtCyDENT1b-R | ND3-MTS-TALE-L-FokI-L_{D450A} + ND3-MTS-TALE-R-FokI-R + |
| for ND3 | MTS-A3A + MTS-γb-Trex2 + MTS-γb-UGI |
| target site | |
| mtCyDENT-L | ND6.2-MTS-TALE-L-FokI-L + ND6.2-MTS-TALE-R-FokI-R_{D450A} + |
| for ND6.2 | MTS-A3A + MTS-Trex2 + MTS-UGI |
| target site | |
| mtCyDENT1b-L | ND6.2-MTS-TALE-L-FokI-L + ND6.2-MTS-TALE-R-FokI-R_{D450A} + |
| for ND6.2 | MTS-A3A + MTS-γb-Trex2 + MTS-γb-UGI |
| target site | |
| mtCyDENT-R | ND6.2-MTS-TALE-L-FokI-L_{D450A} + ND6.2-MTS-TALE-R-FokI-R + |
| for ND6.2 | MTS-A3A + MTS-Trex2 + MTS-UGI |
| target site | |
| mtCyDENT1b-R | ND6.2-MTS-TALE-L-FokI-L_{D450A} + ND6.2-MTS-TALE-R-FokI-R + |
| for ND6.2 | MTS-A3A + MTS-γb-Trex2 + MTS-γb-UGI |
| target site | |
| mtCyDENT1b-L | ND1-MTS-TALE-L-FokI-L + ND1-MTS-TALE-R-FokI-R_{D450A} + |
| for ND1 | MTS-A3A + MTS-γb-Trex2 + MTS-γb-UGI |
| target site | |
| mtCyDENT1b-R | ND1-MTS-TALE-L-FokI-L_{D450A} + ND1-MTS-TALE-R-FokI-R + |
| for ND1 | MTS-A3A + MTS-γb-Trex2 + MTS-γb-UGI |
| target site | |
| mtCyDENT1b-L | ND4-MTS-TALE-L-FokI-L + ND4-MTS-TALE-R-FokI-R_{D450A} + |
| for ND4 | MTS-A3A + MTS-γb-Trex2 + MTS-γb-UGI |
| target site | |
| mtCyDENT1b-R | ND4-MTS-TALE-L-FokI-L_{D450A} + ND4-MTS-TALE-R-FokI-R + |
| for ND4 | MTS-A3A + MTS-γb-Trex2 + MTS-γb-UGI |
| target site | |
| mtCyDENT1b-L | ND5.1-MTS-TALE-L-FokI-L + ND5.1-MTS-TALE-R-FokI-R_{D450A} + |
| for ND5.1 | MTS-A3A + MTS-γb-Trex2 + MTS-γb-UGI |
| target site | |
| mtCyDENT1b-R | ND5.1-MTS-TALE-L-FokI-L_{D450A} + ND5.1-MTS-TALE-R-FokI-R + |
| for ND5.1 | MTS-A3A + MTS-γb-Trex2 + MTS-γb-UGI |
| target site | |
| mtCyDENT1b-L | ND6-MTS-TALE-L-FokI-L + ND6-MTS-TALE-R-FokI-R_{D450A} + |
| for ND6 | MTS-A3A + MTS-γb-Trex2 + MTS-γb-UGI |
| target site | |
| mtCyDENT1b-R | ND6-MTS-TALE-L-FokI-L_{D450A} + ND6-MTS-TALE-R-FokI-R + |
| for ND6 | MTS-A3A + MTS-γb-Trex2 + MTS-γb-UGI |
| target site | |

### Example 12: Improving the Editing Efficiency and Precision of CyDENT

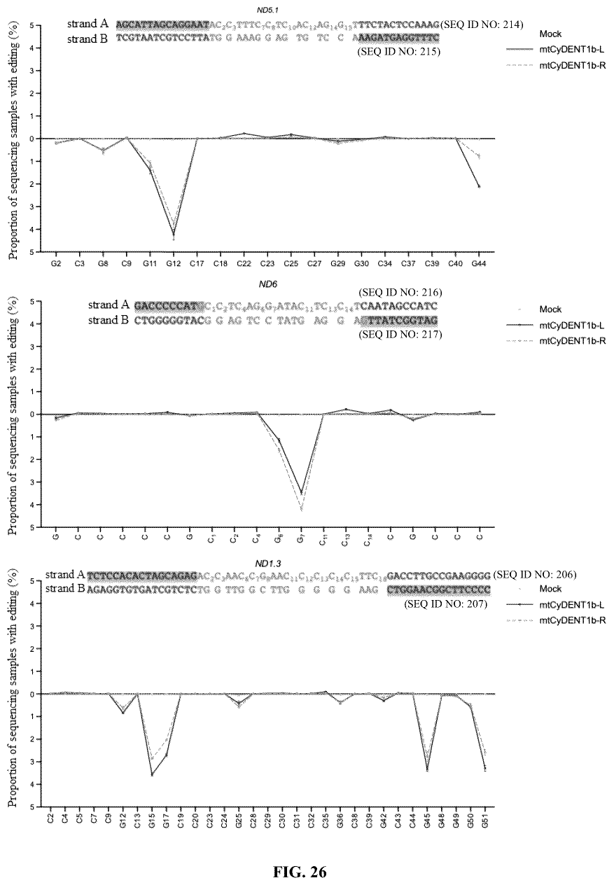

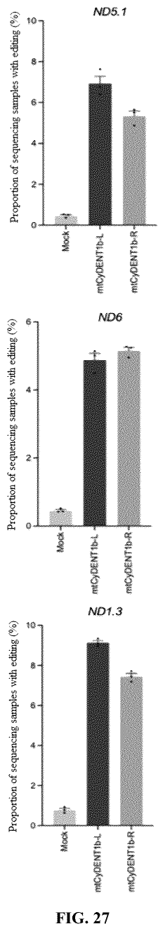

As mentioned in Example 4 above, the base editor of the present disclosure could be formed by the self-assembly of multiple functional modules and was compatible with deaminases of different types. Therefore, the deaminase domain in the base editor could be replaced with a deaminase known in the art to take advantage of the unique characteristics of each deaminase, thereby enhancing the activity or further improving the precision of editing in a strand. A newly discovered single-stranded DNA (ssDNA)-specific cytidine deaminase Sdd7 was found to have higher editing activity than other deaminases (Huang, J. et al. Discovery of new deaminase functions by structure-based protein clustering. bioRxiv (2023).). In this Example, the inventors took the mtCyDENT1b composition as an example and used Sdd7 as the deaminase of this editor, so as to evaluate the editing efficiency at the mtDNA targets ND5.1, ND6 and ND1.3. It was observed by the inventors that 87.5% of the base editing triggered by Sdd7-mtCyDENT1b-L merely occurred in one DNA strand and 93.0% of the base editing triggered by Sdd7-mtCyDENT1b-R merely occurred in one DNA strand. This result further demonstrated that CyDENT had superior strand specificity in base editing (FIG. 26). The average editing efficiency of these two editors on the target bottom strand of DNA ranged between 4.88% and 9.13% (FIG. 27). These results further verified that the deaminase domain in the base editor of the present disclosure could be replaced during modular assembly.

### Example 13: Improvements to Base Editors

In the above-mentioned Examples, the inventors had verified by experiments that the base editor composition of the present disclosure had technical advantages such as having single-strand editing specificity, being able to be formed by modular assembly, achieving efficient, precise and controllable base editing and resulting in low indel frequency. In subsequent Examples, the inventors further optimized the base editor so as to obtain a base editor composition having more superior functions.

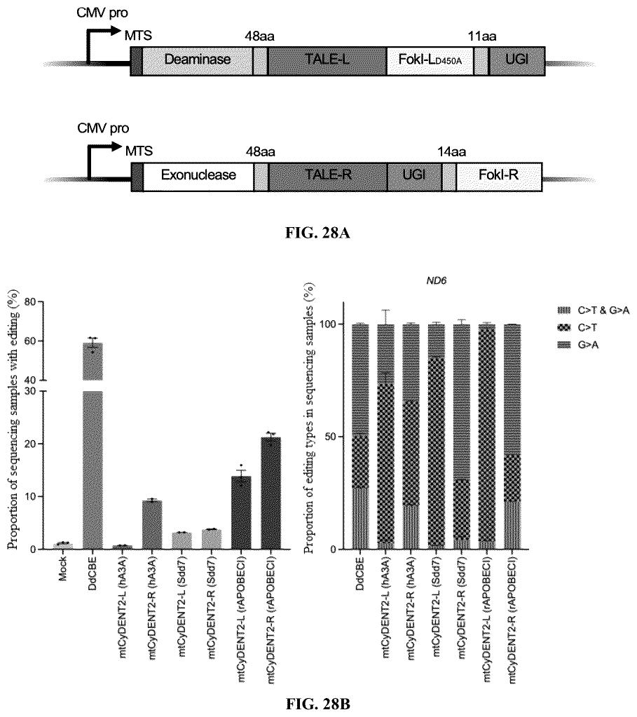

In this Example, the inventors fused the deaminase domain and the exonuclease domain to the N-termini of TALE-L and TALE-R via a 48-amino acid linker peptide (flexible linker), and UGI was fused to the C-terminal and the N-terminal of FokI-L and FokI-R, respectively. This construct architecture was referred to herein as mtCyDENT2 (FIG. 28A). The base editing effect of mtCyDENT2-L (comprising FokI-L_{nickase}) was determined on ND6 (FIG. 28B) and 94.5% of the base editing merely occurred in top strand, thereby reflecting good single-strand specific editing ability of CyDENT system.

In FIGS. 28A to 28B, the experimental treatments or construct combinations involved in figures were as shown below.

| | Experimental treatments or construct |
|---|---|
| | combinations involved in figures |
| mtCyDENT2-L (hA3A) | ND6-MTS-A3A-48aa-TALE-L-FokI-L-11aa-UGI + |
| for ND6 target site | ND6-MTS-Trex2-48aa-TALE-R-UGI-14aa-FokI-R_{D450A} |
| mtCyDENT2-L (hA3A) | ND6-MTS-A3A-48aa-TALE-L-FokI-L_{D450A}-11aa-UGI + |
| for ND6 target site | ND6-MTS-Trex2-48aa-TALE-R-UGI-14aa-FokI-R |
| mtCyDENT2-L (Sdd7) | ND6-MTS-Sdd7-48aa-TALE-L-FokI-L-11aa-UGI + |
| for ND6 target site | ND6-MTS-Trex2-48aa-TALE-R-UGI-14aa-FokI-R_{D450A} |
| mtCyDENT2-R (Sdd7) | ND6-MTS-Sdd7-48aa-TALE-L-FokI-L_{D450A}-11aa-UGI + |
| for ND6 target site | ND6-MTS-Trex2-48aa-TALE-R-UGI-14aa-FokI-R |
| mtCyDENT2-L | ND6-MTS-rAPOBEC1-48aa-TALE-L-FokI-L-11aa-UGI + |
| (rAPOBEC1) for ND6 | ND6-MTS-Trex2-48aa-TALE-R-UGI-14aa-FokI-R_{D450A} |
| target site | |
| mtCyDENT2-L | ND6-MTS-rAPOBEC1-48aa-TALE-L-FokI-L_{D450A}-11aa-UGI + |
| (rAPOBEC1) for ND6 | ND6-MTS-Trex2-48aa-TALE-R-UGI-14aa-FokI-R |
| target site | |

### Example 14 Base Editing on GC-Motifs by mtCyDNET

A DddA-dependent DdCBE system had strict constraints on the context of TC-motifs for cytidine deamination, and researches had found that the frequency of occurrence of editing in the context of GC sequence was relatively low (Nakazato, I. et al. Targeted base editing in the mitochondrial genome of Arabidopsis thaliana. Proc. Natl. Acad. Sci. USA. 119, e2121177119 (2022).). Phage-assisted discontinuous and continuous evolution were used for the evolution of the “wild-type” DddA (Mok, B. Y et al. CRISPR-free base editors with enhanced activity and expanded targeting scope in mitochondrial and nuclear DNA. Nat. Biotechnol. 40, 1378-1387 (2022).), and the evolved DddA11 variant had better compatibility with AC and CC sequence motifs. However, there still remained challenge in the editing on GC sequence motifs by DddA11. In this Example, efficient and strand-selective editing on GC sequence motifs was achieved by using the modular replacement of the deaminase domain of CyDENT.

The inventors introduced a single-stranded DNA-specific cytidine deaminase having editing activity on GC sequence motifs, thereby developing a GC-compatible mtCyDENT base editor. Recently, a newly discovered single-stranded DNA-specific and GC- and AC-compatible cytidine deaminase Sdd3 exhibited higher editing activity on GC sequence motifs than other deaminases (Huang, J. et al. Discovery of new deaminase functions by structure-based protein clustering. bioRxiv (2023).).

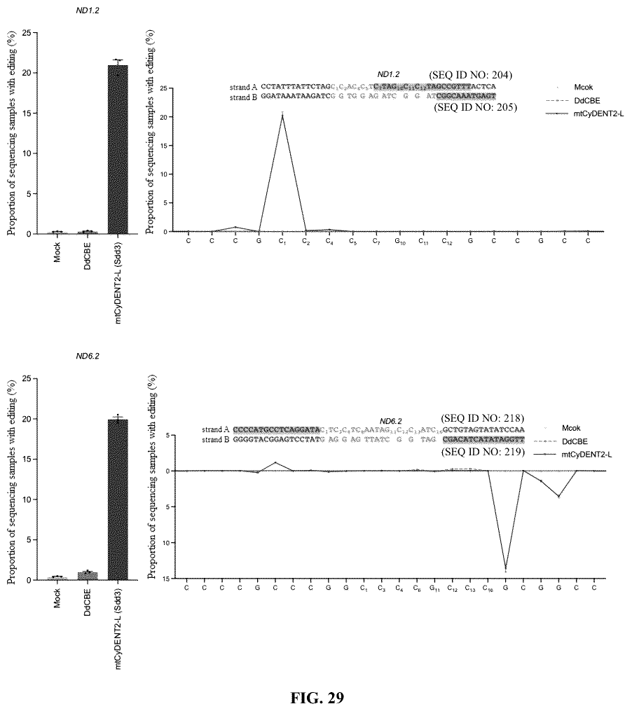

Therefore, a TALE array (FIG. 29) was designed to target ND1.2 and ND6.2 sites in HEK293T cells in the present disclosure, so as to evaluate the editing preference of the sequence motifs that were difficult to edit with prior art. It was worth noting that the efficiency of strand-specific cytosine base editing on the GC sequence motifs at ND1.2 and ND6.2 sites reached 21.0% and 20.0% respectively, which was unachievable by the DdCBE in the prior art at the same target sites. At the ND1.2 site, 96.9% of the editing occurred selectively in the top strand of DNA, while at the ND6.2 site, 92.0% of the editing occurred selectively in the bottom strand of DNA (FIG. 29).

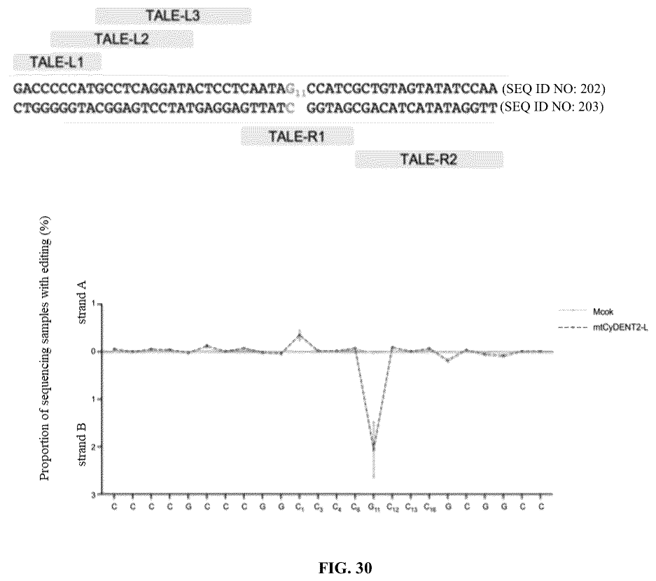

Subsequently, the inventors adjusted the TALE binding site, and observed that Sdd3-mtCyDENT had an editing efficiency of 2.06% at the ND6.2 site (FIG. 30). It was reported that such special mutation (m.14453G>A) was directly associated with the development of Leigh syndrome, and the DdCBE in the prior art, however, could not realize the editing in the context of this same target sequence. Therefore, mtCyDENT and its future optimized products could be used for a superior base editing method capable of performing precise editing on the pathogenic mutation in mtDNA.

In FIGS. 29 and 30, the experimental treatments or construct combinations involved in figures were as shown below.

| | Experimental treatments or construct |
|---|---|
| | combinations involved in the Example |
| mtCyDENT2-L | ND1.2-MTS-Sdd3-48aa-TALE-L-FokI-L-11aa-UGI + |
| (Sdd3) for ND1.2 | ND1.2-MTS-Trex2-48aa-TALE-R-UGI-14aa-FokI-R_{D450A} |
| target site | |
| mtCyDENT2-L | ND6.2-MTS-Sdd3-48aa-TALE-L-FokI-L-11aa-UGI + |
| (Sdd3) for ND6.2 | ND6.2-MTS-Trex2-48aa-TALE-R-UGI-14aa-FokI-R_{D450A} |
| target site | |
| DdCBE for ND1.2 | ND1.2-MTS-TALE-L-DddA_{N}-UGI + |
| target site | ND1.2-MTS-TALE-R-DddA_{C}-UGI |
| DdCBE for ND6.2 | ND6.2-MTS-TALE-L-DddA_{N}-UGI + |
| target site | ND6.2-MTS-TALE-R-DddA_{C}-UGI |

### Example 15: Off-Target Analysis of mtCyDENT

The mitochondrial editing by DdCBE in the prior art could induce a large number of nuclear off-target editing. In order to evaluate the off-target rate of CyDENT in the entire nuclear genome and the entire mitochondrial genome, 2.25 Tb of clean bases were obtained in this Example, with an average of 281.13 Gb for each sample. The average depth of mitochondrial genome sequencing was approximately 6362 fold, and the human reference genome used was hg19.

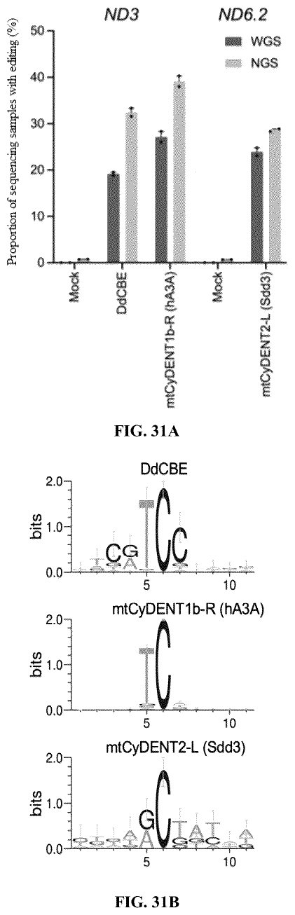

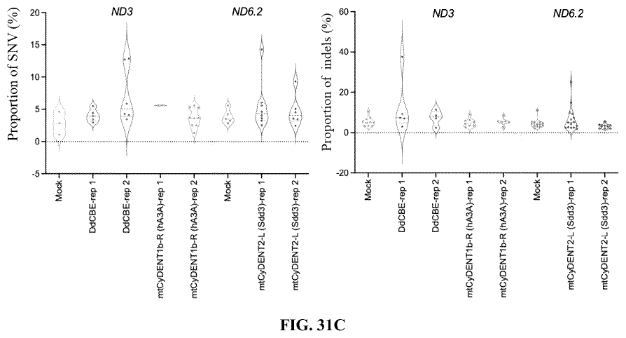

In this Example, the DdCBE plasmid and the mtCyDENT1b-R (hA3A) plasmid targeting ND3 and the mtCyDENT2-L (Sdd3) plasmid targeting ND6.2 were designed to transfect HEK293T cells, and these plasmids were capable of perform editing on GC sequence motifs, as demonstrated by the whole genome sequencing (WGS) and NGS analysis (FIG. 31A). Subsequently, the off-target rates in the whole mitochondrial genome and the whole nuclear genome were analyzed. The results indicated that the average frequencies of C·G-to-T·A and G·C-to-A·T base conversion in the entire mitochondrial genome in the untreated negative control group, DdCBE treatment group, mtCyDENT1b-R (hA3A) treatment group and mtCyDENT2-L (Sdd3) treatment group were 4.8%, 6.9%, 16.5% and 5.9%, respectively. Compared with the control group, the inventors found an average of 32, 678 and 16 single nucleotide variations (SNVs) in the mitochondrial genome in DdCBE treatment group, mtCyDENT1b-R (hA3A) treatment group and mtCyDENT2-L (Sdd3) treatment group, respectively. By analyzing the 5-bp regions upstream and downstream of each potential off-target SNV, conserved TC-motifs were found inDdCBE group and mtCyDENT1b-R (hA3A) group, while conserved GC/AC-motifs were found in mtCyDENT2-L (Sdd3) group (FIG. 31B).

The inventors analyzed the TALE-dependent off-target effects in the nuclear genome. A total of 74963 potential off-target regions (comprising 0 to 3 regions that mismatched with the TALE binding site in ND3 and ND6.2) were identified. It was observed by the inventors that there was no difference in SNV allele frequency and indel frequency at ND3 site or ND6.2 site in the control group, DdCBE treatment group, mtCyDENT1b-R (hA3A) treatment group and mtCyDENT2-L (Sdd3) treatment group (FIG. 31C). These results indicated that the modular assembly and optimization of CyDENT were capable of reducing the off-target effects in mitochondrial and nuclear genomes to the largest extent. mtCyDENT was a valuable tool for mitochondrial genome editing.

In FIGS. 31A to 31C, the experimental treatments or construct combinations involved in figures were as shown below.

| | Experimental treatments or construct |
|---|---|
| | combinations involved in figures |
| Mt CyDENT | ND3-MTS-TALE-L-FokI-L_{D450A} + |
| 1b-R (hA3A) | ND3-MTS-TALE-R-FokI-R + |
| for ND3 | MTS-A3A + MTS-γb-Trex2 + MTS-γb-UGI |
| target site | |
| Mt CyDENT | ND6.2-MTS-Sdd3-48aa-TALE-L-FokI-L-11aa-UGI + |
| 2-L (Sdd3) | ND6.2-MTS-Trex2-48aa-TALE-R-UGI-14aa-FokI-R_{D450A} |
| for ND6.2 | |
| target site | |
| DdCBE for ND3 | ND3-MTS-TALE-L-DddA_{N}-UGI + |
| target site | ND3-MTS-TALE-R-DddA_{C}-UGI |
| DdCBE for ND6.2 | ND6.2-MTS-TALE-L-DddA_{N}-UGI + |
| target site | ND6.2-MTS-TALE-R-DddA_{C}-UGI |

The illustration of the Examples above is merely intended to facilitate the understanding of the methods and the gists of the present disclosure. It should be noted that, a number of improvements and modifications may also be made to the present disclosure by those of ordinary skill in the art under the premise of not departing from the principles of the present disclosure, and these improvements and modifications also fall within the protection scope of the claims of the present disclosure.

What is claimed is:

1. A nucleic acid base editor, comprising the following elements:
a) a sequence-specific DNA binding protein;
b) a nickase;
c) an exonuclease; and
d) a base-specific deaminase.

2. The nucleic acid base editor according to claim 1, wherein each element of the nucleic acid base editor exists alone, or constitutes one or more fusion proteins.

3. The nucleic acid base editor according to claim 1, wherein the sequence-specific DNA binding protein is one or more selected from the group consisting of a TALE protein, a ZFA protein, a Cas protein and a meganuclease.

4. The nucleic acid base editor according to claim 3, wherein the sequence-specific DNA binding protein is a TALE protein.

5. The nucleic acid base editor according to claim 2, wherein the nickase is a dimer of a cleavage domain monomer of FokI (FokICD) or a mutant of the dimer, the dimer of the cleavage domain monomer of FokI (FokICD) or the mutant of the dimer is composed of a pair of interacting cleavage domain monomers of FokI (FokICD), and the dimer of the cleavage domain monomer of FokI (FokICD) or the mutant of the dimer has one and only one cleavage domain monomer of FokI (FokICD) which has DNA endonuclease activity.

6. The nucleic acid base editor according to claim 5, wherein the cleavage domain monomer of FokI is isolated from a mutant of a wild-type FokI protein, the mutant of the wild-type FokI protein has a mutation at position 450 and/or position 467, or has an amino acid sequence which has at least 85%, at least 90%, at least 92%, at least 95%, at least 96%, at least 97%, at least 98%, at least 99%, or at least 99.5% identity with that of the cleavage domain monomer of FokI.

7. The nucleic acid base editor according to claim 6, wherein the mutation causes the cleavage domain monomer of FokI (FokICD) to lose DNA endonuclease activity.

8. The nucleic acid base editor according to claim 5, wherein the cleavage domain monomer of FokI (FokICD) is isolated from a mutant of a wild-type FokI protein, the mutation prevents the cleavage domain monomer of FokI (FokICD) from the self-polymerization with a cleavage domain monomer of FokI (FokICD) containing a mutation at a same site and the formation of a dimer.

9. The nucleic acid base editor according to claim 5, wherein an amino acid sequence of the cleavage domain monomer of FokI (FokICD) is selected from SEQ ID No. 60-63 and 87-88.

10. The nucleic acid base editor according to claim 1, wherein the base-specific deaminase is selected from a cytidine-specific deaminase and an adenosine-specific deaminase.

11. The nucleic acid base editor according to claim 1, wherein the base-specific deaminase is a cytidine-specific deaminase.

12. The nucleic acid base editor according to claim 11, wherein the cytidine-specific deaminase is one or more selected from the group consisting of hAPOBEC3A, rAPOBEC1, hAID, pmCDA1 and Sdd deaminase.

13. The nucleic acid base editor according to claim 11, wherein the nucleic acid base editor further comprises:
e) a uracil glycosylase inhibitor (UGI); and
the uracil glycosylase inhibitor exists alone, or constitutes at least one fusion protein with other elements of the nucleic acid base editor.

14. The nucleic acid base editor according to claim 1, wherein the base-specific deaminase is an adenosine-specific deaminase.

15. The nucleic acid base editor according to claim 14, wherein the adenosine-specific deaminase is TadA-8e.

16. The nucleic acid base editor according to claim 1, wherein the nucleic acid base editor further comprises:
f) γb;
the γb constitutes at least one fusion protein with other elements of the nucleic acid base editor.

17. The nucleic acid base editor according to claim 2, wherein the fusion proteins are selected from any one of the following (1) to (12):
(1) fusion proteins comprise:
a first fusion protein comprising a nuclear localization signal (NLS), a sequence-specific DNA binding protein, a nickase and a base-specific deaminase;
a second fusion protein comprising an exonuclease and a nuclear localization signal (NLS); and
a third fusion protein comprising a uracil glycosylase inhibitor (UGI) and a nuclear localization signal (NLS);
(2) fusion proteins comprise:
a first fusion protein comprising in linear order from the protein's amino terminus a nuclear localization signal (NLS), a base-specific deaminase, a TALE-L protein, an FokI-L_{D450A}/FokI-L_{D467A}/FokI-L protein, a T2A sequence, an NLS, a TALE-R protein and an FokI-R/FokI-R_{D450A}/FokI-R_{D467A} protein;
a second fusion protein comprising an exonuclease and a nuclear localization signal (NLS); and
a third fusion protein comprising a uracil glycosylase inhibitor (UGI) and a nuclear localization signal (NLS);
(3) fusion proteins comprise:
a first fusion protein comprising in linear order from the protein's amino terminus a nuclear localization signal (NLS), a TALE-L protein, an FokI-L_{D450A}/FokI-L_{D467A}/FokI-L protein, a T2A sequence, an NLS, a base-specific deaminase, a 48-amino acid linker peptide, a TALE-R protein and an FokI-R/FokI-R_{D450A}/FokI-R_{D467A} protein;
a second fusion protein comprising an exonuclease and a nuclear localization signal (NLS); and
a third fusion protein comprising a uracil glycosylase inhibitor (UGI) and a nuclear localization signal (NLS);
(4) fusion proteins comprise:
a first fusion protein comprising a nuclear localization signal (NLS), a nickase, a sequence-specific DNA binding protein, a base-specific deaminase and a uracil glycosylase inhibitor (UGI); and
a second fusion protein comprising an exonuclease and a nuclear localization signal (NLS);
(5) fusion proteins comprise:
a first fusion protein comprising in linear order from the protein's amino terminus a nuclear localization signal (NLS), a base-specific deaminase, a 48-amino acid linker peptide, a TALE-L protein, an FokI-L_{D450A}/FokI-L_{D467A}/FokI-L protein, a T2A sequence, an NLS, a TALE-R protein, an FokI-R/FokI-R_{D450A}/FokI-R_{D467A} protein, a 4-amino acid linker peptide and a uracil glycosylase inhibitor (UGI); and
a second fusion protein comprising an exonuclease and a nuclear localization signal (NLS),
or, the fusion proteins comprise:
a first fusion protein comprising in linear order from the protein's amino terminus a nuclear localization signal (NLS), a uracil glycosylase inhibitor (UGI), a 4-amino acid linker peptide, a base-specific deaminase, a 48-amino acid linker peptide, a TALE-L protein, an FokI-L_{D450A}/FokI-L_{D467A}/FokI-L protein, a T2A sequence, an NLS, a TALE-R protein and an FokI-R/FokI-R_{D450A}/FokI-R_{D467A} protein; and
a second fusion protein comprising an exonuclease and a nuclear localization signal (NLS);
(6) fusion proteins comprise:
a first fusion protein comprising in linear order from the protein's amino terminus a mitochondrial targeting sequence (MTS), a TALE-L protein and an FokI-L_{D450A}/FokI-L_{D467A}/FokI-L protein;
a second fusion protein comprising in linear order from the protein's amino terminus a mitochondrial targeting sequence (MTS), a TALE-R protein and an FokI-R/FokI-R_{D450A}/FokI-R_{D467A} protein;
a third fusion protein comprising in linear order from the protein's amino terminus a mitochondrial targeting sequence (MTS) and an exonuclease;
a fourth fusion protein comprising in linear order from the protein's amino terminus a mitochondrial targeting sequence (MTS) and a base-specific deaminase; and
a fifth fusion protein comprising in linear order from the protein's amino terminus mitochondrial targeting sequence (MTS) and a uracil glycosylase inhibitor (UGI);
(7) fusion proteins comprise:
a first fusion protein comprising in linear order from the protein's amino terminus a mitochondrial targeting sequence (MTS), a TALE-L protein and an FokI-L_{D450A}/FokI-L_{D467A}/FokI-L protein;
a second fusion protein comprising in linear order from the protein's amino terminus a mitochondrial targeting sequence (MTS), a TALE-R protein and an FokI-R/FokI-R_{D450A}/FokI-R_{D467A} protein;
a third fusion protein comprising in linear order from the protein's amino terminus a mitochondrial targeting sequence (MTS), γb and an exonuclease;
a fourth fusion protein comprising in linear order from the protein's amino terminus a mitochondrial targeting sequence (MTS) and a base-specific deaminase; and
a fifth fusion protein comprising in linear order from the protein's amino terminus a mitochondrial targeting sequence (MTS), γb and a uracil glycosylase inhibitor (UGI);
(8) fusion proteins comprise:
a first fusion protein comprising a nuclear localization signal (NLS)/a chloroplast translocation peptide (CTP)/a mitochondrial targeting sequence (MTS), a sequence-specific DNA binding protein and a nickase;
a second fusion protein comprising an exonuclease and a nuclear localization signal (NLS)/a chloroplast translocation peptide (CTP)/a mitochondrial targeting sequence (MTS); and
a third fusion protein comprising a base-specific deaminase, a uracil glycosylase inhibitor (UGI), and a nuclear localization signal (NLS)/a chloroplast translocation peptide (CTP)/a mitochondrial targeting sequence (MTS);
(9) fusion proteins comprise:
a first fusion protein comprising in linear order from the protein's amino terminus a nuclear localization signal (NLS)/a chloroplast translocation peptide (CTP)/a mitochondrial targeting sequence (MTS), a TALE-L protein, an FokI-L_{D450A}/FokI-L_{D467A} protein, a T2A sequence, an NLS, a TALE-R protein, and an FokI-R protein, or comprising in linear order from the protein's amino terminus a nuclear localization signal (NLS)/a chloroplast translocation peptide (CTP)/a mitochondrial targeting sequence (MTS), a TALE-L protein, an FokI-L protein, a T2A sequence, an NLS, a TALE-R protein, and an FokI-R_{D450A}/FokI-R_{D467A} protein;
a second fusion protein comprising in linear order from the protein's amino terminus a nuclear localization signal (NLS)/a chloroplast translocation peptide (CTP)/a mitochondrial targeting sequence (MTS) and an exonuclease; and
a third fusion protein comprising in linear order from the protein's amino terminus a nuclear localization signal (NLS)/a chloroplast translocation peptide (CTP)/a mitochondrial targeting sequence (MTS), a base-specific deaminase, an XTEN linker peptide and a uracil glycosylase inhibitor (UGI);
(10) fusion proteins comprise:
a first fusion protein comprising in linear order from the protein's amino terminus a mitochondrial targeting sequence (MTS), a base-specific deaminase, a 48-amino acid linker peptide, a TALE-L protein, an FokI-L_{D450A}/FokI-L_{D467A}/FokI-L protein, an 11-amino acid linker peptide and a uracil glycosylase inhibitor (UGI); and
a second fusion protein comprising in linear order from the protein's amino terminus a mitochondrial targeting sequence (MTS), a 48-amino acid linker peptide, a TALE-R/FokI-R_{D450A}/FokI-R_{D467A} protein, a uracil glycosylase inhibitor (UGI), a 14-amino acid linker peptide and an FokI-R protein;
(11) fusion proteins comprise:
a first fusion protein comprising a nuclear localization signal (NLS), a sequence-specific DNA binding protein and a nickase;
a second fusion protein comprising a nuclear localization signal (NLS), a base-specific deaminase and a uracil glycosylase inhibitor (UGI); and
a third fusion protein comprising an exonuclease and a nuclear localization signal (NLS); and
(12) fusion proteins comprise:
a first fusion protein comprising a nuclear localization signal (NLS), a sequence-specific DNA binding protein and a nickase;
a second fusion protein comprising in linear order from the protein's amino terminus an exonuclease, an XTEN linker peptide, a base-specific deaminase, an XTEN linker peptide, a uracil glycosylase inhibitor (UGI) and a nuclear localization signal, or a second fusion protein comprising in linear order from the protein's amino terminus an exonuclease, a 48-amino acid linker peptide, a base-specific deaminase, an XTEN linker peptide, a uracil glycosylase inhibitor (UGI) and a nuclear localization signal.

18. A recombinant expression construct for nucleic acid base editing, wherein the recombinant expression construct is used to express the nucleic acid base editor of claim 1.

19. A genetically engineered cell for the transformation of the recombinant expression construct of claim 18.

20. A method of performing nucleic acid base editing in a cell, wherein the nucleic acid is base editor of claim 1 is introduced into the cell so as to edit a target gene.

21. The method of nucleic acid base editing according to claim 20, wherein the target gene is selected from a nuclear genomic DNA, a mitochondrial genomic DNA, and a chloroplast genomic DNA.

22. The method of nucleic acid base editing according to claim 20, wherein the target gene is a nuclear genomic DNA, and the nucleic acid base editor further comprises a nuclear localization signal (NLS);
the target gene is a mitochondrial genomic DNA, and the nucleic acid base editor further comprises a mitochondrial targeting sequence (MTS); or
the target gene is a chloroplast genomic DNA, and the nucleic acid base editor further comprises a chloroplast translocation peptide (CTP).

23. The method of nucleic acid base editing according to claim 20, wherein the cell is a mammalian cell, a bacterium, a protist, a fungus, an insect cell, yeast, a non-conventional yeast or a plant cell.

24. The method of nucleic acid base editing according to claim 23, wherein the plant cell is derived from a whole plant of a monocotyledon or a dicotyledon, a seedling, a meristem, a ground tissue, a vascular tissue, a dermal tissue, a seed, a leaf, a root, a bud, a stem, a flower, a fruit, a stolon, a bulb, a tuber, a corm, an asexual terminal branch, a bud, a budlet and a tumor tissue; or
the mammalian cell is selected from a germ cell, a neuron, a muscle cell, an endocrine/exocrine cell, an epithelial cell, a muscle cell, a tumor cell, an embryonic cell, a hematopoietic cell, an osteocyte, a germplasm cell, a somatic cell, a stem cell, a pluripotent stem cell, an induced pluripotent stem cell, a progenitor cell, a meiotic cell and a mitotic cell of human.

25. A method for treating a disease in a subject in need thereof, comprising delivering an effective amount of the nucleic acid base editor of claim 1 to the subject so as to modify a gene related to the disease.

26. A method for producing a genetically modified plant, wherein the method comprises introducing the base editor of claim 1 into at least one of the plants.
